(19) United States
(12) Patent Application Publication
(10) Pub. No.: US 2014/0294834 A1
(43) Pub. Date: Oct. 2, 2014

(54) TANDEM FC BISPECIFIC ANTIBODIES

(71) Applicant: MERRIMACK PHARMACEUTICALS, INC., CAMBRIDGE, MA (US)

(72) Inventors: Brian HARMS, Roslindale, MA (US); Neeraj KOHLI, Brighton, MA (US); Alexey LUGOVSKOY, Woburn, MA (US); Stephen SU, Boston, MA (US); Melissa GEDDIE, Somerville, MA (US)

(73) Assignee: MERRIMACK PHARMACEUTICALS, INC., CAMBRIDGE, MA (US)

(21) Appl. No.: 14/185,628

(22) Filed: Feb. 20, 2014

Related U.S. Application Data
(63) Continuation of application No. PCT/US12/52490, filed on Aug. 27, 2012.
(60) Provisional application No. 61/527,802, filed on Aug. 26, 2011.

(51) Int. Cl. C07K 16/46; C07K 16/18; C07K 16/28

(52) U.S. Cl. CPC C07K 16/468; C07K 16/2863; C07K 16/2866; C07K 16/2869; C07K 16/18
USPC 424/136.1; 530/387.3; 530/389.6; 536/23.53; 435/320.1; 435/328; 435/69.6

(57) ABSTRACT

Provided herein are tandem Fcs and tandem Fc antibodies (“TFcAs”), e.g., tandem Fc bispecific antibodies (“TFcBAs”), which comprise one or at least two binding sites that specifically bind to one or more cell surface receptors. The binding sites are connected through a TFc, which TFc comprises a first Fc region and a second Fc region, wherein the first and the second Fc regions are linked through a TFc linker to form a contiguous polypeptide and dimerize to form an Fc dimer. Exemplary TFcBAs inhibit signal transduction through the cell surface receptor(s) for which the binding sites of the TFcBA are specific.

### RELATED APPLICATIONS

This application claims priority to U.S. Provisional Application No. 61/527,802, filed Aug. 26, 2011. Where permitted, the foregoing applications are incorporated by reference, each in its entirety, for any and all purposes.

### BACKGROUND

It has been established that tumor cells express receptors for growth factors and cytokines that stimulate proliferation of the cells and, moreover, that antibodies to such receptors can be effective in blocking the stimulation of cell proliferation mediated by growth factors and cytokines to inhibit tumor cell growth. Commercially available therapeutic antibodies that target receptors on cancer cells include, for example, trastuzumab (Herceptin®) for the treatment of breast cancer, which targets the HER2 receptor (also known as ErbB2), and cetuximab (Erbitux®) for the treatment of colorectal cancer and head and neck cancer, which targets the epidermal growth factor receptor (EGFR, also known as HER1 or ErbB1).

While this approach of administering a therapeutic agent comprising only a single therapeutic monoclonal antibody (when administered in the absence of administration of another therapeutic antibody, referred to herein as monotherapy) has shown considerable success in cancer treatment, there are a number of factors that can lead to failure of such treatment or recurrence of tumor growth after initial inhibition. For example, certain tumors rely on more than one growth factor-mediated signal transduction pathway for cell proliferation and thus targeting of a single pathway may prove insufficient to significantly affect tumor cell growth. Alternatively, even in cases where one pathway is the only or predominant growth-stimulatory pathway, certain tumors cells are capable of activating another signaling pathway for growth stimulation when the original one is blocked by antibody (innate resistance to treatment). Still further, some tumors exhibit initial responsiveness to antibody monotherapy but later develop resistance to treatment by switching to use of another signaling pathway (acquired resistance to treatment).

Accordingly, additional therapeutic approaches for cancer treatment are needed to overcome limitations of antibody monotherapy and to provide other benefits.

### SUMMARY

Provided herein are engineered antibodies, such as Tandem Fc Antibodies (“TFcAs”). Exemplary TFcAs are Tandem Fc Bispecific Antibodies (TFcBAs). A TFcBA comprises a Tandem Fc, which is a polypeptide moiety that comprises a first Fc region and a second Fc region, each of said first Fc region and second Fc region having a C-terminus and an N-terminus; the first Fc region and the second Fc region are linked as a single polypeptide chain through a TFc linker having a C-terminus and an N-terminus (i.e., the C-terminus of the first Fc region is linked by a peptide bond to the N-terminus of the TFc linker, the C-terminus of which TFc linker is in turn linked by a peptide bond to the N-terminus of the second Fc region). A TFcBA may comprise at least two binding sites (at least a first binding site and a second binding site). Each such binding site binds specifically to a specific part of a cell surface receptor. Exemplary cell surface receptors are those that are expressed or overexpressed by cancer cells. Exemplary binding sites include antibody-derived binding sites that bind immunospecifically to an extracellular domain of a cell surface receptor. The first or the second binding site of a TFcA or TFcBA may bind specifically to a human receptor protein selected from the group consisting of ErbB2, ErbB3 (e.g., a binding site described in U.S. Pat. No. 7,846,440), ErbB4, IGF1R, IGF2R, Insulin receptor, RON, c-Met, EGFR, VEGFR1, VEGFR2, TNFR, FGFR1-4, PDGFR (alpha and beta), c-Kit, EPCAM and EphA2. Typically such binding will be specific to the extracellular portion of the receptor protein. In certain embodiments disclosed herein, one of the at least two binding sites comprised by a TFcBA is a binding site specific to c-Met, e.g., an anti-c-Met Fab or an anti-cMet scFv. In certain exemplified embodiments a TFcBA is provided that comprises a single anti-c-Met binding site and at least one second binding site that does not bind to c-Met, e.g., a binding site specific to ErbB2, ErbB3, ErbB4, IGF1R, IGF2R, Insulin receptor, RON, EGFR, VEGFR1, VEGFR2, TNFR, FGFR1-4, PDGFR (alpha and beta), c-Kit, EPCAM and EphA2, wherein the anti-c-Met binding site and the second binding site are linked through a TFc to form a contiguous polypeptide. TFcBAs are provided that bind to two epitopes (e.g., extracellular epitopes) on a single receptor or to two distinct cell surface receptors and which, upon such binding, strongly inhibit signal transduction that is normally stimulated by a cognate ligand of at least one cell surface receptor to which the TFcBA binds. For example, an anti-c-Met+anti-EGFR TFcBA may inhibit signal transduction induced by either or both of HGF (hepatocyte growth factor, the cognate ligand of C-met) and EGF (epidermal growth factor, the cognate ligand of EGFR), or an anti-c-Kit+anti-RON TFcBA may inhibit signal transduction induced by either or both of Macrophage Stimulating Protein (the cognate ligand of RON) and Stem Cell Factor (the cognate ligand of c-Kit), or an anti-c-Met+anti-EPCAM TFcBA may inhibit signal transduction induced by HGF; each such inhibition being with an IC50 of 10 nM or less or 1 nM or less or 100 pM or less, or with a maximal percent inhibition of at least 70% or at least 80% or at least 90%, as indicated by inhibition of ligand-induced phosphorylation of the receptor(s) that are signal transduction inhibited by the TFcBA. In certain embodiments, expression of the TFcBA in a cell produces (i) more (i.e. a greater percentage of) correctly formed TFcAB molecules relative to the expression of a multivalent antibody that binds to the same receptor(s) but does not comprise a TFc or (ii) more than 80% of correctly formed TFcAB molecules as determined, e.g., by Size Exclusion Chromatography (SEC).

Also provided herein are Abs which are TFcBAs, wherein the TFcBAs comprise a first binding site and a second binding site, wherein the first binding site binds to a first target and the second binding site binds to a second target, and wherein (i) the first and the second binding sites are linked through a TFc; (ii) the TFc comprises a first Fc region and a second Fc region, each said first Fc region and second Fc region having a C-terminus and an N-terminus; (iii) the first Fc region and the second Fc region are linked through a TFc linker having a C-terminus and an N-terminus to form a contiguous polypeptide; (iv) the first and the second Fc regions associate (bind) to form an Fc dimer; and (v) either or both of the first and the second Fc region comprise one or more amino acid (aa) modification to enhance or stabilize the binding between the first and the second Fc region. The TFcBA may inhibit signal transduction through either or both of the first and the second target. In certain embodiments, expression of the TFcBA in a host cell produces (i) more correctly formed TFcAB molecules relative to the expression in a matched host cell of a multivalent antibody that binds to the same receptor(s) but does not comprise a TFc or (ii) more than 80% of correctly formed TFcBA molecules as determined, e.g., by SEC.

Further provided herein are monovalent tandem FC antibodies (TFcAs). A monovalent TFcA may comprise a single binding site that binds to a target, wherein the binding site is linked to a TFc comprising a first Fc region and a second Fc region, each said first Fc region and second Fc region having a C-terminus and an N-terminus; and wherein (i) the first Fc region and the second Fc region are linked through a TFc linker having a C-terminus and an N-terminus to form a contiguous polypeptide; (ii) the first and the second Fc regions associate to form an Fc dimer; and (iii) either or both of the first and the second Fc region comprise one or more aa modification to enhance or stabilize the binding between the first and the second Fc region. The monovalent TFcA may inhibit signal transduction through the target. In certain embodiments, expression of the monovalent TFcA in a host cell produces (i) more correctly formed TFcA molecules relative to the expression in a matched host cell of an antibody that does not comprise a TFc or (ii) more than 80% of correctly formed TFcA molecules as determined, e.g., by SEC.

The first Fc region and the second Fc region of a TFc comprised by a TFcA, such as a TFcBA, may comprise a first and a second CH3 domain, respectively, each said CH3 domain having a C-terminus and an N-terminus. The first and the second Fc regions of a TFc comprised by a TFcA may comprise a first and a second CH2 domain, respectively, each said CH2 domain having a C-terminus and an N-terminus. The first and the second Fc regions of a TFc comprised by a TFcA may comprise a first and a second hinge, respectively, each said first hinge and said second hinge having a C-terminus and an N-terminus. In certain embodiments, the second hinge does not comprise an upper hinge subdomain. The TFc comprised in the TFcA may comprise in amino to carboxyl terminal order: a first CH2 domain, a first CH3 domain, a TFc linker, a second CH2 domain and a second CH3 domain. The TFc comprised in the TFcA may comprise in amino to carboxyl terminal order: a first hinge, a first CH2 domain, a first CH3 domain, a TFc linker, a second CH2 domain and a second CH3 domain. The TFc comprised in the TFcA may comprise in amino to carboxyl terminal order: a first hinge, a first CH2 domain, a first CH3 domain, a TFc linker, a second hinge, a second CH2 domain and a second CH3 domain. The first hinge may comprise an upper hinge subdomain, a core hinge subdomain and a lower hinge subdomain and the second hinge may comprise a core hinge subdomain and a lower hinge subdomain, but not an upper hinge subdomain, each said hinge sub-domain having a C-terminus and an N-terminus. The TFc comprised by the TFcA may comprise in amino to carboxyl terminal order: a first hinge, which is linked at its C-terminus to the N-terminus of a first CH2 domain, which is linked at its C-terminus to the N-terminus of a first CH3 domain, which is linked at its C-terminus to the N-terminus of a TFc linker, which is linked at its C-terminus to the N-terminus of a second hinge, which is linked at its C-terminus to the N-terminus of a second CH2 domain, which is linked at its C-terminus to the N-terminus of a second CH3 domain.

A TFc linker of a TFc comprised by a TFcA may comprise 20-50 aas. A TFc linker may be a Gly-Ser linker, such as (Gly₄Ser)ₙ, wherein n is 4, 5, 6, 7 or 8. A TFc linker may also comprise an aa sequence that is at least about 70%, 80%, 90%, 95%, 97%, 98%, or 99% identical to an aa sequence of a Gly-Ser linker or which differs therefrom in at most 20, 15, 10, 5, 4, 3, 2, or 1 aa addition, deletion or substitution.

A TFc of a TFcA may be an IgG1 TFc. A TFc may be a hybrid TFc, e.g., an IgG1/IgG4 hybrid TFc. A TFc of a TFcA may be an IgG1 TFc and may comprise in amino to carboxyl terminal order: a first IgG1 hinge, a first IgG1 CH2 domain, a first IgG1 CH3 domain, a TFc linker, a second IgG1 hinge, a second IgG1 CH2 domain, and a second IgG1 CH3 domain. A hybrid TFc may comprise in amino to carboxyl terminal order: a first IgG1/IgG4 hinge, a first IgG4 CH2 domain, a first IgG1 CH3 domain, a TFc linker, a second IgG4 hinge, a second IgG4 CH2 domain, and a second IgG1 CH3 domain.

Either or both of the first CH3 domain and the second CH3 domain of a TFc may comprise one or more aa modifications that enhance or stabilize the binding between the first and the second Fc regions, as evidenced, e.g., by an essentially uniform product (or band) on a non-denaturing SDS-Page gel. Each of the first CH3 domain and the second CH3 domain of a TFc may comprise an amino acid modification, which modification is an Association Enhancing Modification (“AEM”) that enhances the association of the first CH3 domain with the second CH3 domain. An AEM may be comprised by a module selected from the group consisting of AEM module 1, AEM module 2, AEM module 3 and AEM module 4. Either or both of the first Fc region and the second Fc region of a TFc may comprise an aa modification that adds a cysteine as an insertion or replacement, which cysteine forms a disulfide bond with a cysteine in the other Fc region (a “DiS” modification). Either or both of the first and the second Fc region of a TFc may comprise a DiS modification in a hinge. In certain embodiments, either or both of the first and the second Fc region comprise a DiS modification in a CH3 domain. The DiS modification may be comprised by DiS module 1 or DiS module 2. Each of the first CH3 domain and the second CH3 domain of a TFc may comprise one or more AEM modifications and one or more DiS modifications.

Either or both of the first and the second CH3 domains of a TFc may comprise an aa sequence that is at least 70%, 80%, 90%, 95%, 97%, 98%, or 99% identical to an aa sequence of a CH3 domain provided herein, e.g., selected from the group consisting of SEQ ID NOs:27-98, or which differs therefrom in at most 30, 25, 20, 15, 10, 5, 4, 3, 2, or 1 aa additions, deletions or substitutions. In certain embodiments, if the aa sequence of a CH3 domain is not identical to a sequence selected from the group of sequences SEQ ID NOs:27-98, then the aa sequence of the CH3 domain nevertheless comprises the particular AEM and/or DiS of the sequence to which it is similar. The first CH3 domain or the second CH3 domain of a TFc may comprises an aa sequence provided herein, e.g., selected from the group consisting of SEQ ID NOs:27-98. The first CH3 and second CH3 domains of a TFc together may comprise a pair of two different members, each member being a CH3 aa sequence, each pair selected from the group of pairs consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, each member aa sequence being at least 70%, 80%, 90%, 95%, 97%, 98%, or 99% identical to, or differing in at most 30, 25, 20, 15, 10, 5, 4, 3, 2, or 1 aa additions, deletions or substitutions from the each sequence of each said pair, wherein the first CH3 domain comprises a different member of the pair than is comprised by the second CH3 domain. The first and the second CH3 domains of a TFc may each comprise an aa sequence that identical to an aa sequence of a member of the pair of CH3 aa sequences selected from the group consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98.

The first hinge of a TFc may comprise an aa sequence that differs in at most 3, 2 or 1 aa deletions, additions or substitutions from an aa sequence of a hinge provided herein, e.g., selected from the group consisting of SEQ ID NOs:4, 18, 19, 20, 21, 22, 263-265 and 267-273. The first hinge of a TFc may comprise an aa sequence that is an aa sequence selected from the group consisting of SEQ ID NOs:4, 18, 19, 20, 21, 22, 263-265 and 267-273. The second hinge of a TFc may comprise an aa sequence that differs in at most 3, 2 or 1 aa deletions, additions or substitutions from an aa sequence of a hinge provided herein, e.g., selected from the group consisting of SEQ ID NOs:23, 24, 263-265 and 267-273. The second hinge may comprise an aa sequence that is an aa sequence selected from the group consisting of SEQ ID NOs:23, 24, 263-265 and 267-273.

A CH2 domain of a TFc may comprise an aa sequence that is at least 70%, 80%, 90%, 95%, 97%, 98%, or 99% identical to an aa sequence of a CH2 domain provided herein, e.g., SEQ ID NO:25, 26, 261 or 262, or which differs therefrom in at most 30, 25, 20, 15, 10, 5, 4, 3, 2, or 1 aa deletions, additions or substitutions.

The TFc may comprise in amino to carboxyl terminal order: a first hinge, a first CH2 domain, a first CH3 domain, a second hinge, a second CH2 domain and a second CH3 domain, wherein (i) the first hinge comprises an aa sequence selected from the group consisting of SEQ ID NOs:4, 18, 19, 263-265 and 267-273; (ii) the first CH2 domain is aglycosylated and comprises the aa sequence set forth as SEQ ID NO:25; (iii) the first CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98; (iv) the second hinge comprises an aa sequence consisting of a sequence selected from the group consisting of SEQ ID NO:23, 263-265 and 267-273; (v) the second CH2 domain is aglycosylated and comprises the aa sequence set forth in SEQ ID NO:25; and (vi) the second CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, wherein if the first CH3 domain comprises a first sequence of a pair of sequences, the second CH3 domain comprises the second sequence of the pair of sequences; and if the first CH3 domain comprises the second sequence of a pair of sequences, the second CH3 domain comprises the first sequence of the pair of sequences.

A TFc may comprise in amino to carboxyl terminal order: a first hinge, a first CH2 domain, a first CH3 domain, a second hinge, a second CH2 domain and a second CH3 domain, wherein (i) the first hinge comprises an aa sequence selected from the group consisting of SEQ ID NOs:20, 21, 22, 263-265 and 267-273; (ii) the first CH2 domain is aglycosylated and comprises the aa sequence set forth in SEQ ID NO:26; (iii) the first CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98; (iv) the second hinge comprises an aa sequence consisting of SEQ ID NO:24, 263-265 and 267-273; (v) the second CH2 domain is aglycosylated and comprises the aa sequence set forth in SEQ ID NO:26; and (vi) the second CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, wherein if the first CH3 domain comprises a first sequence of a pair of sequences, the second CH3 domain comprises the second sequence of the pair of sequences; and if the first CH3 domain comprises the second sequence of a pair of sequences, the second CH3 domain comprises the first sequence of the pair of sequences.

The first or the second Fc region of a TFc may comprise an aa sequence that is at least 70%, 80%, 90%, 95%, 97%, 98%, or 99% identical to an aa sequence of an Fc region provided herein, e.g., selected from the group consisting of SEQ ID NOs:99-166, or differs therefrom in at most 50, 40, 30, 25, 20, 15, 10, 5, 4, 3, 2, or 1 aa deletions, additions or substitutions. The first or the second Fc region comprises an aa sequence selected from the group consisting of SEQ ID NOs:99-166. The first and the second Fc region may comprise an aa sequence that is at least 70%, 80%, 90%, 95%, 97%, 98%, or 99% identical to one aa sequence of a pair of aa sequences selected from the group consisting of SEQ ID NOs:99 and 100; SEQ ID NOs:101 and 102; SEQ ID NOs:103 and 104; SEQ ID NOs:105 and 106; SEQ ID NOs:107 and 108; SEQ ID NOs:109 and 110; SEQ ID NOs:111 and 112; SEQ ID NOs:113 and 114; SEQ ID NOs:115 and 116; SEQ ID NOs:117 and 118; SEQ ID NOs:119 and 120; SEQ ID NOs:121 and 122; SEQ ID NOs:123 and 124; SEQ ID NOs:125 and 126; SEQ ID NOs:127 and 128; SEQ ID NOs:129 and 130; SEQ ID NOs:131 and 132; SEQ ID NOs:133 and 134; SEQ ID NOs:135 and 136; SEQ ID NOs:137 and 138; SEQ ID NOs:139 and 140; SEQ ID NOs:141 and 142; SEQ ID NOs:143 and 144; SEQ ID NOs:145 and 146; SEQ ID NOs:147 and 148; SEQ ID NOs:149 and 150; SEQ ID NOs:151 and 152; SEQ ID NOs:153 and 154; SEQ ID NOs:155 and 156; SEQ ID NOs:157 and 158; SEQ ID NOs:159 and 160; SEQ ID NOs:161 and 162; SEQ ID NOs:163 and 164; and SEQ ID NOs:165 and 166, or which differs therefrom in at most 50, 40, 30, 25, 20, 15, 10, 5, 4, 3, 2, or 1 aa deletions, additions or substitutions, and wherein the first Fc region comprises a different member of the pair than is comprised by the second Fc region. The first Fc region and the second Fc region together may comprise a pair of two different members, each member being an Fc aa sequence, wherein each pair is selected from the group of pairs consisting of SEQ ID NOs:99 and 100; SEQ ID NOs:101 and 102; SEQ ID NOs:103 and 104; SEQ ID NOs:105 and 106; SEQ ID NOs:107 and 108; SEQ ID NOs:109 and 110; SEQ ID NOs:111 and 112; SEQ ID NOs:113 and 114; SEQ ID NOs:115 and 116; SEQ ID NOs:117 and 118; SEQ ID NOs:119 and 120; SEQ ID NOs:121 and 122; SEQ ID NOs:123 and 124; SEQ ID NOs:125 and 126; SEQ ID NOs:127 and 128; SEQ ID NOs:129 and 130; SEQ ID NOs:131 and 132; SEQ ID NOs:133 and 134; SEQ ID NOs:135 and 136; SEQ ID NOs:137 and 138; SEQ ID NOs:139 and 140; SEQ ID NOs:141 and 142; SEQ ID NOs:143 and 144; SEQ ID NOs:145 and 146; SEQ ID NOs:147 and 148; SEQ ID NOs:149 and 150; SEQ ID NOs:151 and 152; SEQ ID NOs:153 and 154; SEQ ID NOs:155 and 156; SEQ ID NOs:157 and 158; SEQ ID NOs:159 and 160; SEQ ID NOs:161 and 162; SEQ ID NOs:163 and 164; and SEQ ID NOs:165 and 166, each member aa sequence being at least 70%, 80%, 90%, 95%, 97%, 98%, or 99% identical to, or differing in at most 50, 40, 30, 25, 20, 15, 10, 5, 4, 3, 2, or 1 aa additions, deletions or substitutions from each sequence of each said pair, wherein the first Fc region comprises a different member of the pair than is comprised by the second Fc region.

A TFc may comprise an aa sequence that is at least 70%, 80%, 90%, 95%, 97%, 98%, or 99% identical to an aa sequence of a TFc provided herein, e.g., selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221, or which differs therefrom in at most 30, 25, 20, 15, 10, 5, 4, 3, 2, or 1 aa additions, deletions or substitutions. The TFc may comprise an aa sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221.

A TFcA, e.g., a TFcBA, e.g., an anti-c-Met+anti-EGFR TFcBA or an anti-c-Kit+anti-RON TFcBA or an anti-FGFR2+anti-EPCAM TFcBA, may comprise a heavy chain that comprises in amino to carboxyl terminal order: a first heavy chain variable (VH) domain, a TFc, a connecting linker and a second VH domain. The heavy chain may comprise in amino to carboxyl terminal order: a first VH domain, a CH1 domain, a TFc, a connecting linker and a second VH domain. The heavy chain may comprise in amino to carboxyl terminal order: a first VH domain, a CH1 domain, a TFc, a connecting linker, a second VH domain, an scFv linker and a second light chain variable (VL) domain, wherein the second VH and VL domains associate to form a second binding site. A TFcA may comprise a light chain that comprises a first VL domain that dimerizes with the first VH domain to form a first binding site. The light chain may comprise a light chain constant (CL) domain that is linked to the carboxyl terminus of the VL domain. The first binding site may be an anti-c-Met, anti-c-Kit, anti-ErbB2, anti-ErbB3, anti-ErbB4, anti-IGF1R, anti-IGF2R, anti-Insulin receptor, anti-RON, anti-EGFR, anti-VEGFR1, anti-VEGFR2, anti-TNFR, anti-FGFR1, anti-FGFR2, anti-FGFR3, anti-FGFR4, anti-PDGFR alpha, anti-PDGFR beta, anti-EPCAM or anti-EphA2 binding site and the second binding site may be an anti-c-Met, anti-c-Kit, anti-ErbB2, anti-ErbB3, anti-ErbB4, anti-IGF1R, anti-IGF2R, anti-Insulin receptor, anti-RON, anti-VEGFR1, anti-VEGFR2, anti-TNFR, anti-FGFR1, anti-FGFR2, anti-FGFR3, anti-FGFR4, anti-PDGFR alpha, anti-PDGFR beta, anti-EphA2 or anti-EGFR binding site. If a TFcA is a monovalent TFcA, the binding site may be an anti-c-Met, anti-c-Kit, anti-ErbB2, anti-ErbB3, anti-ErbB4, anti-IGF1R, anti-IGF2R, anti-Insulin receptor, anti-RON, anti-VEGFR1, anti-VEGFR2, anti-TNFR, anti-FGFR1, anti-FGFR2, anti-FGFR3, anti-FGFR4, anti-PDGFR alpha, anti-PDGFR beta, anti-EPCAM, anti-EphA2 or anti-EGFR binding site. An exemplary anti-c-Met binding site may comprise a VH domain comprising either or both of a) the aa sequence of the VH Complementarity Determining Region (CDR)3 (VHCDR3) in SEQ ID NO:223 or 287 and b) a VLCDR3 comprising the aa sequence of the VLCDR3 in SEQ ID NO:231 or 289. Another exemplary the anti-c-Met binding site may comprise a VH domain comprising a set of three VH CDRs comprising VHCDR1, VCDR2 and VHCDR3, wherein VHCDR1, VHCDR2 and VHCDR3 comprise the aa sequence of the VHCDR1, VHCDR2 and VHCDR3 in SEQ ID NO:223 or 231; and a VL domain comprising a set of three VLCDRs comprising VLCDR1, VLCDR2, and VLCDR3, wherein VLCDR1, VLCDR2 and VLCDR3 comprise the aa sequence of the VLCDR1, VLCDR2 and VLCDR3 in SEQ ID NO:287 or 289, respectively. An exemplary anti-EGFR binding site may comprise either or both of a) a VHCDR3 comprising the aa sequence of the VHCDR3 in SEQ ID NO:233, 237, 258, 275, 277 or 279 and b) a VLCDR3 comprising the aa sequence of the VLCDR3 in SEQ ID NO:233, 237, 258, 275, 277 or 279. An exemplary anti-EGFR binding site may comprise a VH domain comprising a set of three VHCDRs comprising VHCDR1, VCDR2 and VHCDR3, wherein VHCDR1, VHCDR2 and VHCDR3 comprise the aa sequence of the VHCDR1, VHCDR2 and VHCDR3 in SEQ ID NO: 233, 237, 258, 275, 277 or 279; and a VL domain comprising a set of three VLCDRs comprising VLCDR1, VLCDR2, and VLCDR3, wherein VLCDR1, VLCDR2 and VLCDR3 comprise the aa sequence of the VLCDR1, VLCDR2 and VLCDR3 in SEQ ID NO:233, 237, 258, 275, 277 or 279. The anti-c-Met, anti-c-Kit, anti-ErbB2, anti-ErbB3, anti-ErbB4, anti-IGF1R, anti-IGF2R, anti-Insulin receptor, anti-RON, anti-VEGFR1, anti-VEGFR2, anti-TNFR, anti-FGFR1, anti-FGFR2, anti-FGFR3, anti-FGFR4, anti-PDGFR alpha, anti-PDGFR beta, anti-EPCAM, anti-EphA2 or anti-EGFR binding site may comprise an N-terminal portion of the heavy chain and an N-terminal portion of the light chain. The anti-EGFR, anti-c-Kit, anti-ErbB2, anti-ErbB3, anti-ErbB4, anti-IGF1R, anti-IGF2R, anti-Insulin receptor, anti-RON, anti-VEGFR1, anti-VEGFR2, anti-TNFR, anti-FGFR1, anti-FGFR2, anti-FGFR3, anti-FGFR4, anti-PDGFR alpha, anti-PDGFR beta, anti-EPCAM, anti-EphA2 or anti-c-Met binding site may be comprised by a C-terminal scFv that is entirely comprised by the heavy chain to form a contiguous polypeptide.

An anti-c-Met binding site of a TFcA, e.g., a TFcBA, may be comprised by either or both of a VH domain and a VL domain, wherein the VH domain comprises an aa sequence that is at least 70%, 80%, 90%, 95%, 97%, 98%, or 99% identical to the VH domain of an anti-c-Met binding site, e.g., set forth in SEQ ID NOs:223, 231, 287 or 289, or differs therefrom in at most 10, 9, 8, 7, 6, 5, 4, 3, 2, or 1 aas deletions, additions or substitution; and the VL domain comprises an aa sequence that is at least 70%, 80%, 90%, 95%, 97%, 98%, or 99% identical to the VL domain of an anti-c-Met binding site provided herein, e.g., set forth in SEQ ID NOs:223, 231, 287 or 289, or differs therefrom in at most 10, 9, 8, 7, 6, 5, 4, 3, 2, or 1 aas deletions, additions or substitution.

An anti-EGFR binding site of a TFcA, e.g., a TFcBA, may be comprised by either or both of a VH domain and a VL domain, wherein the VH domain comprises an aa sequence that is at least 70%, 80%, 90%, 95%, 97%, 98%, or 99% identical to the VH domain of an anti-EGFR binding site provided herein, e.g., set forth in SEQ ID NOs: 233, 237, 258, 275, 277 or 279, or differs therefrom in at most 10, 9, 8, 7, 6, 5, 4, 3, 2, or 1 aa(s) deletion(s), addition(s) or substitution(s); and the VL domain comprises an aa sequence that is at least 70%, 80%, 90%, 95%, 97%, 98%, or 99% identical to the VL domain of an anti-EGFR binding site provided herein, e.g., set forth in SEQ ID NOs: 233, 237, 258, 275, 277 or 279, or differs therefrom in at most 10, 9, 8, 7, 6, 5, 4, 3, 2, or 1 aa(s) deletion(s), addition(s) or substitution(s).

A TFcA or TFcBA may be a charge-complementary paired TFcA or TFcBA, e.g., wherein: a charge-complementary paired TFcA or TFcBA is a TFcA or TFcBA that comprises a pair of charged amino acids comprising an amino acid selected from group A and an amino acid selected from group B (a charge-complementary pair); wherein group A comprises all natural amino acids with a pI of greater than 7 and group B comprises all natural amino acids with a pI of less than 7, or optionally wherein group A comprises His, Lys, and Arg, and group B comprises Asp, Glu, Asn, Phe, Gln, Tyr, Ser, Met, Thr, Ile, Gly, Val, Trp, Leu, Ala, and Pro; and said charge-complementary pair consists of a first amino acid residue and a second amino acid residue, and said charge-complementary pair is a position 297 charge-complementary pair or a position 299 charge-complementary pair, wherein a position 297 charge-complementary pair is a charge-complementary pair with said first amino acid residue located at EU position 297 of said first Fc region and said second amino acid residue located at EU position 297 of said second Fc region, and a position 299 charge-complementary pair is a charge-complementary pair with said first amino acid residue located at EU position 299 of said first Fc region and said second amino acid residue located at EU position 299 of said second Fc region. The charge-complementary paired TFcA or TFcBA may comprise both a position 297 charge-complementary pair and a position 299 charge-complementary pair, wherein the first and second amino acid residues of the position 297 charge-complementary pair are the same as or different from the first and second amino acid residues of the position 299 charge-complementary pair. The charge-complementary paired TFcA or may comprise a position 297 charge-complementary pair and wherein the charge-complementary paired TFcA or TFcBA is more stable than a TFcA or TFcBA that is not a charge-complementary paired TFcA or TFcBA but that is identical to the charge-complementary paired TFcA or TFcBA except that amino acid residues corresponding to the first and the second amino acid residues are both residues consisting of the same charged amino acid, said same charged amino acid being one of the amino acids of the position 297 charge-complementary pair of the charge-complementary paired TFcA or TFcBA. The charge-complementary paired TFcA or TFcBA may comprise a position 299 charge-complementary pair and wherein the charge-complementary paired TFcA or TFcBA is more stable than a TFcA or TFcBA that is not a charge-complementary paired TFcA or TFcBA but that is identical to the charge-complementary paired TFcA or TFcBA except that amino acid residues corresponding to the first and the second amino acid residues are both residues consisting of the same charged amino acid, said same charged amino acid being one of the amino acids of the position 299 charge-complementary pair of the charge-complementary paired TFcA or TFcBA.

The first or the second binding site of a TFcA or TFcBA may bind specifically to a human receptor protein selected from the group consisting of ErbB2, ErbB3, ErbB4, IGF1R, IGF2R, Insulin receptor, RON, c-Met, EGFR, VEGFR1, VEGFR2, TNFR, FGFR1-4, PDGFR (alpha and beta), c-Kit, EPCAM and EphA2.

Further provided herein are pharmaceutical compositions comprising a TFcA or TFcBA and a pharmaceutically acceptable carrier. Also provided are nucleic acid molecules, e.g., comprising at least one coding sequence, said at least one coding sequence encoding a heavy chain or a light chain of a TFcA or TFcBA. A nucleic acid molecule may comprise at least two coding sequences, wherein one coding sequence encodes a heavy chain of a TFcA or TFcBA and a second coding sequence encodes a light chain of the TFcBA. Also provided are vectors, e.g., comprising one or more nucleic acid molecules provided herein. Further provided are cells, e.g., host cells or isolated cells, comprising one or more vectors and/or nucleic acid molecules provided herein. A cell may comprise a nucleic acid molecule encoding the heavy chain of a TFcA or TFcBA and a nucleic acid molecule encoding the light chain of the TFcA or TFcBA.

Also encompassed herein are methods of producing a TFcA or TFcBA comprising culturing a host cell described herein under conditions in which the nucleic acids are expressed, and isolating the TFcA or TFcBA. A method for producing a TFcA or TFcBA may comprise culturing a cell described herein under conditions suitable for the expression of the TFcA or TFcBA.

Also provided herein are methods of treating a subject having cancer, said method comprising administering to a subject a therapeutically effective amount of a TFcA or TFcBA, nucleic acid molecule, or vector described herein.

### BRIEF DESCRIPTION OF THE DRAWINGS

FIG. 1A and FIG. 1B: Diagram of an exemplary anti-c-Met/anti-EGFR Tandem Fc Bispecific Antibody (“TFcBA”) (FIG. 1A) and exemplary mutations in each of the domains of the Tandem Fc (“TFc”) (FIG. 1B).

FIG. 1A: Diagram of an exemplary anti-c-Met/anti-EGFR TFcBA comprising the following three modules in amino to carboxyl terminal order: 1) a first module consisting of an anti-c-Met binding site; 2) a second module consisting of a TFc; and 3) a third module consisting of an anti-EGFR binding site. In the exemplified TFcBA, the first module is an anti-c-Met Fab and the third module is an anti-EGFR scFv. The TFc comprises two Fc regions that are linked through a TFc linker. In the exemplified TFcBA, the first Fc region comprises a full length IgG1/IgG4 hybrid hinge, an IgG4 CH2 domain, and an IgG1 CH3 domain, and the second Fc region comprises the core and lower hinge of IgG4 (but does not comprise an upper hinge), an IgG4 CH2 domain and an IgG1 CH3 domain. In the exemplified TFcBA, the CH3 domain comprises one or more Association Enhancing Modifications (“AEMs”), which enhance the association between two CH3 domains or two Fc regions. TFcBAs may also comprise one or more disulfide bond forming modifications (“DiSs”), which introduce cysteines allowing for the formation of disulfide bonds between two Fc regions.

FIG. 1B: Diagram of the structure of a TFc showing in amino to carboxyl terminal order: the first hinge, the first CH2 domain, the first CH3 domain, the TFc linker, the second hinge, the second CH2 domain and the second CH3 domain. Exemplary sequences and domain modifications for each of these domains are shown below the diagram. The name of the first or second CH3 modification in each AEM or DiS is indicated in parenthesis after the name of the modification, wherein the first numeral after “AEM” or “DiS” refers to the module number of the AEM or DiS, respectively, and the second numeral refers to the first or the second of the two CH3 domains. For example, “AEM 1.1” is indicated after the substitutions “T366S/L368A/Y407V,” which substitutions are the combination of substitutions in one of the two CH3 domains of a pair of modifications in AEM module 1. A TFc may comprise any combination of each of these domains, with the proviso that when one of the CH3 domain of the TFc comprises one of the two modifications of an AEM and/or DiS, the other CH3 domain comprises the second, i.e., compatible, modification(s) of the AEM and/or DiS. For example, if one CH3 domain of a TFc comprises AEM 1.1, the other CH3 domain comprises AEM 1.2. “C-term. Cys” refers to a modification adding a C-terminal Cysteine to the CH3 domain by substituting the last three aas of the CH3 domain with those shown in the Figure. Aa residue numbers in this Figure and the other Figures are those in an intact antibody heavy chain, according to the EU index in Kabat.

FIG. 2: Alignment of aa sequences of wild type and variant hinges. A dash “-” at a position represents an aa that is identical to that in the first line of the figure at that position. A) Aa sequences of full length (SEQ ID NOs:4, 18 and 19) or partial (SEQ ID NOs:1, 2, 3, 16, 17, 23 and 263-265) IgG1 hinges that are wild type (SEQ ID NOs:1-4 and 23) or modified (SEQ ID NOs:16-19 and 263-265). B) Aas of full length (SEQ ID NOs:20, 21 and 22) or partial (SEQ ID NOs:1, 13, 14 and 24) IgG1/IgG4 hybrid hinges that are wild type (SEQ ID NOs:1, 13, 14, 20 and 24) or modified (SEQ ID NOs:21 and 22). C) Aa sequences of full length wild type mIgG1 hinge (SEQ ID NO:266) and hybrid mIgG1/mIgG2A hinge (SEQ ID NO:267). D) Aa sequence of a full length, wild type hIgG2 hinge (SEQ ID NO:7) and modified hIgG2 hinges (SEQ ID NOs:268 and 269). E) Aa sequence of a full length wild type hIgA2 hinge (SEQ ID NO:270) and modified hIgA2 hinges (SEQ ID NOs:271-273).

FIG. 3: Alignment of IgG1 CH3 aa sequences with or without various aa modifications. Each line is the aa sequence of a different CH3 domain. A dash “-” at a position represents an aa that is identical to that in the first line of the figure at that position. The CH3 modifications are organized according to their module, e.g., AEM module 1. Each module is divided into two groups labeled with two numerals: for example, AEM module 1 is divided into the groups “AEM 11” and “AEM 12,” wherein AEM 11 represents the modifications made to one CH3 domain (domain “1”) of module AEM 1 and AEM 12 represents the modifications made to the second CH3 domain (domain “2”) of the module. Each line within a module represents a CH3 domain having the modifications of the module with or without other modifications. The CH3 aa sequences within one module differ from each other, e.g., in the presence or absence of the carboxyl terminal lysine and/or in the presence of the substitutions D356E and L358M.

FIG. 4: Alignment of exemplary IgG1 Fc regions. Each line is the aa sequence of a different Fc region. A dash “-” at a position represents an aa that is identical to that in the first line of the figure at that position. Each Fc region comprises a hinge (boldface in first sequence), CH2 and CH3 domain (the CH3 domain is underlined in the first sequence). The SEQ ID NOs of the hinge, CH2 and CH3 sequences of each Fc in this Figure are provided in Table 8. The Fcs are organized in pairs, which are separated form other pairs by lines, and wherein each pair represents compatible Fcs, i.e., Fcs that can associate with each other to form an Fc dimer.

FIG. 5: Alignment of exemplary IgG1/IgG4 hybrid Fc regions. Each line is the aa sequence of a different Fc region. A dash “-” at a position represents an aa that is identical to that in the first line of the figure at that position. Each Fc region comprises a hinge (boldface in the first sequence), CH2 and CH3 domain (the CH3 domain is underlined in the first sequence). The SEQ ID NOs of the hinge, CH2 and CH3 sequences of each Fc in this Figure are provided in Table 9. The Fcs are organized in pairs, which are separated form other pairs by lines, and wherein each pair represents compatible Fcs, i.e., Fcs that can associate with each other to form an Fc dimer.

FIG. 6: Aa sequences of the following IgG1 TFcs: 23 (SEQ ID NO:171); 23A (SEQ ID NO:173); 23B (SEQ ID NO:175); 23C (SEQ ID NO:177); 23D (SEQ ID NO:179); 23E (SEQ ID NO:181); 23F (SEQ ID NO:183); 23E(35L) (SEQ ID NO:185); 23E(35L Inverted) (SEQ ID NO:187); 23E(30L) (SEQ ID NO:189); 23E(25L) (SEQ ID NO:191); 23I (SEQ ID NO:193); and 23J (SEQ ID NO:195). Each of these sequences consists of the following domains in amino to carboxyl terminal order: a first IgG1 hinge (double underlined), an IgG1 CH2 domain, an IgG1 CH3 domain (underlined), a (G4S)n linker (in italics), a second IgG1 hinge (doubly underlined, and consisting of the core and lower hinges only), a second IgG1 CH2 domain and a second IgG1 CH3 domain (underlined). The aa changes that are specific to each of these molecules are shown in boldface, and are named above the sequence.

FIG. 7: Aa sequences of the following IgG1/IgG4 hybrid TFcs: 39 (SEQ ID NO:197); 39A (SEQ ID NO:199); 39B (SEQ ID NO:201); 39C (SEQ ID NO:203); 39D (SEQ ID NO:205); 39E (SEQ ID NO:207); 39F (SEQ ID NO:209); 39E(35L) (SEQ ID NO:211); 39 E(35L Inverted) (SEQ ID NO:213); 39E(30L) (SEQ ID NO:215); 39E(25L) (SEQ ID NO:217); 39I (SEQ ID NO:219); 39J (SEQ ID NO:221). Each of these sequences consist of the following domains in amino to carboxyl terminal order: a first IgG1/IgG4 hybrid hinge consisting of the IgG1 upper hinge and IgG4 core and lower hinges (double underlined), an IgG4 CH2 domain, an IgG1 CH3 domain (underlined), a (G4S)n linker (in italics), a second IgG4 hinge (double underlined, and consisting of the core and lower hinges only), a second IgG4 CH2 domain and a second IgG1 CH3 domain (underlined). The IgG1 sequences are in upper case letters and the IgG4 sequences are in lower case letters. The aa changes that are specific to each of these molecules are shown in boldface, and are named above the sequence.

FIG. 8: Samples of TFcs 23A, 23B, 23D, 23E, 39B and 39G separated on a 4-12% SDS-PAGE gel under A) non reducing or B) reducing conditions. Molecular weights of the proteins (in KDas) of the molecular weight marker (Biorad Precision Plus Marker) of lane 1 are shown on the left of the gel.

FIG. 9: Aa sequences of heavy chains of the following exemplary anti-c-Met/anti-EGFR TFcBAs: TFcBAs comprising a humanized 5D5 VH domain and an anti-EGFR scFv comprising the aa sequences of the VH and VL domains of A), B), C), D) E), L) and M) panimumumab (SEQ ID NO:235); F) 2224 (SEQ ID NO:239); G) cetuximab H1L1 (SEQ ID NO:260); H) cetuximab H1L2 (SEQ ID NO:281); I) cetuximab H2L1 (SEQ ID NO:283); and J) cetuximab H2L2 (SEQ ID NO:285). K) Aa sequence of the heavy chain of anti-c-Met/anti-EGFR TFcBA comprising the VH domain of anti-c-Met binding site 2 and humanized anti-EGFR cetuximab scFv H1L1. The aas that were introduced into the cetuximab VH domains for humanization purposes are indicated in lower case. The CDRs of the anti-c-Met Fab are underlined with a dotted line. The CH1 domain is underlined with a wavy line. The hinges are double underlined. The TFc linker is in italics. The CH3 domains are underlined. The AEM and DiS modifications in the CH3 domains are in boldface. The scFv linker is in italics and underlined. The connecting linker is in italics and double underlined.

FIG. 10: Nucleotide sequences encoding the aa sequences set forth in the Figures and in the specification.

FIG. 11: Nucleotide and aa sequences of TFcs used in Examples 1 and 2. Each of the aa sequences consists of the following domains in amino to carboxyl terminal order: a signal peptide (underlined and boldface), a first IgG1 hinge (double underlined), an IgG1 CH2 domain, an IgG1 CH3 domain (underlined), a TFc linker (in italics), a second IgG1 hinge (doubly underlined, and consisting of the core and lower hinges only), a second IgG1 CH2 domain and a second IgG1 CH3 domain (underlined). IgG1 aas are in upper case and IgG4 aas are in lower case. The aa changes that are specific to each of these molecules, e.g., AEMs and DiSs modifications, are shown in boldface, and are named above the sequence.

FIG. 12A: Selection of onartuzumab (OTZM) monoclonal cell line: Lane 1=size standards, Lanes 2-12; 2=OTZM line 1, 3=OTZM line 2, 4=OTZM line 3, 5=OTZM line 4, 6=OTZM line 5, 7=OTZM line 6, 8=OTZM line 7, 9=OTZM line 8, 10=OTZM line 9, 11=OTZM line 10, 12=OTZM line 11.

FIG. 12B: Selection of onartuzumab (OTZM) monoclonal cell line: Lane 1=size standards Lanes 2-9; 2=OTZM line 12, 3=OTZM line 13, 4=OTZM line 14, 5=OTZM line 15, 6=OTZM line 16, 7=OTZM line 17, 8=OTZM line 18, 9=OTZM line 19.

FIG. 13A: Non-reduced SDS-PAGE of charged glycosylation mutants: Lane 1=size standards, Lanes 2-8; 2=glyco wt, 3=glyco 1, 4=glyco 2, 5=glyco 3, 6=glyco 4, 7=glyco 5, 8=glyco 6.

FIG. 13B: Reduced SDS-PAGE of charged glycosylation mutants: Lane 1=size standards, Lanes 2-8; 2=glyco wt, 3=glyco 1, 4=glyco 2, 5=glyco 3, 6=glyco 4, 7=glyco 5, 8=glyco 6.

FIG. 14: Nucleotide and aa sequences of exemplary TFcBAs.

FIG. 15: A graph showing binding to cMet-Fc and EGFR-his of TFcBAs comprising the 39E glycoform 4 backbone, onartuzumab antibody and either 2224 or panitumumab antibody.

FIG. 16: A graph showing inhibition of pMet by TFcs comprising onartuzumab antibody and various backbones including 23, 23E, 39, 39E glycoform 4 backbone, and including TFcBAs comprising 39E glycoform 4 backbone and 2224, cetuximab, or panitumumab antibody.

FIG. 17: Nucleotide and aa sequences of glycosylation mutants of the exemplary TFcBAs set forth in Table 23.

### BRIEF DESCRIPTION OF THE SEQUENCES

The amino acid (“aa”) sequences referred to herein and listed in the sequence listing are identified below.

SEQ ID NOs:1, 2 and 3 are the aa sequences of the wild type IgG1 upper, middle (or core) and lower hinge, respectively (see Table 2).

SEQ ID NO:4 is the aa sequence of the complete wild type IgG1 hinge, consisting of SEQ ID NOs:1, 2 and 3 in a contiguous sequence in amino to carboxyl terminal order (see Table 2).

SEQ ID NOs:5 and 6 are the aa sequences of the wild type IgG2 upper and lower hinge, respectively (see Table 2). The IgG2 middle hinge is the same as that of IgG1, i.e., SEQ ID NO:2.

SEQ ID NO:7 is the aa sequence of the complete wild type IgG2 hinge, consisting of SEQ ID NOs:5, 2 and 6 in a contiguous sequence in amino to carboxyl terminal order (see Table 2).

SEQ ID NOs:8, 9 and 10 are the aa sequences of the wild type IgG3 upper, middle and lower hinge, respectively (see Table 2).

SEQ ID NO:11 is the aa sequence of the complete wild type IgG3 hinge, consisting of SEQ ID NOs:8, 9 and 10 in a contiguous sequence in amino to carboxyl terminal order (see Table 2).

SEQ ID NOs:12, 13 and 14 are the aa sequences of the IgG4 upper, middle and lower hinge, respectively (see Table 2).

SEQ ID NO:15 is the aa sequence of a full length IgG4 hinge, consisting of SEQ ID NOs:12, 13 and 14 in a contiguous sequence in amino to carboxyl terminal order (see Table 2).

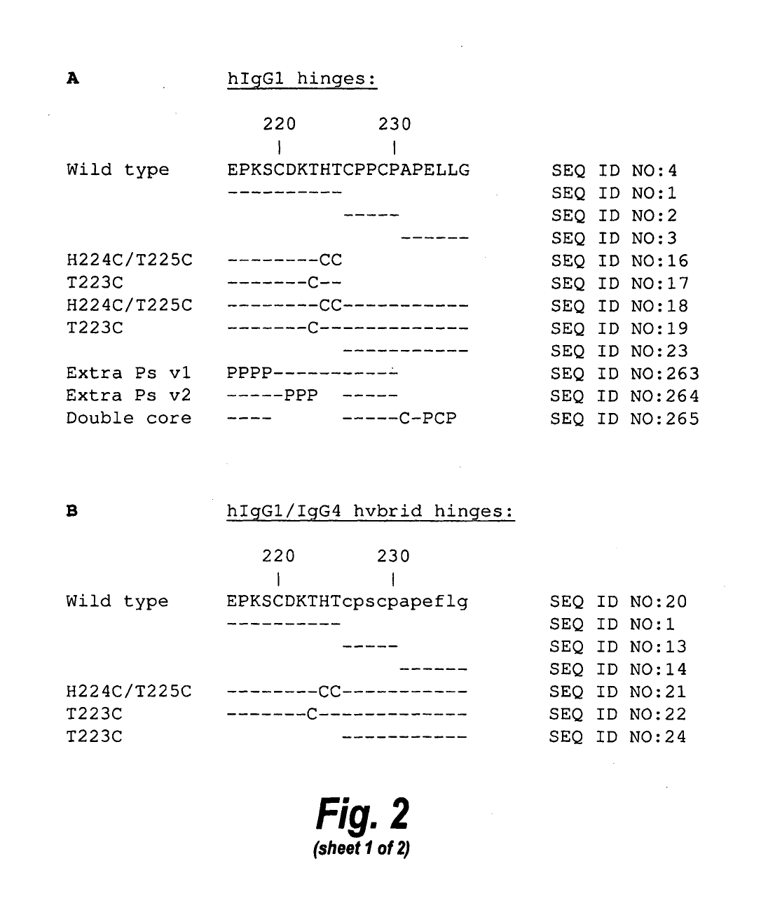

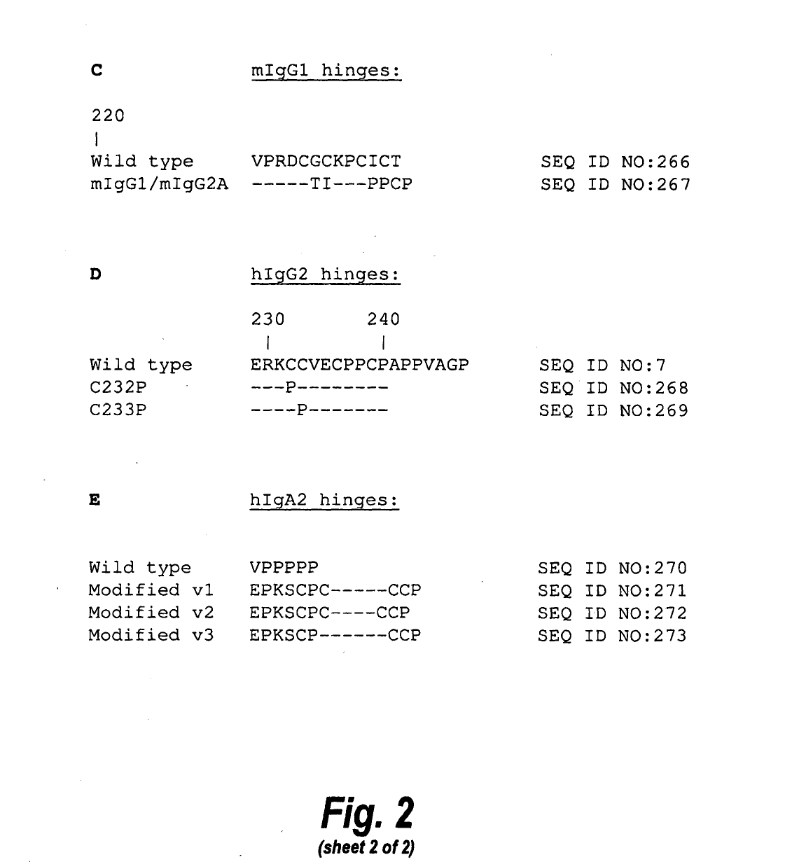

SEQ ID NO:16 is the aa sequence of the IgG1 upper hinge (SEQ ID NO:1) comprising the aa substitutions H224C and T225C (see Table 4 and FIG. 2).

SEQ ID NO:17 is the aa sequence of the IgG1 upper hinge (SEQ ID NO:1) comprising the aa substitution T223C (see Table 4 and FIG. 2).

SEQ ID NO:18 is the aa sequence of the full length IgG1 hinge (SEQ ID NO:4) comprising the aa substitutions H224C and T225C (see Table 4 and FIG. 2).

SEQ ID NO:19 is the aa sequence of the full length IgG1 hinge (SEQ ID NO:4) comprising the aa substitution T223C (see Table 4 and FIG. 2).

SEQ ID NO:20 is the aa sequence of a full length hybrid IgG1/IgG4 hinge, consisting of the upper hinge of IgG1 (SEQ ID NO:1) and the middle and lower hinges of IgG4 (SEQ ID NOs:13 and 14, respectively; see Table 4 and FIG. 2).

SEQ ID NO:21 is the aa sequence of a full length hybrid IgG1/IgG4 hinge, consisting of the upper hinge of IgG1 comprising the aa substitutions H224C and T225C (SEQ ID NO:16) and the middle and lower hinges of IgG4 (SEQ ID NOs:13 and 14, respectively; see Table 4 and FIG. 2).

SEQ ID NO:22 is the aa sequence of a full length hybrid IgG1/IgG4 hinge, consisting of the upper hinge of IgG1 comprising the aa substitution T223C (SEQ ID NO:17) and the middle and lower hinges of IgG4 (SEQ ID NOs:13 and 14, respectively; see Table 4 and FIG. 2).

SEQ ID NO:23 is the aa sequence of a partial IgG1 hinge comprising the middle and lower IgG1 hinges (SEQ ID NOs:2 and 3), but not the upper hinge (see Table 4 and FIG. 2).

SEQ ID NO:24 is the aa sequence of a partial IgG4 hinge comprising the middle and lower IgG4 hinges (SEQ ID NOs:13 and 14), but not the upper hinge (see Table 4 and FIG. 2).

SEQ ID NO:25 is the aa sequence of a full length IgG1 CH2 domain with the aa substitution N297Q reducing glycosylation at aa 297.

SEQ ID NO:26 is the aa sequence of a full length wild type IgG4 CH2 domain with the aa substitution T299K reducing glycosylation at aa 297.

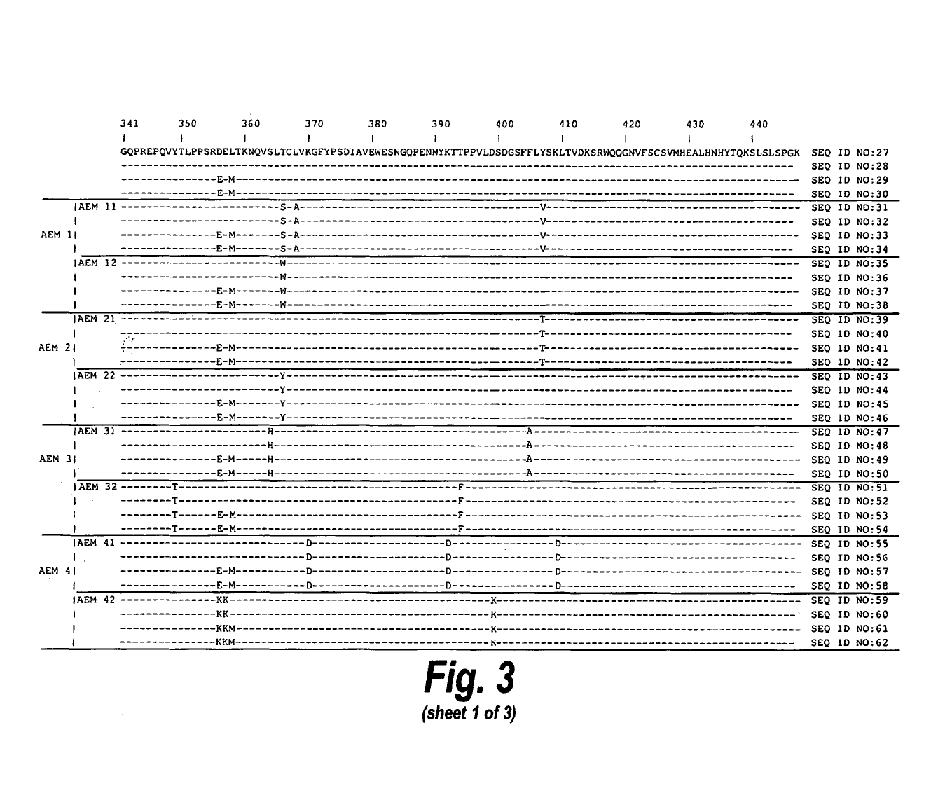

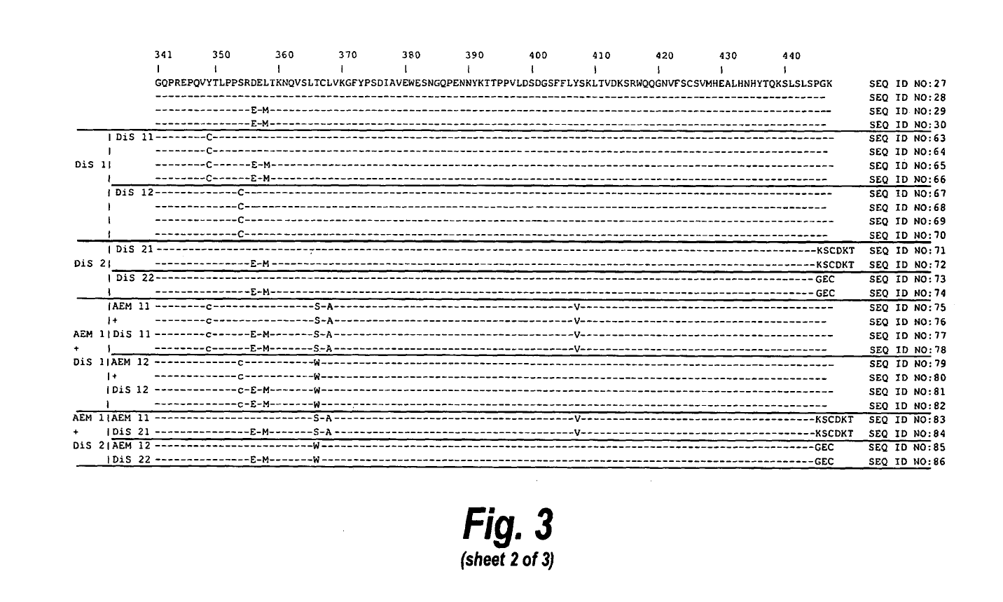

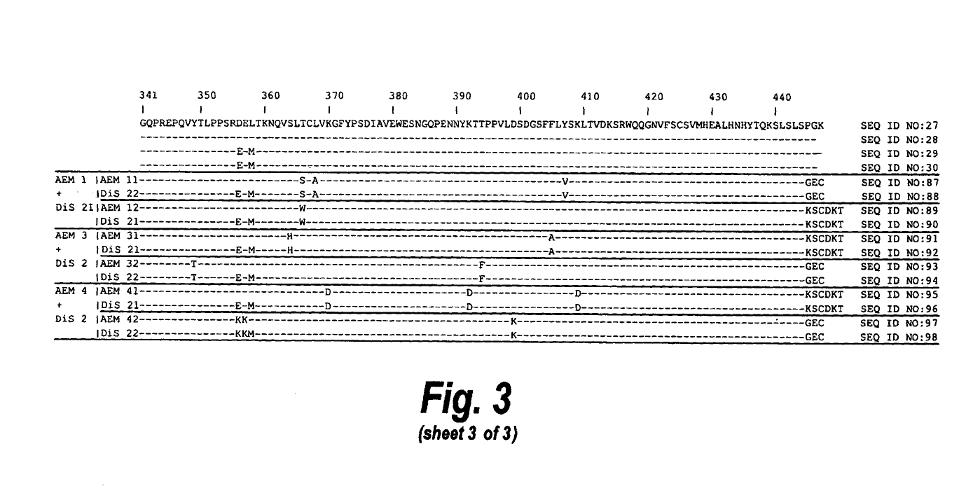

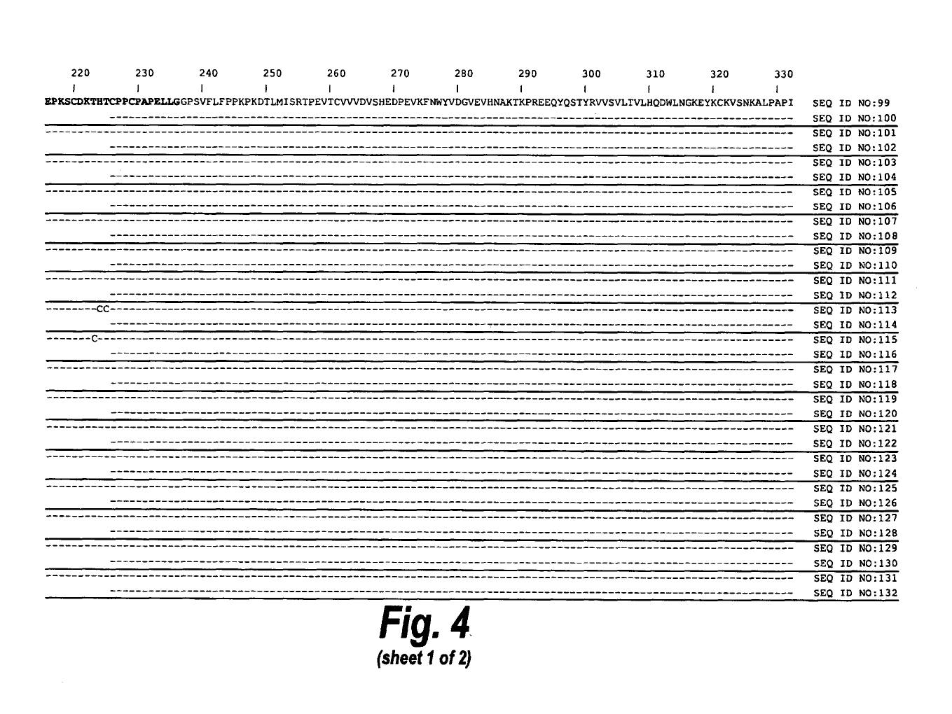

SEQ ID NO:27 is the aa sequence of a full length wild type human IgG1 CH3 domain (see Table 6 and FIG. 3).

SEQ ID NO:28 is the aa sequence of the wild type IgG1 CH3 domain having SEQ ID NO:27, but lacking the C-terminal lysine (see Table 6 and FIG. 3).

SEQ ID NO:29 is the aa sequence of the IgG1 CH3 domain having SEQ ID NO:27 with the substitutions D356E and L358M (see Table 6 and FIG. 3).

SEQ ID NO:30 is the aa sequence of the IgG1 CH3 domain having SEQ ID NO:29, lacking the C-terminal lysine (see Table 6 and FIG. 3).

SEQ ID NO:31 is the aa sequence of the IgG1 CH3 domain having SEQ ID NO:27 with the substitutions T366S, L368A and Y470V, creating a “hole” (Association Enhancing Modification or “AEM” 1.1; see Table 6 and FIG. 3).

SEQ ID NO:32 is the aa sequence of the IgG1 CH3 domain having SEQ ID NO:31, lacking the C-terminal lysine (see Table 6 and FIG. 3).

SEQ ID NO:33 is the aa sequence of the IgG1 CH3 domain having SEQ ID NO:29 with the substitutions T3665, L368A and Y470V, creating a “hole” (AEM 1.1; see Table 6 and FIG. 3).

SEQ ID NO:34 is the aa sequence of the IgG1 CH3 domain having SEQ ID NO:33, lacking the C-terminal lysine (see Table 6 and FIG. 3).

SEQ ID NO:35 is the aa sequence of the IgG1 CH3 domain having SEQ ID NO:27 with the substitution T366W, creating a “bump” or “knob” (AEM 1.2; see Table 6 and FIG. 3).

SEQ ID NO:36 is the aa sequence of the IgG1 CH3 domain having SEQ ID NO:35, lacking the C-terminal lysine (see Table 6 and FIG. 3).

SEQ ID NO:37 is the aa sequence of the IgG1 CH3 domain having SEQ ID NO:29 with the substitution T366W, creating a “bump” or “knob” (AEM 1.2; see Table 6 and FIG. 3).

SEQ ID NO:38 is the aa sequence of the IgG1 CH3 domain having SEQ ID NO:37, lacking the C-terminal lysine (see Table 6 and FIG. 3).

SEQ ID NOs:39-98 are the aa sequences of IgG1 CH3 domains comprising one or more AEM and/or Disulfide bond forming (“DiS”) modifications relative to IgG1 CH3 having SEQ ID NO:27, 28, 29 or 30 (see Table 6 and FIG. 3).

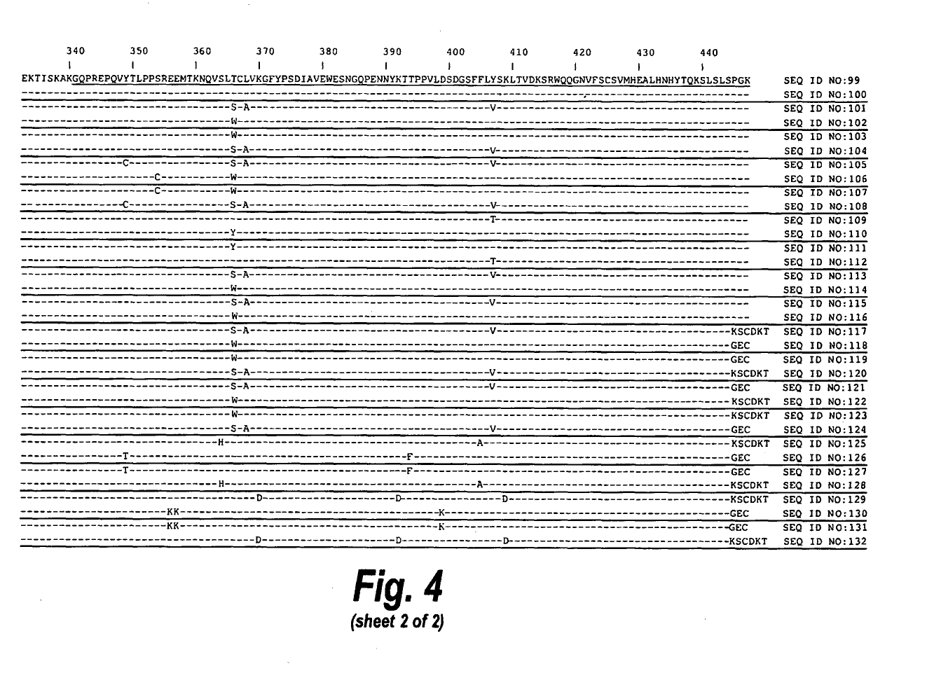

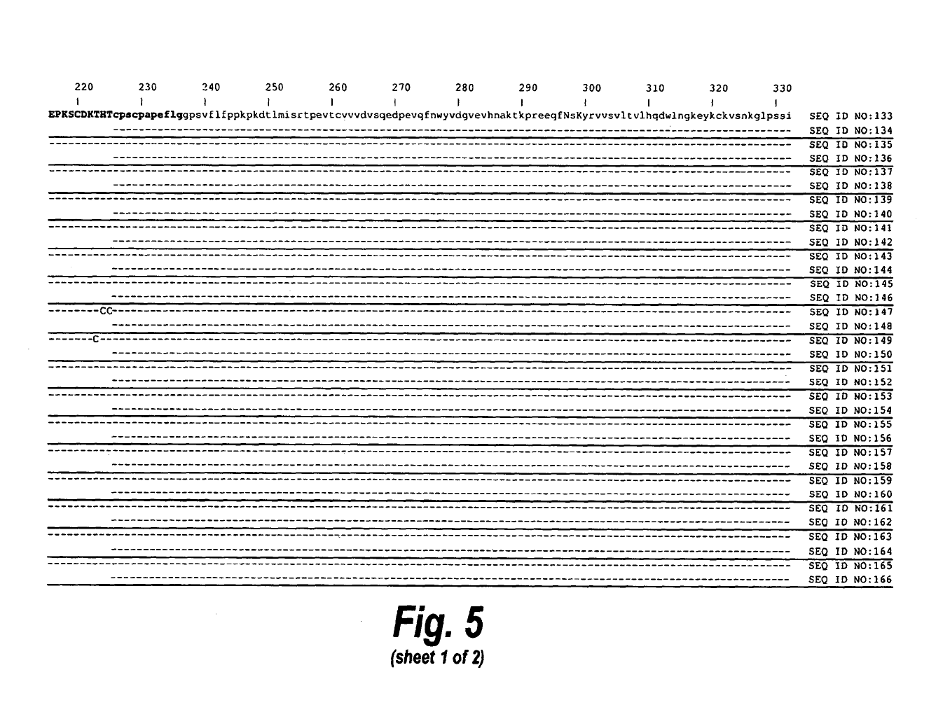

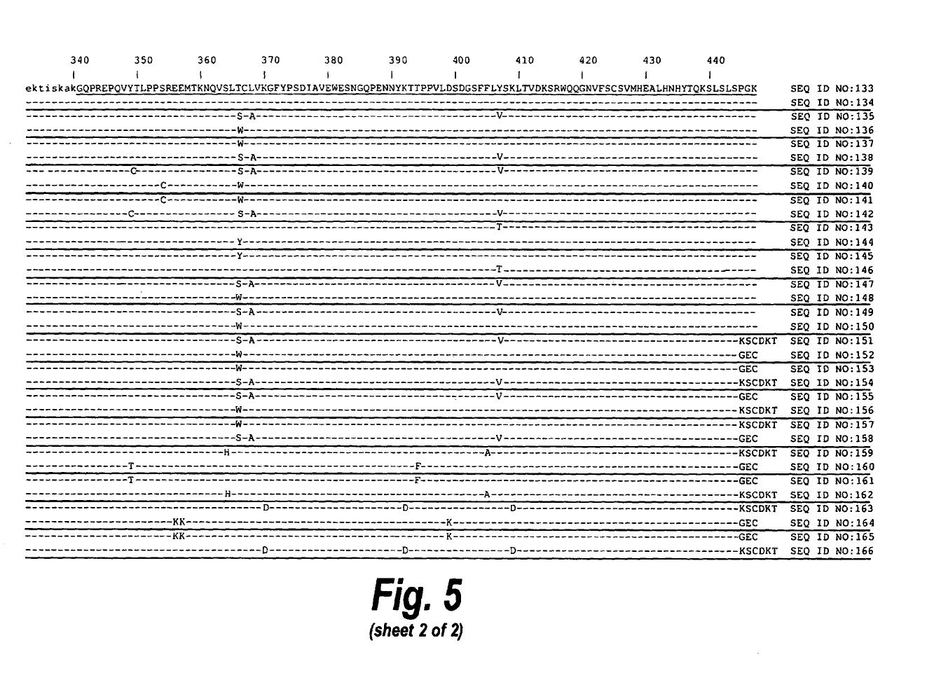

SEQ ID NOs:99-132 are the aa sequences of exemplary IgG1 Fc regions comprising in a contiguous amino to carboxyl terminal order: (a) a hinge selected from the group consisting of an IgG1 hinge, an IgG1 hinge comprising one or more aa substitutions, and a partial IgG1 hinge; (b) an IgG1 CH2 domain with N297Q (SEQ ID NO:25); and (c) an IgG1 CH3 domain selected from the group consisting of SEQ ID NO:29 and SEQ ID NO:29 comprising one or more AEM and/or DiS modifications (FIG. 4). The hinge, CH2 and CH3 domains are covalently linked without intervening sequences. The SEQ ID NOs of each of the domains of SEQ ID NOs:99-132 are set forth in Table 8.

SEQ ID NOs:133-166 are the aa sequences of exemplary IgG1/IgG4 hybrid Fc regions comprising in a contiguous amino to carboxyl terminal order: (a) a hinge selected from the group consisting of an IgG1/IgG4 hybrid hinge, an IgG1/IgG4 hybrid hinge comprising one or more aa substitutions, and a partial IgG4 hinge; (b) an IgG4 CH2 domain with T299K (SEQ ID NO:26); and (c) an IgG1 CH3 domain selected from the group consisting of SEQ ID NO:29 and SEQ ID NO:29 comprising one or more AEM and/or DiS modifications. The hinge, CH2 and CH3 domains are covalently linked without intervening sequences. The SEQ ID NOs of each of the domains of SEQ ID NOs:133-166 are set forth in Table 9.

SEQ ID NO:167 is KSCDKT, which is an exemplary modified carboxyl terminal portion of an IgG1 CH3 domain that introduces a cysteine.

SEQ ID NO:168 is GEC, which is an exemplary modified carboxyl terminal portion of an IgG1 CH3 domain that introduces a cysteine.

SEQ ID NO:169 is the aa sequence of an exemplary non Gly-Ser TFc linker.

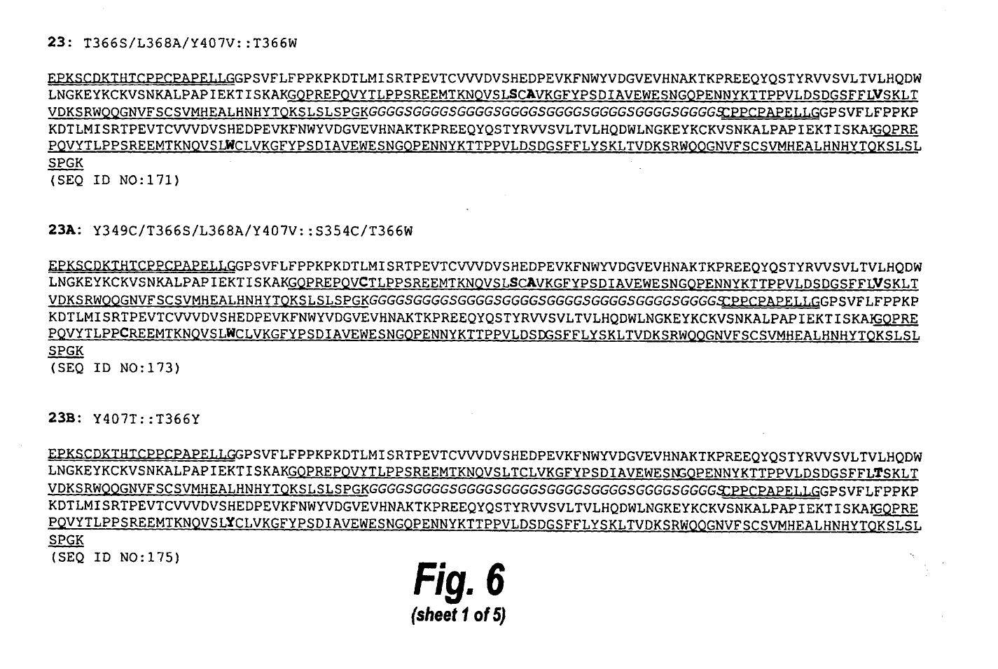

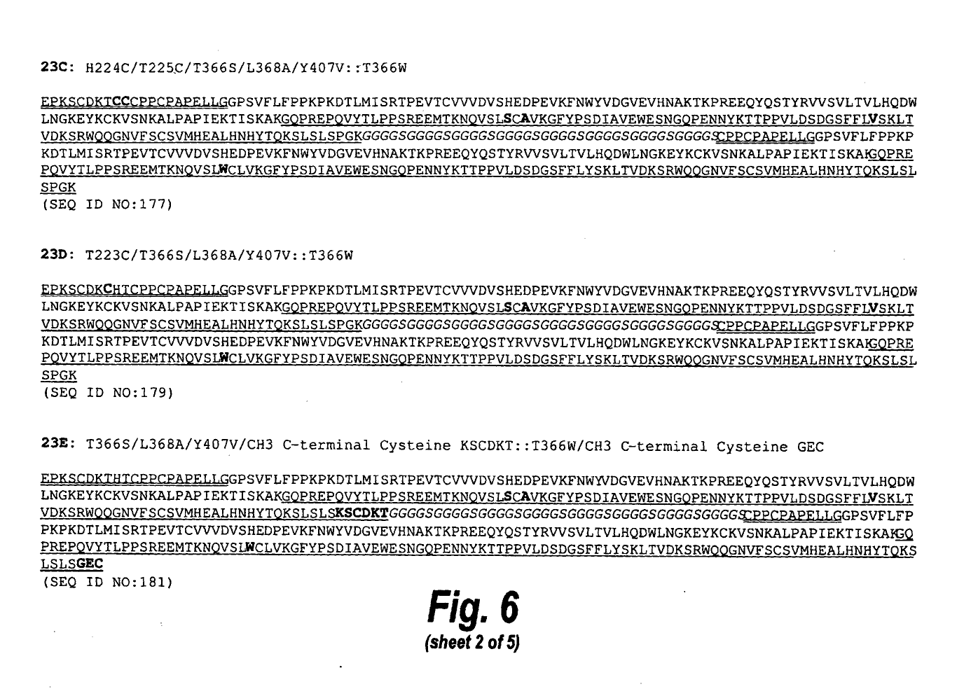

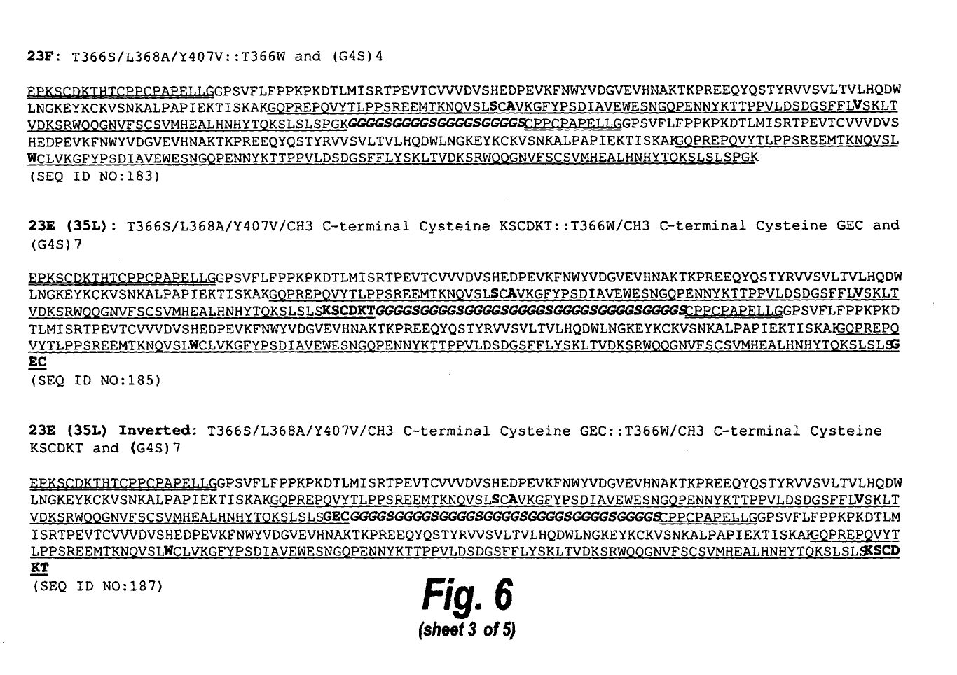

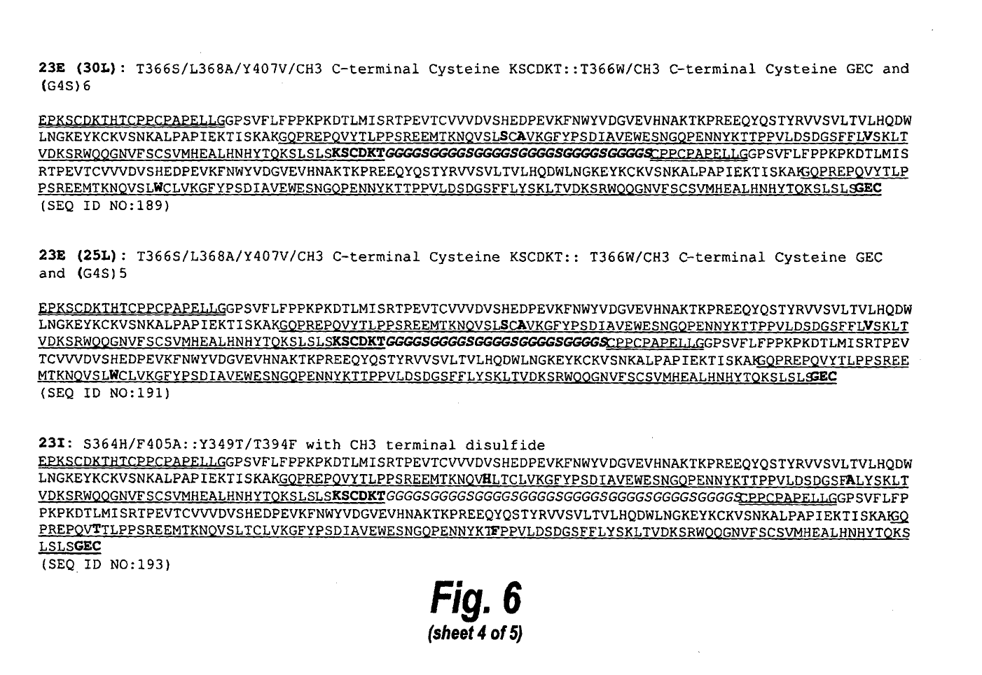

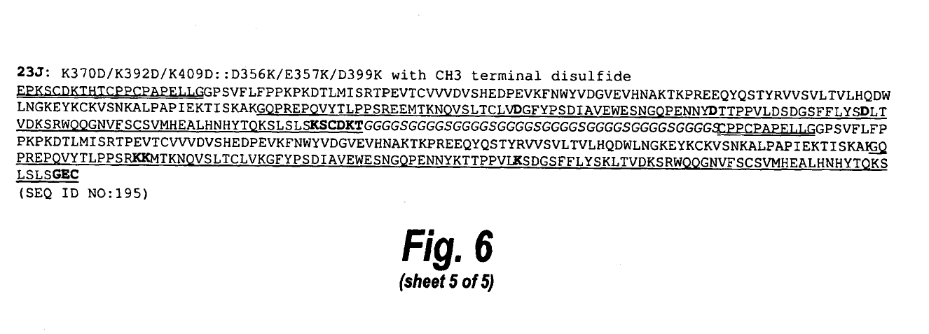

SEQ ID NOs:170-195 are nucleotide sequences (even numbers) and aa sequences (odd numbers) of exemplary IgG1 TFcs, which are set forth in FIG. 6. The SEQ ID NOs of the domains that constitute each of these IgG1 TFcs is set forth in Table 12.

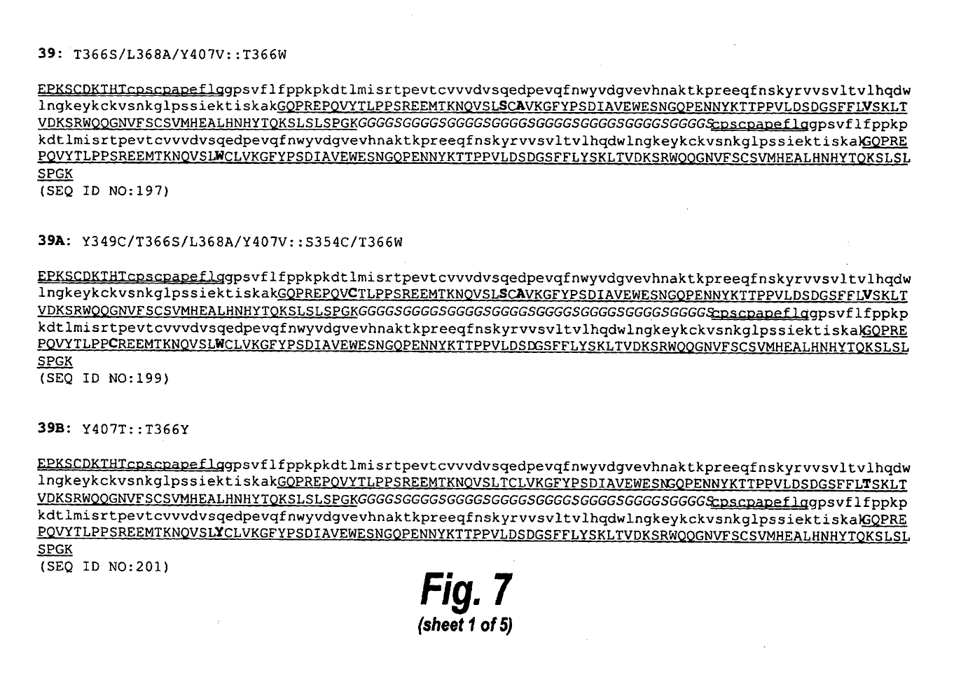

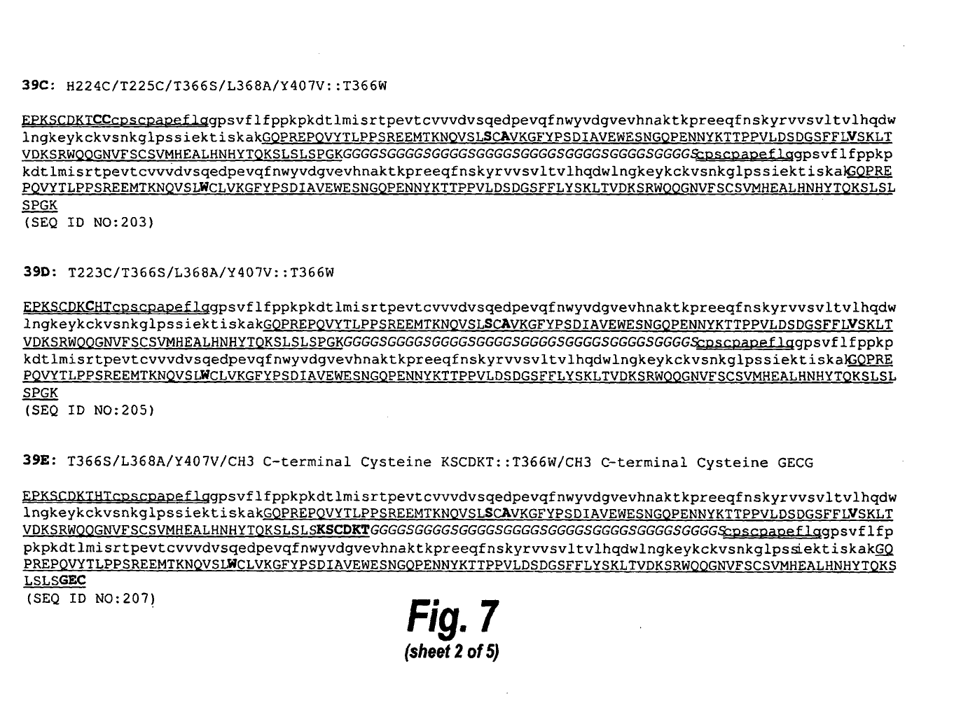

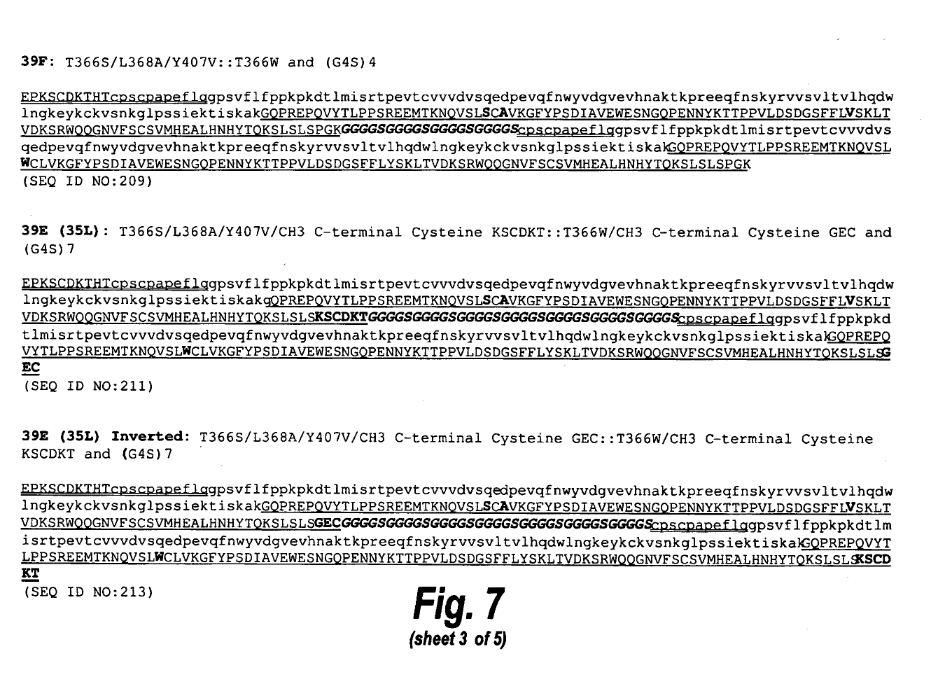

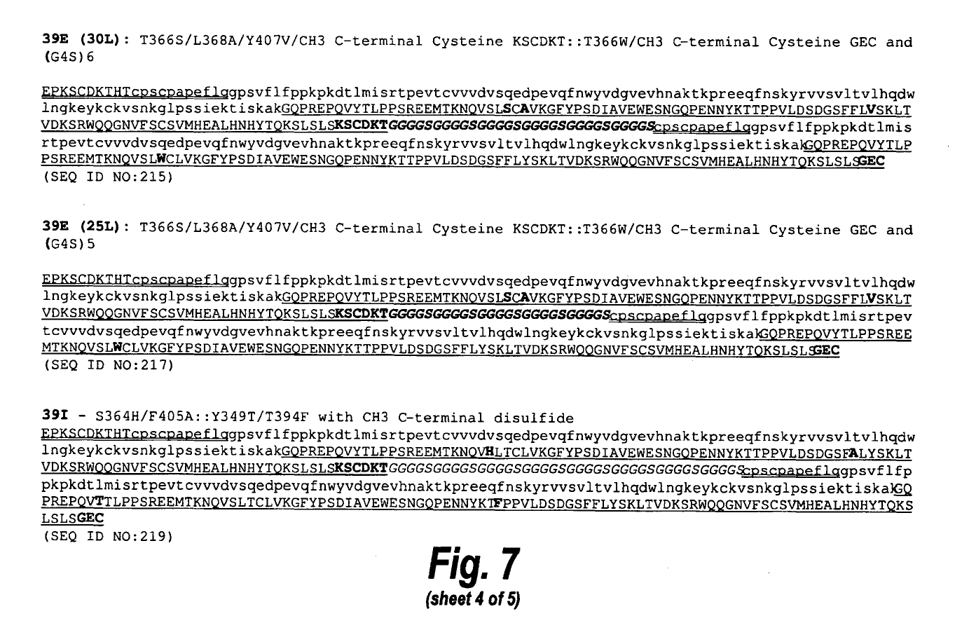

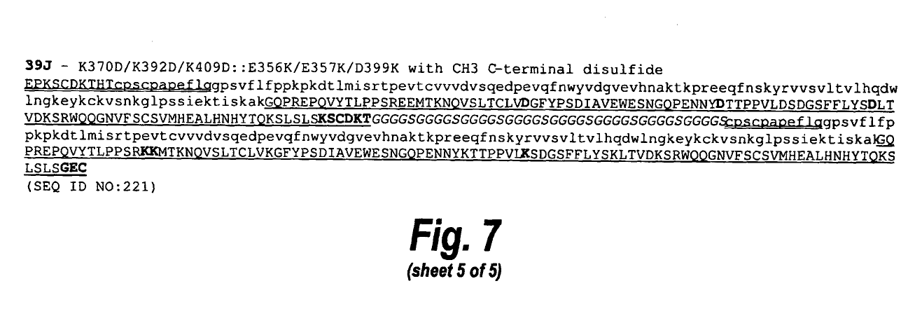

SEQ ID NOs:196-221 are nucleotide sequences (even numbers) and aa sequences (odd numbers) of exemplary TFcs comprising hybrid IgG1/IgG4 Fc regions, which are set forth in FIG. 7. The SEQ ID NOs of the domains that constitute each of these hybrid TFcs is set forth in Table 13.

SEQ ID NOs:222-223 are the nucleotide and aa sequences, respectively, of the heavy chain Fab domain of anti-c-Met Ab 5D5, without signal peptide.

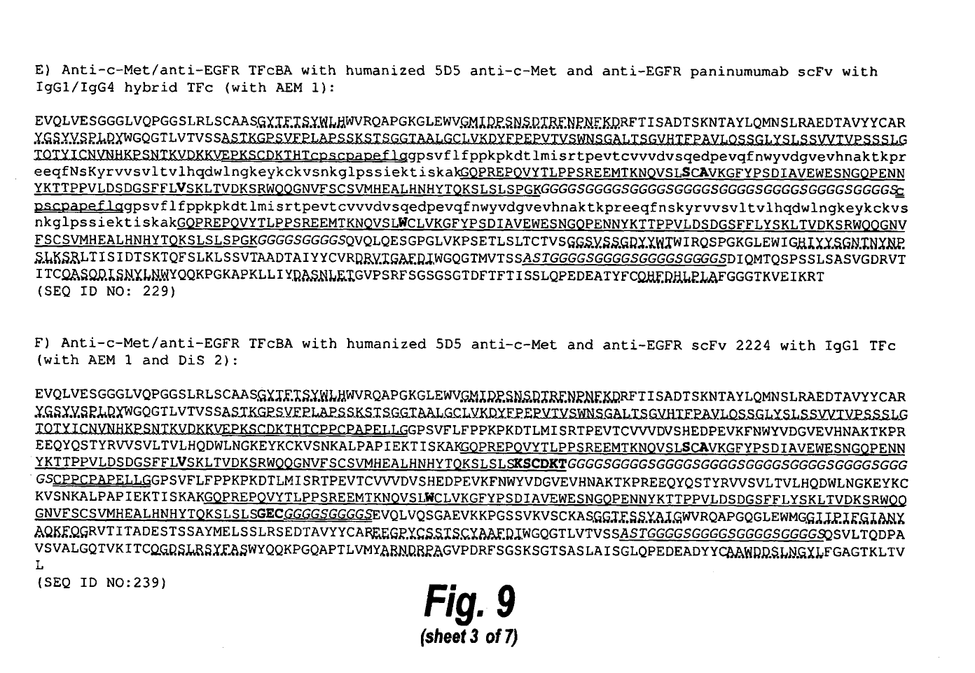

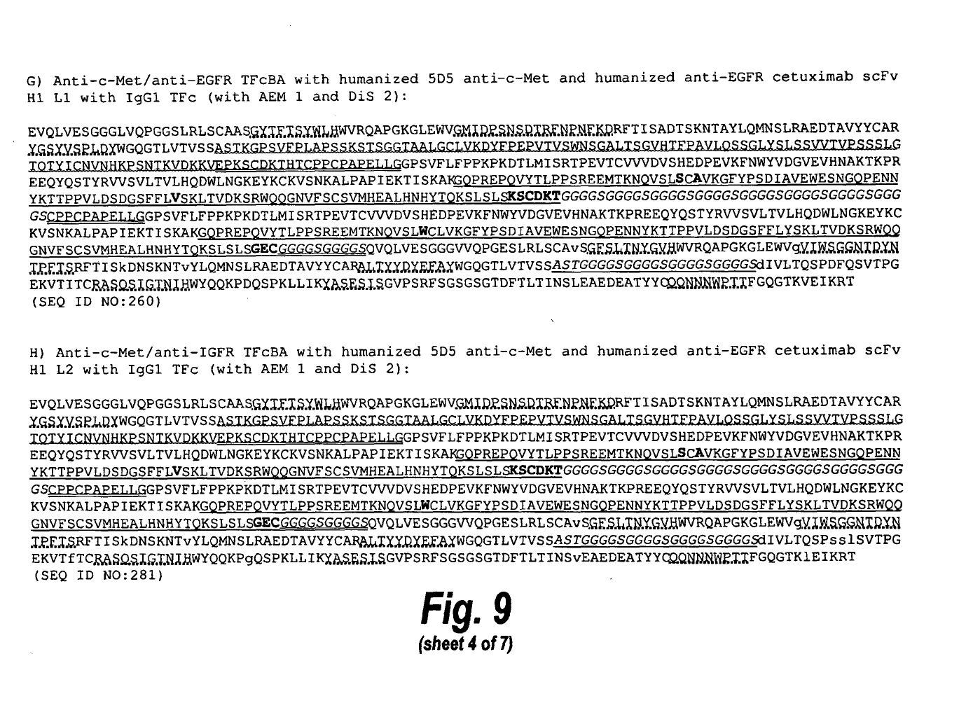

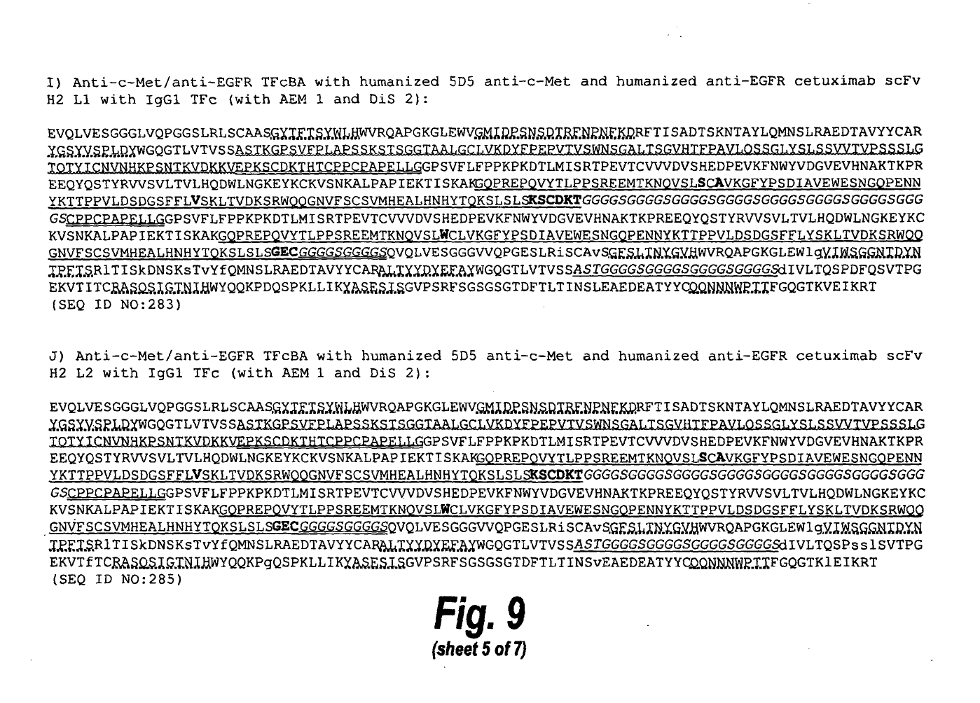

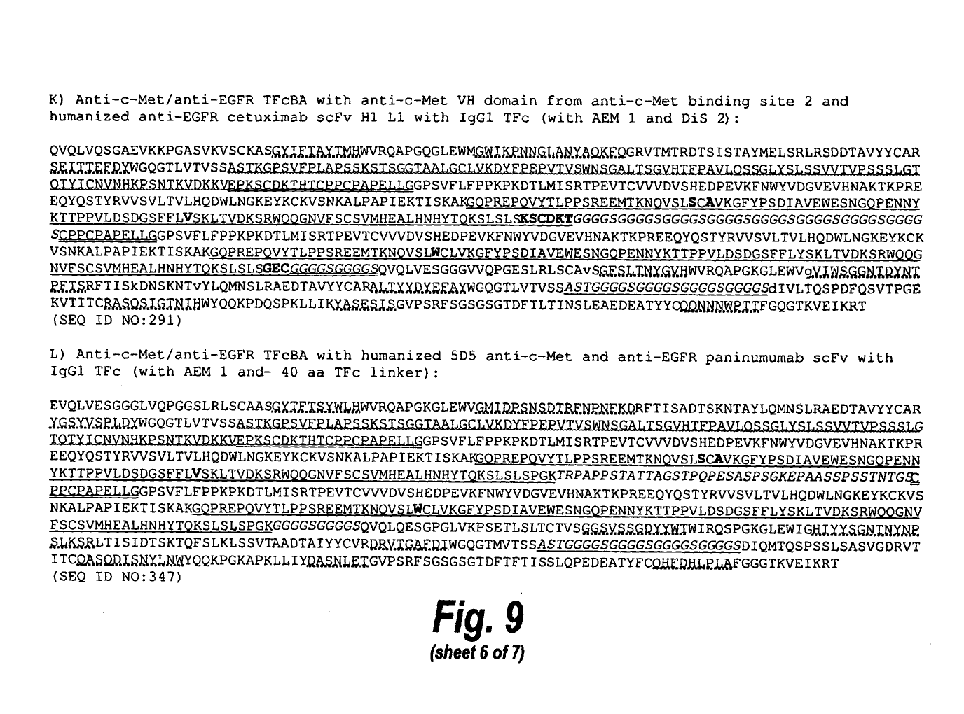

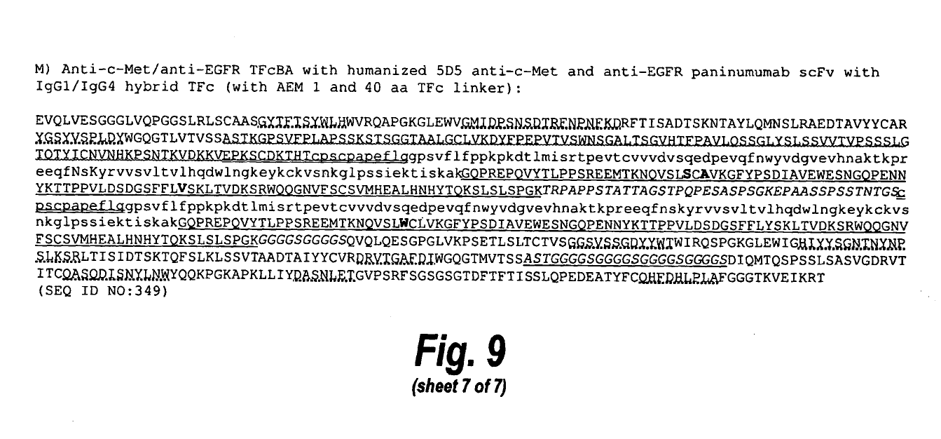

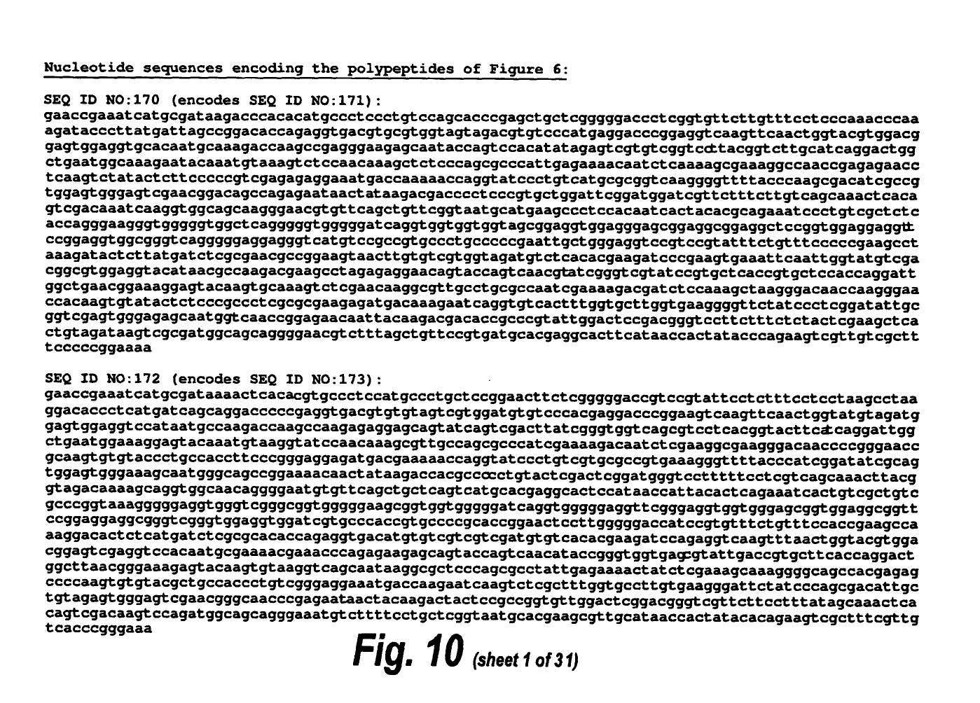

SEQ ID NOs:224-225 are the nucleotide and aa sequences, respectively, of the heavy chain of an IgG1 TFcBA comprising the anti-c-Met 5D5 VH domain, an IgG1 TFc (with AEM 1), and the panitumumab scFv (FIG. 9).

SEQ ID NOs:226-227 are the nucleotide and aa sequences, respectively, of the heavy chain of an IgG1/IgG4 hybrid TFcBA comprising the anti-c-Met 5D5 VH domain, an IgG1/IgG4 hybrid TFc (with AEM 1), and the panitumumab scFv (FIG. 9).

SEQ ID NOs:228-229 are the nucleotide and aa sequences, respectively, of the heavy chain of an IgG1/IgG4 hybrid TFcBA comprising the anti-c-Met 5D5 VH domain, an IgG1/IgG4 hybrid TFc (with AEM 1), and the panitumumab scFv (FIG. 9).

SEQ ID NOs:230 and 231 are the nucleotide and aa sequences, respectively, of a light chain comprising humanized 5D5 anti-c-Met VL domain and CL domain, for use, e.g., with a heavy chain comprising the humanized 5D5 anti-c-Met VH domain, e.g., a heavy chain comprising SEQ ID NO: 225, 227, 229, 244, or 343.

SEQ ID NOs:232 and 233 are the nucleotide and aa sequences of an anti-EGFR scFv comprising the variable regions of panitumumab (VECTIBIX).

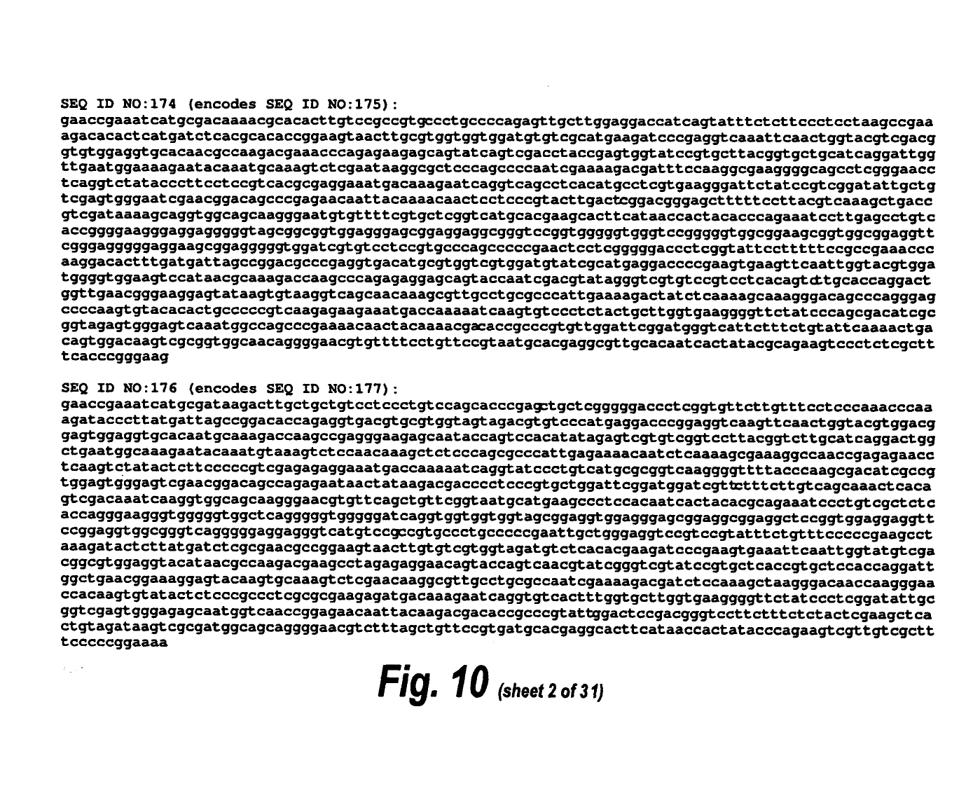

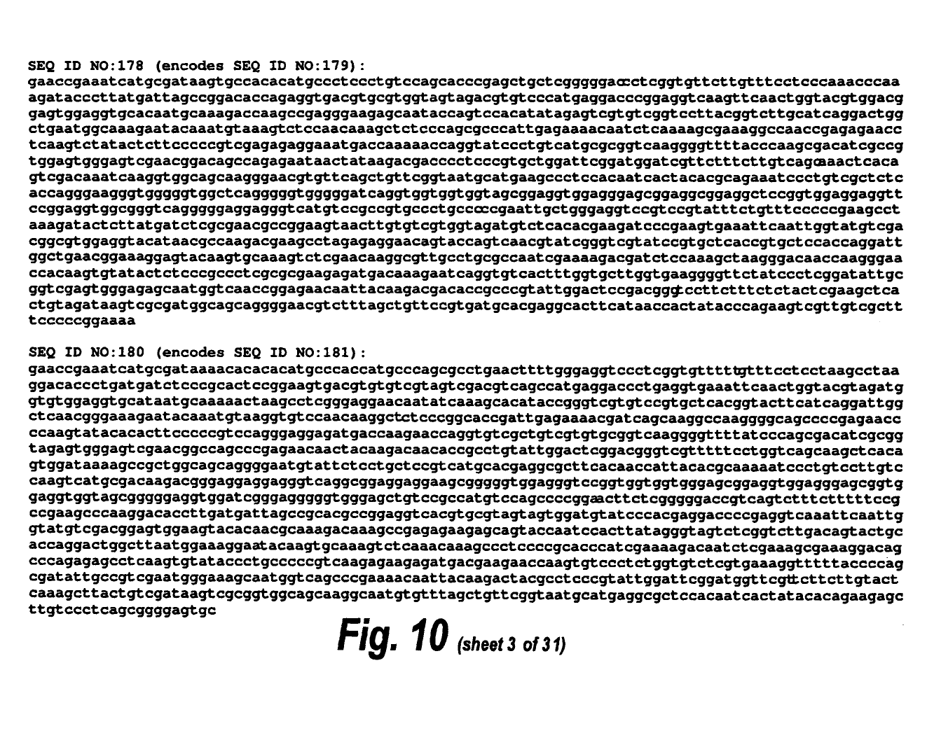

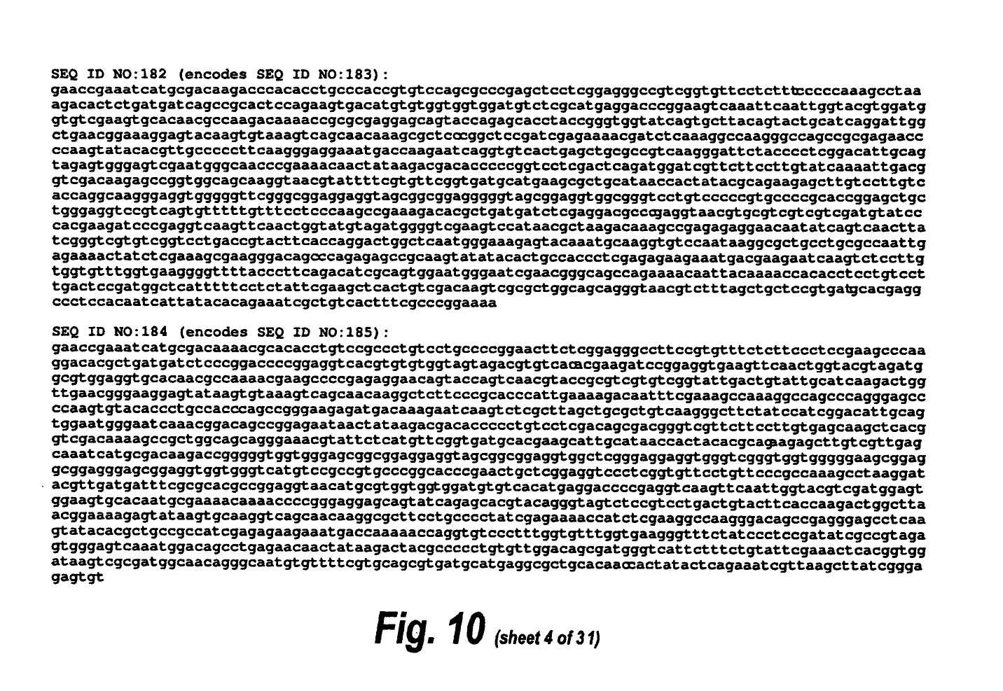

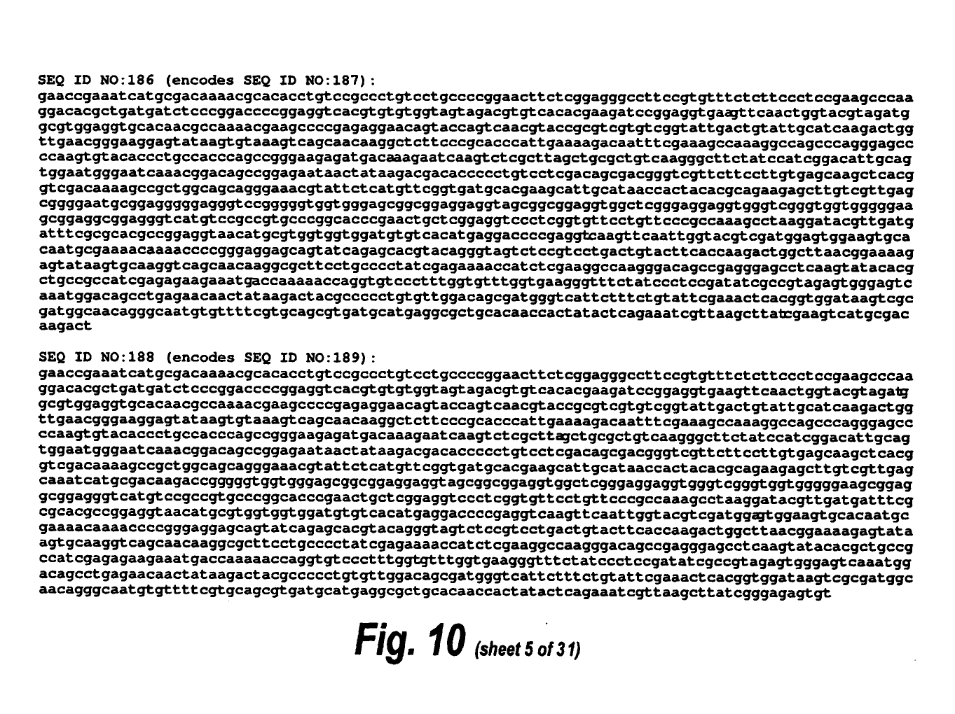

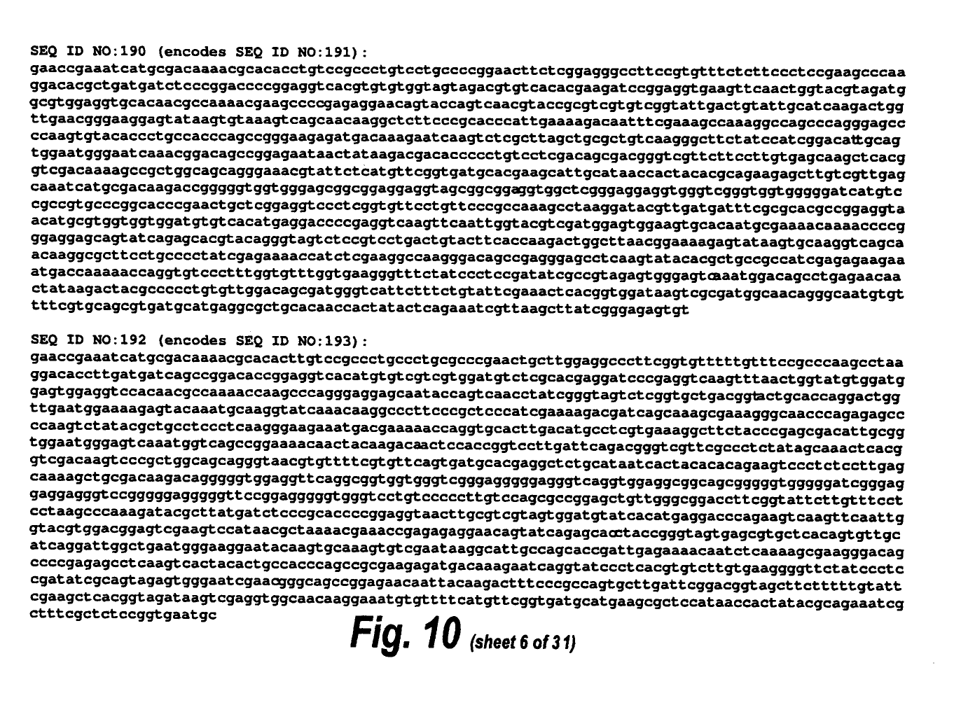

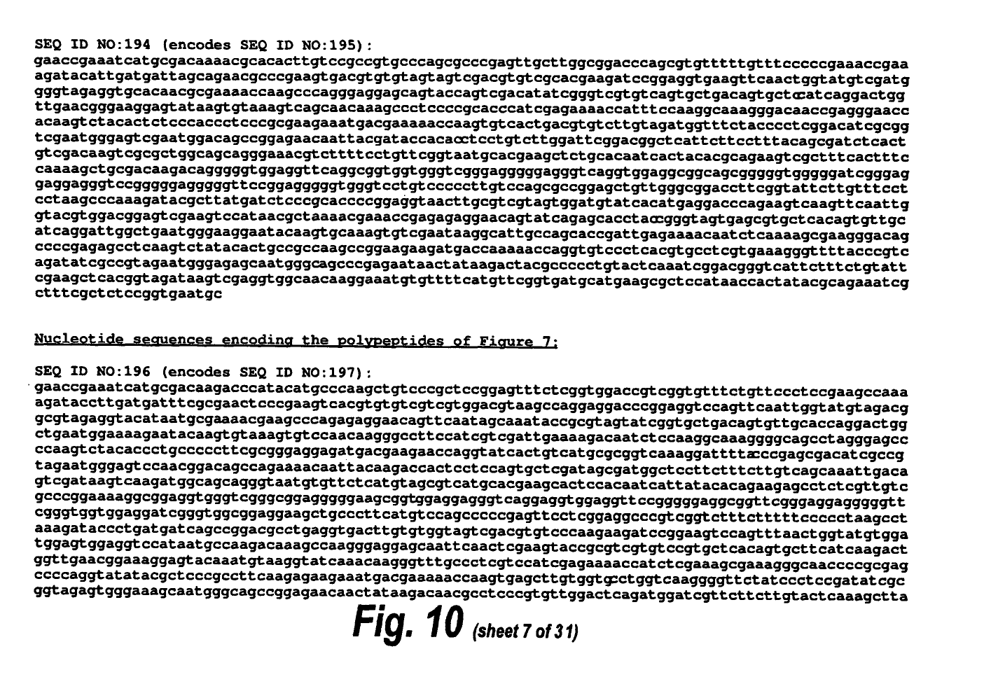

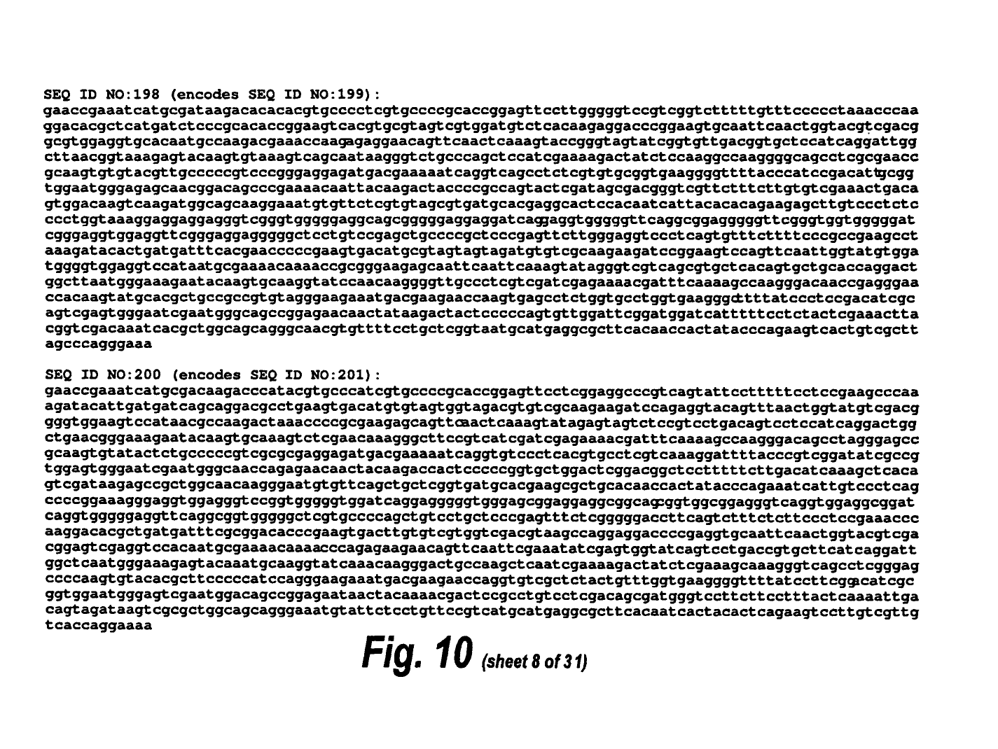

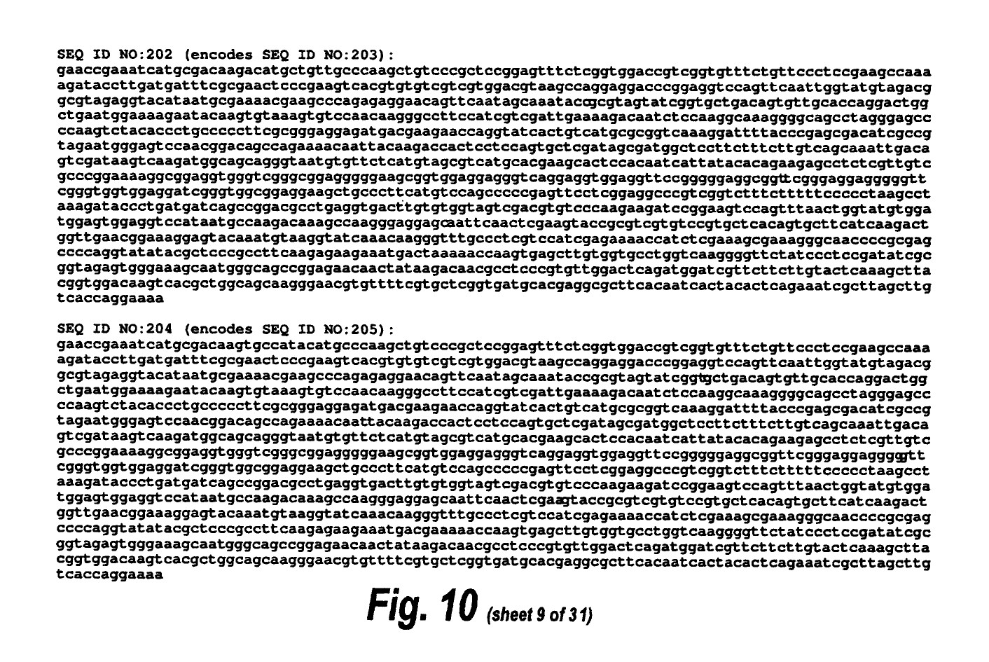

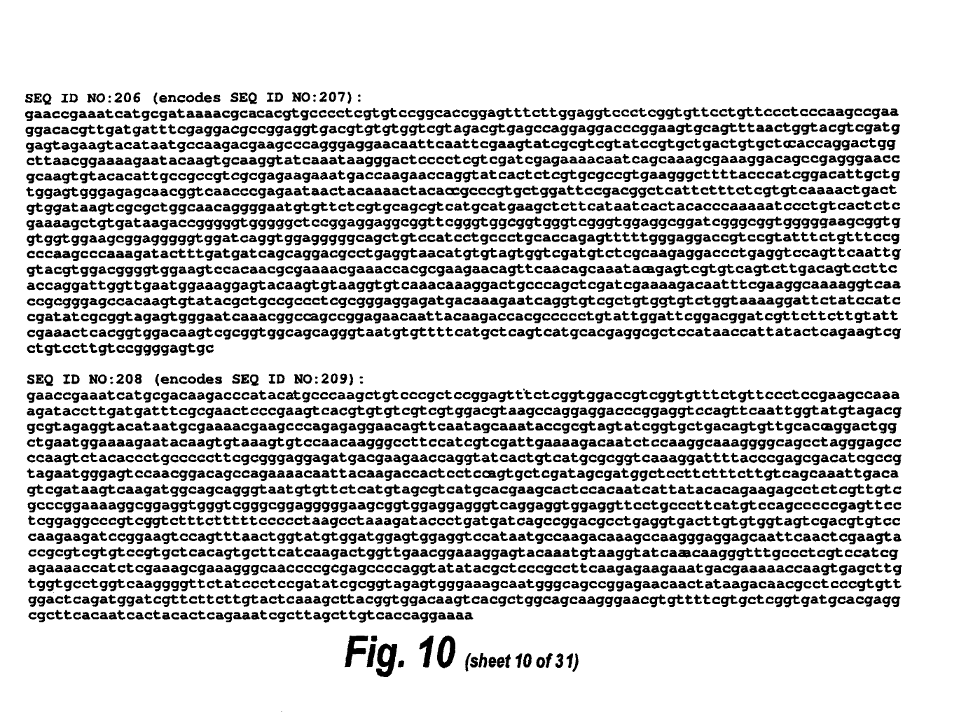

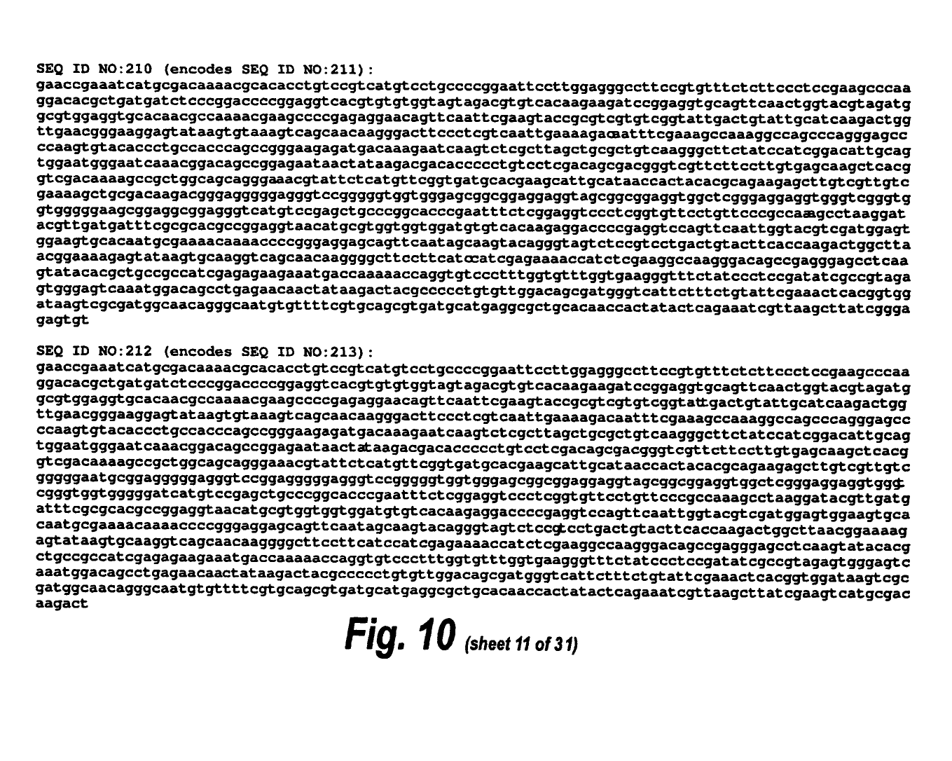

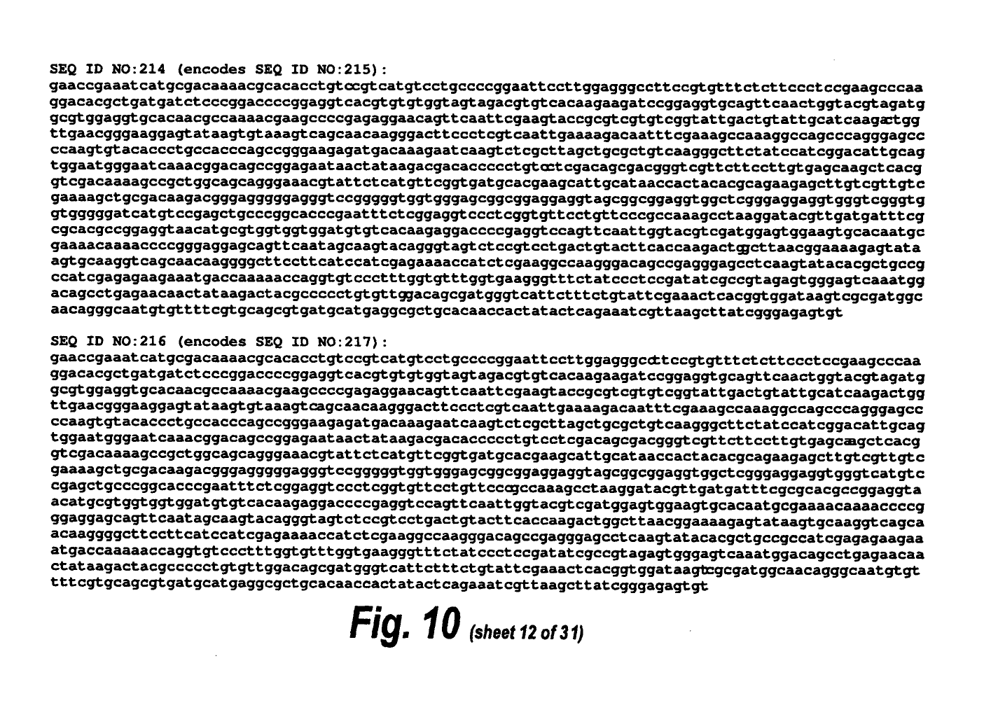

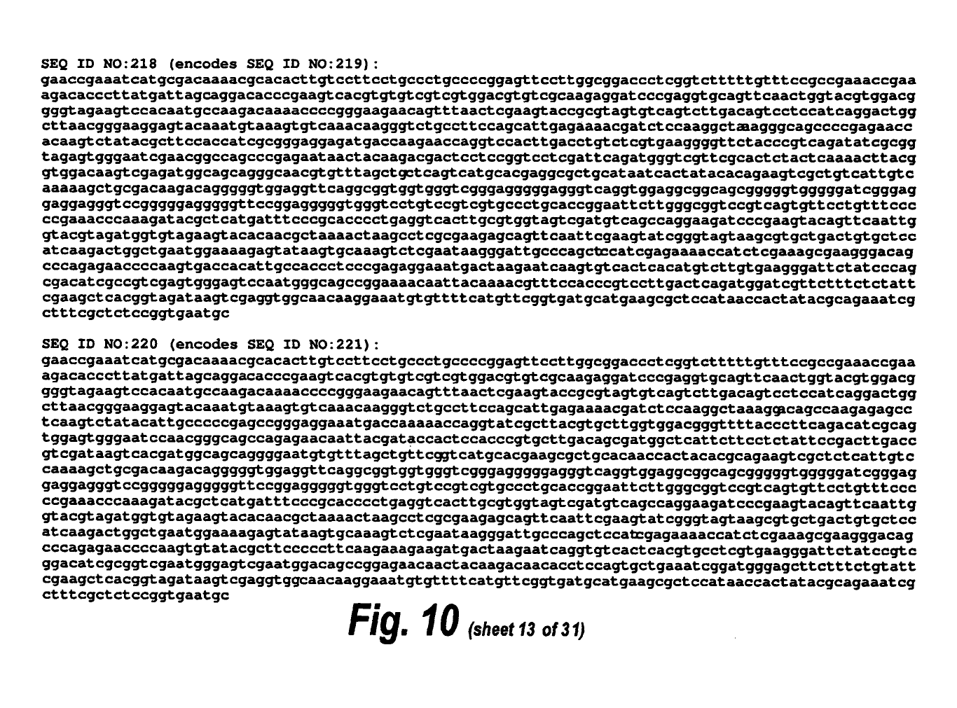

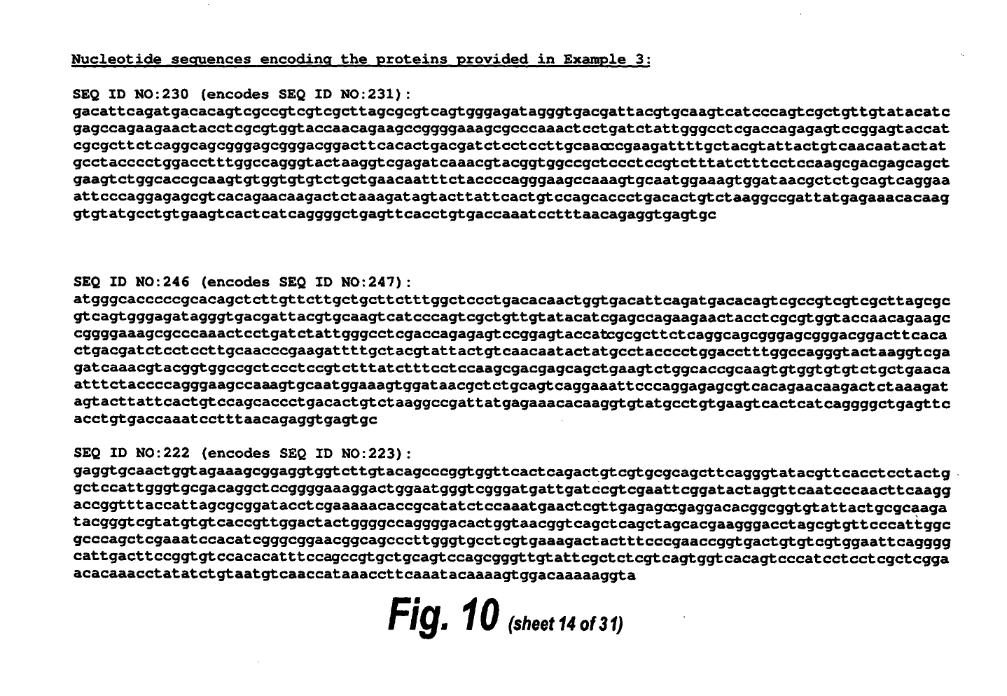

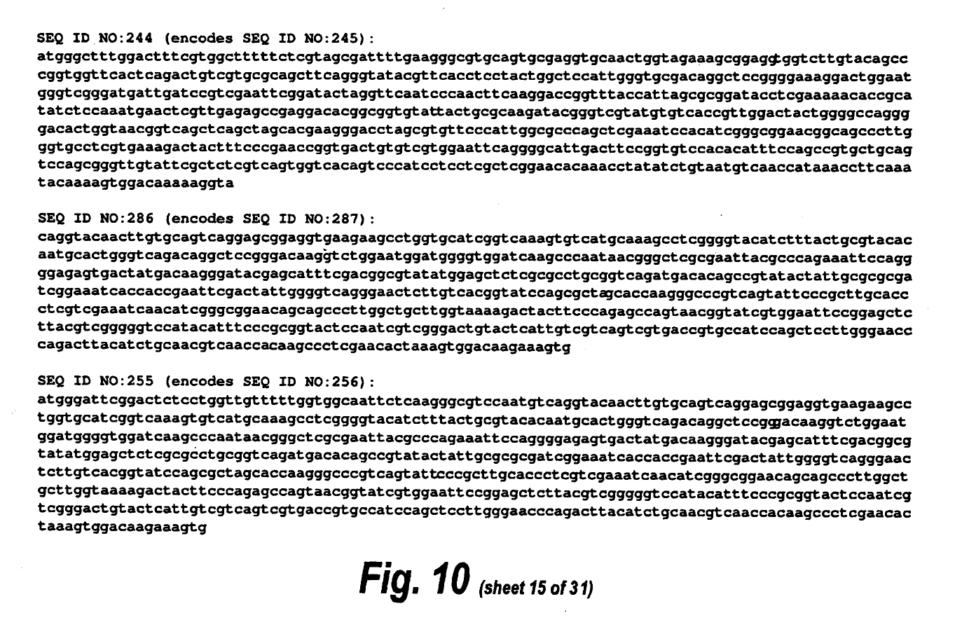

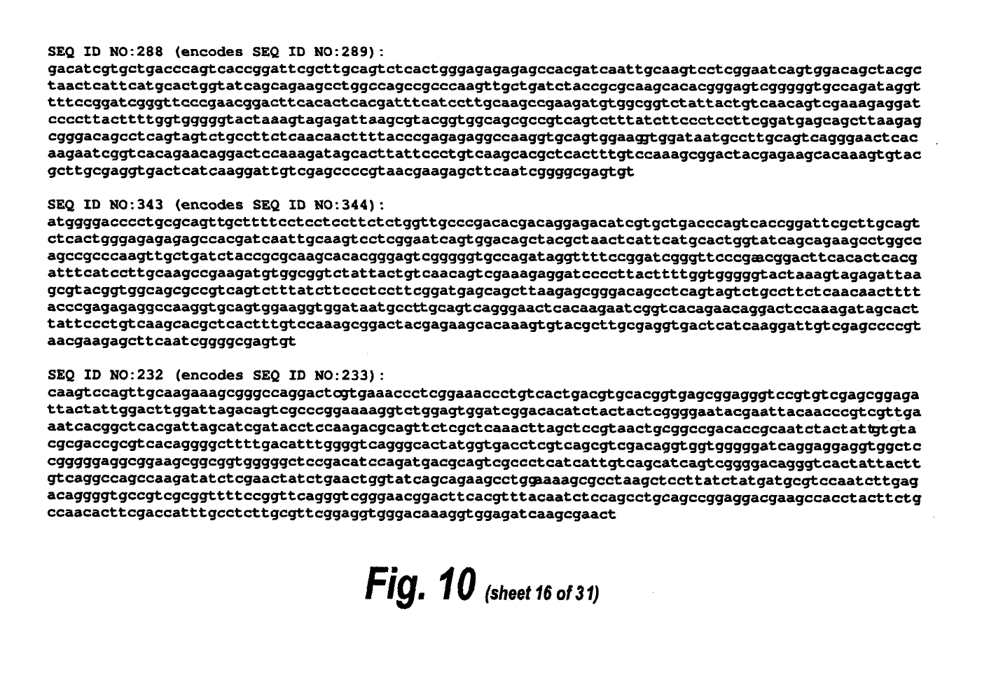

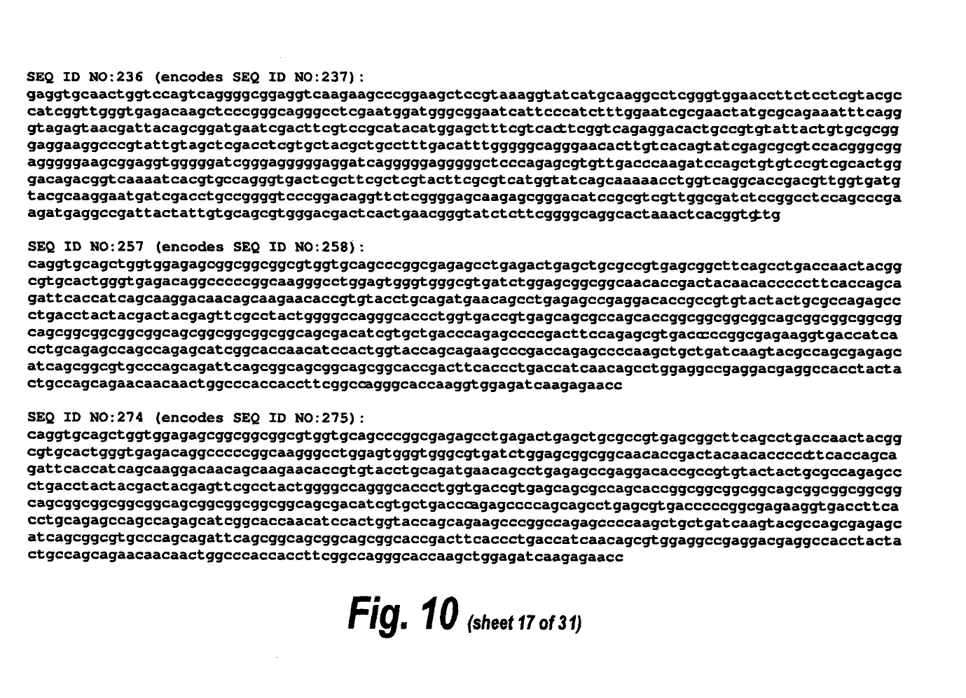

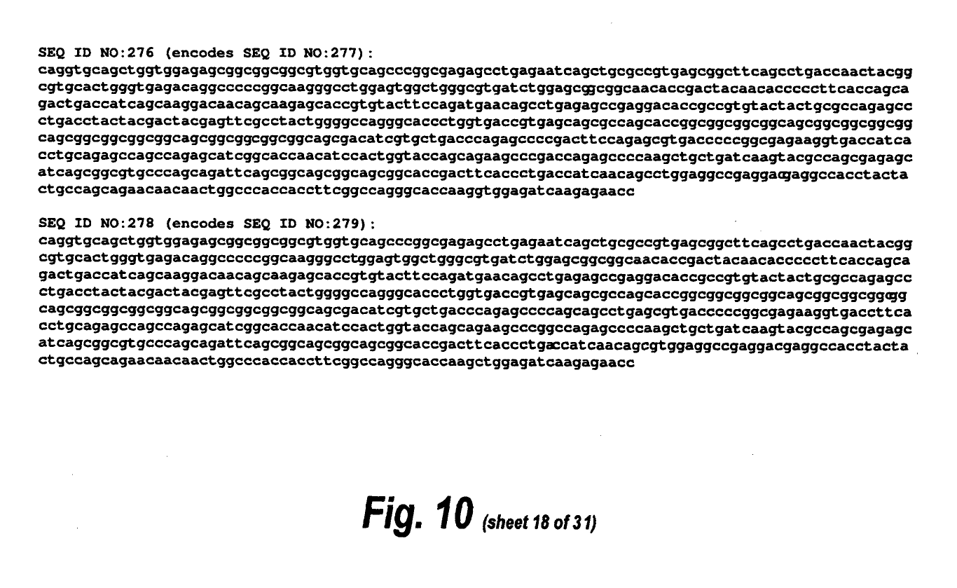

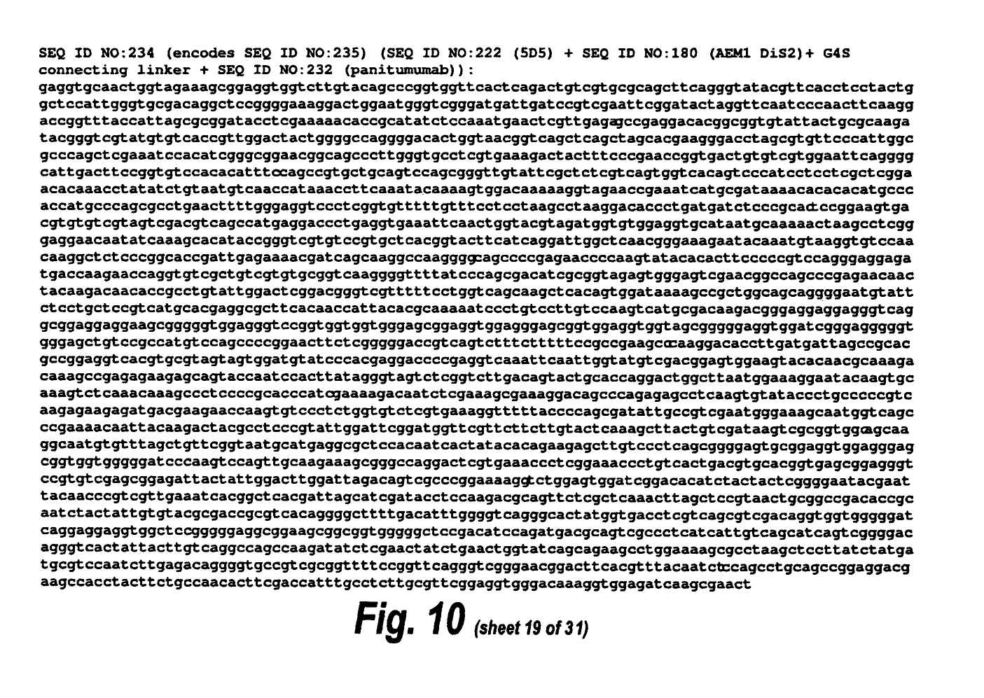

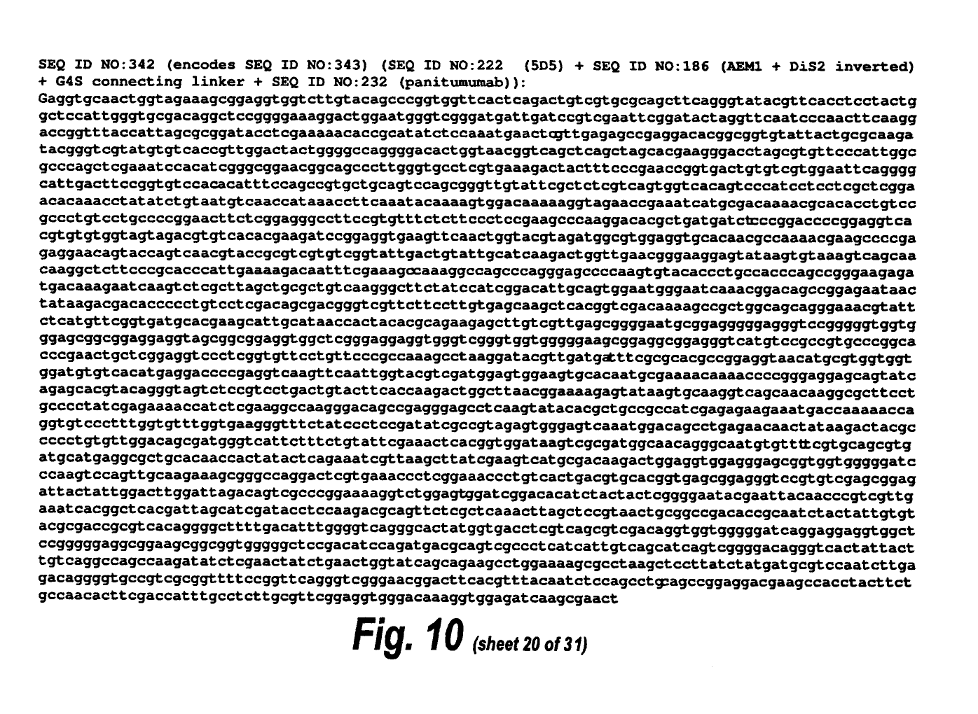

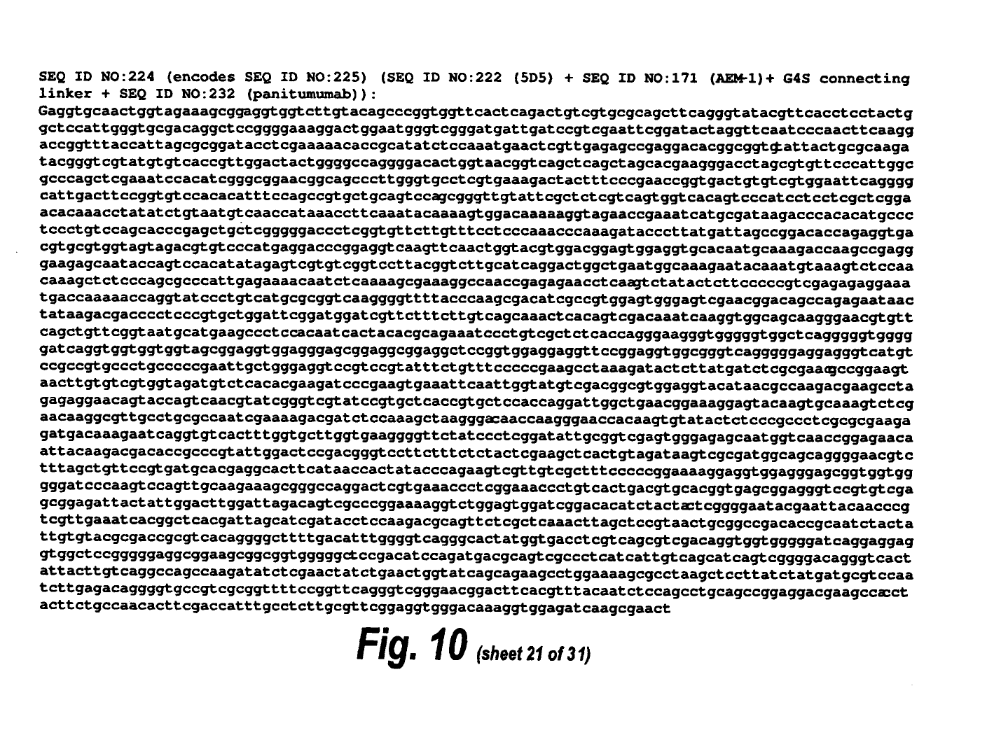

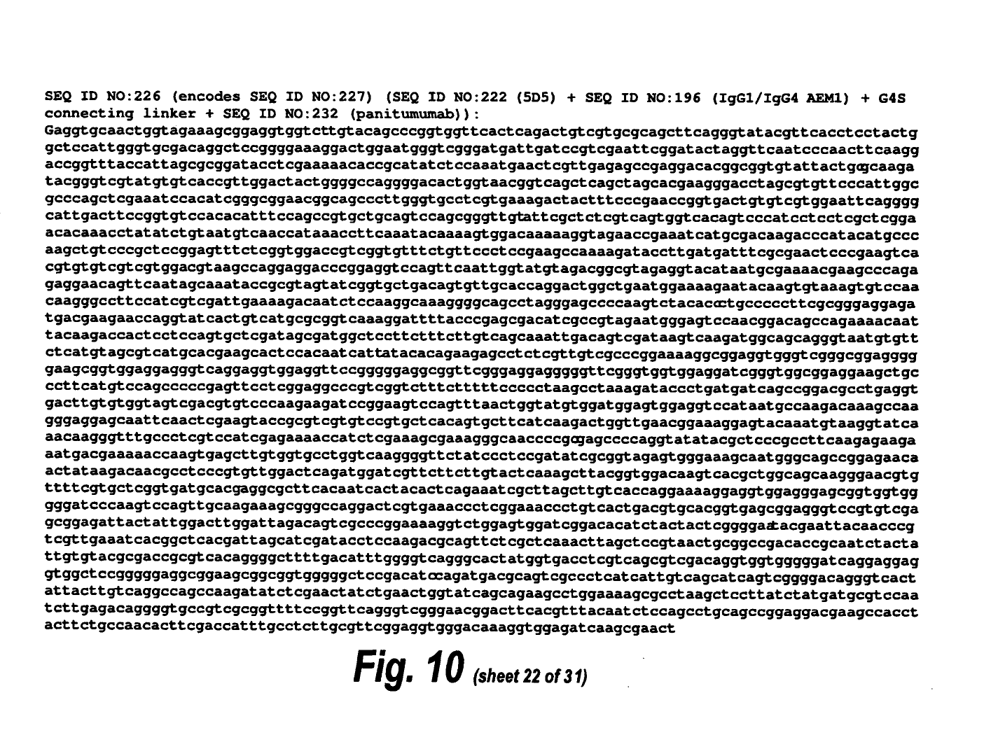

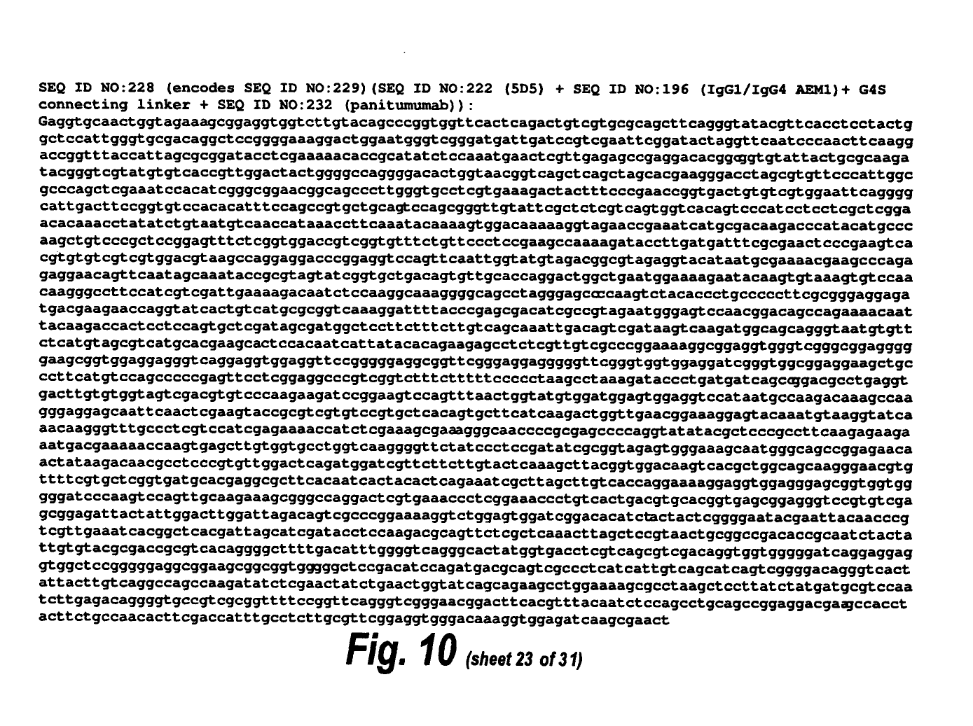

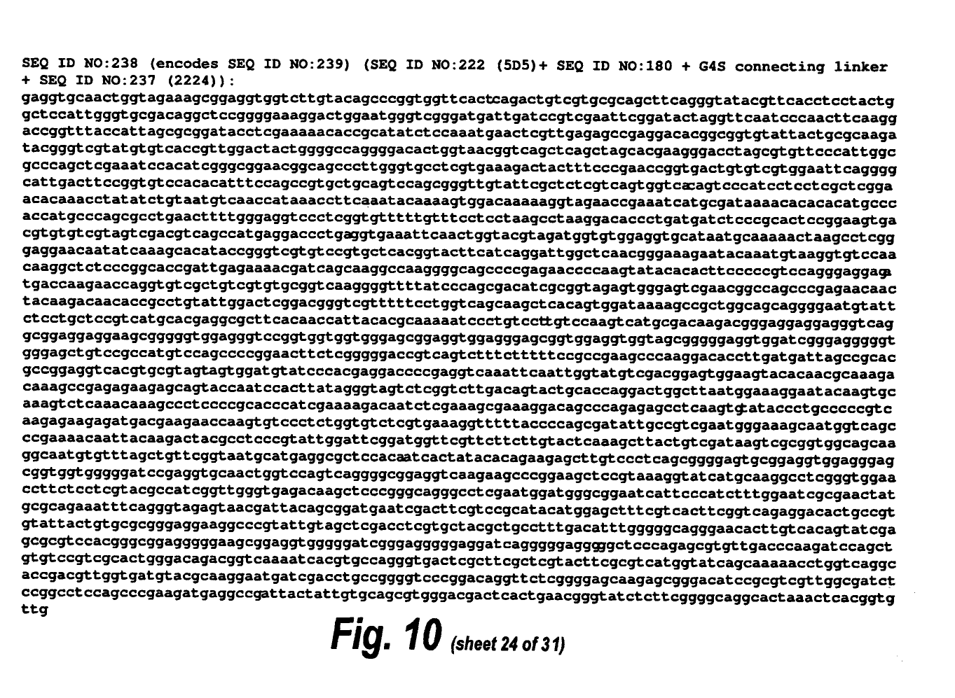

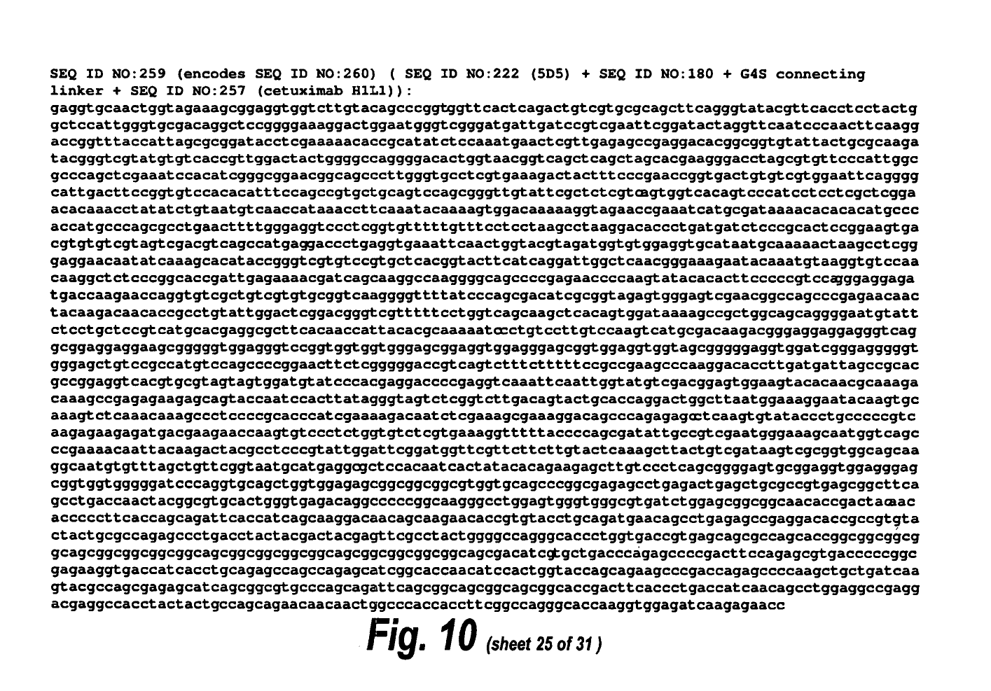

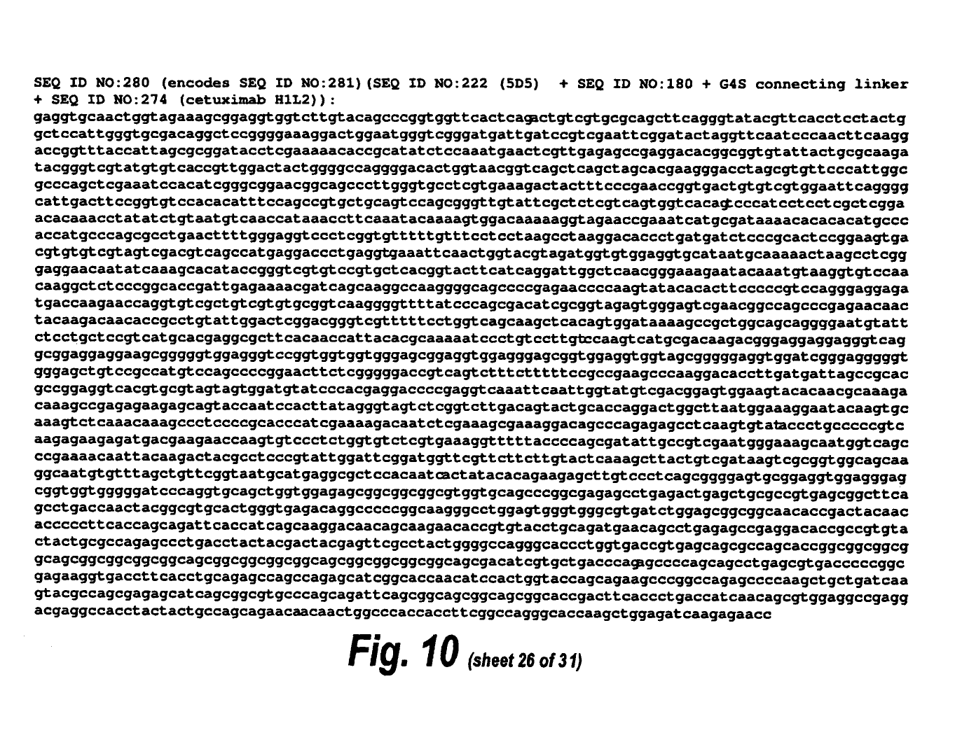

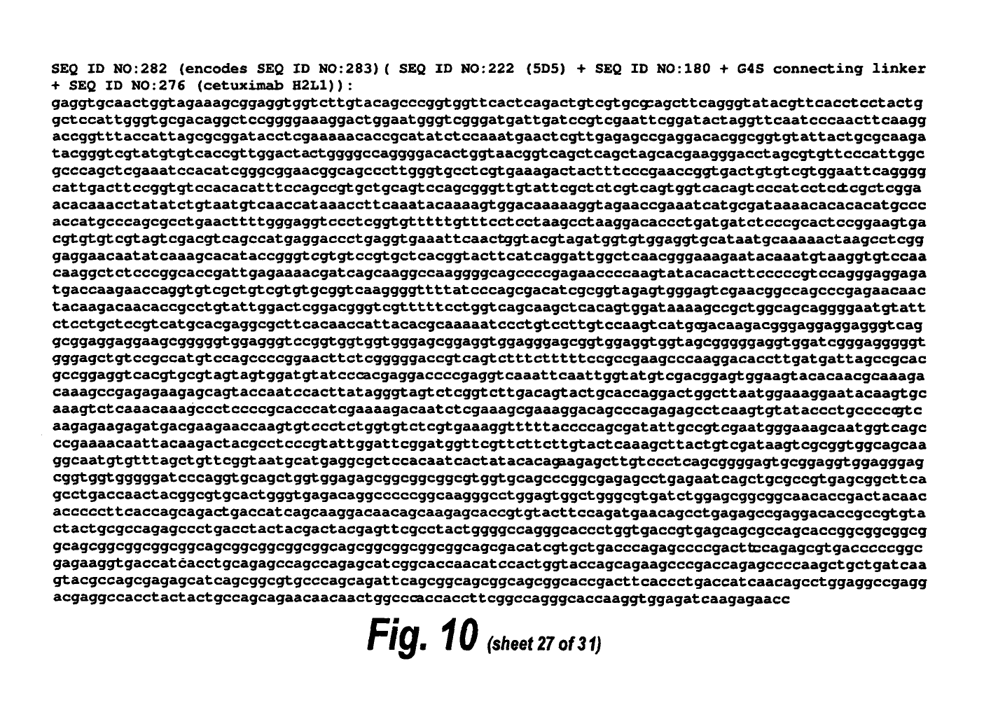

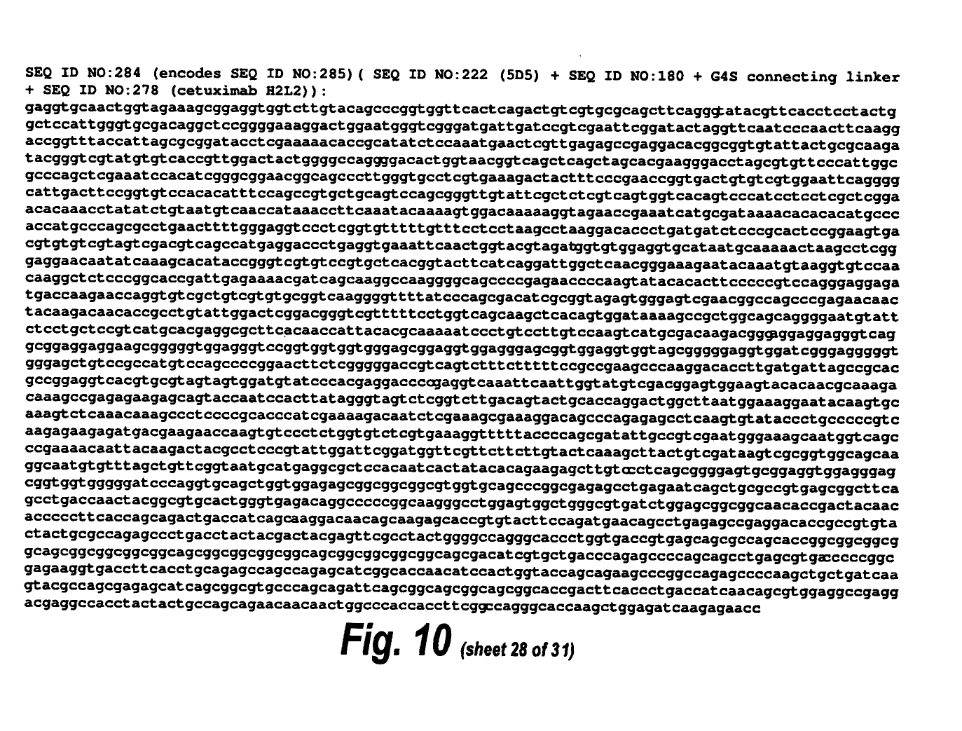

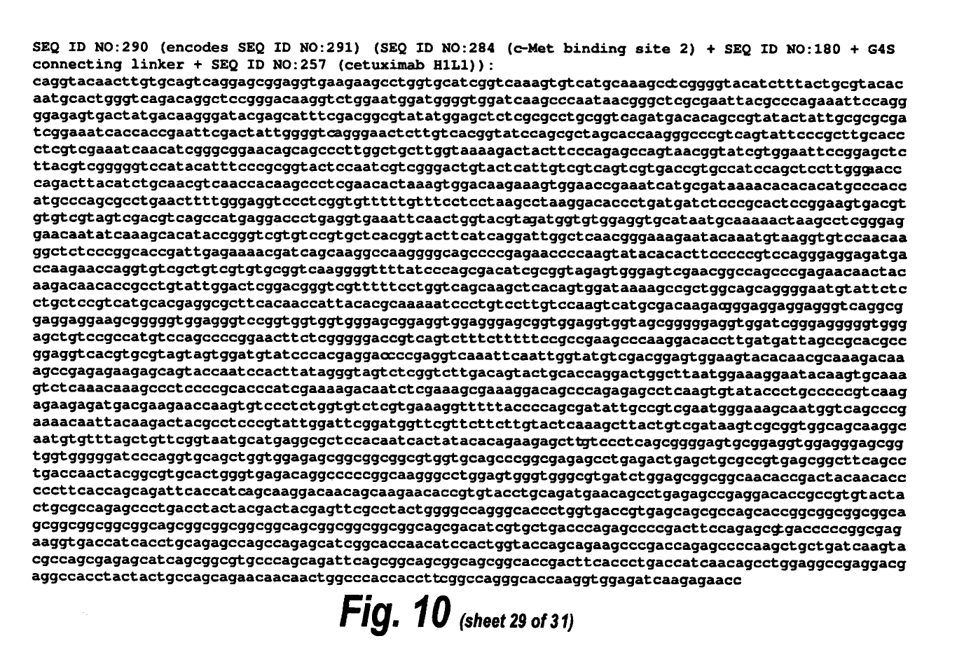

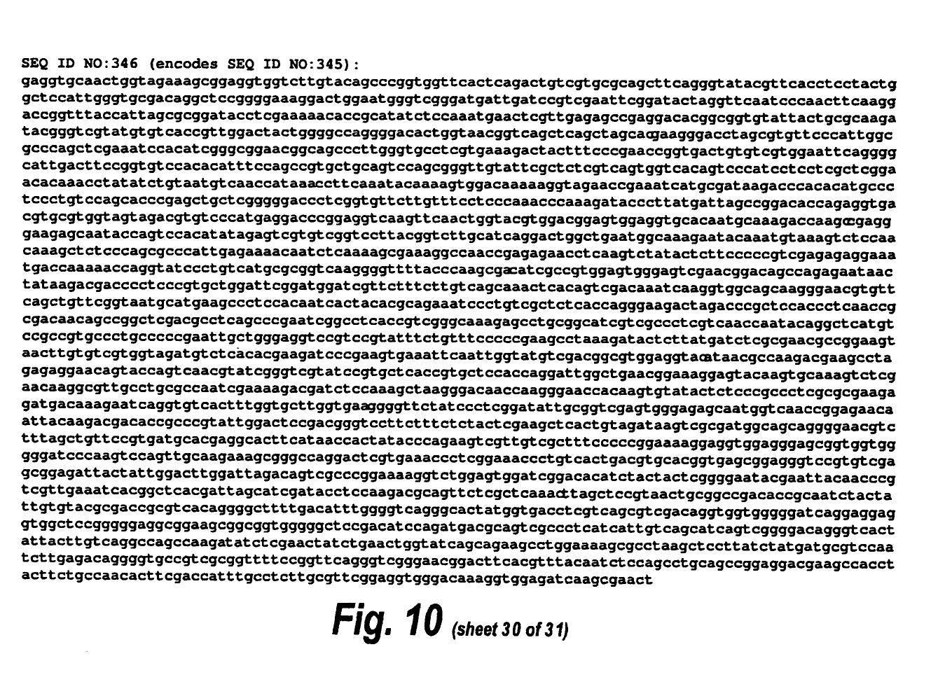

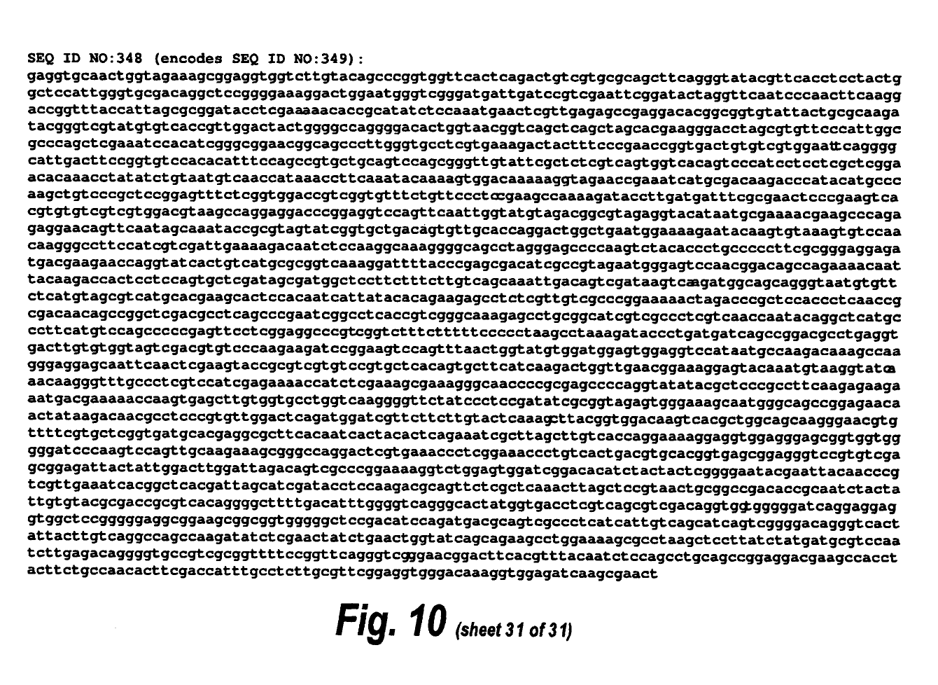

SEQ ID NOs:234 and 235 are the nucleotide and aa sequences, respectively, shown in FIGS. 9 and 10, respectively, of the heavy chain of an anti-c-Met/anti-EGFR TFcBA comprising (a) the anti-c-Met variable domain from humanized 5D5; (b) a TFc with AEM 1 and DiS 2 (SEQ ID NO:181); and (c) an anti-EGFR scFv comprising the variable regions of panitumumab (VECTIBIX) (SEQ ID NO:233).

SEQ ID NOs:236 and 237 are the nucleotide and aa sequences, respectively, of an anti-EGFR scFv comprising the variable regions of Ab 2224.

SEQ ID NOs:238 and 239 are the nucleotide and aa sequences, shown in FIGS. 9 and 10, respectively, of the heavy chain of an anti-c-Met/anti-EGFR TFcBA comprising (a) the anti-c-Met variable domain from humanized 5D5; (b) a TFc with AEM 1 and DiS 2 (SEQ ID NO:181); and (c) an anti-EGFR scFv comprising the variable regions of Ab 2224 (SEQ ID NO:237).

SEQ ID NOs:240 and 241 are the nucleotide and aa sequences, respectively, of an exemplary signal peptide.

SEQ ID NOs:242 and 243 are the nucleotide and aa sequence, respectively, of an exemplary signal peptide.

SEQ ID NOs:244 and 245 are the nucleotide and aa sequences, respectively, of the anti-c-Met VH domain of 5D5 and CL domain with a signal peptide having SEQ ID NO:241.

SEQ ID NO:246 and 247 are the nucleotide and aa sequences of the light chain having SEQ ID NO:231 with a signal peptide having SEQ ID NO:243.

SEQ ID NOs:248-254 are the aa sequences of variant hinges described in the specification.

SEQ ID NO: 255 and 256 are the nucleotide and aa sequences, respectively, of the heavy chain Fab region of the anti-c-Met binding site 2 (SEQ ID NO:287) with the signal peptide consisting of SEQ ID NO:241 and shown in Example 3.

SEQ ID NOs:257 and 258 are the nucleotide and aa sequences, respectively, of an anti-EGFR scFv comprising the variable regions of humanized cetuximab (ERBITUX) H1L1.

SEQ ID NOs:259 and 260 are the nucleotide and aa sequences, shown in FIGS. 9 and 10, respectively, of the heavy chain of an anti-c-Met/anti-EGFR TFcBA comprising (a) the anti-c-Met variable domain from humanized 5D5; (b) a TFc with AEM 1 and DiS 2 (SEQ ID NO:181); and (c) an anti-EGFR scFv comprising the variable regions of humanized cetuximab (ERBITUX) H1L1 (SEQ ID NO:258).

SEQ ID NO:261 is the aa sequence of a full length wild type IgG1 CH2 domain.

SEQ ID NO:262 is the aa sequence of a full length wild type IgG4 CH2 domain. SEQ ID NOs:263, 264 and 265 are aa sequences of variant hIgG1 hinges (FIG. 2).

SEQ ID NO:266 is the aa sequence of the wild type mouse IgG1 hinge (FIG. 2).

SEQ ID NO:267 is the aa sequence of a mouse IgG1/IgG2A hybrid hinge (FIG. 2).

SEQ ID NOs:268 and 269 are the aa sequences of variant hIgG2 hinges (FIG. 2).

SEQ ID NO:270 is the aa sequence of a wild type hIgA2 hinge (FIG. 2).

SEQ ID NOs:271-273 are aa sequences of variant hIgA2 hinges (FIG. 2).

SEQ ID NOs:274-279 are nucleotide (even numbers) and aa (odd numbers) sequences of scFvs comprising variable domains of humanized cetuximab Abs H1L2, H2L1 and H2L2, which are described in Example 3.

SEQ ID NOs:280-285 are nucleotide (even numbers) and aa (odd numbers) sequences of the heavy chain of an anti-c-Met/anti-EGFR TFcBA comprising (a) the anti-c-Met variable domain from humanized 5D5; (b) an anti-EGFR scFv comprising the variable regions of humanized cetuximab (ERBITUX) Abs H1L2, H2L1 and H2L2 (SEQ ID NO:275, 277 or 279, respectively); and (c) a TFc with AEM 1 and DiS 2 (SEQ ID NO:181) (FIG. 9).

SEQ ID NOs:286 and 287 are the nucleotide and aa sequences, respectively, of the heavy chain Fab domain of anti-c-Met binding site 2, which is described in Example 3.

SEQ ID NOs:288 and 289 are the nucleotide and aa sequences, respectively, of the light chain Fab domain of anti-c-Met binding site 2, which is described in Example 3.

SEQ ID NOs:290 and 291 are the nucleotide and aa sequences, shown in FIGS. 9 and 10, respectively, of the heavy chain of an anti-c-Met/anti-EGFR TFcBA comprising (a) the anti-c-Met heavy chain Fab domain from anti-c-Met binding site 2 (SEQ ID NO:287); (b) a TFc with AEM 1 and DiS 2 (SEQ ID NO:181); and (c) an anti-EGFR scFv comprising the variable regions of humanized cetuximab (ERBITUX) H1L1 (SEQ ID NO:258) (FIG. 9). The aa sequence of SEQ ID NO:291 is the same as that having SEQ ID NO:260, wherein the anti-c-Met binding domain has been replaced with that of the anti-c-Met binding site 2.

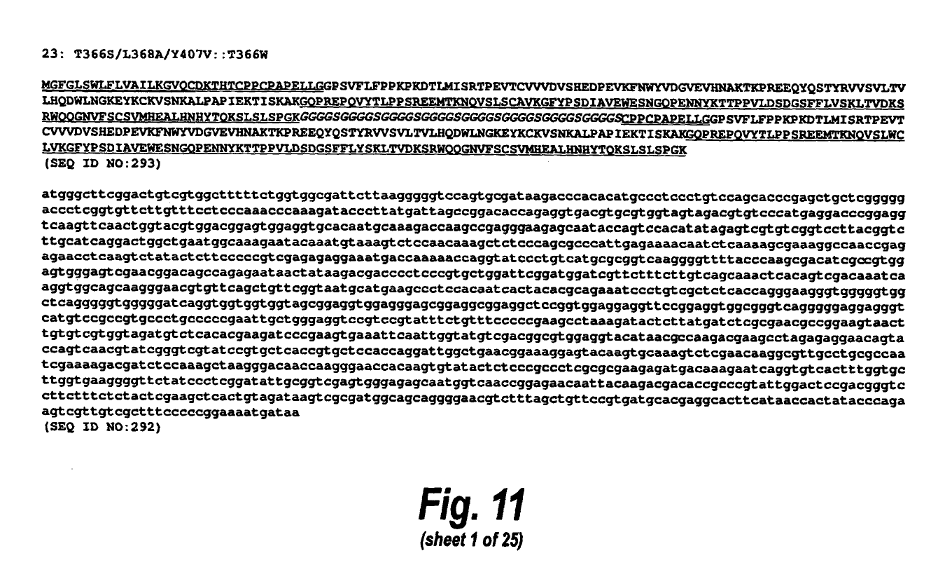

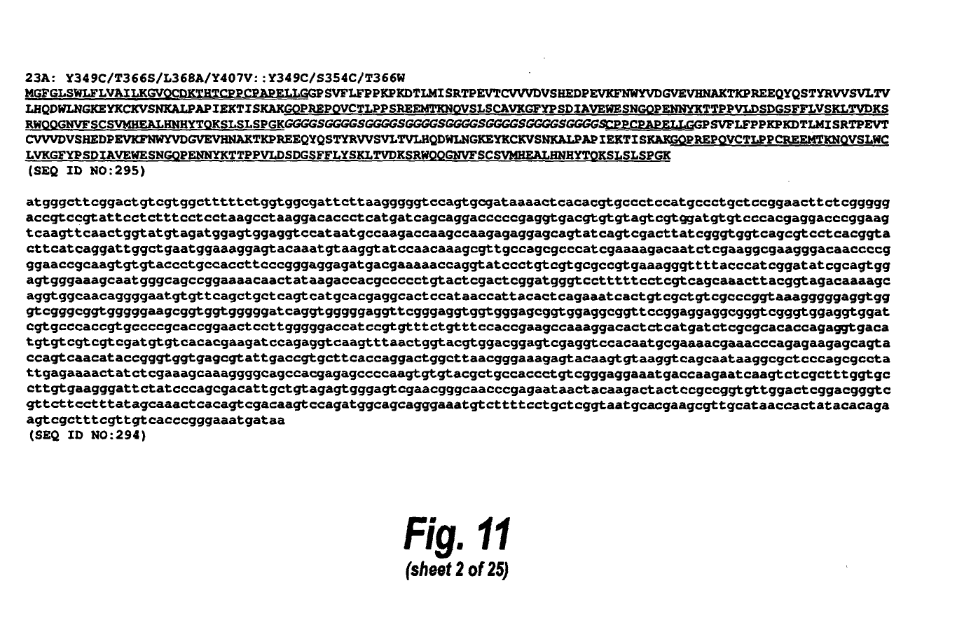

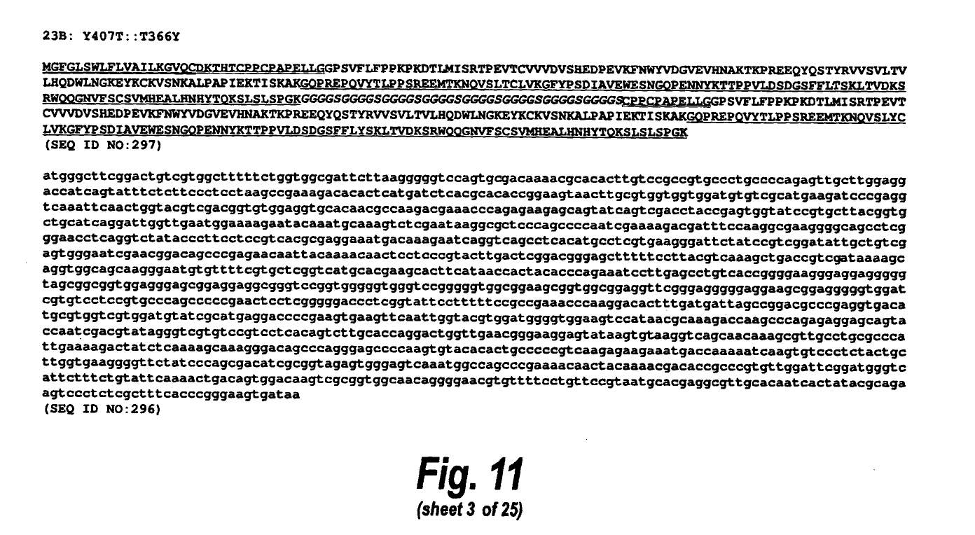

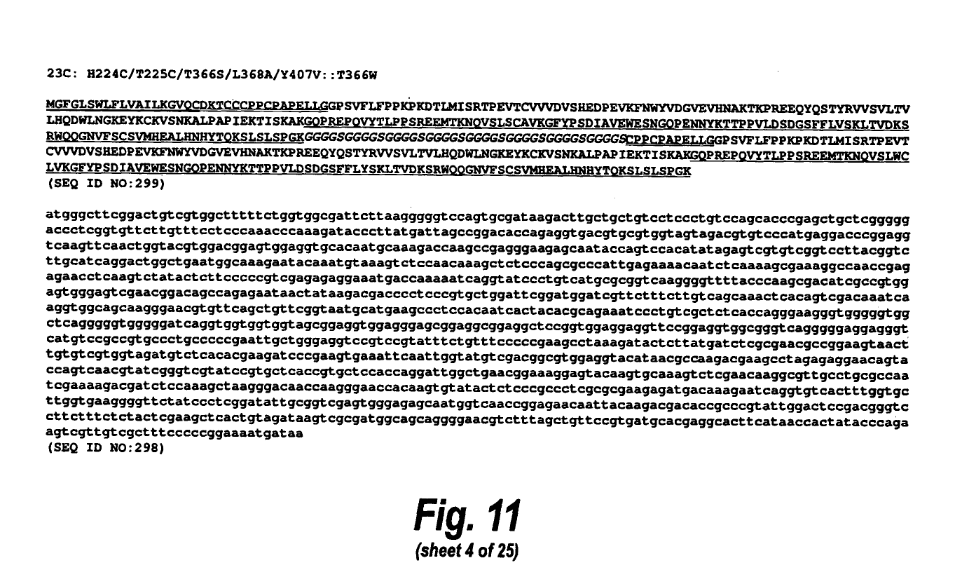

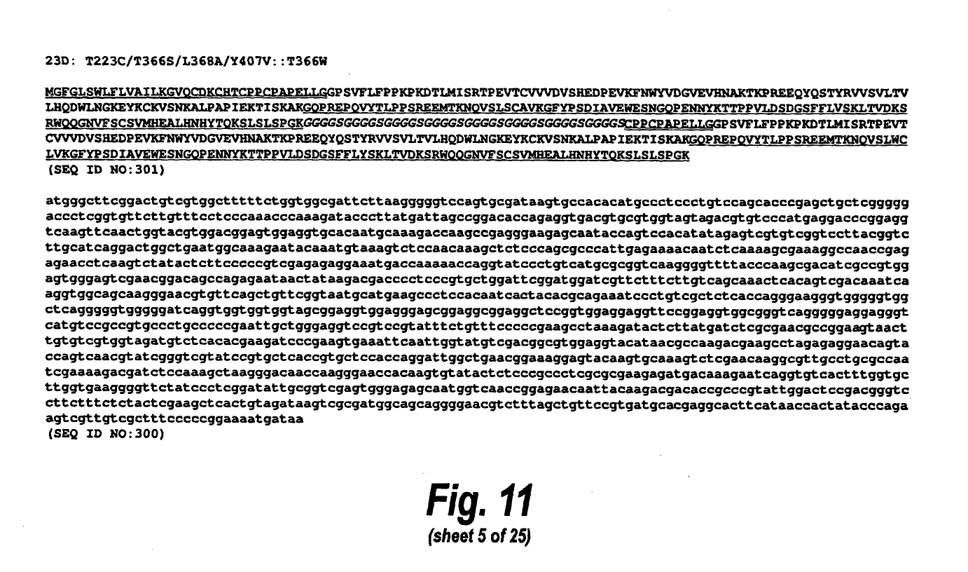

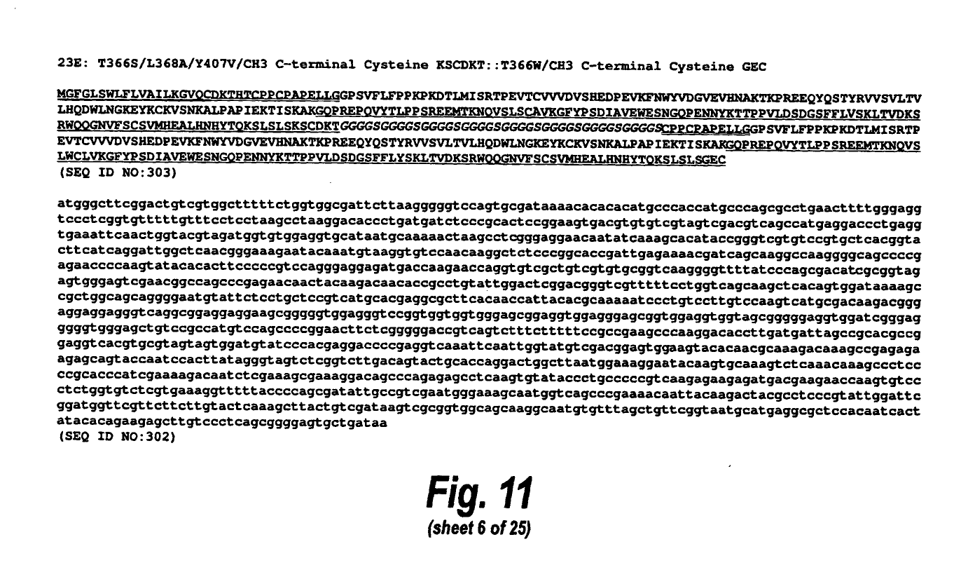

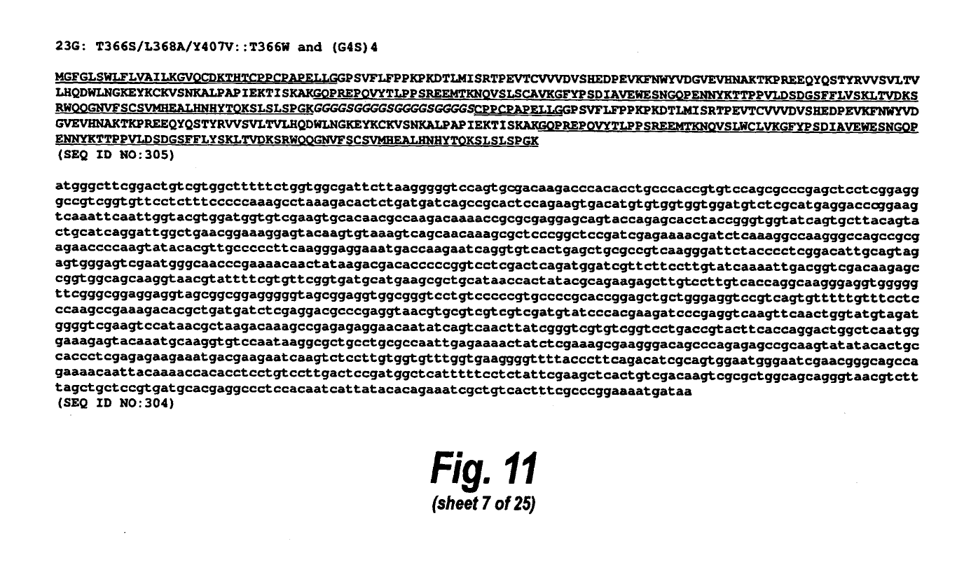

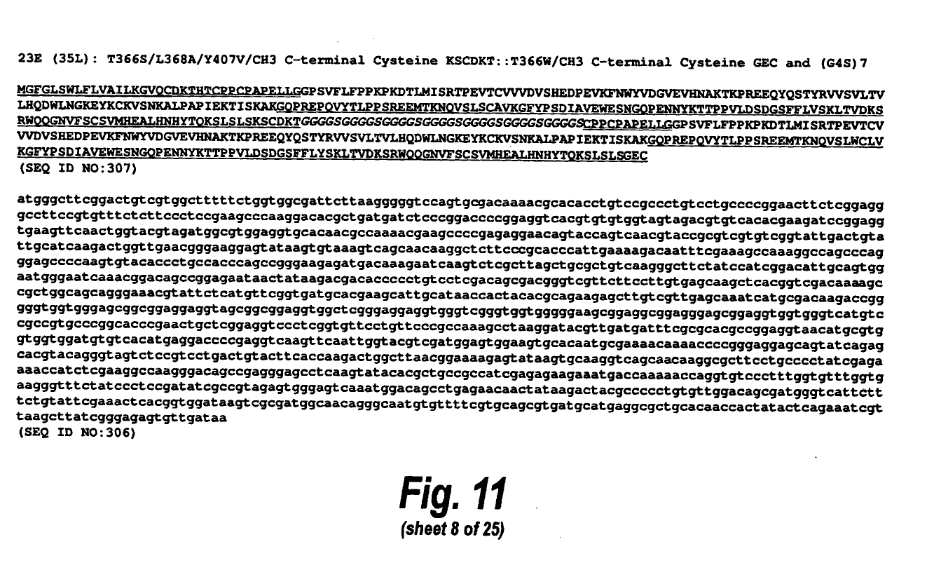

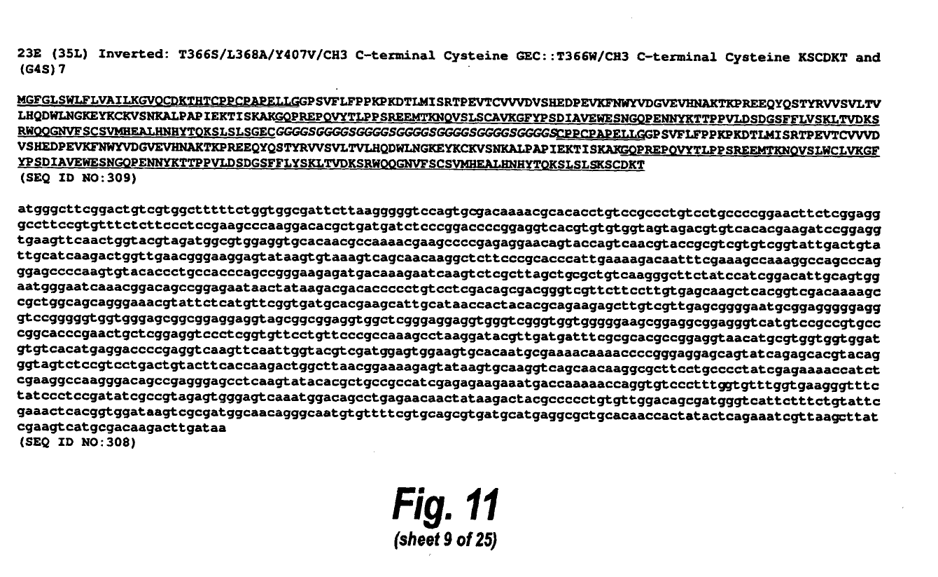

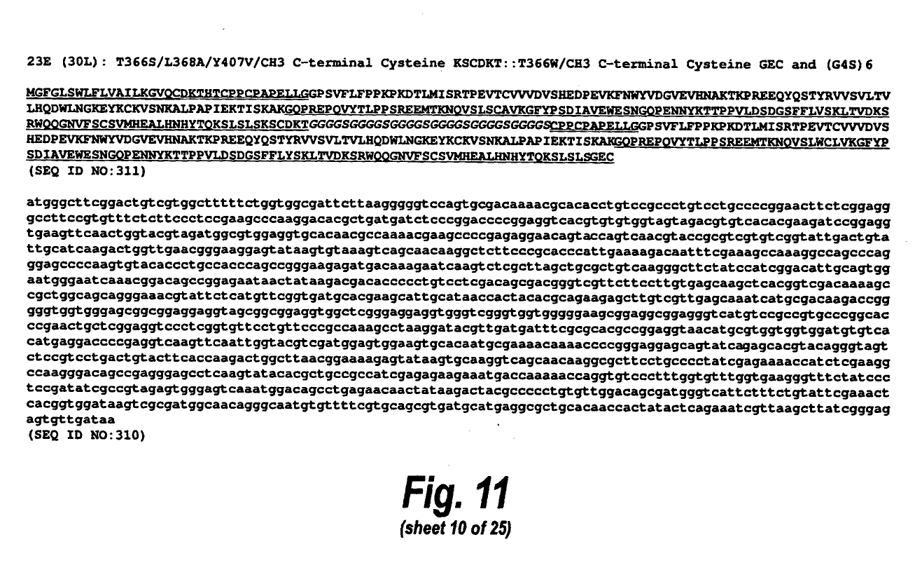

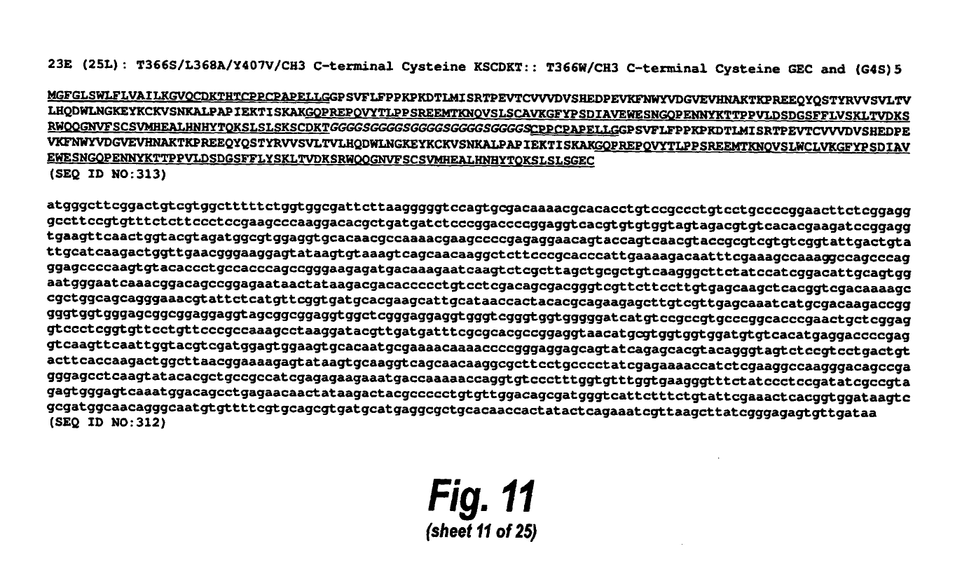

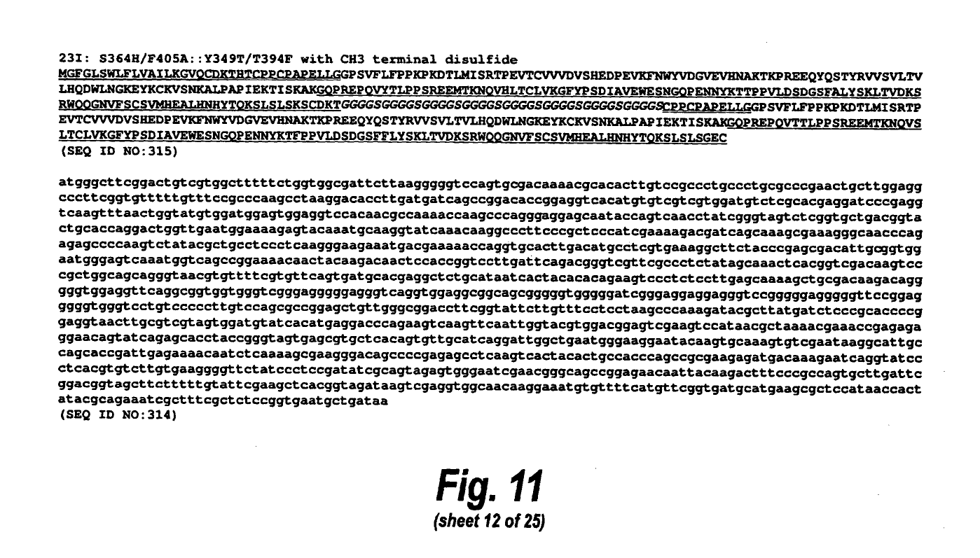

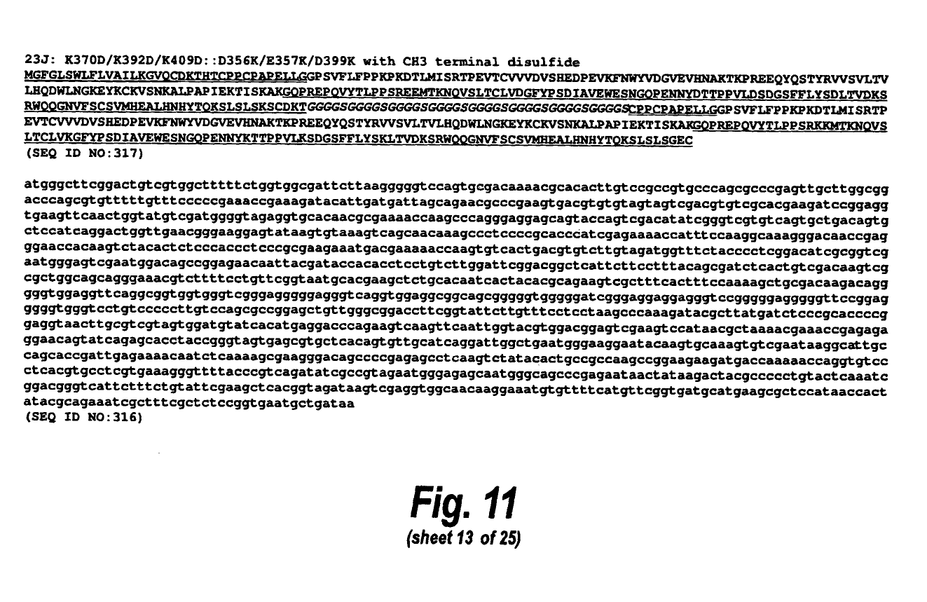

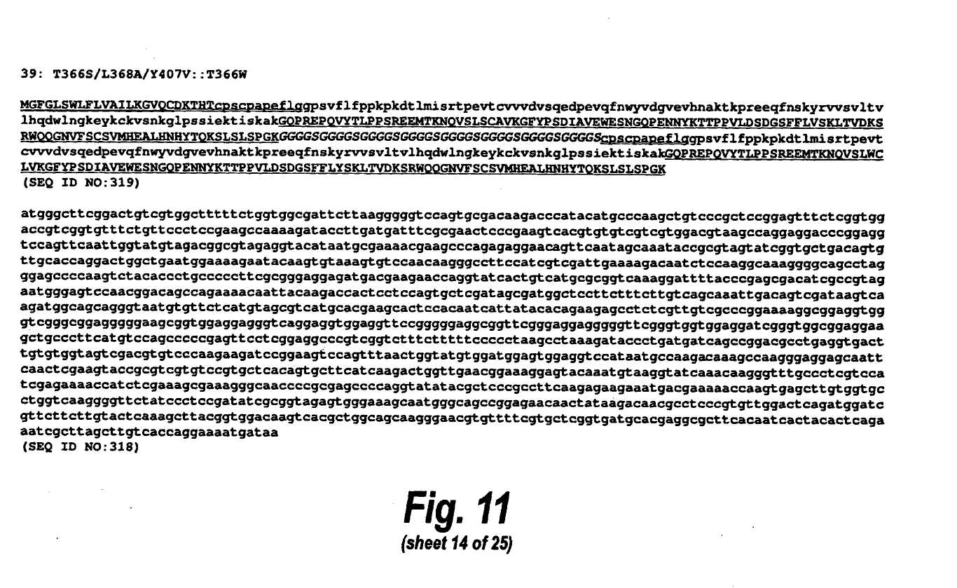

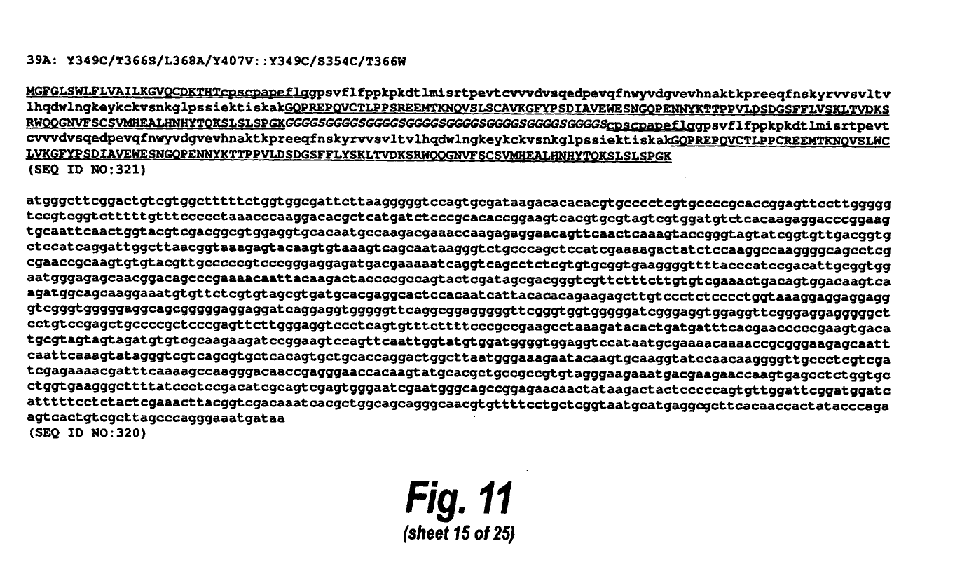

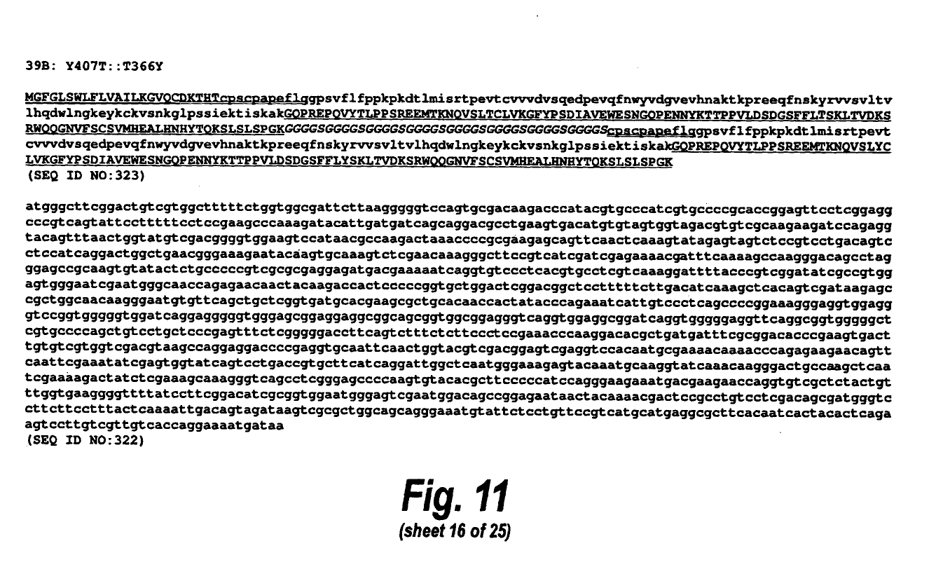

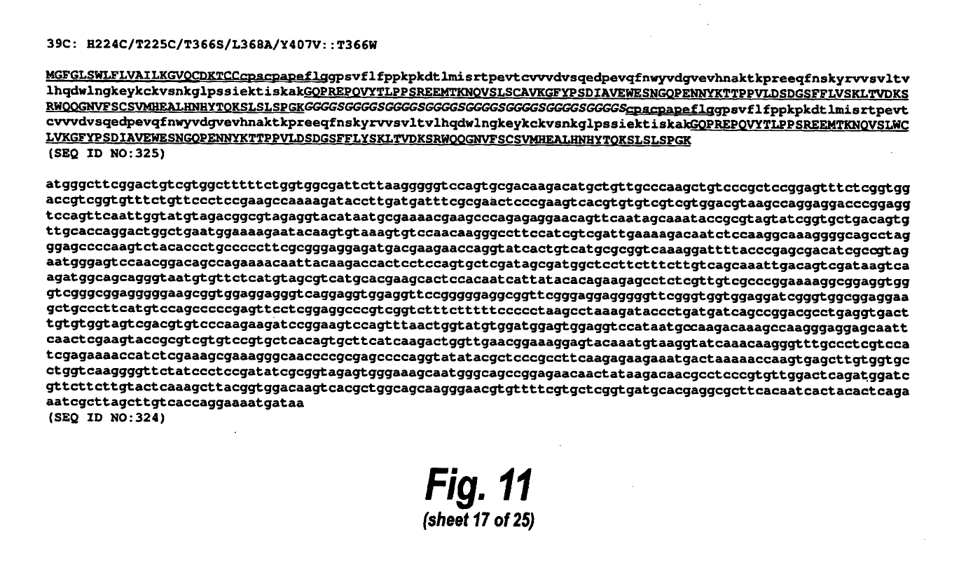

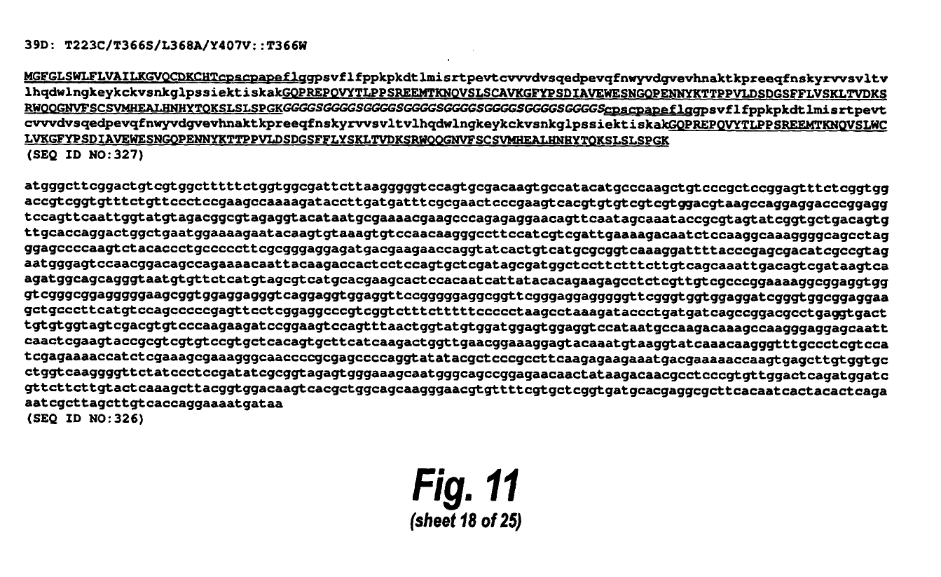

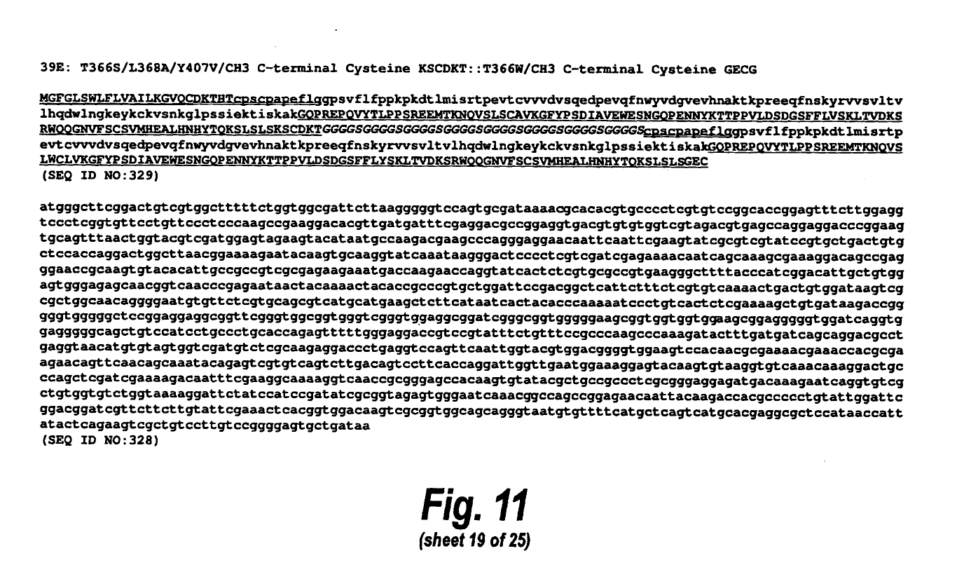

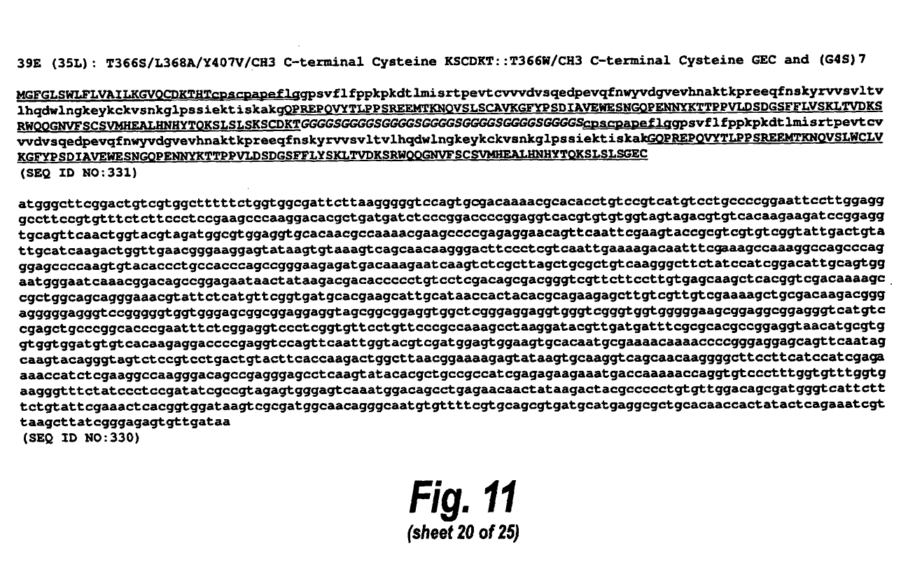

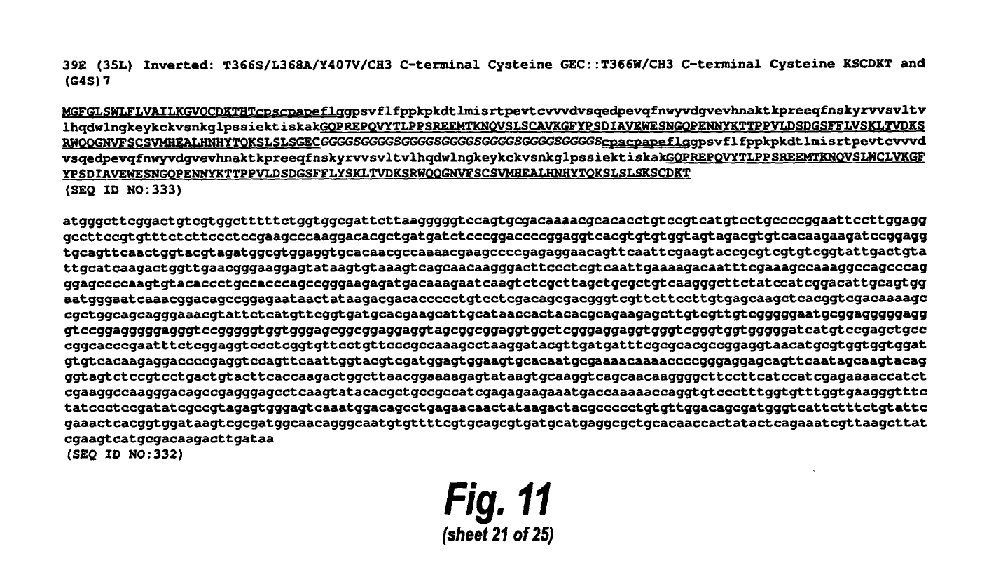

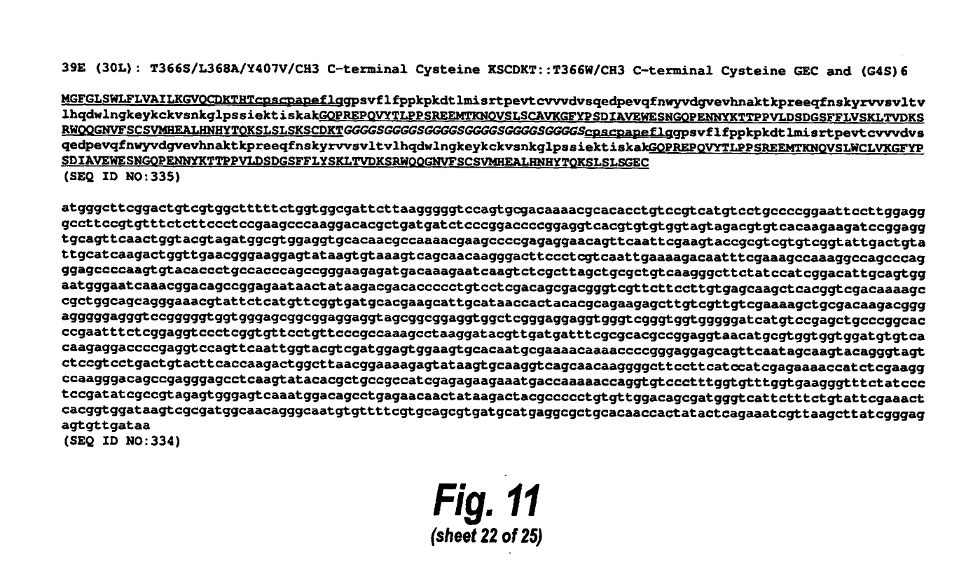

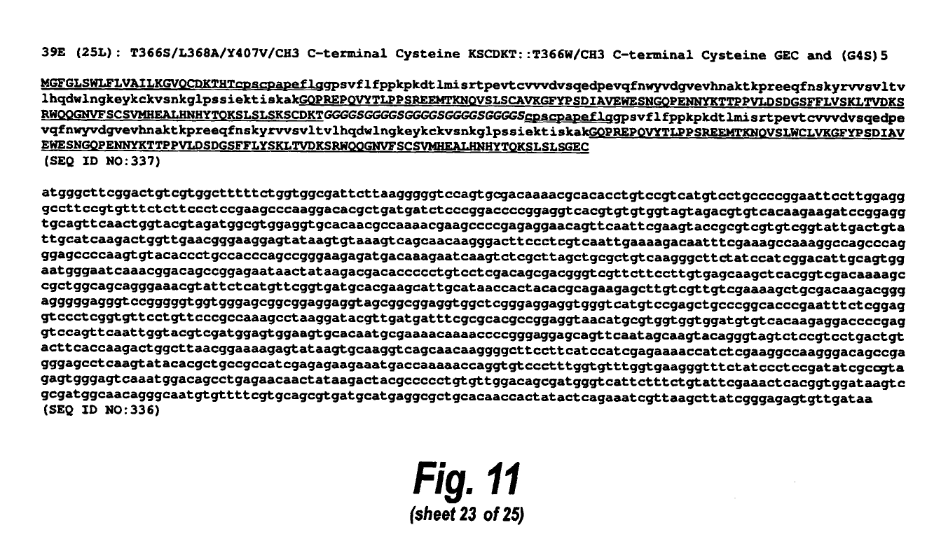

SEQ ID NOs: 292-341 are nucleotide (even numbers) and aa (odd numbers) sequences of TFcs used in Examples 1 and 2 and shown in FIG. 11.

SEQ ID NOs: 342 and 343 are the nucleotide and aa sequences, respectively, of the heavy chain of an IgG1 TFcBA comprising the anti-c-Met 5D5 VH domain, an IgG1 TFc (with AEM 1 and DiS inverted), and the panitumumab scFv (FIG. 9).

SEQ ID NO: 344 and 345 are the nucleotide and aa sequences, respectively, of the light chain Fab region of the anti-c-Met binding site 2 (SEQ ID NO:289) with the signal peptide consisting of SEQ ID NO:243 and shown in Example 3.

SEQ ID NOs: 346 and 347 are the nucleotide and aa sequences, respectively, of the heavy chain of anti-c-met/anti-EGFR TFcBA with humanized 5D5 anti-c-Met and anti-EGFR panitumumab scFv with IgG1 TFc (with AEM 1 and a 40aa TFc linker having SEQ ID NO:169; FIG. 9).

SEQ ID NOs: 348 and 349 are the nucleotide and aa sequences, respectively, of the heavy chain of anti-c-met/anti-EGFR TFcBA with humanized 5D5 anti-c-Met and anti-EGFR panitumumab scFv with IgG1/IgG4 hybrid TFc (with AEM 1 and a 40aa TFc linker having SEQ ID NO:169; FIG. 9).

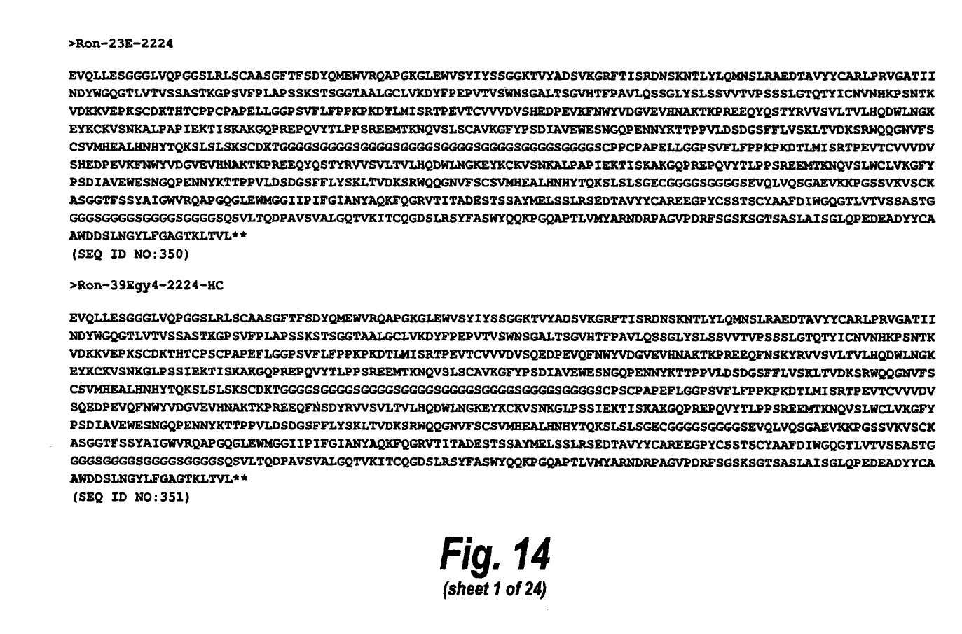

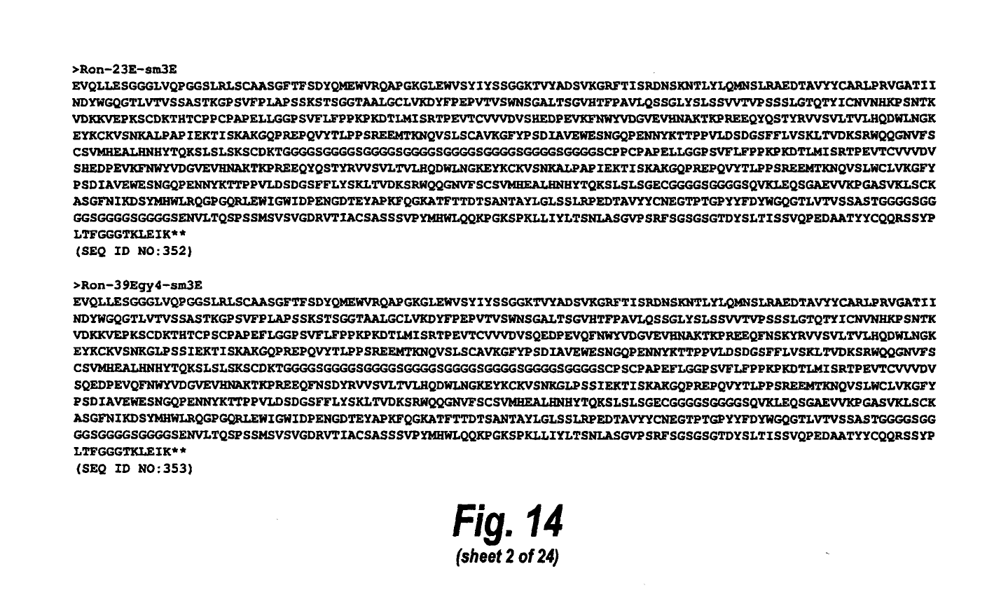

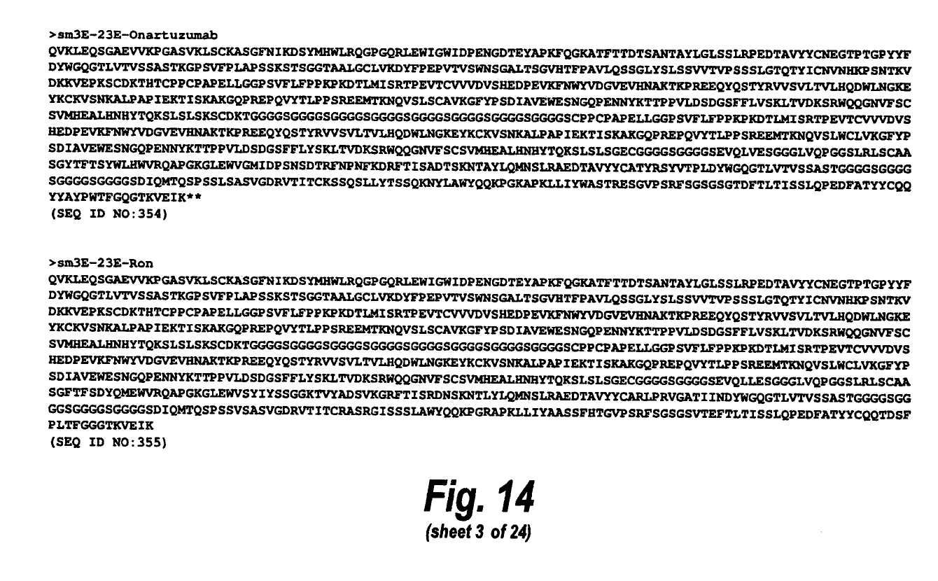

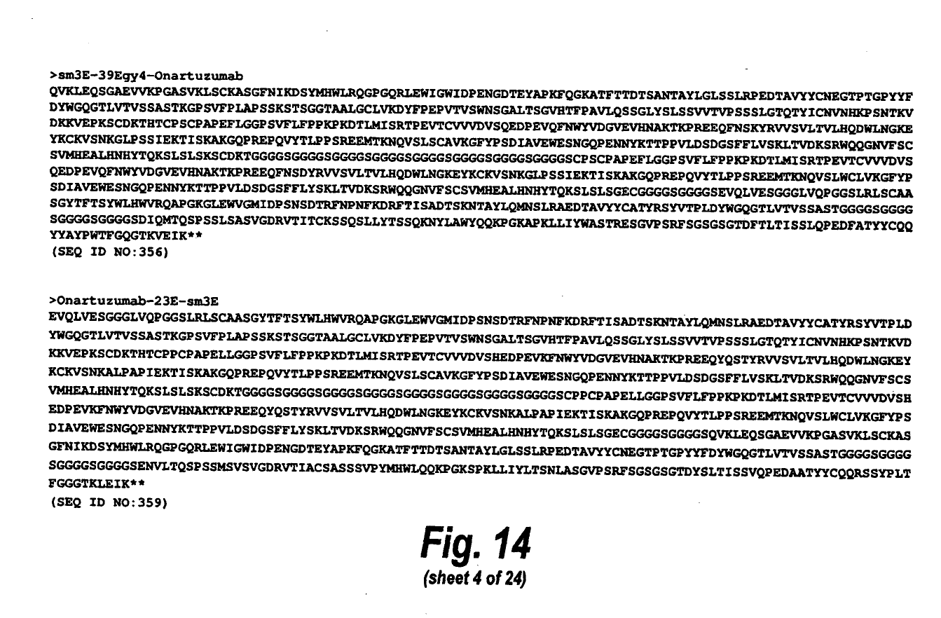

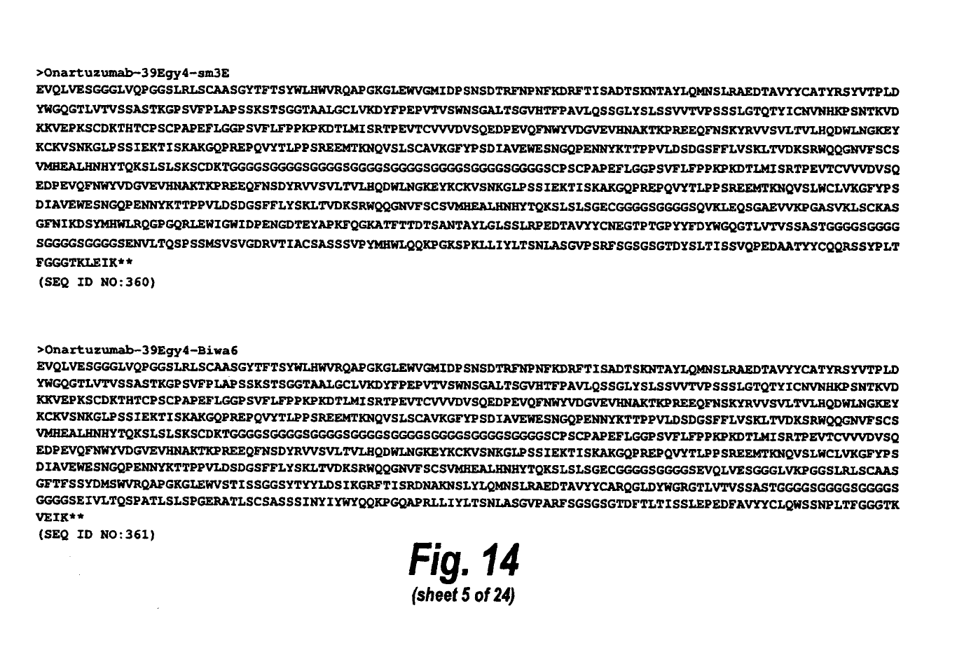

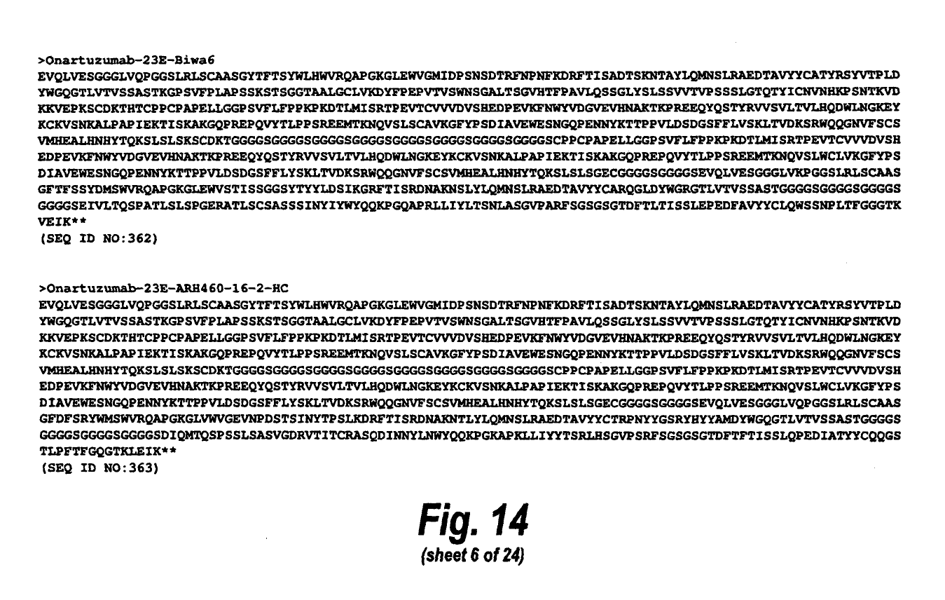

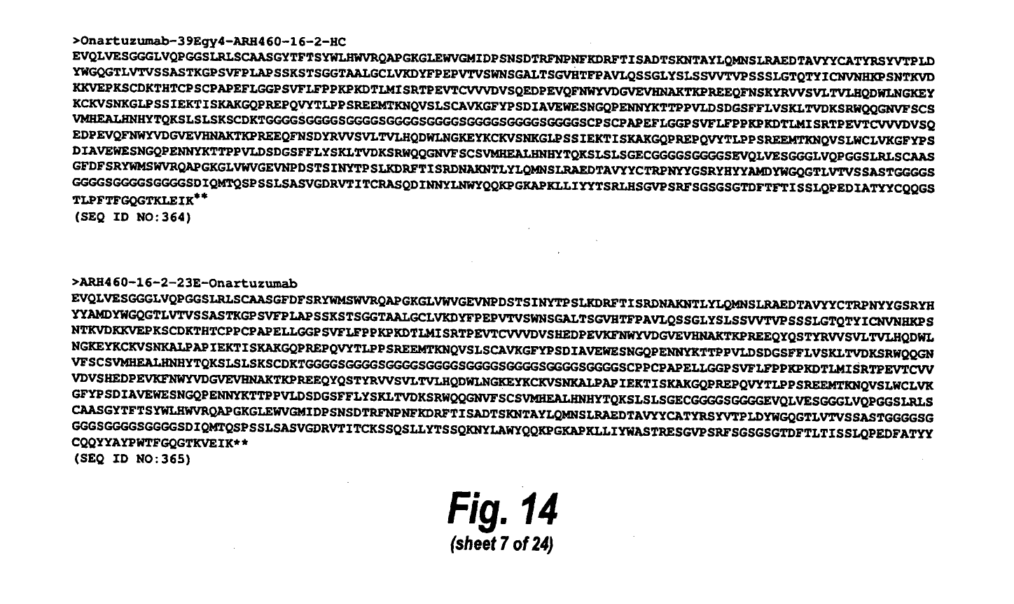

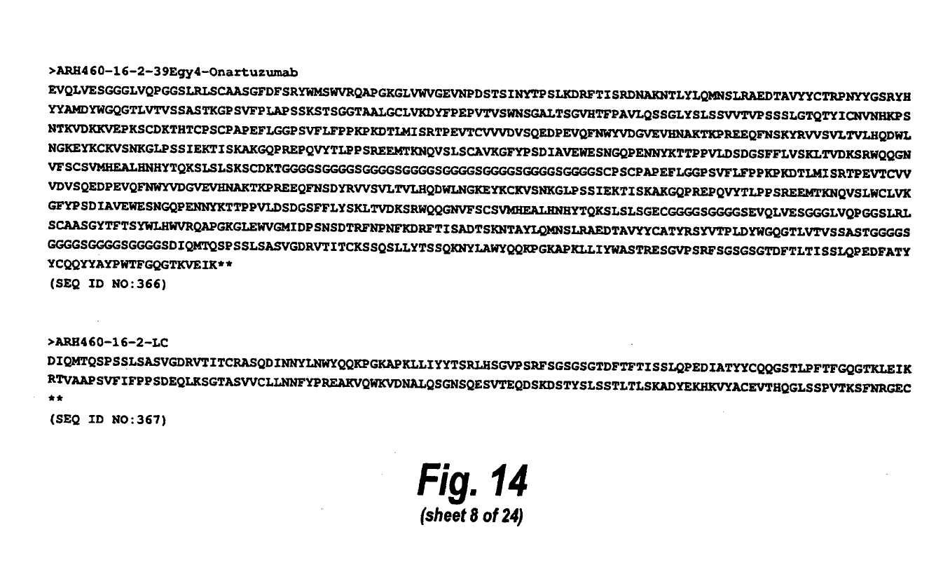

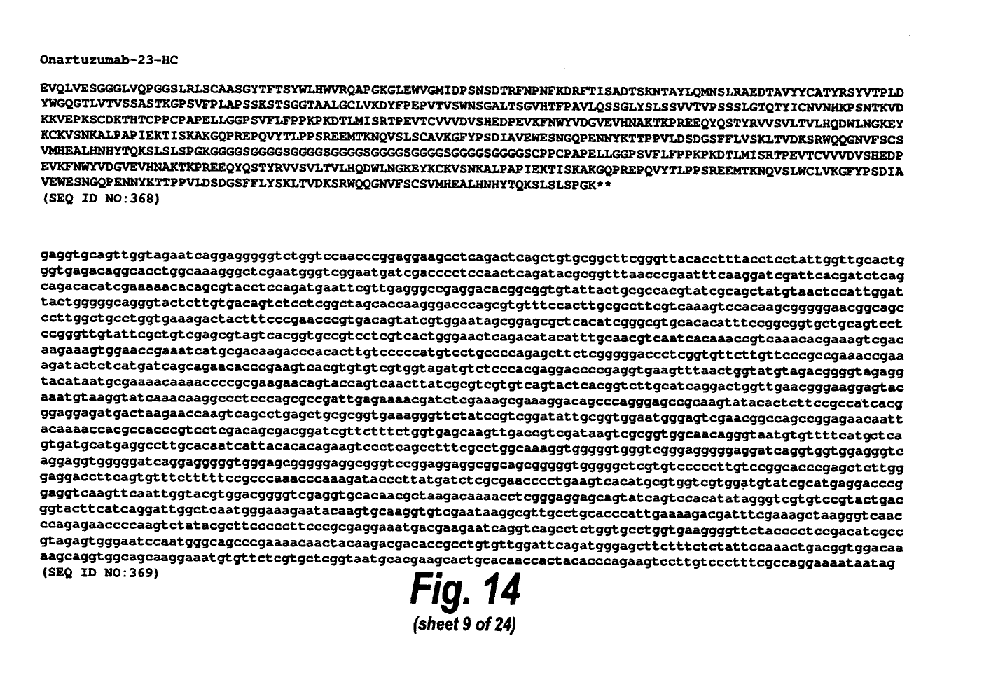

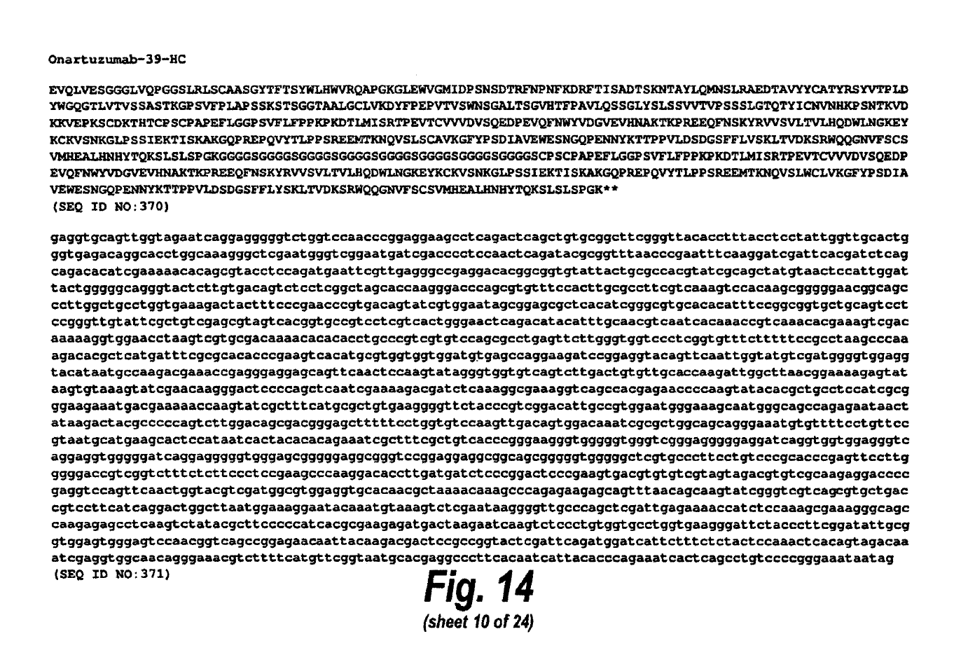

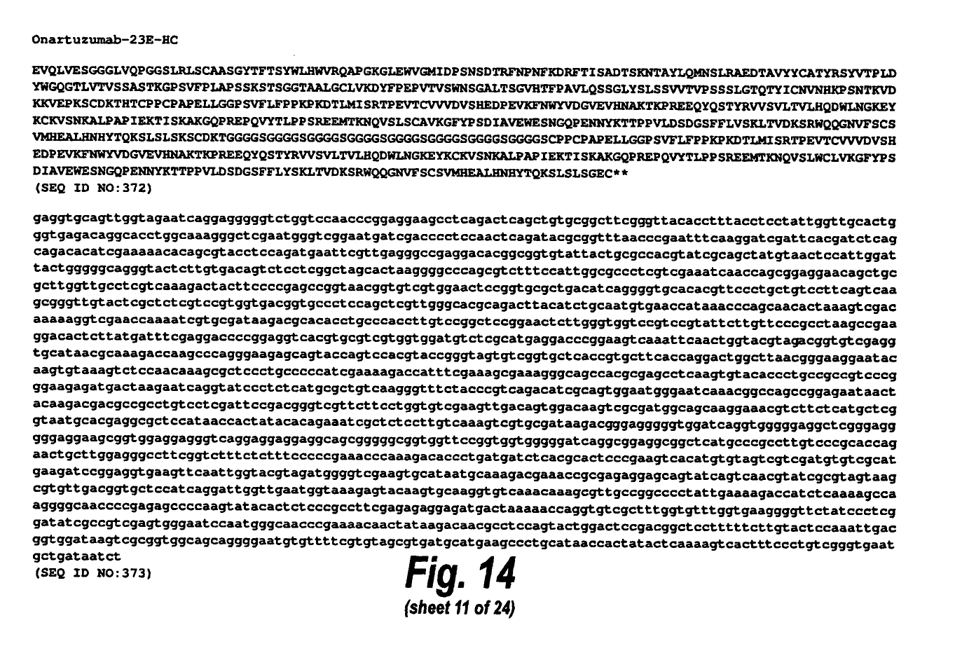

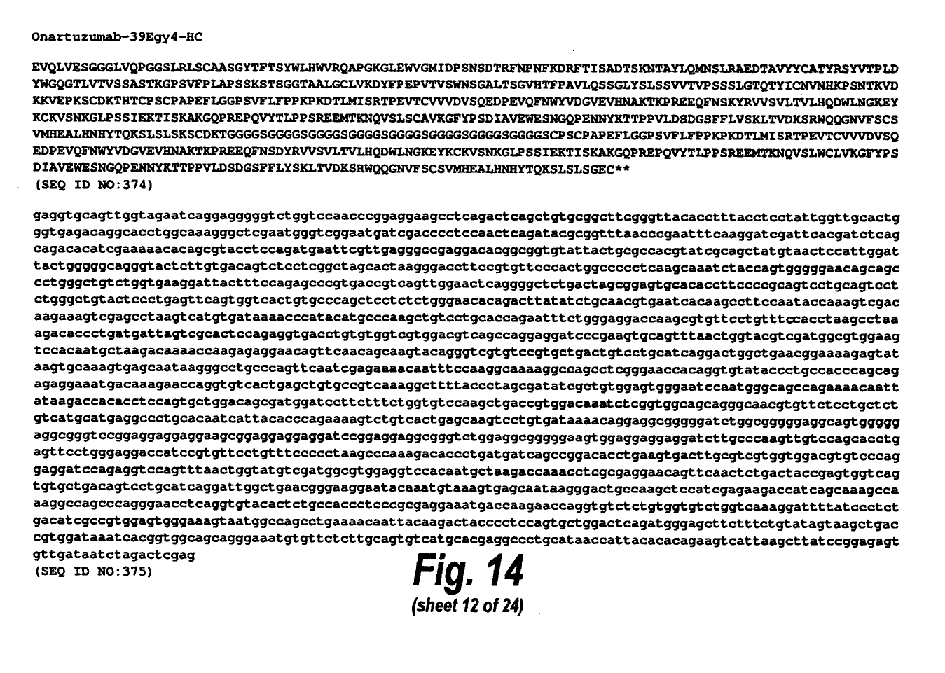

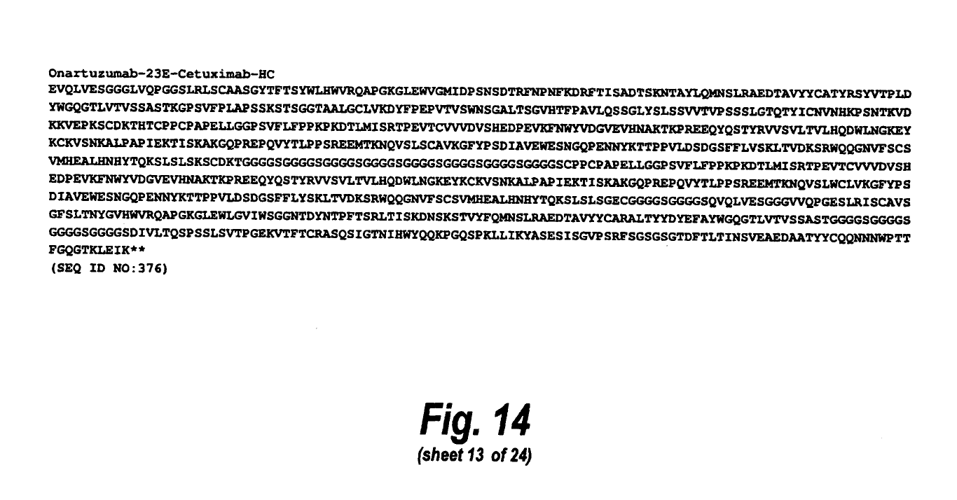

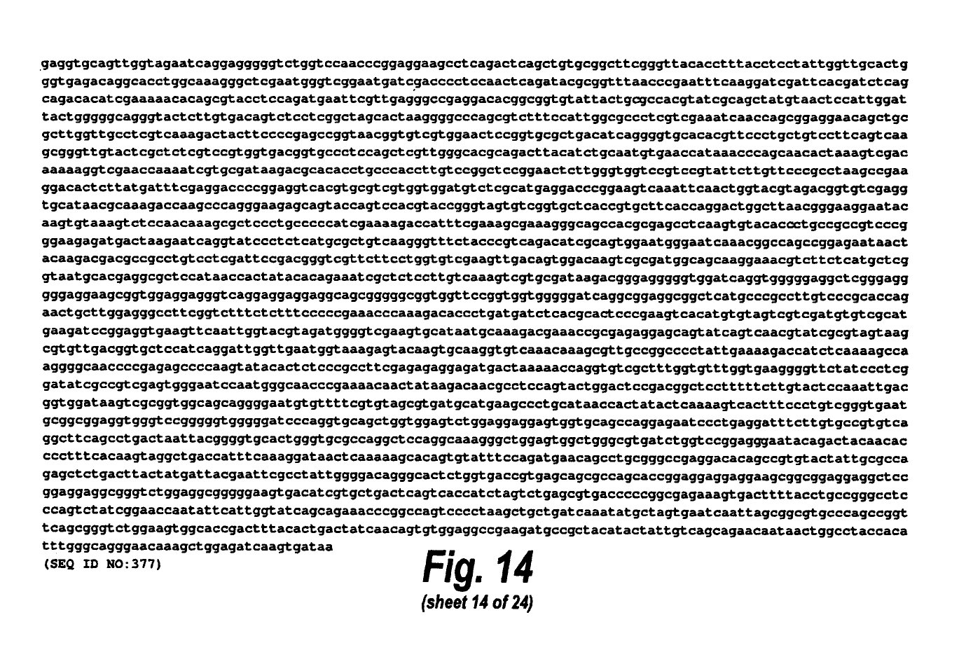

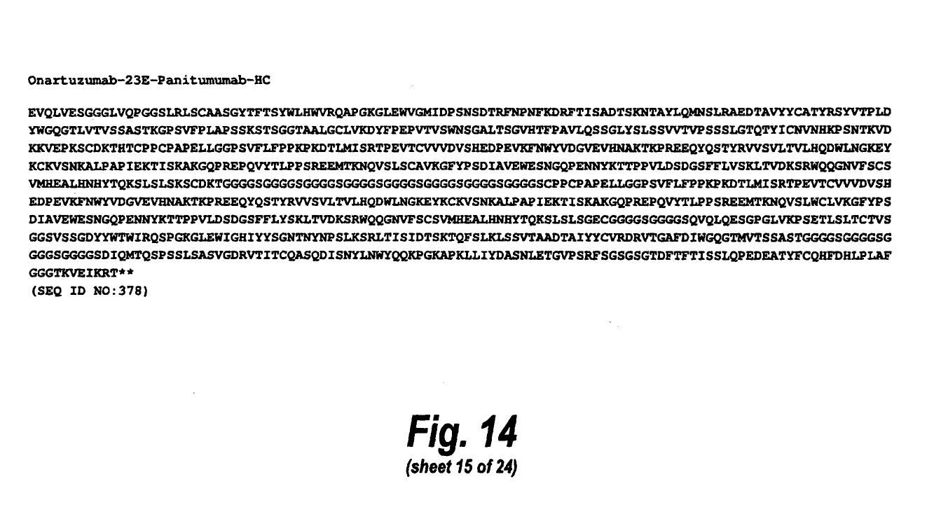

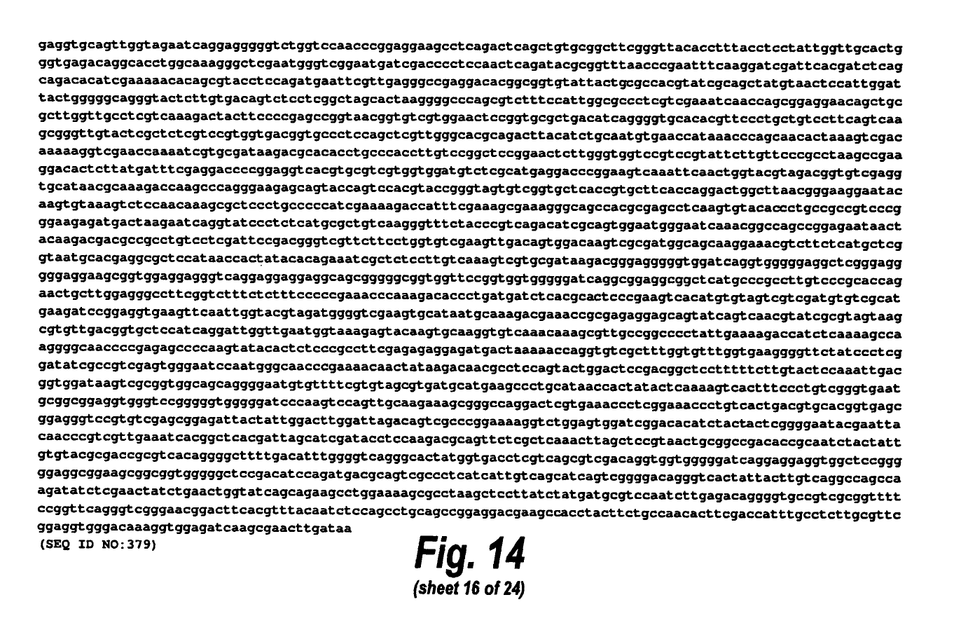

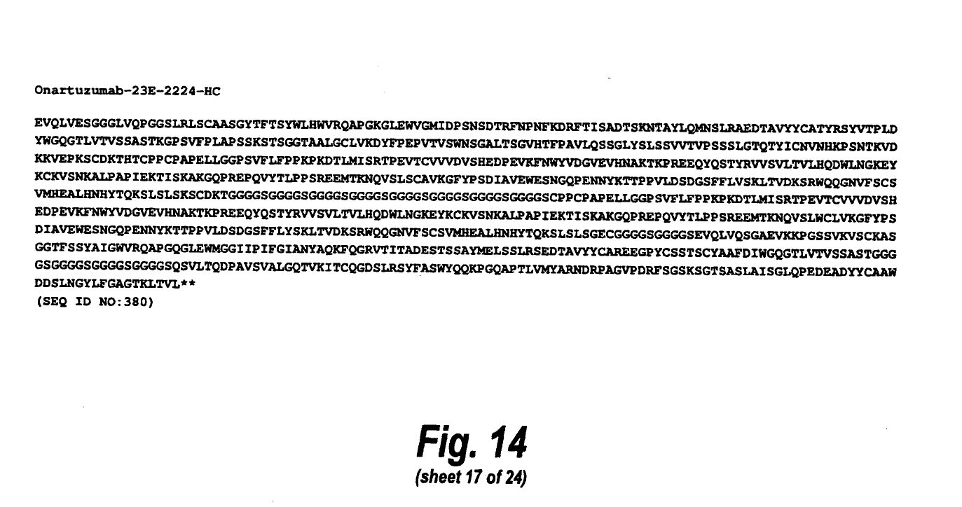

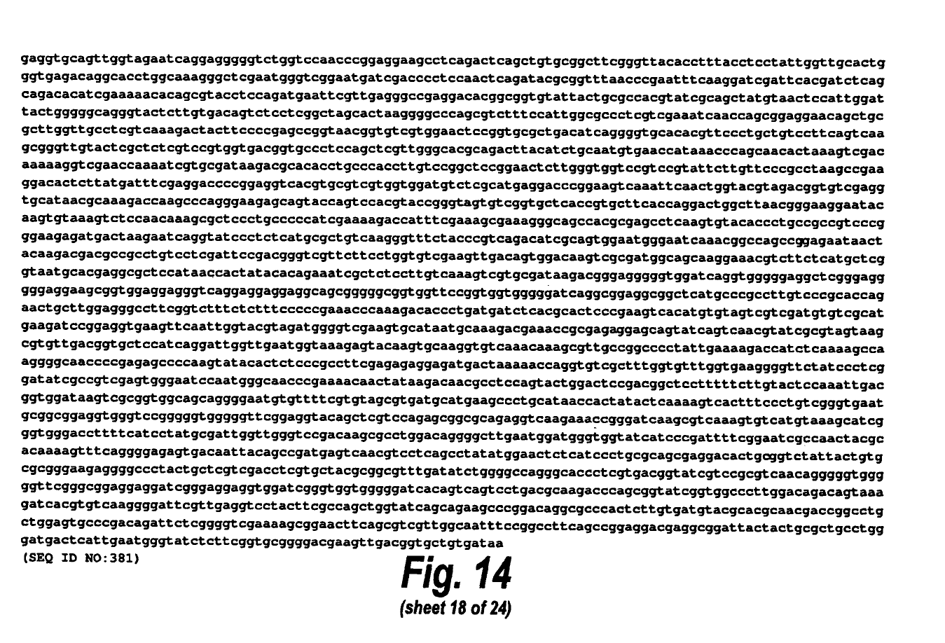

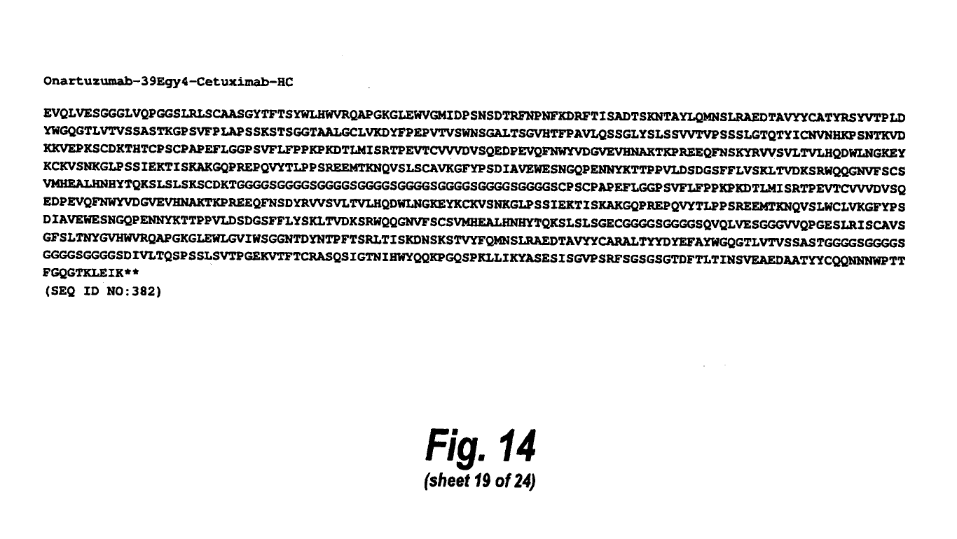

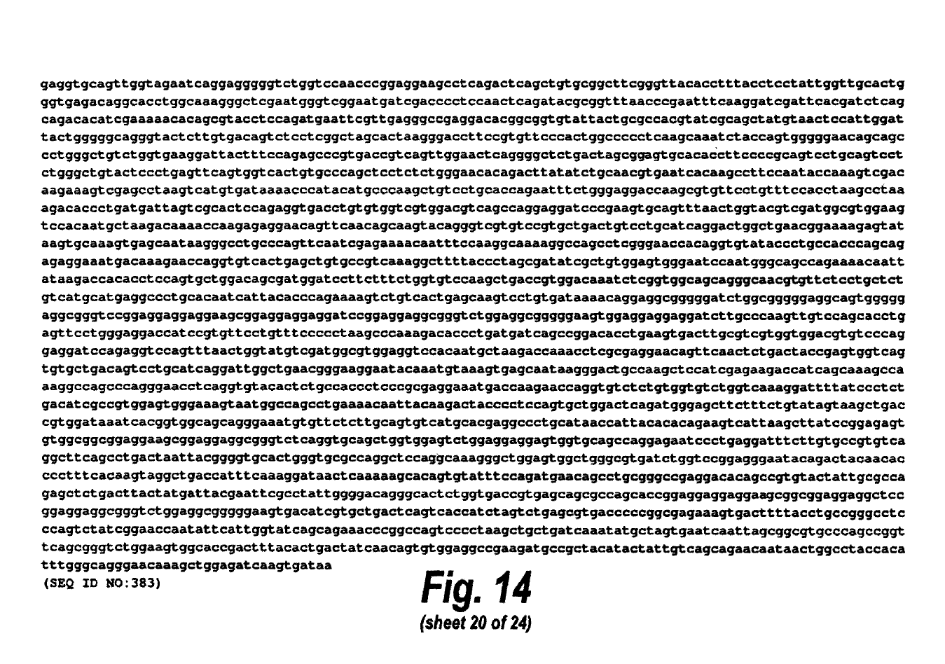

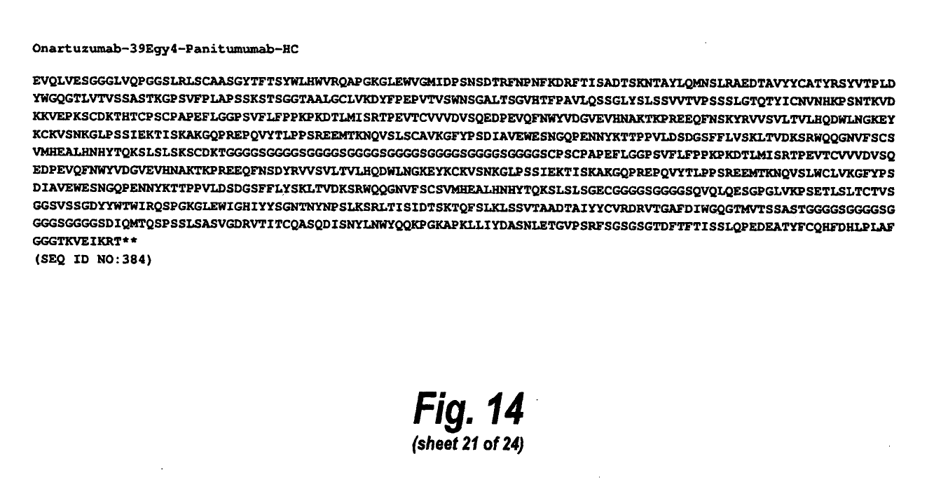

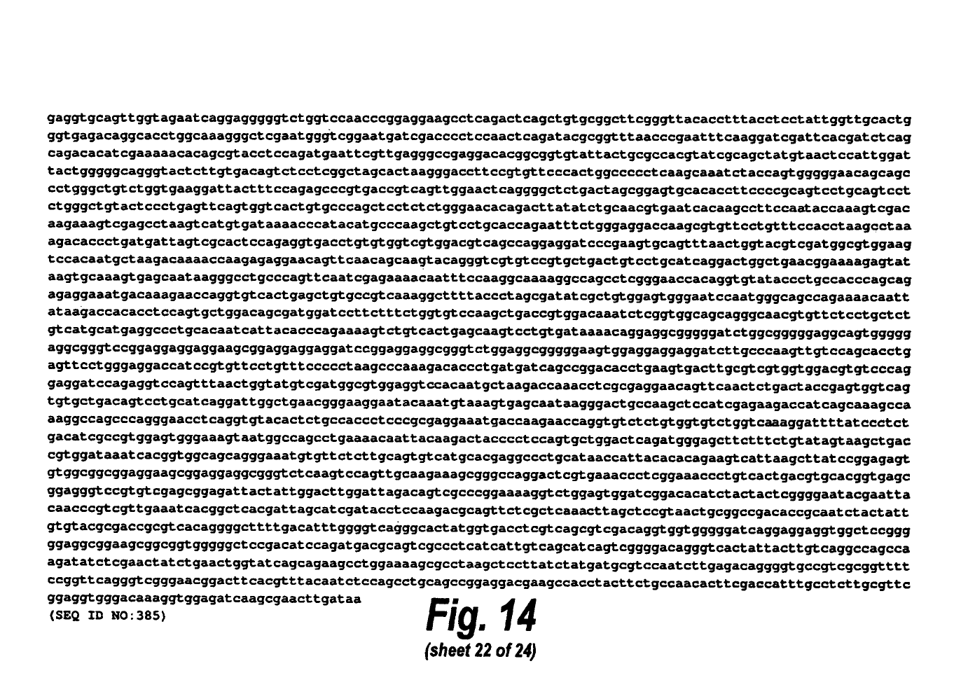

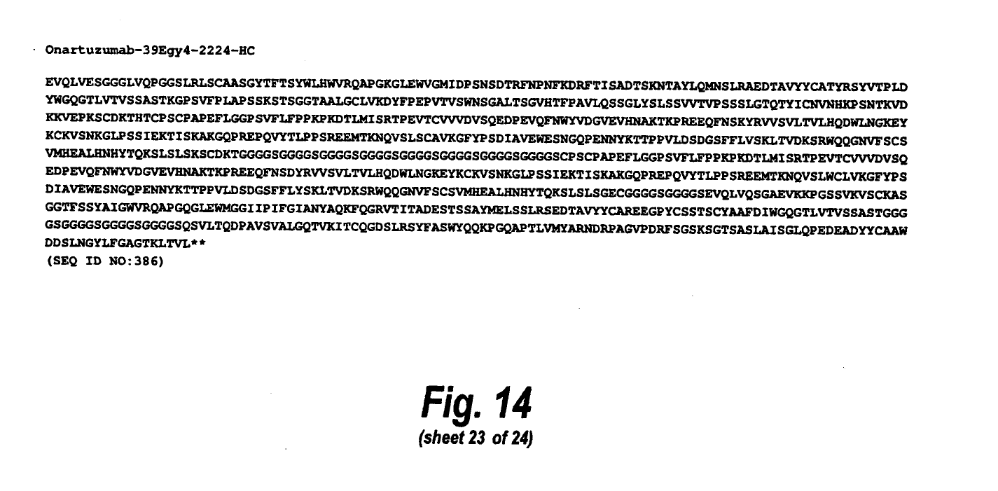

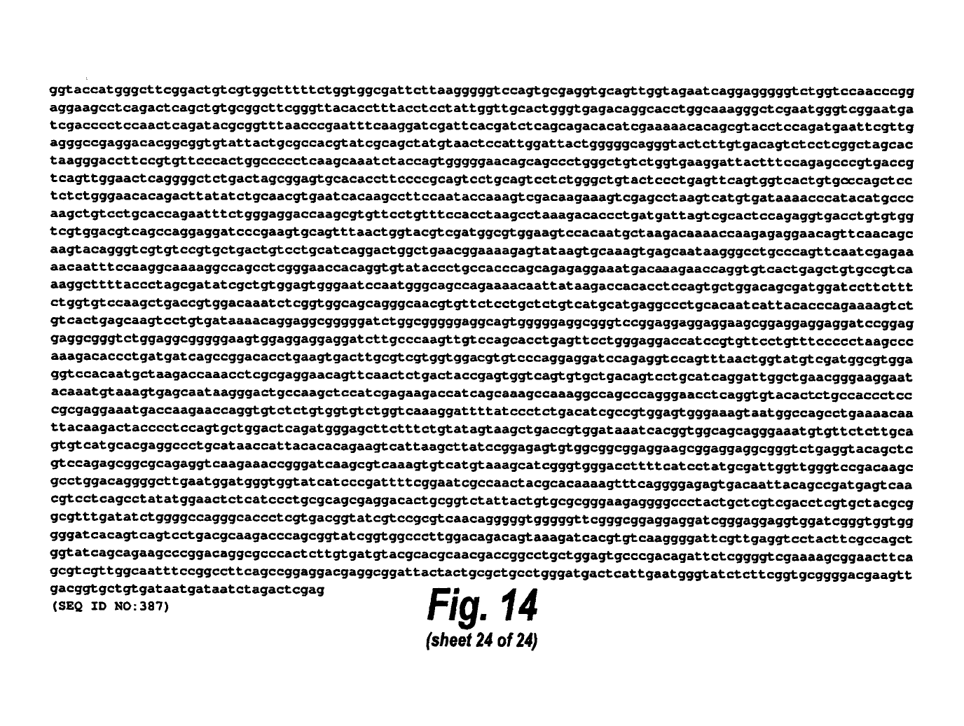

SEQ ID NO: 350 is the aa sequence of the heavy chain of anti-RON/anti-EGFR TFcBA comprising an anti-RON heavy chain Fab domain, anti-EGFR scFv 2224, and TFc 23E (SEQ ID NO:303); FIG. 14.

SEQ ID NO: 351 is the aa sequence of the heavy chain of the anti-RON/anti-EGFR TFcBA comprising an anti-RON heavy chain Fab domain, anti-EGFR scFv 2224, and TFc 39Egy4 (39E glycoform 4) (SEQ ID NO: 394); FIG. 14.

SEQ ID NO: 352 is the aa sequence of the heavy chain of the anti-RON/anti-CEA TFcBA comprising an anti-RON heavy chain Fab domain, anti-CEA scFv, and Tfc 23E (SEQ ID NO: 303); FIG. 14.

SEQ ID NO: 353 is the aa sequence of the heavy chain of the anti-RON/anti-CEA TFcBA comprising an anti-RON heavy chain Fab domain, anti-CEA scFv, and TFc 39Egy4 (SEQ ID NO: 394:); FIG. 14.

SEQ ID NO: 354 is the aa sequence of the heavy chain of the anti-CEA/anti-cMet TFcBA comprising an anti-CEA heavy chain Fab domain, anti-cMet scFv, and TFc 23E (SEQ ID NO: 303); FIG. 14.

SEQ ID NO: 355 is the aa sequence of the heavy chain of the anti-CEA/anti-RON TFcBA comprising an anti-CEA heavy chain Fab domain, anti-RON scFv, and TFc 23E (SEQ ID NO: 303); FIG. 14.

SEQ ID NO: 356 is the aa sequence of the heavy chain of the anti-CEA/anti-scMet TFcBA comprising an anti-CEA heavy chain Fab domain, anti-cMet scFv, and TFc 39Egy4 (SEQ ID NO: 394); FIG. 14.

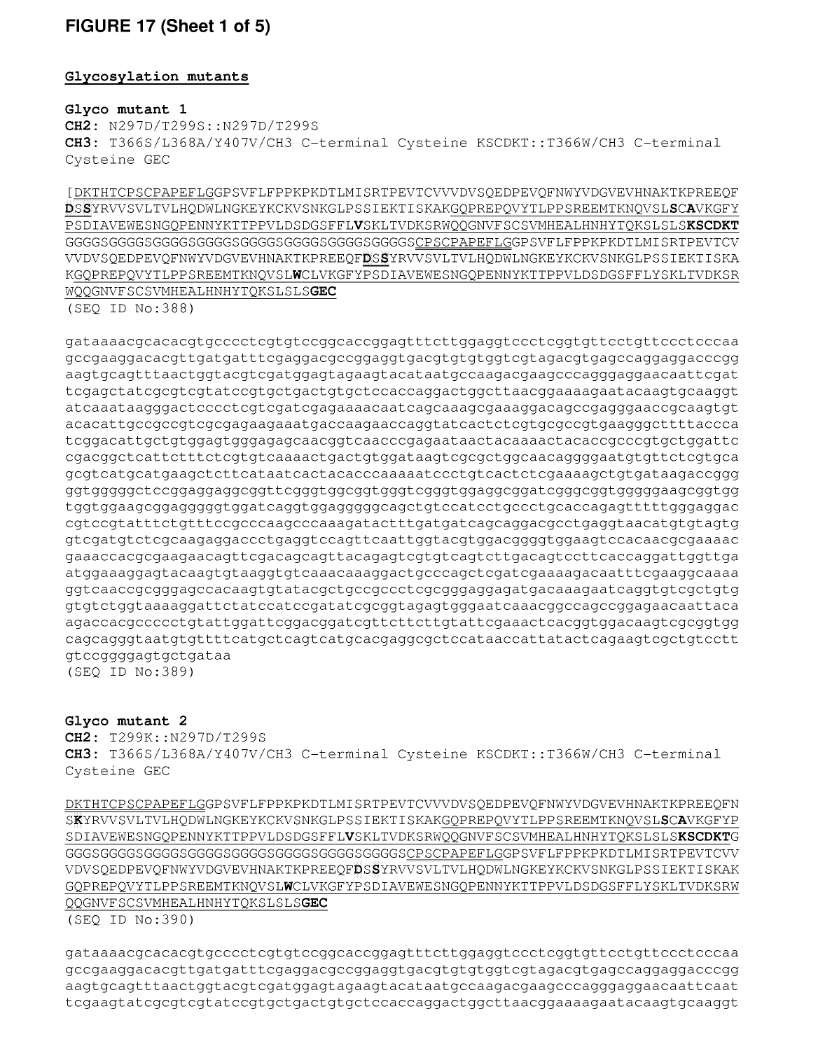

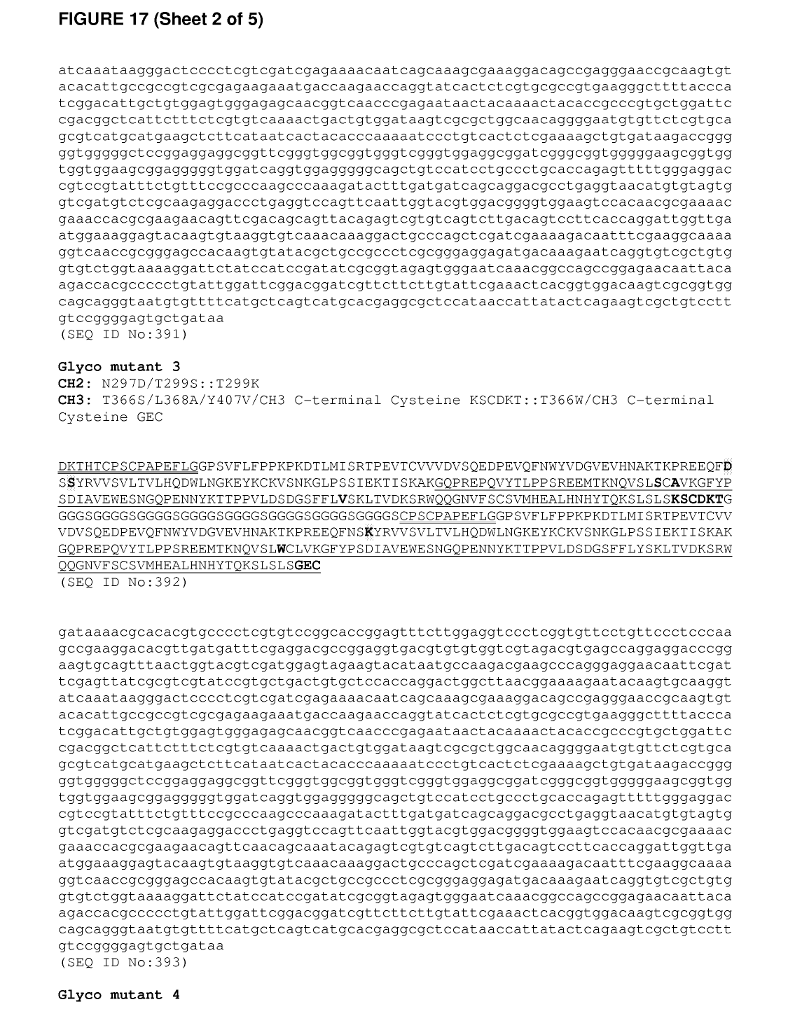

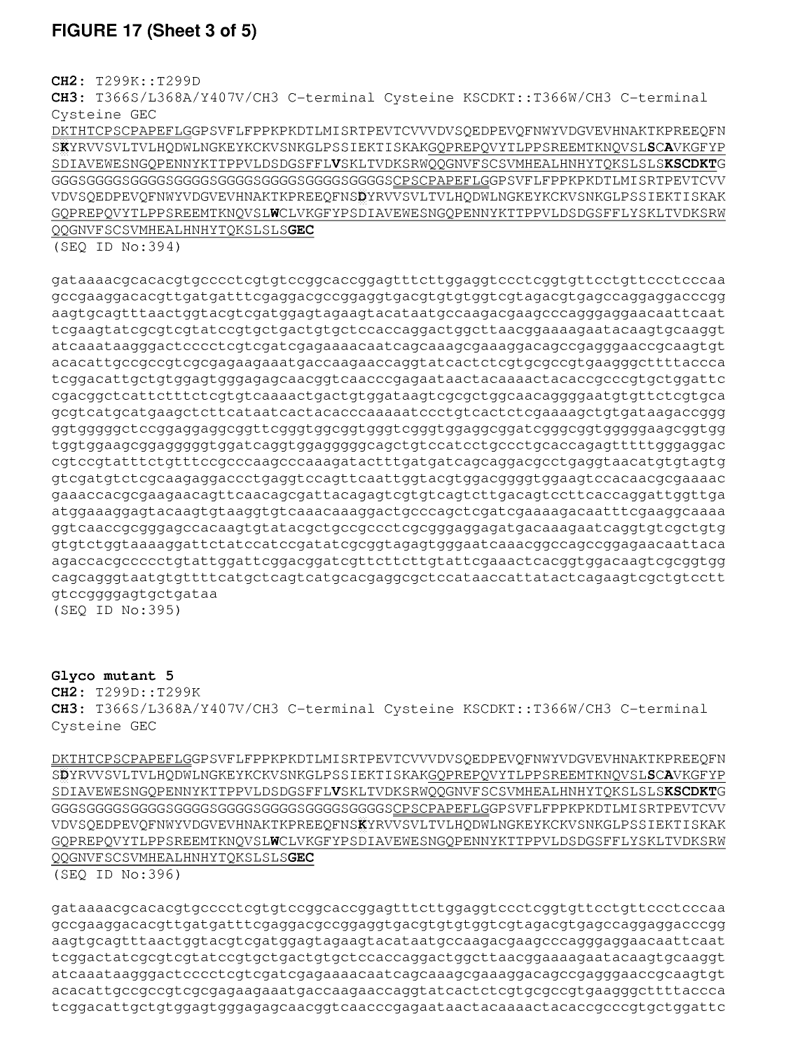

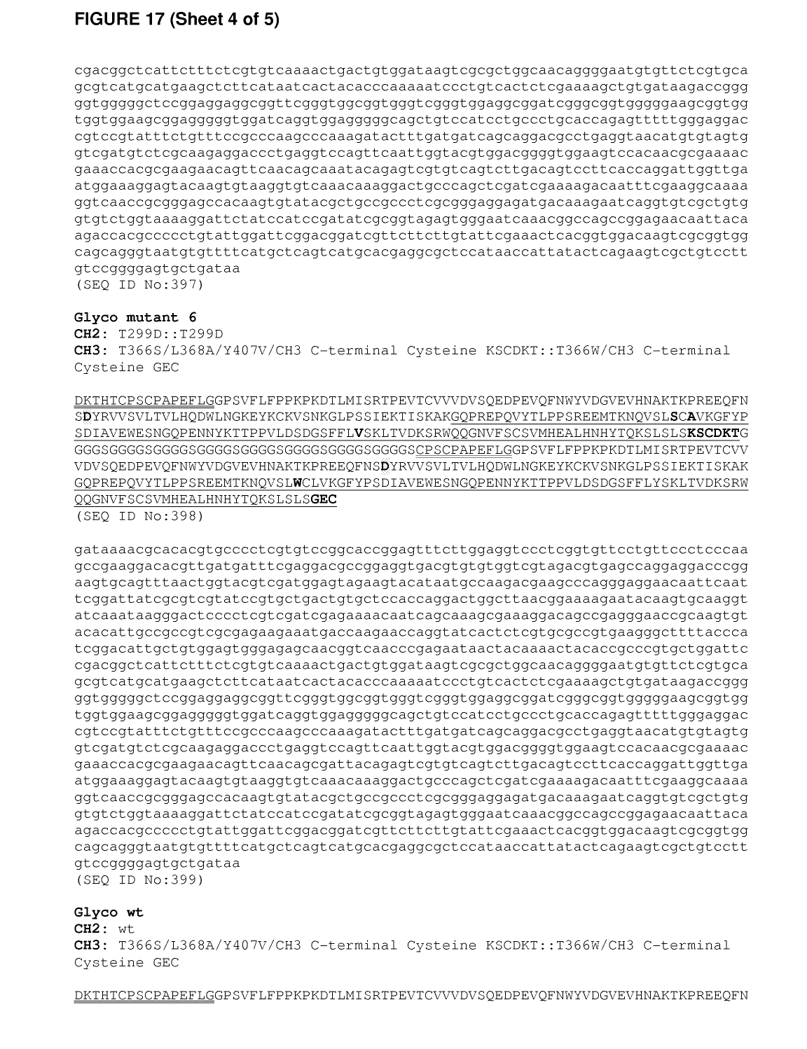

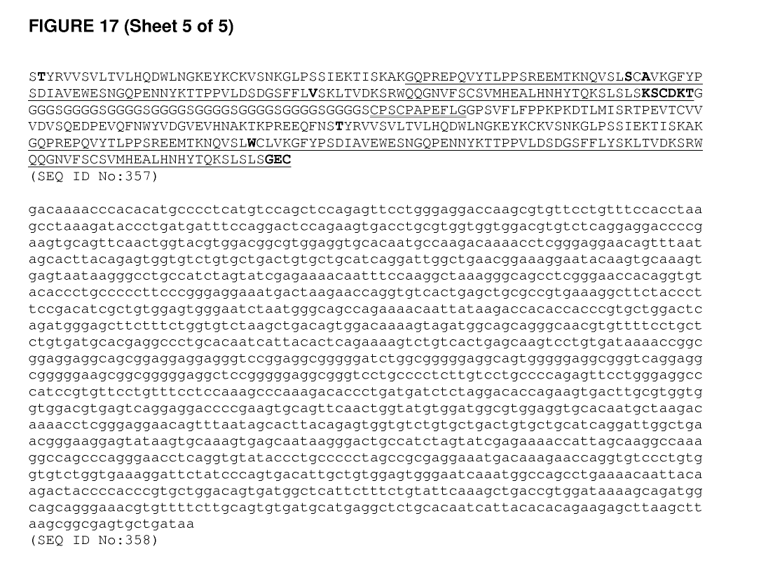

SEQ ID NOs 357-358 are the aa sequence and nucleotide sequence of TFc wild-type CH2 sequence; and T366S/L368A/Y407V/CH3 C-terminal Cysteine KSCDKT::T366W/CH3 C-terminal Cysteine GEC in the CH3 domains; FIG. 17.

SEQ ID NO: 359 is the aa sequence of the heavy chain of the anti-cMet/anti-CEA TFcBA comprising an anti-cMet heavy chain Fab domain, anti-CEA scFv, and TFc 23E (SEQ ID NO: 303); FIG. 14.

SEQ ID NO: 360 is the aa sequence of the heavy chain of the anti-cMet/anti-CEA TFcBA comprising an anti-cMet heavy chain Fab domain, anti-CEA scFv, and TFc 39Egy4 (SEQ ID NO: 394); FIG. 14.

SEQ ID NO: 361 is the aa sequence of the heavy chain of the anti-cMet/anti-CEA CD44 comprising an anti-cMet heavy chain Fab, an anti-CD44 scFv, and TFc 39Egy4 (SEQ ID NO: 394); FIG. 14.

SEQ ID NO: 362 is the aa sequence of the heavy chain of the anti-cMet/anti-CEA CD44 comprising an anti-cMet heavy chain Fab domain, an anti-CD44 scFv, and TFc 23E (SEQ ID NO: 303); FIG. 14.

SEQ ID NO: 363 is the aa sequence of the heavy chain of the anti-cMet/anti-CEA CD44 comprising an anti-cMet heavy chain Fab domain, an anti-CD44 scFv, and TFc 23E (SEQ ID NO: 303); FIG. 14.

SEQ ID NO: 364 is the aa sequence of the heavy chain of the anti-cMet/anti-CEA CD44 comprising an anti-cMet heavy chain Fab domain, an anti-CD44 scFv, and TFc 39Egy4 (SEQ ID NO: 394), FIG. 14.

SEQ ID NO: 365 is the aa sequence of the heavy chain of the anti-CD44/anti-anti-cMet comprising an anti-CD44 heavy chain Fab domain, an anti-cMet scFv, and TFc 23E (SEQ ID NO: 303); FIG. 14.

SEQ ID NO: 366 is the aa sequence of the heavy chain of the anti-CD44/anti-cMet comprising an anti-CD44 heavy chain Fab domain, an anti-cMet scFv, and TFc 39Egy4 (SEQ ID NO: 394); FIG. 14.

SEQ ID NO: 367 is the aa sequence of the anti-CD44 ARH60-16-2 light chain.

SEQ ID NOs 368-369 are the aa sequence and nucleotide sequence of the anti-cMet antibody onartuzumab and TFc 23 light chain; FIG. 14.

SEQ ID NOs 370-371 are the aa sequence and nucleotide sequence of the anti-cMet antibody onartuzumab and TFc 39 heavy chain; FIG. 14.

SEQ ID NOs 372-373 are the aa sequence and nucleotide sequence of the anti-cMet antibody onartuzumab and TFc 23E heavy chain; FIG. 14.

SEQ ID NOs 374-375 are the aa sequence and nucleotide sequence of the anti-cMet antibody onartuzumab and TFc 39Egy4 heavy chain; FIG. 14.

SEQ ID NOs 376-377 are the aa sequence and nucleotide sequence of anti-cMet/anti-EGFR comprising an anti-cMet heavy chain Fab domain, the cetuximab anti-EGFR scFv, and TFc 23E (SEQ ID NO: 303); FIG. 14.

SEQ ID NOs 378-379 are the aa sequence and nucleotide sequence of anti-cMet/anti-EGFR comprising an anti-cMet heavy chain Fab domain, the panitumumab anti-EGFR scFv, and TFc 23E (SEQ ID NO: 303); FIG. 14.

SEQ ID NOs 380-381 are the aa sequence and nucleotide sequence of anti-cMet/anti-EGFR comprising an anti-cMet heavy chain Fab domain, the 2224 anti-EGFR scFv, and TFc 23E (SEQ ID NO: 303); FIG. 14.

SEQ ID NOs 382-383 are the aa sequence and nucleotide sequence of anti-cMet/anti-EGFR comprising an anti-cMet heavy chain Fab domain the cetuximab anti-EGFR scFv, and TFc 39Egy4 (SEQ ID NO: 394); FIG. 14.

SEQ ID NOs 384-385 are the aa sequence and nucleotide sequence of anti-cMet/anti-EGFR comprising an anti-cMet heavy chain Fab domain, the panitumumab anti-EGFR scFv, and TFc 39Egy4 (SEQ ID NO: 394); FIG. 14.

SEQ ID NOs 386-387 are the aa sequence and nucleotide sequence of anti-cMet/anti-EGFR comprising an anti-cMet heavy chain Fab domain, the 2224 anti-EGFR scFv, and TFc 39Egy4 (SEQ ID NO: 394); FIG. 14.

For the sequences disclosed in FIG. 17, the double underline is the hinge, the single underline is the CH3 domain, the second double underline is the second hinge, the second underline is the second CH3.

SEQ ID NOs 388-389 are the aa sequence and nucleotide sequence of glycosylation mutant 1, comprising N297D/T299S::N297D/T299S amino acid changes in the CH2 domains (underlined, bold-face), and T366S/L368A/Y407V/CH3 C-terminal Cysteine KSCDKT::T366W/CH3 C-terminal Cysteine GEC in the CH3 domains; FIG. 17 SEQ ID NOs 390-391 are the aa sequence and nucleotide sequence of glycosylation mutant 2, comprising T299K::N297D/T299S amino acid changes in the CH2 domains (underlined, bold-face), and T366S/L368A/Y407V/CH3 C-terminal Cysteine KSCDKT::T366W/CH3 C-terminal Cysteine GEC in the CH3 domains; FIG. 17.

SEQ ID NOs 392-393 are the aa sequence and nucleotide sequence of glycosylation mutant 3, comprising N297D/T299S::T299K amino acid changes in the CH2 domains (underlined, bold-face), and T366S/L368A/Y407V/CH3 C-terminal Cysteine KSCDKT::T366W/CH3 C-terminal Cysteine GEC in the CH3 domains; FIG. 17.

SEQ ID NOs 394-395 are the aa sequence and nucleotide sequence of glycosylation mutant 4, comprising T299K::T299D amino acid changes in the CH2 domains (underlined, bold-face), and T366S/L368A/Y407V/CH3 C-terminal Cysteine KSCDKT::T366W/CH3 C-terminal Cysteine GEC in the CH3 domains; FIG. 17.

SEQ ID NOs 396-397 are the aa sequence and nucleotide sequence of glycosylation mutant 5, comprising T299D::T299K amino acid changes in the CH2 domains (underlined, bold-face), and T366S/L368A/Y407V/CH3 C-terminal Cysteine KSCDKT::T366W/CH3 C-terminal Cysteine GEC in the CH3 domains; FIG. 17.

SEQ ID NOs 398-399 are the aa sequence and nucleotide sequence of glycosylation mutant 6, comprising T299D::T299D amino acid changes in the CH2 domains (underlined, bold-face), and T366S/L368A/Y407V/CH3 C-terminal Cysteine KSCDKT::T366W/CH3 C-terminal Cysteine GEC in the CH3 domains; FIG. 17.

### DETAILED DESCRIPTION

Provided herein are Tandem Fc Antibodies (“TFcAs”), e.g., Tandem Fc Bispecific

Antibodies (“TFcBAs”). The molecules may be used for treating a cell proliferative disorder, e.g., a cancer.

### DEFINITIONS

For convenience, the meaning of certain terms and phrases used in the specification, examples, and appended claims, are provided below.

“Aa modification” or “aa change” refers to one or more amino acid (aa) deletion, addition or substitution to an aa sequence. Aa sequence insertions include amino- and/or carboxyl-terminal fusions ranging in length from one residue to polypeptides containing a hundred or more residues, as well as intrasequence insertions of single or multiple aa residues. Intrasequence insertions may range generally from about 1 to 10 residues, e.g., 1 to 5, e.g., 1 to 3.

“AEM” or “association enhancing modification” refers to an aa modification made to a CH3 domain to enhance its association with another CH3 domain. An AEM may comprise one or more aa substitutions, deletions or additions in one or both Fcs of a TFc. AEMs are classified in modules, e.g., module 1 (“AEM 1”), wherein the modification to one of the two CH3 domains is referred to as AEM 1.1 and the modification to the other CH3 domain is referred to as AEM 1.2. For example, AEM 1.1 consists of the combination of substitutions T366S/L368A and Y407V and AEM 1.2 consists of the aa substitution T366W. When a CH3 domain comprises two or more aa modifications, e.g., aa substitutions, the modifications are separated from each other by a “/”. When referring to modifications in two CH3 domains, the modifications in each of the CH3 domains are separated by “::”.

“Amino acid substitution” refers to the replacement of one specific amino acid (“aa”) in a protein with another aa. A substitution may be a conservative substitution, as defined below. An “anti-c-Met binding site” refers to a binding site that binds specifically to human c-Met. An “anti-EGFR binding site” refers to a binding site that binds specifically to human EGFR.

“Antigen binding site” refers to a binding site that comprises the VH and/or VL domain of an antibody, or at least one CDR thereof, provided that the antigen binding site binds specifically to its target antigen. For example, an antigen binding site may comprise, consist essentially of, or consist of a VHCDR3 alone or together with a VHCDR2 and optionally a VHCDR1. In certain embodiments, an antigen binding site comprises a VH domain and a VL domain, which may be present on the same polypeptide or on two different polypeptides, e.g., the VH domain is present on a heavy chain and a VL domain is present on a light chain.

“Antigen-binding portion” of an antibody refers to one or more fragments of an antibody that retain the ability to specifically bind to an antigen (e.g., c-met or EGFR). It has been shown that the antigen-binding function of an antibody can be retained by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment which consists of a VH domain; and (vi) an isolated Complementarity Determining Region (“CDR”). Furthermore, although VL and VH are two domains of an Fv fragment, VL and VH are coded for by separate genes, they can be joined, using recombinant methods, by a synthetic linker that enables them to be made as a single protein chain in which the VL and VH regions pair to form monovalent proteins, known as single chain Fvs (scFvs) (see, e.g., U.S. Pat. No. 5,892,019). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

“Binding affinity” refers to the strength of a binding interaction and includes both the actual binding affinity as well as the apparent binding affinity. The actual binding affinity is a ratio of the association rate over the disassociation rate. The apparent affinity can include, for example, the avidity resulting from a polyvalent interaction. Dissociation constant (Kd), is typically the reciprocal of the binding affinity, and may be conveniently measured using a surface plasmon resonance assay (e.g., as determined in a BIACORE 3000 instrument (GE Healthcare) e.g., using recombinant EGFR as the analyte and an anti-EGFR antibody as the ligand) or a cell binding assay, each of which assays is described in Example 3 of U.S. Pat. No. 7,846,440.

“Binding moiety,” “binding domain,” or “binding site,” refers to the portion, region, or site of a binding polypeptide or, when so specified, of a heavy or light chain thereof, that is directly involved in mediating the specific binding of an antibody to a target molecule (i.e., an antigen). Exemplary binding domains include an antigen binding site, a receptor binding domain of a ligand, a ligand binding domain of a receptor or an enzymatic domain. In preferred embodiments, the binding domain comprises or consists of an antigen binding site (e.g., comprising a variable heavy (VH) chain sequence and variable light (VL) chain sequence or six CDRs from an antibody placed into alternative framework regions (e.g., human framework regions optionally comprising one or more aa substitutions). In certain embodiments, a binding site may be comprised essentially only of a VH or a VL chain sequence. A binding site may be entirely from one species, e.g., it has only sequences that derive from the germline sequences of one species. For example, a binding site may be human (i.e., from the human species), mouse, or rat. A binding site may also be humanized, i.e., the CDRs are from one species and the frameworks (FRs) are from another species. For example, a binding site may have CDRs that were derived from a mouse antibody and FRs that are from the human species. Certain humanized binding sites comprise mutations in one or more CDR to make the CDRs look more like the CDRs of the donor antibody. Certain humanized antibodies may also comprise mutations in one or more FR. Generally mutations in a binding site may enhance the affinity of binding of the binding site to its target antigen, and/or they may stabilize the binding site, e.g., to extend its half-life.

“CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of aa residues when compared against each other. The aa residues which encompass the CDRs as defined by each of the above cited references are set forth for comparison. As used herein, and if not otherwise specified, “CDR” is as defined by Kabat.

TABLE 1
CDR definitions

| | CDR Definitions | | |
|---|---|---|---|
| | Kabat¹ | Chothia² | MacCallum³ |
| VHCDR1 | 31-35 | 26-32 | 30-35 |
| VHCDR2 | 50-65 | 53-55 | 47-58 |
| VHCDR3 | 95-102 | 96-101 | 93-101 |
| VLCDR1 | 24-34 | 26-32 | 30-36 |
| VLCDR2 | 50-56 | 50-52 | 46-55 |
| VLCDR3 | 89-97 | 91-96 | 89-96 |

¹Residue numbering follows the nomenclature of Kabat et al., 1991,supra
²Residue numbering follows the nomenclature of Chothia et al., supra
³Residue numbering follows the nomenclature of MacCallum et al., supra

“CH1 domain” refers to the heavy chain immunoglobulin constant domain located between the VH domain and the hinge. It spans EU positions 118-215. A CH1 domain may be a naturally occurring CH1 domain, or a naturally occurring CH1 domain in which one or more amino acids (“aas”) have been substituted, added or deleted, provided that the CH1 domain has the desired biological properties. A desired biological activity may be a natural biological activity, an enhanced biological activity or a reduced biological activity relative to the naturally occurring sequence.

“CH2 domain” refers to the heavy chain immunoglobulin constant domain that is located between the hinge and the CH3 domain. As defined here, it spans EU positions 237-340. A CH2 domain may be a naturally occurring CH2 domain, or a naturally occurring CH2 domain in which one or more aas have been substituted, added or deleted, provided that the CH2 domain has the desired biological properties. A desired biological activity may be a natural biological activity, an enhanced biological activity or a reduced biological activity relative to that of the naturally occurring domain.

“CH3 domain” refers to the heavy chain immunoglobulin constant domain that is located C-terminally of the CH2 domain and spans approximately 110 residues from the N-terminus of the CH2 domain, e.g., about positions 341-446b (EU numbering system). A CH3 domain may be a naturally occurring CH3 domain, or a naturally occurring CH3 domain in which one or more aas have been substituted, added or deleted, provided that the CH3 domain has the desired biological properties. A desired biological activity may be a natural biological activity, an enhanced biological activity or a reduced biological activity relative to that of the naturally occurring domain. A CH3 domain may or may not comprise a C-terminal lysine. “CH4 domain” refers to the heavy chain immunoglobulin constant domain that is located C-terminally of the CH3 domain in IgM and IgE antibodies. A CH4 domain may be a naturally occurring CH4 domain, or a naturally occurring CH4 domain in which one or more aas have been substituted, added or deleted, provided that the CH4 domain has the desired biological properties. A desired biological activity may be a natural biological activity, an enhanced biological activity or a reduced biological activity relative to that of the naturally occurring domain.

“CL domain” refers to the light chain immunoglobulin constant domain that is located C-terminally to the VL domain. It spans about Kabat positions 107A-216. A CL domain may be a naturally occurring CL domain, or a naturally occurring CL domain in which one or more aas have been substituted, added or deleted, provided that the CL domain has the desired biological properties. A desired biological activity may be a natural biological activity, an enhanced biological activity or a reduced biological activity relative to that of the naturally occurring domain. A CL domain may or may not comprise a C-terminal lysine.

“c-Met” or “c-MET” refers to Mesenchymal-Epithelial Transition (MET) factor, which is also known as Hepatocyte Growth Factor Receptor (HGFR), Scatter Factor (SF) receptor, AUTS9, RCCP2, corresponds to Gene ID 4233, and has tyrosine-kinase activity. The primary single chain precursor protein is post-translationally cleaved to produce the alpha and beta subunits, which are disulfide linked to form the mature receptor. Two transcript variants encoding different isoforms have been found for this gene. HGF is the only known ligand for c-Met. The aa sequence of the human c-Met isoform a precursor is provided at Genbank Accession No. NP_{—}001120972.1 and isoform b precursor is provided at Genbank Accession No. NP_{—}000236.2.

“Conservative substitution” or “conservative amino acid substitution” refers to the replacement of one or more aa residues in a protein or a peptide with, for each particular pre-substitution aa residue, a specific replacement aa that is known to be unlikely to alter either the confirmation or the function of a protein or peptide in which such a particular aa residue is substituted for by such a specific replacement aa. Such conservative substitutions typically involve replacing one aa with another that is similar in charge and/or size to the first aa, and include replacing any of isoleucine (I), valine (V), or leucine (L) for each other, substituting aspartic acid (D) for glutamic acid (E) and vice versa; glutamine (Q) for asparagine (N) and vice versa; and serine (S) for threonine (T) and vice versa. Other substitutions are known in the art to be conservative in particular sequence or structural environments. For example, glycine (G) and alanine (A) can frequently be substituted for each other to yield a conservative substitution, as can be alanine and valine (V). Methionine (M), which is relatively hydrophobic, can frequently conservatively substitute for or be conservatively substituted by leucine or isoleucine, and sometimes valine. Lysine (K) and arginine (R) are frequently interchangeable in locations in which the significant feature of the aa residue is its charge and the differing pK's of these two basic aa residues are not expected to be significant. The effects of such substitutions can be calculated using substitution score matrices such PAM120, PAM-200, and PAM-250. Other such conservative substitutions, for example, substitutions of entire regions having similar hydrophobicity characteristics (e.g., transmembrane domains), are well known.

A “constant region” or domain of a light chain of an immunoglobulin is referred to interchangeably as a “CL,” “light chain constant region domain,” “CL region” or “CL domain.” A constant domain on a heavy chain (e.g. hinge, CH1, CH2 or CH3 domains) of an immunoglobulin is referred to interchangeably as a “CH,” “heavy chain constant domain,” “CH” region or “CH domain.” A variable domain on an immunoglobulin light chain is referred to interchangeably as a “VL,” “light chain variable domain,” “VL region” or “VL domain.” A variable domain on an immunoglobulin heavy chain is referred to interchangeably as a “VH,” “heavy chain variable domain,” “VH region” or “VH domain.”

“DiS” refers to the modification of a domain, e.g., a hinge or CH3 domain, that results in the addition of a Cysteine, which can form a disulfide bond with another Cysteine. A DiS may comprise one or more aa substitutions, deletions or additions in one or both Fcs of a TFc. DiSs are classified in modules, e.g., module 1 (“DiS 1”), wherein the modification to one of the two Fcs is referred to as DiS 1.1 and the modification to the other Fc is referred to as DiS 1.2. For example, DiS 1.1 consists of the substitution Y349C and DiS 1.2 consists of the aa substitution S354C.

“Domain” refers generally to a region, e.g., an independently folding, globular region or a non-globular region (e.g., a linker domain), of a heavy or light chain polypeptide which may comprise peptide loops (e.g., 1 to 4 peptide loops) that may be stabilized, for example, by a β-pleated sheet and/or an intrachain disulfide bond. The constant and variable regions of immunoglobulin heavy and light chains are typically folded into domains. In particular, each one of the CH₁, CH2, CH3, CH4, CL, VH and VL domains typically form a loop structure.

“EC₅₀” or “EC50” refers to the concentration of a molecule, e.g., a TFcA, that provides 50% of the maximal effect of the protein on a particular system such as a binding assay or a signal transduction pathway.

“EGFR” refers to Epidermal Growth Factor Receptor, which is also known as ErbB1, HER-1, mENA, and PIG61. EGFR is known to bind ligands including epidermal growth factor (EGF), transforming growth factor α (TGf-α), amphiregulin, heparin-binding EGF (hb-EGF), betacellulin, epiregulin and has Gene ID 1956 (Herbst, R. S., and Shin, D. M., Cancer 94 (2002) 1593-1611; Mendelsohn, J., and Baselga, J., Oncogene 19 (2000) 6550-6565). EGFR is transmembrane glycoprotein that is a member of the protein kinase superfamily that regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al., Ann. Oncology 14 (2003) 1346-1363; Tsao, A. S., and Herbst, R. S., Signal 4 (2003) 4-9; Herbst, R. S., and Shin, D. M., Cancer 94 (2002) 1593-1611; Modjtahedi, H., et al., Br. J. Cancer 73 (1996) 228-235). Binding of the ligand to EGFR induces receptor dimerization and tyrosine autophosphorylation, which leads to cell proliferation. Multiple alternatively spliced transcript variants that encode different protein isoforms have been found for this gene. The aa sequences for human EGFR isoforms a-d precursors are provided at Genbank Accession Nos. NP_{—}005219.2, NP_{—958439.1}, NP_{—}958440.1 and NP_{—}958441.1.

“ErbB2” or “HER2” refers to a putative tyrosine kinase growth factor receptor EGFR2, p185 HER2/NEU antigen, similar to the EGF receptor. The aa sequences for ErbB2 isoforms are provided at Genbank Accession Nos. NP_{—}004439.2 and NP_{—}001005862.1, and the nucleotide sequence has GeneID 2064.

“ErbB3” or “HER3” refers to a receptor tyrosine-protein kinase that is encoded by the human ERBB3 gene and has a role in protein amino acid phosphorylation. The aa sequences for ErbB3 isoforms are provided at Genbank Accession Nos. NP_{—}001973.2 and NP_{—}001005915.1, and the nucleotide sequence has GeneID 2065.

“ErbB4” or “HER4” plays a role in receptor tyrosine kinase signal transduction that regulates cellular proliferation and differentiation. The aa sequences for ErbB3 isoforms are provided at Genbank Accession Nos. NP_{—}001973.2 and NP_{—}001005915.1, and the nucleotide sequence has GeneID 2066.

The ERBB2, ERBB3, and ERBB4 genes encode heregulin/neuregulin receptors, members of the EGFR-related type I receptor tyrosine kinase subfamily. The encoded proteins form homo- and heterodimers, which complicates assignment of function: ERBB2 homodimers do not bind heregulin, but ERBB2/ERBB3 heterodimers do. Herstatin is a secreted alternative ERBB2 product, of the extracellular domain, that binds to p185ERBB2, disrupts ERBB2 dimers, reduces p185 phosphorylation, and inhibits growth. Human ERBB2 gene is located at 17p12-21. Overexpression of HER-2 correlates with poor prognosis in breast carcinoma.

“IGF1R” refers to the insulin-like growth factor 1 receptor. Exemplary human IGF1R nucleic acid and protein sequences are set forth in RefSeqGene Gene ID: 3480 and GenBank Accession Number: NP_{—}000866.1, respectively.

“IGF2R” refers to the insulin-like growth factor 2 receptor. Exemplary human IGF2R nucleic acid and protein sequences are set forth in RefSeqGene Gene ID: 3482 and GenBank Accession Number: NP_{—}000867.2, respectively.

“Insulin receptor” refers to the cellular receptor for insulin. Exemplary human insulin receptor nucleic acid and protein sequences are set forth in RefSeqGene Gene ID: 3643 and GenBank Accession Number: NP_{—}000199.2, respectively.

“c-MET” refers to the receptor for Hepatocyte Growth Factor. Exemplary human c-Met nucleic acid and protein sequences are set forth in RefSeqGene Gene ID: 4233 and GenBank Accession Number: NP_{—}001120972.1, respectively.

“RON” refers to the receptor for Macrophage-stimulating protein receptor. Exemplary human RON nucleic acid and protein sequences are set forth in RefSeqGene Gene ID: 4486 and GenBank Accession Number: NP_{—}002438.2, respectively.

“c-Kit” refers to v-kit Hardy-Zuckerman 4 feline sarcoma viral oncogene homolog. Exemplary human c-Kit nucleic acid and protein sequences are set forth in RefSeqGene Gene ID: 3815 and GenBank Accession Number: NP_{—}001087241.1, respectively.

“VEGFR1” refers to vascular endothelial growth factor 1. Exemplary human VEGFR1 nucleic acid and protein sequences are set forth in RefSeqGene Gene ID: 2321 and GenBank Accession Number: NP_{—}002010.2, respectively.

“VEGFR2” refers to vascular endothelial growth factor 2. Exemplary human VEGFR2 nucleic acid and protein sequences are set forth in RefSeqGene Gene ID: 3791 and GenBank Accession Number: NP_{—}002244.1, respectively.

“TNFR” refers to tumor necrosis factor receptor. Exemplary human TNFR nucleic acid and protein sequences are set forth in RefSeqGene Gene ID: 7132 and GenBank Accession Number: NP_{—}001056.1, respectively.

“FGFR1” refers to fibroblast growth factor receptor 1. Exemplary human FGFR1 nucleic acid and protein sequences are set forth in RefSeqGene Gene ID: 2260 and GenBank Accession Number: NP_{—}001167537.1, respectively.

“FGFR2” refers to fibroblast growth factor receptor 2. Exemplary human FGFR2 nucleic acid and protein sequences are set forth in RefSeqGene Gene ID: 2263 and GenBank Accession Number: NP_{—}001138390.1, respectively.

“FGFR3” refers to fibroblast growth factor receptor 3. Exemplary human FGFR3 nucleic acid and protein sequences are set forth in RefSeqGene Gene ID: 2261 and GenBank Accession Number: NP_{—}000133.1, respectively.

“FGFR4” refers to fibroblast growth factor receptor 4. Exemplary human FGFR4 nucleic acid and protein sequences are set forth in RefSeqGene Gene ID: 2264 and GenBank Accession Number: NP_{—}075252.2, respectively.

“PDGFR-alpha” refers to platelet-derived growth factor receptor alpha. Exemplary human PDGFR-alpha nucleic acid and protein sequences are set forth in RefSeqGene Gene ID: 5156 and GenBank Accession Number: NP_{—}006197.1, respectively.

“PDGFR-beta” refers to platelet-derived growth factor receptor beta. Exemplary human PDGFR-beta nucleic acid and protein sequences are set forth in RefSeqGene Gene ID: 5159 and GenBank Accession Number: NP_{—}002600.1, respectively.

“EpCAM” refers to epithelial cell adhesion molecule. Exemplary human EpCAM nucleic acid and protein sequences are set forth in RefSeqGene Gene ID: 4072 and GenBank Accession Number: NP_{—}002345.2, respectively.

“EphA2” refers to EPH receptor A2. Exemplary human EphA2 nucleic acid and protein sequences are set forth in RefSeqGene Gene ID: 1969 and GenBank Accession Number: NP_{—}004422.2, respectively.

“CEA” refers to carcinoembryonic antigen-related cell adhesion molecule 5. Exemplary human CEA nucleic acid and protein sequences are set forth in RefSeqGene Gene ID: 1048 and GenBank Accession Number: NP_{—}004354.2, respectively.

“CD44” refers to the cell-surface glycoprotein CD44. Exemplary human CD44 nucleic acid and protein sequences are set forth in RefSeqGene Gene ID: 960 and GenBank Accession Number: NP_{—}001189486.1, respectively.

“ALK” refers to the anaplastic lymphoma receptor tyrosine kinase. Exemplary human ALK nucleic acid and protein sequences are set forth in RefSeqGene Gene ID: 238 and GenBank Accession Number: NP_{—}004295.2, respectively.

“AXL” refers to the AXL receptor tyrosine kinase. Exemplary human AXL nucleic acid and protein sequences are set forth in RefSeqGene Gene ID: 558 and GenBank Accession Number: NP_{—}068713.2, respectively.

“EU” indicates that aa positions in a heavy chain constant region, including aa positions in the CH1, hinge, CH2, and CH3 domains, are numbered herein according to the EU index numbering system (see Kabat et al., in “Sequences of Proteins of Immunological Interest”, U.S. Dept. Health and Human Services, 5^{th} edition, 1991).

“Fab” refers to the antigen binding portion of an antibody, comprising two chains: a first chain that comprises a VH domain and a CH1 domain and a second chain that comprises a VL domain and a CL domain. Although a Fab is typically described as the N-terminal fragment of an antibody that was treated with papain and comprises a portion of the hinge region, it is also used herein as referring to a binding domain wherein the heavy chain does not comprise a portion of the hinge.

“Fc region” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge, a CH2 domain, and a CH3 domain. Two Fc regions that are dimerized are referred to as “Fc” or “Fc dimer.” An Fc region may be a naturally occurring Fc region, or a naturally occurring Fc region in which one or more aas have been substituted, added or deleted, provided that the Fc region has the desired biological properties. A desired biological activity may be a natural biological activity, an enhanced biological activity or a reduced biological activity relative to that of the naturally occurring domain.

“Framework region” or “FR” or “FR region” includes the aa residues that are part of the variable region, but are not part of the CDRs (e.g., using the Kabat definition of CDRs). Therefore, a variable region framework is between about 100-120 aas in length but includes only those aas outside of the CDRs. For the specific example of a heavy chain variable region and for the CDRs as defined by Kabat et al., 1991, ibid., framework region 1 corresponds to the domain of the variable region encompassing aas 1-30; framework region 2 corresponds to the domain of the variable region encompassing aas 36-49; framework region 3 corresponds to the domain of the variable region encompassing aas 66-94, and framework region 4 corresponds to the domain of the variable region from aas 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat.

“Full length antibody” or “full length Ab” is an antibody (“Ab”) that comprises one or more heavy chains and one or more light chains, which optionally may be connected. Each heavy chain is comprised of a heavy chain variable region (abbreviated herein as VH) and a heavy chain constant region. The heavy chain constant region is comprised of three domains CH1, CH2, and CH3, and optionally a fourth domain, CH4. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is typically composed of three CDRs and four FRs, arranged from amino-terminus to carboxyl-terminus in the following order: FR1, CDR1, FR2, CDR2, FR3, CDR3, and FR4. Immunoglobulin proteins can be of any type or class (e.g., IgG, IgE, IgM, IgD, IgA and IgY) or subclass (e.g., IgG1, IgG2, IgG3, IgG4, IgA1 and IgA2).

“Gly-Ser linker” or “Gly-Ser peptide” refers to a peptide that consists of glycine and serine residues. An exemplary Gly-Ser peptide comprises the aa sequence (Gly4 Ser)n, wherein n=1, 2, 3, 4, 5, 6, 7, 8, 9, 10, 11, 12, 13, 14, 15, 16, 17, 18, 19, 20 or more. In certain embodiments, n is a number between 1 and 5, n is a number between 6 and 10, n is a number between 11 and 15, n is a number between 16 and 20, n is a number between 21 and 25, or n is a number between 26 and 30.

“Hinge” or “hinge region” or “hinge domain” refers to the flexible portion of a heavy chain located between the CH1 domain and the CH2 domain. It is approximately 25 aas long, and is divided into an “upper hinge,” a “middle hinge” or “core hinge,” and a “lower hinge.” A hinge may be a naturally occurring hinge, or a naturally occurring hinge in which one or more aas have been substituted, added or deleted, provided that the hinge has the desired biological properties. A desired biological activity may be a natural biological activity, an enhanced biological activity or a reduced biological activity relative to the naturally occurring sequence.

A “hinge subdomain” refers to the upper hinge, middle (or core) hinge or the lower hinge. The aa sequences of the hinge subdomains of an IgG1, IgG2, IgG3 and IgG4 are set forth in Table 2:

TABLE 2
Listing of IgG hinge subdomains

| | Upper | Middle | Lower | Complete |
|---|---|---|---|---|
| IgG | hinge | hinge | hinge | hinge |
| IgG1 | EPKSCDKTHT | CPPCP | APELLG | SEQ ID |
| | (SEQ ID | (SEQ ID | (SEQ ID | NO: 4 |
| | NO: 1) | NO: 2) | NO: 3) | |
| IgG2 | ERKCCVE | CPPCP | APPVAGP | SEQ ID |
| | (SEQ ID | (SEQ ID | (SEQ ID | NO: 7 |
| | NO: 5) | NO: 2) | NO: 6) | |
| IgG3 | ELKTPL | CPRCP(EPKSC | APELLG | SEQ ID |
| | GDTTHT | DTPPPCPRCP)₃ | (SEQ ID | NO: 11 |
| | (SEQ ID | (SEQ ID | NO: 10) | |
| | NO: 8) | NO: 9) | | |
| IgG4 | ESKYGPP | CPSCP | APEFLG | SEQ ID |
| | (SEQ ID | (SEQ ID | (SEQ ID | NO: 15 |
| | NO: 12) | NO: 13) | NO: 14) | |

The complete hinge consists of the upper hinge subdomain, middle hinge subdomain and lower hinge subdomain in amino to carboxy terminal order and without intervening sequences.

“IC₅₀,” or “IC50” refers to the concentration of a molecule, e.g., a TFcA, that provides a 50% inhibition of a maximal activity (e.g., a response to a stimulus or a constitutive activity), i.e., a concentration that reduces the activity to a level halfway between the maximal activity and the baseline. The IC₅₀ value may be converted to an absolute inhibition constant (Ki) using, e.g., the Cheng-Prusoff equation. In a system that is inhibited by a binding agent, such as an antibody or a TFcA provided herein, the IC50 may be indistinguishable from the EC50.

“Inhibition” of a biological activity by a binding protein refers to any reproducibly detectable decrease in biological activity mediated by the binding protein. In some embodiments, inhibition provides a statistically significant decrease in biological activity, e.g., a decrease of about 5%, 10%, 20%, 30%, 40%, 50%, 60%, 70%, 80%, 90%, or 100% in biological activity relative to the biological activity determined in he absence of the binding protein.

“Isolated,” in reference to polynucleotides, polypeptides or proteins, means that the polynucleotide, polypeptide or protein is substantially removed from polynucleotides, polypeptides, proteins or other macromolecules with which it, or its analogues, occurs in nature. Although the term “isolated” is not intended to require a specific degree of purity, typically, the protein will be at least about 75% pure, more preferably at least about 80% pure, more preferably at least about 85% pure, more preferably at least about 90% pure, more preferably still at least about 95% pure, and most preferably at least about 99% pure. In certain embodiments, a TFcA, e.g., a TFcBA, is an isolated TFcA. In certain embodiments, a TFcA is a monoclonal TFcA.

“Kabat” in conjunction with designation of immunoglobulin aa sequence positions indicates that amino acid positions in a light chain constant region (e.g. CL domain) are numbered according to the Kabat index numbering system (see Kabat et al., 1991., op. cit.).

“Linked to” refers to direct or indirect linkage or connection of, in context, amino acids or nucleotides. An “indirect linkage” refers to a linkage that is mediated through a linker or a domain, comprising, e.g., one or more aas or nucleotides. A “direct linkage” or “linked directly” when referring to two polypeptide segments refers to the presence of covalent bond between the two polypeptide segments, e.g., the two polypeptide segments are joined contiguously without intervening sequences.

“Linker” refers to one or more aas connecting two domains or regions together. A linker may be flexible to allow the domains being connected by the linker to form a proper three dimensional structure thereby allowing them to have the required biological activity. A linker connecting the VH and the VL of an scFv is referred to herein as an “scFv linker” A linker connecting the N-terminus of a VH domain or the C-terminus of the CH3 domain to a second VH or VL domain, e.g., that of an scFv, is referred to as a “connecting linker”

“Module” refers to a structurally and/or functionally distinct part of a TFcA, such a binding site (e.g., an scFv domain or a Fab domain) and the TFc. Modules provided herein can be rearranged (by recombining sequences encoding them, either by recombining nucleic acids or by complete or fractional de novo synthesis of new polynucleotides) in numerous combinations with other modules to produce a wide variety of TFcAs, e.g., as disclosed herein. “Module” is also used to refer to the type of AEM or DiS modifications. In this context, and as further described herein, a “module” is one or a combination of two or more aa substitutions, additions or deletions that are made to enhance or favor the association or dimerization of the Fc regions comprising these modifications.

“Percent identical” or “% identical” refers to two or more nucleic acid or polypeptide sequences or subsequences that are the same (100% identical) or have a specified percentage of nucleotide or aa residues that are the same, when the two sequences are aligned for maximum correspondence and compared. To align for maximum correspondence, gaps may be introduced into one of the sequences being compared. The aa residues or nucleotides at corresponding positions are then compared and quantified. When a position in the first sequence is occupied by the same residue as the corresponding position in the second sequence, then the sequences are identical at that position. The percent identity between the two sequences is a function of the number of identical positions shared by the sequences (e.g., % identity=# of identical positions/total # of positions (e.g., overlapping positions)×100). In certain embodiments, the two sequences are the same length. The determination that one sequence is a measured % identical with another sequence can be determined using a mathematical algorithm. A non-limiting example of a mathematical algorithm utilized for such comparison of two sequences is incorporated in the ALIGN program (version 2.0) which is part of the GCG sequence alignment software package. When utilizing the ALIGN program e.g., for comparing aa sequences, a PAM120 weight residue table, a gap length penalty of 12, and a gap penalty of 4 may be used. Additional algorithms for sequence analysis are well known in the art and many are available online.

“Portion” or “fragment” (e.g., of a domain) of a reference moiety refers to a discrete part of the whole reference moiety (e.g., domain, e.g., a naturally occurring domain) that is at least, or at most 10% 20%, 30%, 40%, 50%, 60%, 70%, 80%, 90%, 95%, 98%, or 99% of the size of the reference moiety.

“scFv linker” refers to a peptide or polypeptide domain interposed between the VL and VH domains of an scFv. scFv linkers preferably allow orientation of the VL and VH domains in a antigen binding conformation. In one embodiment, an scFv linker comprises or consists of a peptide or polypeptide linker that only comprises glycines and serines (a “Gly-Ser linker”). In certain embodiments, an scFv linker comprises a disulfide bond.

“Similarity” or “percent similarity” in the context of two or more polypeptide sequences, refer to two or more sequences or subsequences that have a specified percentage of aa residues that are the same or conservatively substituted when compared and aligned for maximum correspondence. By way of example, a first aa sequence can be considered similar to a second aa sequence when the first aa sequence is at least 50%, 60%, 70%, 75%, 80%, 90%, 95%, 97%, 98% or even 99% identical, or conservatively substituted, to the second aa sequence when compared to an equal number of aas as the number contained in the first sequence, or when compared to an alignment of polypeptides that has been aligned by a computer similarity program known in the art. These terms are also applicable to two or more polynucleotide sequences.

“Specific binding,” “specifically binds,” “selective binding,” and “selectively binds,” as well as “binds specifically” “binds selectively,” when referring to the binding of a binding site to its target epitope or a combination of binding sites to their target epitopes, means that the binding site(s) exhibit(s) immunospecific binding to the target epitope(s). A binding site that binds specifically to an epitope exhibits appreciable affinity for a target epitope and, generally, does not exhibit cross-reactivity with other epitopes in that it does not exhibit appreciable affinity to any unrelated epitope and preferably does not exhibit affinity for any unrelated epitope that is equal to, greater than, or within two orders of magnitude lower than the affinity for the target epitope. “Appreciable” or preferred binding includes binding with a dissociation constant (Kd) of 10⁻⁸, 10⁻⁹ M, 10⁻¹⁰, 10⁻¹¹, 10⁻¹² M, 10⁻¹³ M or an even lower Kd value. Note that lower values for Kd (dissociation constant) indicate higher binding affinity, thus a Kd of 10⁻⁷ is a higher Kd value than a Kd of 10⁻⁸, but indicates a lower binding affinity than a Kd of 10⁻⁸. Dissociation constants with values of about 10⁻⁷M, and even as low as about 10⁻⁸M, are at the high end of dissociation constants suitable for therapeutic antibodies. Binding affinities may be indicated by a range of dissociation constants, for example, 10⁻⁶ to 10⁻¹² M, 10⁻⁷ to 10⁻¹²M, 10⁻⁸ to 10⁻¹²M or better (i.e., or lower value dissociation constant). Dissociation constants in the nanomolar (10⁻⁹ M) to picomolar (10⁻¹²M) range or lower are typically most useful for therapeutic antibodies. Suitable dissociation constants are Kds of 50 nM or less (i.e., a binding affinity of 50 nM or higher—e.g., a Kd of 45 nM) or Kds of 40 nM, 30 nM, 20 nM, 10 nM, 1 nM, 100 pM, 10 pM or 1 pM or less. Specific or selective binding can be determined according to any art-recognized means for determining such binding, including, for example, according to Scatchard analysis and/or competitive binding assays.

A “TFc” or “tandem Fc” refers to an entity comprising in an amino to carboxyl terminal order: a first Fc region, which is linked at its C-terminus to the N-terminus of a TFc linker, which is linked at its C-terminus to the N-terminus of a second Fc region, wherein the first and the second Fc regions associate to form an Fc.

“TFcA” refers to a tandem Fc antibody. A TFcA may be a monovalent or monospecific TFcA, e.g., comprising a single binding site. A TFcA may also be a bispecific TFcA, which is referred to herein as a TFcBA. A TFcA may be monoclonal.

“TFcBA” refers to a tandem Fc bispecific antibody, an artificial hybrid protein comprising at least two different binding moieties or domains and thus at least two different binding sites (e.g., two different antibody binding sites), wherein one or more of the pluralities of the binding sites are covalently linked, e.g., via peptide bonds, to each other. An exemplary TFcBA described herein is an anti-c-Met+anti-EGFR TFcBA, which is a polyvalent bispecific antibody that comprises a first binding site binding specifically to a c-Met protein, e.g., a human c-Met protein, and one or more second binding sites binding specifically to an EGFR protein, e.g., a human EGFR protein. When a TFcBA name comprises two antigens separated by a plus sign (+) this indicates that the binding sites for the two antigens may be in either relative amino to carboxy orientation in the molecule, whereas when the TFcBA name comprises two antigen binding site names separated by a slash (/) the antigen binding site to the left of the slash is amino terminal to the antigen binding site to the right of the slash. A TFcBA may be a bivalent binding protein, a trivalent binding protein, a tetravalent binding protein or a binding protein with more than 4 binding sites. An exemplary TFcBA is a bivalent bispecific antibody, i.e., an antibody that has 2 binding sites, each binding to a different antigen or epitope. In certain embodiments, the N-terminal binding site of a TFcBA is a Fab and the C-terminal binding site is an scFv.

### Tandem Fc Abs

Provided herein are Tandem Fc Antibodies (“TFcAs”), which may be monovalent or polyvalent, e.g., bivalent, trivalent, or tetravalent. TFcAs which are polyvalent may be monospecific, bispecific (“Tandem Fc Bispecific Abs” or “TFcBAs”) trispecific or tetraspecific TFcBAs. When a TFcBA is multispecific, it may be monovalent for one or more specificities.

In certain embodiments, a TFcA is a TFcBA. Exemplary TFcBAs inhibit ligand-induced signal transduction through one or both of the receptors targeted by the TFcBA and may thereby inhibit tumor cell proliferation or tumor growth. TFcBAs may also induce receptor downregulation or block receptor dimerization. Exemplary anti c-Met/anti-EGFR TFcBAs comprise a single anti-c-Met binding site (monovalent for anti-c-Met) and one or more anti-EGFR binding sites (monovalent or polyvalent for anti-EGFR). A TFc typically comprises a first Fc region linked to a second Fc region through a TFc linker, wherein the first and the second Fc regions dimerize to form an Fc.

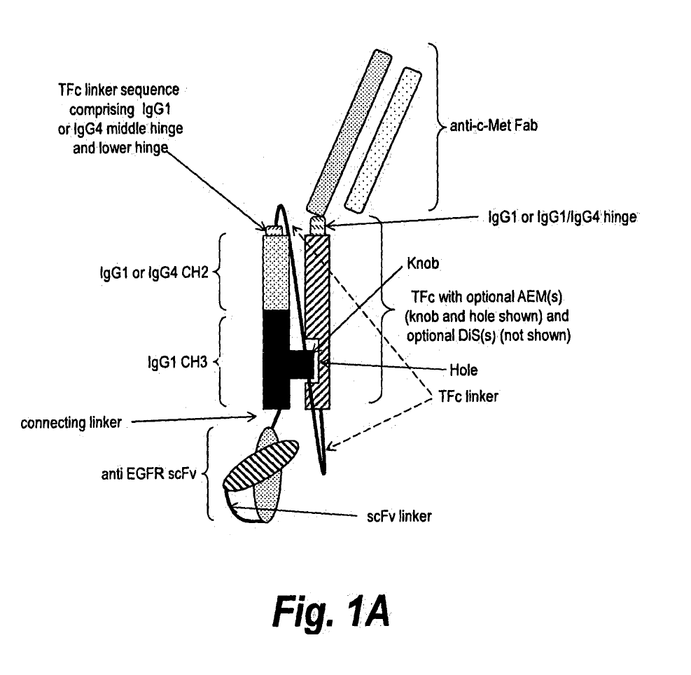

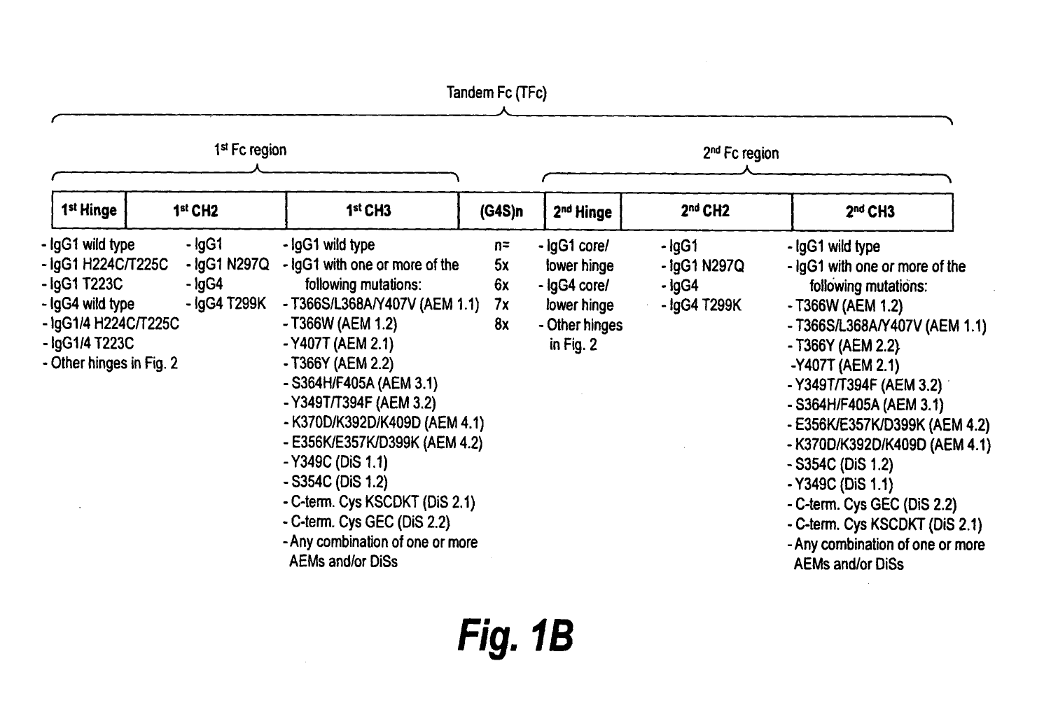

FIG. 1A and FIG. 1B show a diagram of an exemplary TFcBA showing the various elements of the molecule. As shown in FIGS. 1A and 1B, a TFcBA comprises a first binding site (e.g., an anti-c-Met Fab), a second binding site (e.g., an anti-EGFR scFv), and a tandem Fc (“TFc”) that links the first and the second binding sites together. A TFcBA may be described as containing three modules, wherein the first module comprises the first binding site, the second module comprises the TFc and the third module comprises the second binding site. A TFc generally comprises in a contiguous aa sequence a first Fc region, a TFc linker, and a second Fc region, wherein the TFc linker links the first Fc region to the second Fc region and allows the association of the two Fc regions. As illustrated in the exemplary TFcBA in FIG. 1A and FIG. 1B, each of the two Fc regions of a TFc may comprise a hinge, a CH2 domain and a CH3 domain. Each of these regions may be from the same immunoglobulin isotype, or from different isotypes. For example, the hinge, CH2 and CH3 domains may all be from IgG1, IgG2, IgG3 or IgG4, or certain domains or portions thereof may be from one immunoglobulin isotype and another domain or portion may be from another immunoglobulin isotype. For example, the TFcBA that is pictured in FIGS. 1A and 1B comprises all domains from IgG1, or alternatively, it may comprise an IgG1/IgG4 hybrid hinge, an IgG4 CH2 domain and an IgG1 CH3 domain. An Fc region preferably comprises human Fc domains, however, sequences from other mammals or animals may also be used, provided that the TFcBA retains its biological activity and is preferably not significantly immunogenic in a human subject.

In preferred embodiments, the first and/or the second Fc region comprise one or more modifications to enhance their association and/or to stabilize such association. In certain embodiments, the first and/or the second CH3 domains of a TFcA comprises one or more modification to enhance the association of the CH3 domains or Fcs comprising such. Such modifications are referred to herein as Association Enhancing Modifications or “AEMs.” Exemplary modifications include aa substitutions in both CH3 domains to enhance their interaction, e.g., knob/hole mutations.

In certain embodiments, the first and/or the second Fc region comprises an aa modification that results in the addition of one or more cysteines to the Fc region, to thereby form a disulfide bond with the other Fc region of the TFc. Such modifications are referred to herein as disulfide forming modifications or “DiS” modifications. DiS modifications may be present in the hinge, CH2 and/or CH3 domains.

A TFc may comprise one or more AEM and/or one or more DiS modifications. FIG. 1B shows exemplary modifications that can be made to either the CH3 region or the hinge. Fc regions may also comprise additional modifications, e.g., modifications that modulate a biological activity that is mediated through the Fc region, such as ADCC.

Although generally, the first and the second Fc regions comprise a hinge, a CH2 domain and a CH3 domain, in certain embodiments, an Fc region may comprise a CH3 domain and a CH2 domain, but no hinge. In other embodiments, an Fc region comprises a CH3 domain and a hinge, but does not comprise a CH2 domain. In other embodiments, an Fc region may comprise a CH3 domain and a CH4 domain, but does not comprise a CH2 domain nor a hinge. In other embodiments, an Fc region may comprise a CH3 domain, a CH4 domain, a CH2 domain, but does not comprise a hinge. In other embodiments, an Fc region may comprise a CH3 domain, a CH4 domain, a hinge, but does not comprise a CH2 domain. In certain embodiments, a portion of one or more domains is absent.

In certain embodiments, the first Fc region comprises an aa sequence that differs from that of the second Fc region in one or more aa addition, deletion or substitution (a “heterodimeric Fc”). This is often the case as AEM and DiS modifications, which typically introduce different modifications to the first and the second Fc region. In other embodiments, the first Fc region comprises the same aa sequence as the second Fc region (a “homodimeric Fc”).

In certain embodiments, an Fc domain (hinge, CH2 or CH3 domain) is directly linked to another Fc domain. For example, a hinge may be directly linked to a CH2 domain and/or a CH2 domain may be directly linked to a CH3 domain. In other embodiments, an Fc domain is linked to another Fc domain through a linker, which may be one or more aas long, provided that the TFcA comprising these domains has the desired biological activity and stability and any other desired characteristics.

In certain embodiments, a binding site is an antigen binding site, which comprises, e.g., a heavy chain variable (VH) domain and a light chain variable (VL) domain. The VH and VL domains generally contain 3 Complementarity Determining Regions (CDRs) each, although in certain embodiments, fewer than 6 CDRs may be sufficient for providing specific binding to an antigen. In certain embodiments, the VH domain is part of a Fab, in which case, the VH domain is linked to a CH1 domain, generally in the natural order, i.e., the VH domain is linked to the N-terminus of the CH1. When the antigen binding site is part of a Fab, the VL domain may be linked to a light chain constant (CL) domain, generally in the natural order, i.e., the VL domain is linked to the N-terminus of the CL domain.

The variable domains (VH and VL) may be linked directly or indirectly to the constant domains (CH1 and CL), e.g., through a linker, which may be one or more aas long, provided that the TFcA comprising these domains has the desired biological activity and stability and any other desired characteristics.

In certain embodiments, the VH domain is part of an scFv, in which case, the VH domain is linked to the VL domain through an scFv linker, and the scFv is linked to the N- and/or C-terminus of a TFc. When a binding site is an scFv, the variable regions are generally not linked to a CH1 or CL domain.

In certain embodiments, a TFcA is monovalent and monospecific. A monovalent TFcA may comprise a binding site at the amino terminus or at the C-terminus of the TFc. The binding site of a monovalent TFcA may be a Fab or an scFv. Exemplary heavy chains of monovalent TFcAs comprise in amino to carboxyl terminal order:
i) a VH domain and a TFc;
ii) a VH domain, a CH1 domain, and a TFc;
iii) a VH domain, an scFv linker, a VL domain, and a TFc;
iv) a TFc, a connecting linker and a VH domain;
v) a TFc, a connecting linker, a VH domain and a CH1 domain; and
vi) a TFc, a connecting linker, a VH domain, an scFv linker and a VL domain.

When a TFcA comprises a Fab, the TFcA also comprises a light chain comprising the VL domain of the Fab and optionally a CL domain.

In certain embodiments, a TFcA is a TFcBA. TFcBAs may comprise one Fab binding specifically to a first antigen and a second Fab binding specifically to a second antigen. TFcBAs may also comprise a first scFv binding specifically to a first antigen and a second scFv binding specifically to a second antigen. TFcBAs may also comprise a Fab binding specifically to a first antigen and an scFv binding specifically to a second antigen. In certain embodiments, the amino terminus of a TFc is connected to a Fab and the carboxyl terminus of the TFc is connected to an scFv. Alternatively, the amino terminus of a TFc is connected to an scFv and the carboxyl terminus of the TFc is connected to a Fab. Exemplary molecules have the following format: Fab-TFc-scFv; Fab-TFc-Fab; scFv-TFc-scFv; and scFv-TFc-Fab.

In one embodiment, a TFcBA comprises a heavy chain, which comprises in amino to carboxyl-terminal order:
(i) a first VH domain, a TFc, a connecting linker, and a second VH domain;
(ii) a first VH domain, a CH1 domain, a TFc, a connecting linker, and a second VH domain;
(iii) a first VH domain, a CH1 domain, a TFc, a connecting linker, a second VH domain, an scFv linker and a second VL domain, wherein the second VH and VL domains associate to form a second binding site;
(iv) a first VH domain, a TFc, a connecting linker, a second VH domain, and a CH1 domain;
(v) a first VH domain, a first CH1 domain, a TFc, a connecting linker, a second VH domain and a second CH1 domain;
(vi) a first VH domain, a first scFv linker, a first VL domain, a TFc, a connecting linker, and a second VH domain, wherein the first VL and VH domains associate to form a first binding site;
(vii) a first VH domain, a first scFv linker, a first VL domain, a TFc, a connecting linker, a second VH domain, and a CH1 domain, wherein the first VL and VH domains associate to form a first binding site; and
(viii) a first VH domain, a first scFv linker, a first VL domain, a TFc, a connecting linker, a second VH domain, a second scFv linker, and a second VL domain, wherein the first VH and VL domains form a first binding site and the second VH and VL domains form a second binding site.

A TFcBA of (i)-(v) may further comprise a light chain comprising a first VL domain and optionally a CL domain located at the C-terminus of the VL domain, wherein the first VH and VL domains associate to form a first binding site. A TFcBA of (i), (ii), (iv)-(vii) may comprise a light chain comprising a second VL domain and optionally a CL domain located at the C-terminus of the VL domain, wherein the first VH and VL domains associate to form a second binding site.

In certain embodiments, a heavy chain comprises a first VH domain, which is linked at its C-terminus to the N-terminus of a TFc, which is linked at its C-terminus to the N-terminus of a connecting linker, which is linked at its C-terminus to the N-terminus of a second VH domain. In certain embodiments, a heavy chain comprises a first VH domain, which is linked at its C-terminus to the N-terminus of a CH1 domain, which is linked at its C-terminus to the N-terminus of a TFc, which is linked at its C-terminus to the N-terminus of a connecting linker, which is linked at its C-terminus to the N-terminus of a second VH domain. In certain embodiments, a heavy chain comprises a first VH domain, which is linked at its C-terminus to the N-terminus of a CH1 domain, which is linked at its C-terminus to the N-terminus of a TFc, which is linked at its C-terminus to the N-terminus of a connecting linker, which is linked at its C-terminus to the N-terminus of a second VH domain, which is linked at its C-terminus to the N-terminus of an scFv linker, which is linked at its C-terminus to the N-terminus of a second VL domain, wherein the second VH and VL domains associate to form a second binding site. In certain embodiments, a heavy chain comprises a first VH domain, which is linked at its C-terminus to the N-terminus of a TFc, which is linked at its C-terminus to the N-terminus of a connecting linker, which is linked at its C-terminus to the N-terminus of a second VH domain, which is linked at its C-terminus to the N-terminus of a CH1 domain. In certain embodiments, a heavy chain comprises a first VH domain, which is linked at its C-terminus to the N-terminus of a first CH1 domain, which is linked at its C-terminus to the N-terminus of a TFc, which is linked at its C-terminus to the N-terminus of a connecting linker, which is linked at its C-terminus to the N-terminus of a second VH domain, which is linked at its C-terminus to the N-terminus of a second CH1 domain. In certain embodiments, a heavy chain comprises a first VH domain, which is linked at its C-terminus to the N-terminus of a first scFv linker, which is linked at its C-terminus to the N-terminus of a first VL domain, which is linked at its C-terminus to the N-terminus of a TFc, which is linked at its C-terminus to the N-terminus of a connecting linker, which is linked at its C-terminus to the N-terminus of a second VH domain, wherein the first VH and VL domains associate to form a first binding site. In certain embodiments, a heavy chain comprises a first VH domain, which is linked at its C-terminus to the N-terminus of a first scFv linker, which is linked at its C-terminus to the N-terminus of a first VL domain, which is linked at its C-terminus to the N-terminus of a TFc, which is linked at its C-terminus to the N-terminus of a connecting linker, which is linked at its C-terminus to the N-terminus of a second VH domain, which is linked at its C-terminus to the N-terminus of a CH1 domain, wherein the first VH and VL domains associate to form a first binding site. In certain embodiments, a heavy chain comprises a first VH domain, which is linked at its C-terminus to the N-terminus of a first scFv linker, which is linked at its C-terminus to the N-terminus of a first VL domain, which is linked at its C-terminus to the N-terminus of a TFc, which is linked at its C-terminus to the N-terminus of a connecting linker, which is linked at its C-terminus to the N-terminus of a second VH domain, which is linked at its C-terminus to the N-terminus of a second scFv linker, which is linked at its C-terminus to the N-terminus of a second VL domain, wherein the first VH and VL domains form a first binding site and the second VH and VL domains form a second binding site.

In certain embodiments, a VL domain is substituted for the VH domain and the VH domain is substituted for the VL domain in the constructs above.

When the heavy chain does not comprise either a first or a second VL domain, a VL domain may be provided by a light chain. A light chain may comprise a first or a second VL domain and optionally a CL domain. For example, an scFv may comprise in amino to carboxy terminal order a VL domain, an scFv linker and a VH domain.

In certain embodiments, a TFcBA comprises a first antigen binding site that binds specifically to a first receptor and a second antigen binding site that binds specifically to a second receptor. In certain embodiments, the first antigen binding site that binds specifically to the first receptor is a Fab and the second antigen binding site that binds specifically to the second receptor is an scFv. Exemplary combinations of binding sites are set forth in Table 3, wherein a “yes” indicates a possible combination and anti-c-Met+anti-EGFR TFcBAs are used to illustrate the possible combinations:

TABLE 3
Exemplary combinations of binding sites of anti-c-Met + anti-EGFR
TFcBAs

| | | Binding site linked to the N-terminus | | | |
|---|---|---|---|---|---|
| | | of the TFc | | | |
| | | Anti-c- | Anti-c- | Anti- | Anti- |
| | | Met | Met | EGFR | EGFR |
| | | scFv | Fab | scFv | Fab |
| Binding site | Anti-c-Met | yes | yes | yes | yes |
| linked to the | scFv | | | | |
| C-terminus | Anti-c-Met | yes | yes | yes | yes |
| of the TFc | Fab | | | | |
| | Anti-EGFR | yes | yes | yes | yes |
| | scFv | | | | |
| | Anti-EGFR | yes | yes | yes | yes |
| | Fab | | | | |

In certain embodiments, a TFcBA comprises more than 2 binding sites. A TFcBA may comprise 3, 4, 5, 6 or more binding sites. Additional binding sites may be linked, e.g., to the N- and/or C-terminus of a TFcA or TFcBA. For example, a heavy chain may comprise one or more Fabs and/or scFvs linked to the amino- or carboxyl-terminus of the TFc.

Exemplary domains of TFcBAs are further described below.

### Exemplary Hinges

In one embodiment, the first and/or the second Fc region of a TFcA, e.g., a TFcBA, comprises an IgG upper hinge, an IgG middle hinge and/or an IgG lower hinge. For example, an Fc region may comprise one or more IgG1 upper, middle and lower hinge, e.g., set forth in SEQ ID NOs:1, 2 and 3, respectively (see Table 2). Fc regions may also comprise one or more of an IgG2 upper, middle and lower hinge, e.g., set forth in SEQ ID NOs:5, 2 and 6, respectively (the middle hinge of IgG1 and IgG2 have the same aa sequence/see Table 2). Fc regions may also comprise one or more of an IgG3 upper, middle and lower hinge, e.g., set forth in SEQ ID NOs:8, 9 and 10, respectively (Table 2). Fc regions may also comprise one or more of an IgG4 upper, middle and lower hinge, e.g., set forth in SEQ ID NOs:12, 13 and 14, respectively (Table 2). Fc regions may also comprise one or more mouse Ig sequences or IgA1 or IgA2 sequences.

A first and/or a second Fc region of a TFcA may also comprise an aa sequence of an upper, middle, or lower hinge having an aa sequence that differs from a naturally occurring sequence, such as a sequence set forth herein (e.g., SEQ ID NOs:1-14) comprising up to 1, 2, 3, 4, or 5 aa modifications, e.g., aa substitutions, deletions or additions. For example, the following IgG1 upper hinges may be used:

EPKSCDKTCC (SEQ ID NO:16; corresponds to SEQ ID NO:1 with the aa substitutions H224C and T225C (underlined)) and
EPKSCDKCHT (SEQ ID NO:17; corresponds to SEQ ID NO:1 with the aa substitution T223C (underlined)).

The amino acid numbering of the hinge residues referred to herein is according to their numbering in a full length antibody (EU numbering; see FIG. 2).

In one embodiment, the first and/or the second hinge of a TFcA is a full length wild type IgG1 hinge comprising the following aa sequence:

(SEQ ID NO: 4)
EPKSCDKTHTCPPCPAPELLG.

The first and/or the second hinge of a TFcBA may also consist of an IgG1 hinge comprising up to 1, 2, 3, 4, or 5 aa modifications, e.g., aa substitutions, deletions or additions, relative to SEQ ID NO:4. For example, the following IgG1 hinges may be used:

EPKSCDKTCCCPPCPAPELLG (SEQ ID NO:18; corresponds to SEQ ID NO:4 with the aa substitutions H224C and T225C); and
EPKSCDKCHTCPPCPAPELLG (SEQ ID NO:19; corresponds to SEQ ID NO: 4 with the aa substitution T223C).

In one embodiment, the first and/or the second hinge of a TFcA is a hybrid hinge, i.e., a hinge that comprises portions from different IgG subclasses. In one embodiment, a hinge comprises an upper hinge from IgG1 and a middle and lower hinge from IgG4, and may, e.g., consist of the following aa sequence:

EPKSCDKTHTcpscpapeflg (SEQ ID NO:20; upper case residues represents IgG1 sequences and lower case residues represent IgG4 sequences).

The first and/or the second hinge of a TFcBA may also be a hybrid hinge comprising the aa sequence set forth in SEQ ID NO:20, comprising up to 1, 2, 3, 4, or 5 aa modifications, e.g., aa substitutions, deletions or additions. For example, the following IgG1/IgG4 hybrid hinges may be used:

EPKSCDKTCCcpscpapeflg (SEQ ID NO:21; corresponds to SEQ ID NO:20 with the aa substitutions H224C and T225C; upper case residues represent IgG1 sequences and lower case residues represent IgG4 sequences); and

EPKSCDKCHTcpscpapeflg (SEQ ID NO:22; corresponds to SEQ ID NO:20 with the aa substitution T223C).

In certain embodiments, the first and/or the second Fc region comprises a portion of a hinge instead of a full length hinge. For example, a first and/or a second Fc region of a TFcBA may comprise a hinge lacking the upper, middle and/or lower hinge. In certain embodiments, an Fc region comprises a middle and lower hinge, but does not comprise an upper hinge. An exemplary aa sequence of an IgG1 middle and lower hinge is the following:

(SEQ ID NO: 23)
CPPCPAPELLG.

An exemplary aa sequence of an IgG4 middle and lower hinge is the following:

(SEQ ID NO: 24)
CPSCPAPEFLG.

A summary of the aa numbers of the hinges and portions thereof provided above is set forth in Table 4. Alignments of the IgG1 and IgG1/IgG4 hybrid hinges are set forth in FIG. 2.

TABLE 4
SEQ ID NOs of exemplary
hinges and sub domains thereof

| | | | IgG1/IG4 |
|---|---|---|---|
| | | | hybrid |
| | IgG1 | IgG4 | hinge |
| Upper | SEQ ID | SEQ ID | SEQ ID |
| hinge | NO: 1 | NO: 12 | NO: 1 |
| Upper | SEQ ID | — | SEQ ID |
| hinge with | NO: 16 | | NO: 16 |
| H224C/T225C | | | |
| Upper hinge | SEQ ID | — | SEQ ID |
| with T223C | NO: 17 | | NO: 17 |
| Middle | SEQ ID | SEQ ID | SEQ ID |
| hinge | NO: 2 | NO: 13 | NO: 13 |
| Lower | SEQ ID | SEQ ID | SEQ ID |
| hinge | NO: 3 | NO: 14 | NO: 14 |
| Middle and | SEQ ID | SEQ ID | SEQ ID |
| lower hinge | NO: 23 | NO: 24 | NO: 24 |
| Complete | SEQ ID | SEQ ID | SEQ ID |
| hinge | NO: 4 | NO: 15 | NO: 20 |
| Complete | SEQ ID | — | SEQ ID |
| hinge with | NO: 18 | | NO: 21 |
| H224C/T225C | | | |
| Complete | SEQ ID | — | SEQ ID |
| hinge | NO: 19 | | NO: 22 |
| with T223C | | | |

Cysteines may also be introduced at positions other than T223, H224 and T225 in a hinge, e.g., by the substitution K222C, described in WO2010/064090.

Additional hinges that may be used in TFcAs include hIgG1 hinge variants comprising one of the following aa sequences (FIG. 2):

(SEQ ID NO: 263; hIgG1 Extra Prolines v1)
PPPPCDKTHTCPPCP
(SEQ ID NO: 264; hIgG1 Extra Prolines v2)
EPKSCPPPCPPCP
(SEQ ID NO: 265; hIgG1-like double core)
EPKSCPPCPCPPCP

Hinges that may be used in TFcAs may also include mouse hinge sequences, e.g., mIgG1 and mIgG2 sequences, and hybrids thereof. An exemplary mIgG1/mIgG2A hinge comprises the aa sequence

### VPRDCTIKPCPPCP (SEQ ID NO:267).

Other hinges that may be used in TFcAs comprise an IgG2 hinge or variant thereof, such as a variant comprising one of the following amino acid sequences (FIG. 2):

(SEQ ID NO: 268; hIgG2 C232P)
ERKPCVECPPCP
(SEQ ID NO: 269; hIgG2 C233P)
ERKCPVECPPCP

In certain embodiments, a TFcA comprises an IgA, e.g., IgA2, hinge or variant thereof. Exemplary IgA2 hinge variants include those comprising one of the following aa sequences (FIG. 2):

(SEQ ID NO: 271; hIgA2 Modified v1)
EPKSCPCPPPPPCCP
(SEQ ID NO: 272; hIgA2 modified v2)
EPKSCPCPPPPCCP
(SEQ ID NO: 273; hIgA2 Modified v3)
EPKSCPVPPPPPCCP

Other variations, e.g., aa modifications, may also be introduced into a hinge. For example, the substitution S228P may be made in the middle hinge of IgG4 to stabilize the interaction between two Fc regions comprising IgG4 middle hinges.

A TFcA comprising IgG2 sequences in its Fab domain may comprise the mutation C129S in the heavy chain portion of the Fab domain, which is a mutation of the cysteine that normally links the heavy chain to the light chain. Such a mutation will encourage the formation of a disulfide bridge between the light chain cysteine and C232 in the heavy chain, and C233 will pair with C233 of the neighboring hinge (in addition to the two disulfides in the CPPCP motif).

In certain embodiments, the following variant hinges are used: PRDCGCKPCICT (SEQ ID NO:248), PKSCGCKPCICT (SEQ ID NO:249), PKSCGCKPCICP (SEQ ID NO:250), PRDCGCKPCPPCP (SEQ ID NO:251), PRDCGCHTCPPCP (SEQ ID NO:252), PKSCDCHCPPCP (SEQ ID NO:253), and RKCCVECPPCP (SEQ ID NO:254).

In certain embodiments, a TFcBA does not comprise a first or a second hinge. For example, instead of a first hinge, a TFcBA may comprise a connecting linker that links the first binding site to the first CH2 domain. Such a linker may be a Gly-Ser linker as further described herein in the context of TFc linkers. In certain embodiments, a connecting linker comprises a (G₄S) 2 or (G₄S)₃ or (G₄S)₄ sequence. Other peptide sequences may also be used as a connecting linker provided that they provide the required flexibility and rigidity of certain parts of the linker. In certain embodiments, a TFcBA does not comprise a second hinge, but comprises a connecting linker instead, which may be a Gly-Ser linker similar to that of the TFc linker.

### Exemplary CH2 Domains

In certain embodiments, an Fc region comprises a CH2 domain. A CH2 domain may be from a human IgG1, IgG2, IgG3 or IgG4 or from a combination thereof (a “hybrid” CH2 domain). An exemplary full length wild type IgG1 CH2 domain consists of the following aa sequence:

(SEQ ID NO: 261)
GPSVFLFPPKPKDTLMISRTPEVTCVVVDVSHEDPEVKFNVVYVDG
VEVHNAKTKPREEQYNSTYRVVSVLTVLHQDWLNGKEYKCKVSNKA
LPAPIEKTISKAK.

An exemplary full length IgG1 CH2 domain with an N297Q substitution to reduce the glycosylation at that residue, such that the variant is substantially aglycosylated when expressed in a mammalian cell, consists of the following amino acid sequence:

(SEQ ID NO: 25)
GPSVFLFPPKPKDTLMISRTPEVTCVVVDVSHEDPEVKFNVVYVDG
VEVHNAKTKPREEQYQSTYRVVSVLTVLHQDWLNGKEYKCKVSNKA
LPAPIEKTISKAK.

An exemplary full length wild type IgG4 CH2 domain comprises the following aa sequence:

(SEQ ID NO: 262)
GPSVFLFPPKPKDTLMISRTPEVTCVVVDVSQEDPEVQFNVVYVDG
VEVHNAKTKPREEQFNSTYRVVSVLTVLHQDWLNGKEYKCKVSNKG
LPSSIEKTISKAK.

An exemplary full length IgG4 CH2 domain with a T299K substitution to reduce the glycosylation at residue 297, such that the variant is substantially aglycosylated when expressed in a mammalian cell, consists of the following amino acid sequence:

(SEQ ID NO: 26)
GPSVFLFPPKPKDTLMISRTPEVTCVVVDVSQEDPEVQFNVVYVDG
VEVHNAKTKPREEQFNSKYRVVSVLTVLHQDWLNGKEYKCKVSNKG
LPSSIEKTISKAK.

A CH2 domain may also comprise an aa sequence that differs from that of IgG1, IgG2, IgG3 or IgG4 in one or more aa modifications, e.g., aa deletions, additions or substitutions. In certain embodiments, a CH2 domain comprises an aa sequence that differs from that of a naturally occurring (or wild type) CH2 domain (e.g., SEQ ID NO:261 and 262) or from SEQ ID NOs:25 or 26 in at most, 1, 2, 3, 4, 5, 6, 7, 8, 9, 10, 15, 20, 25 or 30 aas. In certain embodiments, a CH2 domain comprises an aa sequence that is at least 70%, 75%, 80%, 85%, 90%, 95%, 97%, 98% or 99% identical or similar to that of a naturally occurring CH2 domain (e.g., SEQ ID NO:261 and 262) or SEQ ID NOs:25 or 26. Exemplary modifications include other modifications to reduce or remove glycosylation at aa 297. A modification may generally comprise an amino acid substitution in any of EU positions 297-299 (aa motif NXT) such that the variant is substantially aglycosylated when expressed in a mammalian cell. In addition to T299K, other substitutions that may be made at aa 299 to reduce glycosylation at aa 297 include T299S, T299A, T299N, T299G, T299Y, T299C, T299H, T299E, T299D, T299R, T299G, T299I, T299L, T299M, T299F, T299P, T299W, and T299V, as described, e.g., in WO/2005/018572.

Other aa changes may affect antibody effector functions, e.g., ADCC and CDC, or stability or other desired antibody characteristic. For example, FcγRI binding to an IgG1 Fc region may be modulated by modifying Leu235 and/or G1y237. Binding to Clq for CDC may be modulated by substitution of Ala330 and/or Pro331. Other modifications that may be made to CH2 domains to modulate the effector functions include substitutions at one or more aas at positions 234 to 238, 253, 279, 310, 318, 320, and 322.

### Exemplary CH3 Domains

In certain embodiments, the first and/or the second Fc region of a TFcA, e.g., a TFcBA, comprises a CH3 domain. A CH3 domain may be from a human immunoglobulin, e.g., an IgG1, IgG2, IgG3 or IgG4, or from a combination thereof (a “hybrid” CH3 domain). An exemplary full length wild type IgG1 CH3 domain comprises the following aa sequence:

(SEQ ID NO: 27)
GQPREPQVYTLPPSRDELTKNQVSLTCLVKGFYPSDIAVEWESNGQ
PENNYKTTPPVLDSDGSFFLYSKLTVDKSRWQQGNVFSCSVMHEAL
HNHYTQKSLSLSPGK.

In certain embodiments, variations of SEQ ID NO:27 may be used. For example, the C-terminal lysine of the CH3 domain may be deleted (see SEQ ID NO:28 in FIG. 3). In other embodiments, a CH3 domain comprises the aa substitutions D356E and L358M and the C-terminal lysine may be present or absent (SEQ ID Nos:29 and 30 respectively, and shown in FIG. 3).

In certain embodiments, the first and/or the second CH3 domains of a TFcA are modified to enhance the association of the first and the second Fc comprising the first and the second CH3 domains, respectively. Such CH3 modifications are referred to herein as Association Enhancing Modifications (“AEMs”). As further described in the Examples, it has been unexpectedly found that the addition of a TFc linker joining the two Fc regions of an Ab further enhances the ability of Abs having AEMs to properly assemble and increases their stability.

Exemplary AEM modifications that can be used are modifications that create “knobs-into-holes” and which are described, e.g., in U.S. Pat. No. 7,183,076. In this strategy, the CH3 domains are engineered to give one a protruding “knob” or “bump” and the other a complementary “hole,” thereby favoring the association of the CH3 domains. An exemplary aa modification to a CH3 domain that creates a “hole” is the combination of aa substitutions T366S, L368A and Y407V (e.g., in SEQ ID NOs:31-34; FIG. 3). Such a CH3 domain with a “hole” will dimerize favorably with a CH3 domain having a “knob” or “bump,” e.g., a CH3 domain comprising the amino acid substitution T366W (e.g., in SEQ ID NOs:35-38; FIG. 3). This pair of knob/hole mutations is referred to herein as “AEM module 1” or “AEM 1,” of which the first and the second CH3 domains are referred to as “AEM 1.1” and “AEM 1.2,” respectively.

In another embodiment, one of the two CH3 domains of a TFcA comprises a hole created by the substitution Y407T (e.g., in SEQ ID NOs:39-42; FIG. 3) and the other CH3 domain comprises a knob created by the substitution T366Y (e.g., in SEQ ID NOs:43-46; FIG. 3). This second pair of knob/hole mutations is referred to as “AEM module 2” or “AEM 2,” of which the first and the second CH3 domains are referred to as “AEM 2.1” and “AEM 2.2,” respectively.

The association between two CH3 domains may also be enhanced by mechanisms other than those creating typical knob/holes, e.g., by electrostatic modifications. In one embodiment, one of the two CH3 domains of a TFcA comprises the combination of substitutions S364H and F405A (e.g., in SEQ ID NOs:47-50; FIG. 3) and the other CH3 domain comprises the combination of substitutions Y349T and T394F (e.g., in SEQ ID NOs:51-54; FIG. 3). This third pair of modifications is referred to herein as “AEM module 3” or “AEM 3,” of which the first and the second CH3 domains are referred to as “AEM 3.1” and “AEM 3.2,” respectively.

In one embodiment, one of the two CH3 domains of a TFcA comprises the combination of substitutions K370D, K392D and K409D (e.g., in SEQ ID NOs:55-58; FIG. 3) and the other CH3 domain comprises the combination of substitutions E(or D)356K, E357K and D399K (e.g., in SEQ ID NOs:59-62; FIG. 3). This fourth pair of modifications is referred to herein as “AEM module 4” or “AEM 4,” of which the first and the second CH3 domains are referred to as “AEM 4.1” and “AEM 4.2,” respectively. The aa at position 356 may be either an E or a D, depending on the sequence that is used, and therefore the substitution at that position is referred to as “E (or D)356.”

In certain embodiments, the first and/or the second CH3 domains of a TFcA comprise one or more aa modifications resulting in the addition of one or more Cysteines that allow the formation of one or more disulfide bonds between the two CH3 or Fc domains. In one embodiment, one of the two CH3 domains of a TFcA comprises the substitution Y349C (e.g., in SEQ ID NOs:63-66; FIG. 3) and the other CH3 domain comprises the substitution S354C (e.g., in SEQ ID NOs:67-70; FIG. 3). This pair of disulfide forming modifications is referred to herein as “DiS module 1” or “DiS 1,” of which the first and the second CH3 domains are referred to as “DiS 1.1” and “DiS 1.2,” respectively.

In other embodiments, a cysteine is added to the C-terminus of each of the two CH3 domains of a TFcA, to thereby form a disulfide bond between the two CH3 domains. For example, one of the two CH3 domains may comprise the substitution of the carboxyl terminal aas “PGK” with “KSCDKT” (e.g., in SEQ ID NOs:71-72; FIG. 3) and the other CH3 domain may comprise the substitution of the carboxyl-terminal aas “PGK” with “GEC” (e.g., in SEQ ID NOs:73-74; FIG. 3).

In certain embodiments, a CH3 domain comprises a combination of two or more aa change(s). For example, one or more AEMs may be combined with one or more DiS modifications. In an exemplary embodiment, a CH3 domain comprises the hole mutations T366S, L368A, Y407V and the disulfide bond generating mutation Y349C (AEM 1.1+DiS 1.1). Such a CH3 domain may be combined in a TFc with a CH3 domain comprising the knob mutation T366W and the disulfide bond generating mutation 5354C (AEM 1.2+DiS 1.2). Exemplary aa sequences comprising this combination of substitutions include SEQ ID NOs:75-82 (FIG. 3).

Exemplary combinations of AEMs and DiSs that are made in CH3 domains to favor the association of CH3 domains or Fc regions comprising these are set forth in Table 5, wherein a “yes” indicates a combination that may be used.

TABLE 5
Exemplary combinations of AEM and DiS modifications

| | | | AEM module 1 | | | | | | AEM module 4 | |
|---|---|---|---|---|---|---|---|---|---|---|
| | | | 1.1 | | | | AEM module 3 | | 4.1 | 4.2 |
| | | | T366S/ | | AEM module 2 | | 3.1 | 3.2 | K370D/ | D356K*/ |
| | | | L368A/ | 1.2 | 2.1 | 2.2 | S364H/ | Y349T/ | K392D/ | E357K/ |
| | | | Y470V | T366W | Y407T | T366Y | F405A | T394F | K409D | D399K |
| DiS 1 | 1.1 | Y349C | yes | yes | yes | yes | yes | yes | yes | yes |
| | 1.2 | S354C | yes | yes | yes | yes | yes | yes | yes | yes |
| DiS 2 | 2.1 | C. term. | yes | yes | yes | yes | yes | yes | yes | yes |
| | | KSCDKT | | | | | | | | |
| | 2.2 | C term. | yes | yes | yes | yes | yes | yes | yes | yes |
| | | GEC | | | | | | | | |

*With respect to the sequences that have an E at position 356 (e.g., SEQ ID NOs: 29 and 30), this mutation is E356K.

Aa sequences of exemplary IgG1 CH3 domains with an AEM and/or DiS comprise SEQ ID NOs:31-98. An alignment of these aa sequences is provided in FIG. 3, and a description of these sequences is provided in Table 6. The CH3 domains in Table 6 and FIG. 3 are organized according to their AEM module (number 1, 2, 3 or 4) and their DiS module (number 1 or 2). Compatible CH3 domains are listed as “1” and “2” preceded by the module number.

TABLE 6
SEQ ID NOs for IgG1 CH3 domains with an AEM and/or a DiS

| | | | | | Wild type with |
|---|---|---|---|---|---|
| | | | | | D356E and |
| | | | | | L358M; |
| | | | Wild type without | Wild type with | without a |
| | | Wild type | terminal lysine | D356E and L358M | terminal lysine |
| | — | SEQ ID | SEQ ID NO:28 | SEQ ID NO:29 | SEQ ID NO:30 |
| | | NO:27 | | | |
| AEM | AEM 1.1 T366S/L368A/ | SEQ ID | SEQ ID NO:32 | SEQ ID NO:33 | SEQ ID NO:34 |
| 1 | Y407V | NO:31 | | | |
| | AEM 1.2 T366W | SEQ ID | SEQ ID NO:36 | SEQ ID NO:37 | SEQ ID NO:38 |
| | | NO:35 | | | |
| AEM | AEM 2.1 Y407T | SEQ ID | SEQ ID NO:40 | SEQ ID NO:41 | SEQ ID NO:42 |
| 2 | | NO:39 | | | |
| | AEM 2.2 T366Y | SEQ ID | SEQ ID NO:44 | SEQ ID NO:45 | SEQ ID NO:46 |
| | | NO:43 | | | |
| AEM | AEM 3.1 S364H/F405A | SEQ ID | SEQ ID NO:48 | SEQ ID NO:49 | SEQ ID NO:50 |
| 3 | | NO:47 | | | |
| | AEM 3.2 Y349T/T394F | SEQ ID | SEQ ID NO:52 | SEQ ID NO:53 | SEQ ID NO:54 |
| | | NO:51 | | | |
| AEM | AEM 4.1 K370D/K392D/ | SEQ ID | SEQ ID NO:56 | SEQ ID NO:57 | SEQ ID NO:58 |
| 4 | K409D | NO:55 | | | |
| | AEM 4.2 D356K*/E357K/ | SEQ ID | SEQ ID NO:60 | SEQ ID NO:61 | SEQ ID NO:62 |
| | D399K | NO:59 | | | |
| DiS 1 | DiS 1.1 Y349C | SEQ ID | SEQ ID NO:64 | SEQ ID NO:65 | SEQ ID NO:66 |
| | | NO:63 | | | |
| | DiS 1.2 S354C | SEQ ID | SEQ ID NO:68 | SEQ ID NO:69 | SEQ ID NO:70 |
| | | NO:67 | | | |
| DiS 2 | DiS 2.1 | SEQ ID | — | SEQ ID NO:72 | — |
| | C. term. KSCDKT | NO:71 | | | |
| | DiS 2.2 | SEQ ID | — | SEQ ID NO:74 | — |
| | C. term. GEC | NO:73 | | | |
| AEM | AEM 1.1 + DiS 1.1 | SEQ ID | SEQ ID NO:76 | SEQ ID NO:77 | SEQ ID NO:78 |
| 1 + | Y349C/T366S/ | NO:75 | | | |
| DiS 1 | L368A/Y407V | | | | |
| | AEM 1.2 + DiS 1.2 | SEQ ID | SEQ ID NO:80 | SEQ ID NO:81 | SEQ ID NO:82 |
| | S354C/T366W | NO:79 | | | |
| AEM | AEM 1.1 + DiS 2.1 | SEQ ID | — | SEQ ID NO:84 | — |
| 1 + | T366S/L368A/ | NO:83 | | | |
| DiS 2 | Y407V/C. term. KSCDKT | | | | |
| | AEM 1.2 + DiS 2.2 | SEQ ID | — | SEQ ID NO:86 | — |
| | T366W/C term. GEC | NO:85 | | | |
| AEM | AEM 1.1 + DiS 2.2 | SEQ ID | — | SEQ ID NO:88 | — |
| 1 + | T366S/L368A/ | NO:87 | | | |
| DiS | Y407V/C term. GEC | | | | |
| 2Inv | AEM 2.1 + DiS 2.1 | SEQ ID | — | SEQ ID NO:90 | — |
| | T366W/C. term. KSCDKT | NO:89 | | | |
| AEM | AEM 3.1 + DiS 2.1 | SEQ ID | — | SEQ ID NO:92 | — |
| 3 + | S364H/F405A/C term. | NO:91 | | | |
| DiS 2 | KSCDKT | | | | |
| | AEM 3.2 + DiS 2.2 | SEQ ID | — | SEQ ID NO:94 | — |
| | Y349T/T394F/C term. GEC | NO:93 | | | |
| AEM | AEM 4.1 + DiS 2.1 | SEQ ID | — | SEQ ID NO:96 | — |
| 4 + | K370D/K392D/ | NO:95 | | | |
| DiS 2 | K409D/C. term. KSCDKT | | | | |
| | AEM 4.2 + DiS 2.2 | SEQ ID | — | SEQ ID NO:98 | — |
| | D356K*/E357K/ | NO:97 | | | |
| | D399K/C term. GEC | | | | |

*With respect to the sequences that have an E at position 356 (e.g., SEQ ID NOs:29 and 30), this mutation is E356K.

Other CH3 AEMs that may be used in TFcAs include the following pairs of aa modifications, wherein the substitution(s) to the first and the second member of a pair of AEM modifications are separated by “and”:

1) F405A and T394E; S364D and Y349K; S364E and L368K; S364E Y349K; S364F and K3700; S364H and Y349K; S364H and Y349T; S364Y and K370G; T411K and K370E; V397S/F405A and T394F; K370/T411K and K370E/T411E; L351E/S364D and Y349K/L351K; L351E/S364E and Y349K/L351K; L351E/T366D and L351K/F366K; P395T/V397S/F405A and T394E; S364D/K370G and S364Y/K370R; S364D/T394F and Y349K/F405A; S364E/F405A and Y349K/T394E; S364E/F405S and Y349K/T394Y; S364E/F411E and Y349K/D401K; S364H/D401K and Y349T/F411E; S364H/T394F and Y349T/F405A; Y349C/S364E and Y349K/S354C; L351E/S364D/F405A and Y349K/L351K/T394F; L351K/S364H/D401K and Y349T/L351E/T411E; S364E/T411E/F405A and Y349K/T394F/D401K; S364H/D401K/F405A and Y349T/T394F/T411E; S364H/F405A/T411E and Y349T/F394F/D401K (WO2011/028952).
2) T366W and Y407A; T366W and T366S; L368A and Y407Y; K409E and D399K; K409E and D399R; and K409D and D399K; K409D and D399; K392E and D399R; K392E and D399K; K392D and D399R; and K392D and D399R (WO2009/089004).
3) T366W and Y407A; F405A and T394W; Y407T and T366Y; T366Y/F405A and T394W/Y407T; T366W/F405W and T394S/Y407A; F405W/Y407A and T366W/T394S; and F405W and T394S (U.S. Pat. No. 7,642,228).

Generally, any other AEM or DiS described in the art may be used.

A CH3 domain may also comprise an aa sequence that differs from that of a CH3 aa sequence provided herein, e.g., SEQ ID NOs:27-98, in at most, 1, 2, 3, 4, 5, 6, 7, 8, 9, 10, 15, 20, or 30 aas. In certain embodiments, a CH3 domain comprises an aa sequence that is at least 70%, 75%, 80%, 85%, 90%, 95%, 97%, 98% or 99% identical to a CH3 aa sequence provided herein, e.g., SEQ ID NOs:27-98. As several antibody effector functions, e.g., ADCC and CDC are mediated at least in part through areas in CH3 domains, aa changes that can be made to CH3 domains include changes affecting the effector function(s) of Fc regions. Exemplary modifications that may be made to CH3 domains are further described herein.

### Exemplary Fc Regions

An Fc region of a TFcA comprises one or more of a hinge, CH2 domain, CH3 domain and CH4 domain, which may be full length or not and which may be wild type or with aa modifications. The Fc domains may be from human immunoglobulins (“Igs”) or non-human Igs, e.g., mouse Igs, and may be from any type or isotype of an Ig, such as an IgG (e.g., IgG1, IgG2, IgG3 and IgG4) or IgA (e.g., IgA1 and IgA2).

In certain embodiments, a TFcA comprises a TFc that comprises a first and/or a second Fc region from a human immunoglobulin, e.g., IgG1. An Fc region preferably comprises in a contiguous amino to carboxyl terminal order: an IgG1 hinge or portion thereof (e.g., a core and lower hinge), an IgG1 CH2 domain and an IgG1 CH3 domain. Fc regions may comprise any combination of an IgG1 hinge (or portion thereof), IgG1 CH2 domain and IgG1 CH3 domains set forth herein, provided that the TFcA has the desired activity and stability.

In certain embodiments, a TFc comprises a first and/or a second Fc region that is a hybrid Fc region. A hybrid Fc region may comprise Fc domains from two or more IgG subclasses IgG1, IgG2, IgG3 and IgG4. In one embodiment, a hybrid Fc region comprises in a contiguous amino to carboxyl terminal order: an IgG1 upper hinge, an IgG4 middle and lower hinge, an IgG4 CH2 domain and an IgG1 CH3 domain. Exemplary IgG1/IgG4 hybrid Fc regions may comprise any combination of an IgG1 upper hinge, IgG4 core hinge, IgG4 lower hinge, IgG 4 CH2 domain and IgG1 CH3 domains set forth herein, provided that the TFcA comprising the TFc has the desired activity and stability.

In certain embodiments, an Fc region does not comprise a full length hinge. For example, a TFcBA may comprise a first Fc region comprising a full length hinge and a second Fc region comprising a hinge that consists of the core and/or lower hinge and does not comprise an upper hinge.

In certain embodiments, a TFc comprises a hinge that has been modified to comprise a cysteine for forming a disulfide bond with another cysteine in the other Fc region, to thereby stabilize the TFc. In one embodiment, an IgG1 hinge comprises the substitutions H224C and T225C (e.g., SEQ ID NO:18; FIG. 2). In another embodiment, a hinge comprises the substitution T223C (e.g., SEQ ID NO: 19; FIG. 2).

In one embodiment, a TFcA comprises an IgG1 TFc comprising in amino to carboxyl terminal order: i) an IgG1 hinge selected from the group of hinges consisting of the aa sequences set forth in SEQ ID NO:4 (full length IgG1 hinge), SEQ ID NO:18 (SEQ ID NO:4 with H224C/T225C), SEQ ID NO:19 (SEQ ID NO:4 with T223C) and SEQ ID NO:23 (middle and lower IgG1 hinge only); ii) an IgG1 CH2 domain comprising SEQ ID NO:261 or 25; and iii) a CH3 domain comprising an aa sequence selected from the group of CH3 domains consisting of an aa sequence set forth in SEQ ID NOs:31-98 (FIG. 3). In another embodiment, a TFcA comprises an IgG1/IgG4 hybrid TFc comprising in amino to carboxyl terminal order: i) a hinge selected from the group of hinges consisting of an aa sequence set forth in SEQ ID NO:20 (IgG1 upper hinge and IgG4 core and lower hinge), SEQ ID NO:21 (SEQ ID NO:20 with H224C/T225C), SEQ ID NO:22 (SEQ ID NO:20 with T223C) and SEQ ID NO:24 (middle and lower IgG4 hinge only); ii) an IgG4 CH2 domain comprising SEQ ID NO:262 or 26; and iii) a CH3 domain comprising an aa sequence selected from the group of CH3 domains consisting of the aa sequences SEQ ID NOs:31-98 (FIG. 3). Exemplary combinations of hinges and CH3 domains are set forth in Table 7, wherein a “yes’ indicates a combination that may be used, and wherein compatible modifications are separated from others by a blank row.

TABLE 7
Exemplary combinations of hinges and CH3 domains for forming Fc regions

| | | Hinge | | | | | | | |
|---|---|---|---|---|---|---|---|---|---|
| | | | IgG1 | IgG1 | Partial | | Hybrid | Hybrid | Partial |
| | | IgG1 | H224C/T225C | T223C | IgG1 | Hybrid | H224C/T225C | T223C | IgG4 |
| | | SEQ ID | SEQ ID | SEQ ID | SEQ ID | SEQ ID | SEQ ID | SEQ ID | SEQ ID |
| | | NO: 1 | NO: 18 | NO: 19 | NO: 23 | NO: 20 | NO: 21 | NO: 22 | NO: 24 |
| CH3 | AEM 1.1 | yes | yes | yes | yes | yes | yes | yes | yes |
| | SEQ ID | | | | | | | | |
| | NOs: 31-34 | | | | | | | | |
| | AEM 1.2 | yes | yes | yes | yes | yes | yes | yes | yes |
| | SEQ ID | | | | | | | | |
| | NOs: 35-38 | | | | | | | | |
| | AEM 2.1 | yes | yes | yes | yes | yes | yes | yes | yes |
| | SEQ ID | | | | | | | | |
| | NOs: 39-42 | | | | | | | | |
| | AEM 2.2 | yes | yes | yes | yes | yes | yes | yes | yes |
| | SEQ ID | | | | | | | | |
| | NOs: 43-46 | | | | | | | | |
| | AEM 3.1 | yes | yes | yes | yes | yes | yes | yes | yes |
| | SEQ ID | | | | | | | | |
| | NOs: 47-50 | | | | | | | | |
| | AEM 3.2 | yes | yes | yes | yes | yes | yes | yes | yes |
| | SEQ ID | | | | | | | | |
| | NOs: 51-54 | | | | | | | | |
| | AEM 4.1 | yes | yes | yes | yes | yes | yes | yes | yes |
| | SEQ ID | | | | | | | | |
| | NOs: 55-58 | | | | | | | | |
| | AEM 4.2 | yes | yes | yes | yes | yes | yes | yes | yes |
| | SEQ ID | | | | | | | | |
| | NOs: 59-62 | | | | | | | | |
| | DiS 1.1 | yes | yes | yes | yes | yes | yes | yes | yes |
| | SEQ ID | | | | | | | | |
| | NOs: 63-66 | | | | | | | | |
| | DiS 1.2 | yes | yes | yes | yes | yes | yes | yes | yes |
| | SEQ ID | | | | | | | | |
| | Nos: 67-70 | | | | | | | | |
| | DiS 2.1 | yes | yes | yes | yes | yes | yes | yes | yes |
| | SEQ ID | | | | | | | | |
| | NOs: 71-72 | | | | | | | | |
| | DiS 2.2 | yes | yes | yes | yes | yes | yes | yes | yes |
| | SEQ ID | | | | | | | | |
| | NOs: 73-74 | | | | | | | | |
| | AEM 1.1 | yes | yes | yes | yes | yes | yes | yes | yes |
| | DiS 1.1 | | | | | | | | |
| | SEQ ID | | | | | | | | |
| | NOs: 75-78 | | | | | | | | |
| | AEM 1.2 | yes | yes | yes | yes | yes | yes | yes | yes |
| | DiS 1.2 | | | | | | | | |
| | SEQ ID | | | | | | | | |
| | NOs: 79-82 | | | | | | | | |
| | AEM 1.1 | yes | yes | yes | yes | yes | yes | yes | yes |
| | DiS 2.1 | | | | | | | | |
| | SEQ ID | | | | | | | | |
| | NOs: 83-84 | | | | | | | | |
| | AEM 1.2 | yes | yes | yes | yes | yes | yes | yes | yes |
| | DiS 2.2 | | | | | | | | |
| | SEQ ID | | | | | | | | |
| | NOs: 85-86 | | | | | | | | |
| | AEM 1.1 | yes | yes | yes | yes | yes | yes | yes | yes |
| | DiS 2.2 | | | | | | | | |
| | SEQ ID | | | | | | | | |
| | NOs: 87-88 | | | | | | | | |
| | AEM 1.2 | yes | yes | yes | yes | yes | yes | yes | yes |
| | DiS 2.1 | | | | | | | | |
| | SEQ ID | | | | | | | | |
| | NOs: 89-90 | | | | | | | | |
| | AEM 3.1 | yes | yes | yes | yes | yes | yes | yes | yes |
| | DiS 2.1 | | | | | | | | |
| | SEQ ID | | | | | | | | |
| | NOs: 91-92 | | | | | | | | |
| | AEM 3.2 | yes | yes | yes | yes | yes | yes | yes | yes |
| | DiS 2.2 | | | | | | | | |
| | SEQ ID | | | | | | | | |
| | NOs: 93-94 | | | | | | | | |
| | AEM 4.1 | yes | yes | yes | yes | yes | yes | yes | yes |
| | DiS 2.1 | | | | | | | | |
| | SEQ ID | | | | | | | | |
| | NOs: 95-96 | | | | | | | | |
| | AEM 4.2 | yes | yes | yes | yes | yes | yes | yes | yes |
| | DiS 2.2 | | | | | | | | |
| | SEQ ID | | | | | | | | |
| | NOs: 97-98 | | | | | | | | |

In one embodiment, a TFcA comprises a TFc that comprises an IgG1 Fc region comprising a hinge comprising SEQ ID NO:4, a CH2 domain comprising SEQ ID NO:25 and a CH3 domain comprising SEQ ID NO:29, and may form, e.g., an IgG1 Fc region comprising SEQ ID NO:99 (see Table 8 and FIG. 4). Other combinations of IgG1 hinges, IgG1 CH2 domain, and IgG1 CH3 domains and exemplary IgG1 Fcs created by such combinations are provided in Table 8. The aa sequences of the exemplary IgG1 Fcs listed in Table 8 (SEQ ID NOs:99-132) are provided in FIG. 4. Compatible IgG1 Fcs in Table 8 are separated from other IgG1 Fcs by a blank row.

TABLE 8
SEQ ID NOs of exemplary combinations of IgG1 hinges, CH2 domain and CH3 domain
forming exemplary IgG1 Fcs

| IgG1 Hinge | IgG1 CH2 | IgG1 CH3 | IgG1 Fc |
|---|---|---|---|
| SEQ ID NO:4 | SEQ ID NO:25 | SEQ ID NO:29 | SEQ ID NO:99 |
| SEQ ID NO:23 | SEQ ID NO:25 | SEQ ID NO:29 | SEQ ID NO:100 |
| SEQ ID NO:4 | SEQ ID NO:25 | SEQ ID NO:33 | SEQ ID NO:101 |
| SEQ ID NO:23 | SEQ ID NO:25 | SEQ ID NO:37 | SEQ ID NO:102 |
| SEQ ID NO:4 | SEQ ID NO:25 | SEQ ID NO:37 | SEQ ID NO:103 |
| SEQ ID NO:23 | SEQ ID NO:25 | SEQ ID NO:33 | SEQ ID NO:104 |
| SEQ ID NO:4 | SEQ ID NO:25 | SEQ ID NO:77 | SEQ ID NO:105 |
| SEQ ID NO:23 | SEQ ID NO:25 | SEQ ID NO:81 | SEQ ID NO:106 |
| SEQ ID NO:4 | SEQ ID NO:25 | SEQ ID NO:81 | SEQ ID NO:107 |
| SEQ ID NO:23 | SEQ ID NO:25 | SEQ ID NO:77 | SEQ ID NO:108 |
| SEQ ID NO:4 | SEQ ID NO:25 | SEQ ID NO:41 | SEQ ID NO:109 |
| SEQ ID NO:23 | SEQ ID NO:25 | SEQ ID NO:45 | SEQ ID NO:110 |
| SEQ ID NO:4 | SEQ ID NO:25 | SEQ ID NO:45 | SEQ ID NO:111 |
| SEQ ID NO:23 | SEQ ID NO:25 | SEQ ID NO:41 | SEQ ID NO:112 |
| SEQ ID NO:4 | SEQ ID NO:25 | SEQ ID NO:33 | SEQ ID NO:113 |
| SEQ ID NO:23 | SEQ ID NO:25 | SEQ ID NO:77 | SEQ ID NO:114 |
| SEQ ID NO:4 | SEQ ID NO:25 | SEQ ID NO:33 | SEQ ID NO:115 |
| SEQ ID NO:23 | SEQ ID NO:25 | SEQ ID NO:77 | SEQ ID NO:116 |
| SEQ ID NO:4 | SEQ ID NO:25 | SEQ ID NO:84 | SEQ ID NO:117 |
| SEQ ID NO:23 | SEQ ID NO:25 | SEQ ID NO:86 | SEQ ID NO:118 |
| SEQ ID NO:4 | SEQ ID NO:25 | SEQ ID NO:86 | SEQ ID NO:119 |
| SEQ ID NO:23 | SEQ ID NO:25 | SEQ ID NO:84 | SEQ ID NO:120 |
| SEQ ID NO:4 | SEQ ID NO:25 | SEQ ID NO:88 | SEQ ID NO:121 |
| SEQ ID NO:23 | SEQ ID NO:25 | SEQ ID NO:90 | SEQ ID NO:122 |
| SEQ ID NO:4 | SEQ ID NO:25 | SEQ ID NO:90 | SEQ ID NO:123 |
| SEQ ID NO:23 | SEQ ID NO:25 | SEQ ID NO:88 | SEQ ID NO:124 |
| SEQ ID NO:4 | SEQ ID NO:25 | SEQ ID NO:92 | SEQ ID NO:125 |
| SEQ ID NO:23 | SEQ ID NO:25 | SEQ ID NO:94 | SEQ ID NO:126 |
| SEQ ID NO:4 | SEQ ID NO:25 | SEQ ID NO:94 | SEQ ID NO:127 |
| SEQ ID NO:23 | SEQ ID NO:25 | SEQ ID NO:92 | SEQ ID NO:128 |
| SEQ ID NO:4 | SEQ ID NO:25 | SEQ ID NO:96 | SEQ ID NO:129 |
| SEQ ID NO:23 | SEQ ID NO:25 | SEQ ID NO:98 | SEQ ID NO:130 |
| SEQ ID NO:4 | SEQ ID NO:25 | SEQ ID NO:98 | SEQ ID NO:131 |
| SEQ ID NO:23 | SEQ ID NO:25 | SEQ ID NO:96 | SEQ ID NO:132 |

In one embodiment, a TFcA comprises a TFc that comprises an IgG1/IgG4 Fc region comprising a hinge comprising SEQ ID NO:20, a CH2 domain comprising SEQ ID NO:26 and a CH3 domain comprising SEQ ID NO:29, and may form, e.g., an IgG1/IgG4 hybrid Fc region comprising SEQ ID NO:133 (see Table 9 and FIG. 4). Other combinations of IgG1/IgG4 hinges, IgG4 CH2 domain, and IgG1 CH3 domains and exemplary IgG1/IgG4 hybrid Fcs created by such combinations are provided in Table 9. The aa sequences of the exemplary IgG1/IgG4 hybrid Fcs listed in Table 9 (SEQ ID NOs:133-166) are provided in FIG. 5. Compatible IgG1 Fcs in Table 9 are separated from other IgG1 Fcs by a blank row.

TABLE 9
Exemplary combinations of IgG1/IgG4 hinges, CH2 domain and CH3 domain forming
exemplary IgG1/IgG4 hybrid Fcs

| Hinge | IgG4 CH2 | IgG1 CH3 | IgG1/IgG4 Fc |
|---|---|---|---|
| SEQ ID NO:20 | SEQ ID NO:26 | SEQ ID NO:29 | SEQ ID NO:133 |
| SEQ ID NO:24 | SEQ ID NO:26 | SEQ ID NO:29 | SEQ ID NO:134 |
| SEQ ID NO:20 | SEQ ID NO:26 | SEQ ID NO:33 | SEQ ID NO:135 |
| SEQ ID NO:24 | SEQ ID NO:26 | SEQ ID NO:37 | SEQ ID NO:136 |
| SEQ ID NO:20 | SEQ ID NO:26 | SEQ ID NO:37 | SEQ ID NO:137 |
| SEQ ID NO:24 | SEQ ID NO:26 | SEQ ID NO:33 | SEQ ID NO:138 |
| SEQ ID NO:20 | SEQ ID NO:26 | SEQ ID NO:77 | SEQ ID NO:139 |
| SEQ ID NO:24 | SEQ ID NO:26 | SEQ ID NO:81 | SEQ ID NO:140 |
| SEQ ID NO:20 | SEQ ID NO:26 | SEQ ID NO:81 | SEQ ID NO:141 |
| SEQ ID NO:24 | SEQ ID NO:26 | SEQ ID NO:77 | SEQ ID NO:142 |
| SEQ ID NO:20 | SEQ ID NO:26 | SEQ ID NO:41 | SEQ ID NO:143 |
| SEQ ID NO:24 | SEQ ID NO:26 | SEQ ID NO:45 | SEQ ID NO:144 |
| SEQ ID NO:20 | SEQ ID NO:26 | SEQ ID NO:45 | SEQ ID NO:145 |
| SEQ ID NO:24 | SEQ ID NO:26 | SEQ ID NO:41 | SEQ ID NO:146 |
| SEQ ID NO:20 | SEQ ID NO:26 | SEQ ID NO:33 | SEQ ID NO:147 |
| SEQ ID NO:24 | SEQ ID NO:26 | SEQ ID NO:77 | SEQ ID NO:148 |
| SEQ ID NO:20 | SEQ ID NO:26 | SEQ ID NO:33 | SEQ ID NO:149 |
| SEQ ID NO:24 | SEQ ID NO:26 | SEQ ID NO:77 | SEQ ID NO:150 |
| SEQ ID NO:20 | SEQ ID NO:26 | SEQ ID NO:84 | SEQ ID NO:151 |
| SEQ ID NO:24 | SEQ ID NO:26 | SEQ ID NO:86 | SEQ ID NO:152 |
| SEQ ID NO:20 | SEQ ID NO:26 | SEQ ID NO:86 | SEQ ID NO:153 |
| SEQ ID NO:24 | SEQ ID NO:26 | SEQ ID NO:84 | SEQ ID NO:154 |
| SEQ ID NO:20 | SEQ ID NO:26 | SEQ ID NO:88 | SEQ ID NO:155 |
| SEQ ID NO:24 | SEQ ID NO:26 | SEQ ID NO:90 | SEQ ID NO:156 |
| SEQ ID NO:20 | SEQ ID NO:26 | SEQ ID NO:90 | SEQ ID NO:157 |
| SEQ ID NO:24 | SEQ ID NO:26 | SEQ ID NO:88 | SEQ ID NO:158 |
| SEQ ID NO:20 | SEQ ID NO:26 | SEQ ID NO:92 | SEQ ID NO:159 |
| SEQ ID NO:24 | SEQ ID NO:26 | SEQ ID NO:94 | SEQ ID NO:160 |
| SEQ ID NO:20 | SEQ ID NO:26 | SEQ ID NO:94 | SEQ ID NO:161 |
| SEQ ID NO:24 | SEQ ID NO:26 | SEQ ID NO:92 | SEQ ID NO:162 |
| SEQ ID NO:20 | SEQ ID NO:26 | SEQ ID NO:96 | SEQ ID NO:163 |
| SEQ ID NO:24 | SEQ ID NO:26 | SEQ ID NO:98 | SEQ ID NO:164 |
| SEQ ID NO:20 | SEQ ID NO:26 | SEQ ID NO:98 | SEQ ID NO:165 |
| SEQ ID NO:24 | SEQ ID NO:26 | SEQ ID NO:96 | SEQ ID NO:166 |

Fc regions for use in TFcAs may also comprise aa sequences that differ from those described herein, e.g., SEQ ID NOs:99-166, in one or more aa modifications, e.g., aa deletions, additions or substitutions. In certain embodiments, an Fc region comprises an aa sequence that differs from a sequence set forth herein, e.g., from a sequence selected from the group consisting of SEQ ID NOs:99-166, in at most, 1, 2, 3, 4, 5, 6, 7, 8, 9, 10, 15, 20, 25, 30, 35, 40, 45 or 50 aas. In certain embodiments, an Fc region comprises an aa sequence that is at least 70%, 75%, 80%, 85%, 90%, 95%, 97%, 98% or 99% identical to that of a sequence set forth herein, e.g., a sequence selected from the group consisting of SEQ ID NOs:99-166. For example, the CH3 domain of an Fc region consisting of an aa sequence selected from the group consisting of SEQ ID NOs:99-166 may comprise a deletion of the C-terminal lysine and/or E356D and/or M358L. As several antibody effector functions, e.g., ADCC and CDC, are mediated through Fc regions, aa changes that can be made to Fc regions include changes affecting the effector function(s) of Fc regions. Exemplary mutations to these domains are set forth herein. Any of these aa modifications are permitted provided that the Fc region retains the desired properties, e.g., biological activity, stability and low immunogenicity.

### Exemplary Fc Modifications Affecting Effector Activity

TFcAs may comprise TFcs comprising aa modifications affecting the effector activity of Fc regions. Exemplary aa modifications are set forth below.

Replacements of aa residues in the Fc portion to alter antibody effector function are known in the art (see, e.g., U.S. Pat. Nos. 5,648,260 and 5,624,821). The Fc portion of an antibody mediates several important effector functions e.g., cytokine induction, antibody-dependent cellular cytotoxicity (“ADCC”), phagocytosis, complement dependent cytotoxicity (CDC) and half-life/clearance rate of antibody and antigen-antibody complexes. In some cases these effector functions are desirable for a therapeutic antibody but in other cases might be unnecessary or even deleterious, depending on the therapeutic objectives. Certain human IgG isotypes, particularly IgG1 and IgG3, mediate ADCC and CDC via binding to FcγRs and complement CIq, respectively. Neonatal Fc receptors (FcRn) are the critical components determining the circulating half-life of antibodies.

In one embodiment, a TFcA retains one or more of, and preferably all of the following attributes: ADCC and antibody-dependent cellular phagocytosis (ADCP) that in humans are determined by interactions with activating FcγRI, FcγRIIa/c, FcγRIIIa and inhibitory FcγRIIb receptors; CDC that is triggered by antibody binding to the components of the complement system; and long half-life that is mediated via active recycling by the neonatal Fc receptor (FcRn). When desired, all of these functions can be tuned to optimize the effectiveness of an anti-cancer therapy.

Certain aa modifications, e.g., the addition, deletion and/or substitution of one or more aas may be made to an immunoglobulin constant region to reduce or increase the natural biological activities of the constant domains, such as those set forth above.

In certain embodiments, a TFcA (e.g., a TFcBA) comprises an aa modification (e.g., an aa substitution, addition or deletion) in an Fc region that alters one or more antigen-independent effector functions of the domain, e.g., the circulating half-life of a protein comprising the domain. Exemplary antibodies exhibit either increased or decreased binding to FcRn when compared to antibodies lacking such aa changes and, therefore, have an increased or decreased half-life in serum, respectively. Antibodies comprising Fc variants with improved affinity for FcRn are anticipated to have longer serum half-lives, whereas those comprising Fc variants with decreased FcRn binding affinity are expected to have shorter half-lives. In one embodiment, a TFcA with altered FcRn binding comprises at least one Fc region having one or more aa changes within the “FcRn binding loop” of an Fc region. The FcRn binding loop is comprised of aa residues 280-299 (EU) of a wild type, full length, Fc. In certain embodiments, a TFcA having altered FcRn binding affinity comprises at least one Fc region having one or more aa substitutions within the 15 Å FcRn “contact zone.” The term 15 Å FcRn “contact zone” includes residues at the following positions of a wild type, full-length Fc domain: 243-261, 275-280, 282-293, 302-319, 336-348, 367, 369, 372-389, 391, 393, 408, 424-440 (EU). In certain embodiments, a TFcA having altered FcRn binding affinity comprises at least one Fc region (e.g., one or two Fc moieties) having one or more aa changes at an aa position corresponding to any one of the following EU positions: 256, 277-281, 283-288, 303-309, 313, 338, 342, 376, 381, 384, 385, 387, 434 (e.g., N434A or N434K), and 438. Exemplary aa changes that alter FcRn binding activity are disclosed in International PCT Publication No. WO05/047327.

Additional Fc modifications that enhance FcRn binding include substitutions at positions 259, 308, 428, and 434, e.g., 259I, 308F, 428L, 428M, 434S, 434H, 434F, 434Y, 434M, 428L/434S, 259I/308F and 259I/308F/428L. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604, entirely incorporated by reference), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524, entirely incorporated by reference). Other modifications for modulating FcRn binding are described in Yeung et al., 2010, J Immunol, 182:7663-7671.

In some embodiments, a TFcA comprises an Fc variant comprising an aa change that alters the antigen-dependent effector functions of the polypeptide, in particular ADCC or complement activation, e.g., as compared to a wild type Fc region. In exemplary embodiment, said antibodies exhibit altered binding to an Fc gamma receptor (e.g., CD16). Such antibodies exhibit either increased or decreased binding to FcγRs when compared to wild type polypeptides and, therefore, mediate enhanced or reduced effector function, respectively. Fc variants with improved affinity for FcγRs are anticipated to enhance effector function, and such proteins may have useful applications in methods of treating mammals where target molecule destruction is desired, e.g., in tumor therapy. In contrast, Fc variants with decreased FcγR binding affinity are expected to reduce effector function. In one embodiment, a TFcA comprises at least one altered antigen-dependent effector function selected from the group consisting of opsonization, phagocytosis, complement dependent cytotoxicity, antigen-dependent cellular cytotoxicity (ADCC), or effector cell modulation as compared to a TFcA comprising a wild type Fc region.

In certain embodiments, a TFcA exhibits altered binding to an activating FcγR (e.g. FcγRI, FcγRIIa, or FcγRIIIa). In certain embodiments, a TFcA exhibits altered binding affinity to an inhibitory FcγR (e.g. FcγRIIb). In other embodiments, a TFcA having increased FcγR binding affinity (e.g. increased FcγRIIIa binding affinity) comprises at least one Fc domain having an aa change at an aa position corresponding to one or more of the following positions: 239, 268, 298, 332, 334, and 378 (EU). In certain embodiments, a TFcA having decreased FcγR binding affinity (e.g. decreased FcγRI, FcγRII, or FcγRIIIa binding affinity) comprises at least one Fc domain having an aa substitution at an aa position corresponding to one or more of the following positions: 234, 236, 239, 241, 251, 252, 261, 265, 268, 293, 294, 296, 298, 299, 301, 326, 328, 332, 334, 338, 376, 378, and 435 (EU).

In certain embodiments, a TFcA having increased complement binding affinity (e.g. increased Clq binding affinity) comprises an Fc domain having an aa change at an aa position corresponding to one or more of the following positions: 251, 334, 378, and 435 (EU). In certain embodiments, a TFcA having decreased complement binding affinity (e.g. decreased Clq binding affinity) comprises an Fc domain having an aa substitution at an aa position corresponding to one or more of the following positions: 239, 294, 296, 301, 328, 333, and 376 (EU). Exemplary aa changes that alter FcγR or complement binding activity are disclosed in International PCT Publication No. WO05/063815. In certain embodiments, a TFcA may comprise one or more of the following specific Fc region substitutions: S239D, S239E, M252T, H268D, H268E, 1332D, I332E, N434A, and N434K (EU).

Other Fc variants that reduce binding to FcγRs and/or complement include variants comprising one or more of the following aa substitutions: 34G, 235G, 236R, 237K, 267R, 269R, 325L, 328R, 236R/328R, 297A, 234A, 235A, 237A, 318A, 228P, 236E, 268Q, 309L, 330S, 331 S, 220S, 226S, 229S, 238S, 233P, and 234V. Removal of the glycosylation at position 297 (see below) also reduces binding to FcγRs.

Fc modifications that improve binding to FcγRs and/or complement include variants comprising one or more of the following aa substitutions: 236A, 239D, 239E, 268D, 267E, 268E, 268F, 324T, 332D, and 332E. Preferred variants include but are not limited to 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 305I, and 396L.

Variants that improve binding to FcγRIIb include variants comprising one or more of the following aa substitutions: 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y and 332E, 235Y/267E, 236D/267E, 239D/268D, 239D/267E, 267E/268D, 267E/268E, and 267E/328F.

Fc modifications modulating Fc are described in Strohl, 2009, Current Opinion in Biotechnology 20:685-691.

A TFcA may also comprise an aa substitution that alters the glycosylation of the TFcA. For example, an immunoglobulin constant region of a TFcA may comprise an Fc domain having a mutation leading to reduced glycosylation (e.g., N- or O-linked glycosylation) or may comprise an altered glycoform of the wild type Fc domain (e.g., a low fucose or fucose-free glycan). An “engineered glycoform” refers to a carbohydrate composition that is covalently attached to an Fc region, wherein said carbohydrate composition differs chemically from that of a parent Fc region. Engineered glycoforms may be useful for a variety of purposes, including but not limited to enhancing or reducing effector function. Engineered glycoforms may be generated by a variety of methods known in the art (U.S. Pat. No. 6,602,684; US Pat Pub No. 2010-0255013; US Pat Pub No. 2003-0003097; WO 00/61739A1; WO 01/29246A1; WO 02/31140A1; WO 02/30954A1); (Potelligent™ technology (Biowa, Inc., Princeton, N.J.); and GlycoMAb™ glycosylation engineering technology (Glycart Biotechnology AG, Zurich, Switzerland). Many of these techniques are based on controlling the level of fucosylated and/or bisecting oligosaccharides that are covalently attached to the Fc region, for example by expressing an Fc polypeptide in various organisms or cell lines, engineered or otherwise (for example Lec-13 CHO cells or rat hybridoma YB2/0 cells), by regulating enzymes involved in the glycosylation pathway (for example FUT8 [al, 6-fucosyltranserase] and/or (31-4-N-acetylglucosaminyl, transferase III [GnTIII]), or by modifying carbohydrate(s) after the Fc polypeptide has been expressed.

In exemplary embodiments, an aa change, e.g., an aa substitution, results in an Fc region comprising reduced glycosylation of the N-linked glycan normally found at aa position 297 (EU). The Fc region may also comprise a low fucose or fucose free glycan at aa position 297 (EU). In certain embodiments, the TFcA has an aa substitution near or within a glycosylation motif, for example, an N-linked glycosylation motif that contains the aa sequence NXT or NXS. In a particular embodiment, a TFcA comprises an aa substitution at an aa position corresponding to 297 or 299 of Fc (EU) as further described herein. Exemplary aa substitutions that reduce or alter glycosylation are disclosed in International PCT Publication No. WO05/018572 and US Pat Pub No. 2007/0111281.

In other embodiments, a TFcA comprises at least one Fc domain having one or more engineered cysteine residues or analog thereof that are located at the solvent-exposed surface. Preferably the engineered cysteine residue or analog thereof does not interfere with the desired biological activity of the TFcA. For example, it may be desirable that the alteration does not interfere with the ability of the Fc to bind to Fc receptors (e.g. FcγRI, FcγRII, or FcγRIII) or complement proteins (e.g. Clq), or to trigger immune effector function (e.g., antibody-dependent cytotoxicity (ADCC), phagocytosis, or CDC). In certain embodiments, TFcAs comprise an Fc domain comprising at least one engineered free cysteine residue or analog thereof that is substantially free of disulfide bonding with a second cysteine residue. TFcAs may comprise an Fc region having engineered cysteine residues or analogs thereof at one or more of the following positions in the CH3 domain: 349-371, 390, 392, 394-423, 441-446, and 446b (EU), and more specifically positions 350, 355, 359, 360, 361, 389, 413, 415, 418, 422, 441, 443, and EU position 446b.

Desired effector functions may also be obtained by choosing an Fc from a particular immunoglobulin class or subclass, or by combining particular regions from particular immoglobulin classes or subclasses, e.g., IgG1, IgG2, etc. For example, since ADCC and CDC (through binding of IgG to the FcγRs and Clq, respectively) is mediated by residues located in the hinge and CH2 domain, and since IgG4 essentially lacks effector functions, an Fc constructed by combining the hinge and CH2 domain of IgG4 and the CH3 domain of IgG1 has much reduced effector functions. An lgG1/lgG3 hybrid variant may be constructed by substituting lgG1 positions in the CH2 and/or CH3 region with the amino acids from lgG3 at positions where the two isotypes differ. Thus a hybrid variant IgG antibody may be constructed that comprises one or more of the following substitutions: 274Q, 276K, 300F, 339T, 356E, 358M, 384S, 392N, 397M, 422I, 435R, and 436F. In certain embodiments, an lgG1/lgG2 hybrid variant may be constructed by substituting lgG2 positions in the CH2 and/or CH3 region with amino acids from lgG1 at positions where the two isotypes differ. Thus a hybrid variant IgG antibody may be constructed that comprises one or more of the following amino acid substations: 233E, 234L, 235L, −236G (referring to an insertion of a glycine at position 236), and 327A.

### Exemplary TFc Linkers

A TFcA may comprise a TFc comprising a first Fc region that is linked to a second Fc region through a TFc linker A wide variety of linkers may be used provided that they are sufficiently flexible to allow proper folding of the TFc and of a TFcA comprising the TFc. In certain embodiments, a linker is biologically inert, e.g., mostly incapable of inducing a biological response, e.g., an immune response.

A TFc linker may be 1-10, 10-20, 20-30, 30-40, 40-50, 50-60, 60-70, 70-80, 80-90 or at least 90-100 aas long. The size of a TFc linker may depend on whether the second Fc region comprises a hinge, a portion thereof or no hinge at all. For example, when the second Fc region comprises a hinge, a shorter TFc linker may be used than when the second Fc region does not comprise a hinge. For example, when a second Fc region does not comprise a hinge, a TFc linker may be longer by a number of aas corresponding to the length of a hinge. When a second Fc region does not comprise an upper hinge, a TFc linker may be longer by a number of aas corresponding to the length of the upper hinge. In a preferred embodiment, a TFcA, e.g., a TFcA that comprises a second hinge consisting of a middle and lower hinge, comprises a TFc linker comprising from 35 to 45 aas, such as from 37 to 43 aas, such as from 38 to 42 aas, such as from 39 to 41 aas, and more particularly, 40 aas.

A TFc linker may comprise a Gly-Ser linker. A “Gly-Ser linker” refers to a peptide that consists of glycine and serine residues. An exemplary Gly-Ser linker comprises an aa sequence having the formula (Gly₄Ser)ₙ, wherein n is a positive integer (e.g., 1, 2, 3, 4, 5, 6, 7, 8, 9, 10, 11, 12, 13, 14, 15, 16, 17, 18, 19, 20). For example, in certain embodiments, a TFc linker comprises or consists of (Gly₄Ser)₃ or (Gly₄Ser)₄ or (Gly₄Ser)₅ or (Gly₄Ser)₆ or (Gly₄Ser)₇ or (Gly₄Ser)₈. In a preferred embodiment, the TFc linker is (Gly₄Ser)₈.

Other linkers that may be used include those that comprise Gly and Ser, but not in a (G4S)n structure. For example, linkers may comprise (Gly-Gly-Ser)n or (Gly-Ser-Gly-Ser)n, wherein n is 1, 2, 3, 4, 5, 6, 7, 8, 9, 10, 11, 12, 13, 14, 15 or more. Other linkers may comprise Pro or Thr. Suitable linkers may be found in the Registry of Standard Biological Parts at http://partsregistry.org/Protein_domains/Linker (see also, e.g., Crasto CJ and Feng JA. LINKER: a program to generate linker sequences for fusion proteins. Protein Eng 2000 May; 13(5) 309-12 and George RA and Hering a J. An analysis of protein domain linkers: their classification and role in protein folding. Protein Eng 2002 November; 15(11) 871-9).

In certain embodiments, a TFc linker comprises the following aa sequence:

(SEQ ID NO: 169)
TRPAPPSTATTAGSTPQPESASPSGKEPAASSPSSTNTGS

TFc linkers comprising an aa sequence that differs from SEQ ID NO:169 or from a (G4S)n sequence in at most 1, 2, 3, 4, 5, 6, 7, 8, 9 or 10 aas may also be used.

A TFc linker may also be a non-peptide linker, such as a non-peptide polymer. The term “non-peptide polymer,” refers to a biocompatible polymer including two or more repeating units linked to each other by a covalent bond excluding the peptide bond. Examples of the non-peptide polymer include poly (ethylene glycol), poly (propylene glycol), copolymers of ethylene glycol and propylene glycol, polyoxyethylated polyols, polyvinyl alcohol, polysaccharides, dextran, polyvinyl ether, biodegradable polymers such as PLA (poly (lactic acid) and PLGA (poly (lactic-glycolic acid), lipid polymers, chitins, and hyaluronic acid. The most preferred is poly (ethylene glycol) (PEG).

### Exemplary TFcs

TFcAs may comprise a TFc comprising a first Fc region that is linked to a second Fc region through a TFc linker. In certain embodiments, a TFc comprises a first and a second Fc region that are identical to each other. In other embodiments, the first and the second Fc regions differ from each other in at least one aa (“heteromeric TFc”). A first and a second Fc region may be any Fc region disclosed herein or a variation thereof. For example, a TFc may comprise a first Fc region that comprises a full length hinge, e.g., a full length IgG1 or IgG1/IgG4 hybrid hinge, and a second Fc region that comprises a partial hinge, e.g., a hinge that is devoid of the upper hinge.

First and second Fc regions may be combined with any TFc linker described herein. As set forth above, generally the length of the TFc linker may depend on whether the second Fc region comprises a hinge, a portion thereof, or no hinge at all.

In certain embodiments, an IgG1 TFc comprises a first Fc region comprising SEQ ID NO:99 and a second Fc region comprising SEQ ID NO:100. Combinations of first and second Fc regions that may be used in IgG1 TFcs are set forth in Table 10.

TABLE 10
Exemplary combinations of first and second Fc regions (shown in FIG. 4)
for use in IgG1 TFcs

| First Fc region | Second Fc region |
|---|---|
| SEQ ID NO:99 | SEQ ID NO:100 |
| SEQ ID NO:101 | SEQ ID NO:102 |
| SEQ ID NO:103 | SEQ ID NO:104 |
| SEQ ID NO:105 | SEQ ID NO:106 |
| SEQ ID NO:107 | SEQ ID NO:108 |
| SEQ ID NO:109 | SEQ ID NO:110 |
| SEQ ID NO:111 | SEQ ID NO:112 |
| SEQ ID NO:113 | SEQ ID NO:114 |
| SEQ ID NO:115 | SEQ ID NO:116 |
| SEQ ID NO:117 | SEQ ID NO:118 |
| SEQ ID NO:119 | SEQ ID NO:120 |
| SEQ ID NO:121 | SEQ ID NO:122 |
| SEQ ID NO:123 | SEQ ID NO:124 |
| SEQ ID NO:125 | SEQ ID NO:126 |
| SEQ ID NO:127 | SEQ ID NO:128 |
| SEQ ID NO:129 | SEQ ID NO:130 |
| SEQ ID NO:131 | SEQ ID NO:132 |

In certain embodiments, an IgG1/IgG4 hybrid TFc comprises a first Fc region comprising SEQ ID NO:133 and a second Fc region comprising SEQ ID NO:134. Combinations of first and second Fc regions that may be used in IgG1/IgG4 hybrid TFcs are set forth in Table 11.

TABLE 11
Exemplary combinations of first and second Fc regions (shown in FIG. 5)
for use in IgG1/IgG4 TFcs

| First Fc region | Second Fc region |
|---|---|
| SEQ ID NO:133 | SEQ ID NO:134 |
| SEQ ID NO:135 | SEQ ID NO:136 |
| SEQ ID NO:137 | SEQ ID NO:138 |
| SEQ ID NO:139 | SEQ ID NO:140 |
| SEQ ID NO:141 | SEQ ID NO:142 |
| SEQ ID NO:143 | SEQ ID NO:144 |
| SEQ ID NO:145 | SEQ ID NO:146 |
| SEQ ID NO:147 | SEQ ID NO:148 |
| SEQ ID NO:149 | SEQ ID NO:150 |
| SEQ ID NO:151 | SEQ ID NO:152 |
| SEQ ID NO:153 | SEQ ID NO:154 |
| SEQ ID NO:155 | SEQ ID NO:156 |
| SEQ ID NO:157 | SEQ ID NO:158 |
| SEQ ID NO:159 | SEQ ID NO:160 |
| SEQ ID NO:161 | SEQ ID NO:162 |
| SEQ ID NO:163 | SEQ ID NO:164 |
| SEQ ID NO:165 | SEQ ID NO:166 |

A TFc may comprise a combination of two Fcs set forth in Table 10 or 11, which are linked together through a TFc linker to form a contiguous polypeptide comprising in amino to carboxyl terminal order: a first Fc region, which is linked at its C-terminus to the N-terminus of a TFc linker, which is linked at its C-terminus to the N-terminus of the second Fc region. The TFc linker may comprise or consist of 20 to 50 amino acids in length.

Exemplary TFcs may comprise: i) a first Fc region comprising a hinge comprising SEQ ID NO:4, a CH2 domain comprising SEQ ID NO:25, a CH3 domain comprising SEQ ID NO:33; ii) a TFc linker comprising (G₄S)₈; and iii) a second Fc region comprising a hinge comprising SEQ ID NO:23, a CH2 domain comprising SEQ ID NO:25 and a CH3 domain comprising SEQ ID NO:37. An exemplary IgG1 TFc comprising this set of elements is a TFc comprising SEQ ID NO:171. Additional combinations of domains or elements forming IgG1 and IgG1/IgG4 hybrid TFcs are provided in Table 12 and 13, respectively. Each of the elements or domains in Tables 12 and 13 is referred to by its SEQ ID NO and the specific AEM and/or DiS that it comprises. Each of the domains or elements in Tables 12 and 13 may be linked directly or indirectly.

The aa sequence of each of the TFcs listed in Table 12 (SEQ ID NOs:171, 173, 175, 177, 179, 181, 183, 185, 187, 189, 191, 193 and 195) is provided in FIG. 6. The aa sequence of each of the TFcs listed in Table 13 (SEQ ID NOs:197, 199, 201, 203, 205, 207, 209, 211, 213, 215, 217, 219 and 221) is provided in FIG. 7. The first column of Tables 12 and 13 lists the name and SEQ ID NO of an exemplary TFc comprising the elements listed in the corresponding row of the Table.

TABLE 12
IgG1 TFcs set forth in FIG. 6
IgG1 TFc

| | First Fc | | | TFc | Second Fc | | |
|---|---|---|---|---|---|---|---|
| | Hinge | CH2 | CH3 | linker | Hinge | CH2 | CH3 |
| 23 | SEQ ID | SEQ ID | SEQ ID | (G₄S)₈ | SEQ ID | SEQ ID | SEQ ID |
| SEQ ID | NO: 4 | NO: 25 | NO: 33 | | NO: 23 | NO: 25 | NO: 37 |
| NO: 171 | wild type | | T366S/L368A/ | | partial | | T366W |
| | | | Y407V | | | | |
| 23A | SEQ ID | SEQ ID | SEQ ID | (G₄S)₈ | SEQ ID | SEQ ID | SEQ ID |
| SEQ ID | NO: 4 | NO: 25 | NO: 77 | | NO: 23 | NO: 25 | NO: 81 |
| NO: 173 | wild type | | Y349C/T366S/ | | partial | | S354C/T366W |
| | | | L368A/Y407V | | | | |
| 23B | SEQ ID | SEQ ID | SEQ ID | (G₄S)₈ | SEQ ID | SEQ ID | SEQ ID |
| SEQ ID | NO: 4 | NO: 25 | NO: 41 | | NO: 23 | NO: 25 | NO: 45 |
| NO: 175 | wild type | | Y407T | | partial | | T366Y |
| 23C | SEQ ID | SEQ ID | SEQ ID | (G₄S)₈ | SEQ ID | SEQ ID | SEQ ID |
| SEQ ID | NO: 18 | NO: 25 | NO: 33 | | NO: 23 | NO: 25 | NO: 37 |
| NO: 177 | H224C/ | | T366S/L368A/ | | partial | | T366W |
| | T225C | | Y407V | | | | |
| 23D | SEQ ID | SEQ ID | SEQ ID | (G₄S)₈ | SEQ ID | SEQ ID | SEQ ID |
| SEQ ID | NO: 19 | NO: 25 | NO: 33 | | NO: 23 | NO: 25 | NO: 37 |
| NO: 179 | T223C | | T366S/L368A/ | | partial | | T366W |
| | | | Y407V | | | | |
| 23E | SEQ ID | SEQ ID | SEQ ID | (G₄S)₈ | SEQ ID | SEQ ID | SEQ ID |
| SEQ ID | NO: 4 | NO: 25 | NO: 84 | | NO: 23 | NO: 25 | NO: 86 |
| NO: 181 | wild type | | T366S/L368A/ | | partial | | T366W/C-term |
| | | | Y407V/C-term | | | | Cysteine |
| | | | Cysteine | | | | |
| 23G | SEQ ID | SEQ ID | SEQ ID | (G₄S)₄ | SEQ ID | SEQ ID | SEQ ID |
| SEQ ID | NO: 4 | NO: 25 | NO: 33 | | NO: 23 | NO: 25 | NO: 37 |
| NO: 183 | wild type | | T366S/L368A/ | | partial | | T366W |
| | | | Y407V | | | | |
| 23E | SEQ ID | SEQ ID | SEQ ID | (G₄S)₇ | SEQ ID | SEQ ID | SEQ ID |
| (35L) | NO: 4 | NO: 25 | NO: 84 | | NO: 23 | NO: 25 | NO: 86 |
| SEQ ID | wild type | | T366S/L368A/ | | partial | | T366W/C-term |
| NO: 185 | | | Y407V/C-term | | | | Cysteine |
| | | | Cysteine | | | | |
| 23E | SEQ ID | SEQ ID | SEQ ID | (G₄S)₇ | SEQ ID | SEQ ID | SEQ ID |
| (35L) Inv | NO: 4 | NO: 25 | NO: 88 | | NO: 23 | NO: 25 | NO: 90 |
| SEQ ID | wild type | | T366S/L368A/ | | partial | | T366W/C-term |
| NO: 187 | | | Y407V/C-term | | | | Cysteine Inv |
| | | | Cysteine Inv | | | | |
| 23E | SEQ ID | SEQ ID | SEQ ID | (G₄S)₆ | SEQ ID | SEQ ID | SEQ ID |
| (30L) | NO: 4 | NO: 25 | NO: 84 | | NO: 23 | NO: 25 | NO: 86 |
| SEQ ID | wild type | | T366S/L368A/ | | partial | | T366W/C-term |
| NO: 189 | | | Y407V/C-term | | | | Cysteine |
| | | | Cysteine | | | | |
| 23E | SEQ ID | SEQ ID | SEQ ID | (G₄S)₅ | SEQ ID | SEQ ID | SEQ ID |
| (25L) | NO: 4 | NO: 25 | NO: 84 | | NO: 23 | NO: 25 | NO: 86 |
| SEQ ID | wild type | | T366S/L368A/ | | partial | | T366W/C-term |
| NO: 191 | | | Y407V/C-term | | | | Cysteine |
| | | | Cysteine | | | | |
| 23I | SEQ ID | SEQ ID | SEQ ID | (G₄S)₈ | SEQ ID | SEQ ID | SEQ ID |
| SEQ ID | NO: 4 | NO: 25 | NO: 92 | | NO: 23 | NO: 25 | NO: 94 |
| NO: 193 | wild type | | S364H/F405A | | partial | | Y349T/T394F |
| 23J | SEQ ID | SEQ ID | SEQ ID | (G₄S)₈ | SEQ ID | SEQ ID | SEQ ID |
| SEQ ID | NO: 4 | NO: 25 | NO: 96 | | NO: 23 | NO: 25 | NO: 98 |
| NO: 195 | wild type | | K370D/K392D/ | | partial | | D356K/E357K/ |
| | | | K409D | | | | D399K |

TABLE 13
IgG1/IgG4 hybrid TFcs set forth in FIG. 7
IgG1/IgG4 hybrid TFc

| | First Fc | | | TFc | Second Fc | | |
|---|---|---|---|---|---|---|---|
| | Hinge | CH2 | CH3 | linker | Hinge | CH2 | CH3 |
| 39 | SEQ ID | SEQ ID | SEQ ID | (G₄S)₈ | SEQ ID | SEQ ID | SEQ ID |
| SEQ ID | NO: 20 | NO: 26 | NO: 33 | | NO: 24 | NO: 26 | NO: 37 |
| NO: 197 | wild type | | T366S/L368A/ | | partial | | T366W |
| | | | Y407V | | | | |
| 39A | SEQ ID | SEQ ID | SEQ ID | (G₄S)₈ | SEQ ID | SEQ ID | SEQ ID |
| SEQ ID | NO: 20 | NO: 26 | NO: 77 | | NO: 24 | NO: 26 | NO: 81 |
| NO: 199 | wild type | | Y349C/T366S/ | | partial | | S354C/T366W |
| | | | L368A/Y407V | | | | |
| 39B | SEQ ID | SEQ ID | SEQ ID | (G₄S)₈ | SEQ ID | SEQ ID | SEQ ID |
| SEQ ID | NO: 20 | NO: 26 | NO: 41 | | NO: 24 | NO: 26 | NO: 45 |
| NO: 201 | wild type | | Y407T | | partial | | T366Y |
| 39C | SEQ ID | SEQ ID | SEQ ID | (G₄S)₈ | SEQ ID | SEQ ID | SEQ ID |
| SEQ ID | NO: 21 | NO: 26 | NO: 33 | | NO: 24 | NO: 26 | NO: 37 |
| NO: 203 | H224C/ | | T366S/L368A/ | | partial | | T366W |
| | T225C | | Y407V | | | | |
| 23D | SEQ ID | SEQ ID | SEQ ID | (G₄S)₈ | SEQ ID | SEQ ID | SEQ ID |
| SEQ ID | NO: 22 | NO: 26 | NO: 33 | | NO: 24 | NO: 26 | NO: 37 |
| NO: 205 | T223C | | T366S/L368A/ | | partial | | T366W |
| | | | Y407V | | | | |
| 39E | SEQ ID | SEQ ID | SEQ ID | (G₄S)₈ | SEQ ID | SEQ ID | SEQ ID |
| SEQ ID | NO: 20 | NO: 26 | NO: 84 | | NO: 24 | NO: 26 | NO: 86 |
| NO: 207 | wild type | | T366S/L368A/ | | partial | | T366W/C-term |
| | | | Y407V/C-term | | | | Cysteine |
| | | | Cysteine | | | | |
| 39G | SEQ ID | SEQ ID | SEQ ID | (G₄S)₄ | SEQ ID | SEQ ID | SEQ ID |
| SEQ ID | NO: 20 | NO: 26 | NO: 33 | | NO: 24 | NO: 26 | NO: 37 |
| NO: 209 | wild type | | T366S/L368A/ | | partial | | T366W |
| | | | Y407V | | | | |
| 39E | SEQ ID | SEQ ID | SEQ ID | (G₄S)₇ | SEQ ID | SEQ ID | SEQ ID |
| (35L) | NO: 20 | NO: 26 | NO: 84 | | NO: 24 | NO: 26 | NO: 86 |
| SEQ ID | wild type | | T366S/L368A/ | | partial | | T366W/C-term |
| NO: 211 | | | Y407V/C-term | | | | Cysteine |
| | | | Cysteine | | | | |
| 39E | SEQ ID | SEQ ID | SEQ ID | (G₄S)₇ | SEQ ID | SEQ ID | SEQ ID |
| (35L) Inv | NO: 20 | NO: 26 | NO: 88 | | NO: 24 | NO: 26 | NO: 90 |
| SEQ ID | wild type | | T366S/L368A/ | | partial | | T366W/C-term |
| NO: 213 | | | Y407V/C-term | | | | Cysteine Inv |
| | | | Cysteine Inv | | | | |
| 39E | SEQ ID | SEQ ID | SEQ ID | (G₄S)₆ | SEQ ID | SEQ ID | SEQ ID |
| (30L) | NO: 20 | NO: 26 | NO: 84 | | NO: 24 | NO: 26 | NO: 86 |
| SEQ ID | wild type | | T366S/L368A/ | | partial | | T366W/C-term |
| NO: 215 | | | Y407V/C-term | | | | Cysteine |
| | | | Cysteine | | | | |
| 39E | SEQ ID | SEQ ID | SEQ ID | (G₄S)₅ | SEQ ID | SEQ ID | SEQ ID |
| (25L) | NO: 20 | NO: 26 | NO: 84 | | NO: 24 | NO: 26 | NO: 86 |
| SEQ ID | wild type | | T366S/L368A/ | | partial | | T366W/C-term |
| NO: 217 | | | Y407V/C-term | | | | Cysteine |
| | | | Cysteine | | | | |
| 39I | SEQ ID | SEQ ID | SEQ ID | (G₄S)₈ | SEQ ID | SEQ ID | SEQ ID |
| SEQ ID | NO: 20 | NO: 26 | NO: 92 | | NO: 24 | NO: 26 | NO: 94 |
| NO: 219 | wild type | | S364H/F405A | | partial | | Y349T/T394F |
| 39J | SEQ ID | SEQ ID | SEQ ID | (G₄S)₈ | SEQ ID | SEQ ID | SEQ ID |
| SEQ ID | NO: 20 | NO: 26 | NO: 96 | | NO: 24 | NO: 26 | NO: 98 |
| NO: 221 | wild type | | K370D/K392D/ | | partial | | D356K/E357K/ |
| | | | K409D | | | | D399K |

In certain embodiments, a TFc comprises an aa sequence that differs from that of a TFc described herein, e.g., an aa sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221, in at most 10, 20, 30, 40, 50, 60, 70, 80, 90, 100, 150, 200, 250 or 300 aas, provided that the TFc has the desired biological activity, such as effector function or lack thereof, proper folding, sufficient stability and solubility. Differences may be one or more aa insertions, deletions and/or substitutions. In certain embodiments, a TFc comprises an aa sequence that is at least about 70%, 75%, 80%, 85%, 90%, 95%, 97%, 98%, or 99% identical to that of a TFc described herein, e.g., an aa sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221, provided that the TFcA comprising the TFc has the desired biological activity, such as effector function or lack thereof, proper folding, sufficient stability and/or sufficient solubility.

### Exemplary Binding Sites

A TFcA may be a monovalent TFcA that comprises a single binding site. The single binding site may be located a the amino terminus or carboxyl terminus of the TFc. The single binding site may be a Fab or an scFv. When the single binding site is a Fab, the monovalent TFcA comprises a heavy chain comprising the VH domain and optionally a CH1 domain and a light chain comprising the VL domain and optionally a CL domain.

A TFcA may also be a TFcBA comprising two binding sites, e.g., wherein each binding site binds to the same or to a different epitope or antigen (a bivalent monospecific of bispecific TFcA). The binding sites of a TFcBA may be of the same type or of a different type. For example, both binding sites may be TFcs, both binding sites may be Fabs, one binding site may be a Fab and the other binding site may be an scFv. Single domain binding sites may also be used. A Fab will generally comprise a VH domain, which may be linked to a CH1 domain on the heavy chain of a TFcBA and a VL domain, which may be linked to a CL domain on the light chain of the molecule. An scFv will generally comprise a VH domain linked to an scFv linker that is linked to a VL domain.

An scFv may be connected to a TFc by a connecting linker. A connecting linker may be about 1-5, 1-10, 1-15, 1-20 aas long or longer. A connecting linker is preferably chemically inert, non immunogenic and has the required flexibility and rigidity for allowing the proper conformation of a TFcBA comprising the scFv. In one embodiment, a connecting linker comprises a Gly-Ser sequence, e.g., the aa sequence (G₄S)ₙ, wherein n is 1, 2, 3, 4, or 5 or more. In one embodiment, a connecting linker comprises (G₄S)₂ (see, e.g., FIG. 9).

An scFv comprises an scFv linker that links together the VH and the VL domains. An scFv linker may be 15-30 or 20-25 aa long. An scFv linker is preferably chemically inert, non immunogenic and has the required flexibility and rigidity for allowing the proper conformation of a TFcBA comprising the scFv. In one embodiment, an scFv linker comprises a Gly-Ser sequence, e.g., the aa sequence (G₄S)ₙ, wherein n is 1, 2, 3, 4, or 5 or more. However, other sequences may also be used. In certain embodiments, an scFv linker may comprise a portion of a hinge or a full length hinge alone or together with other aas, such as a (G₄S)ₙ sequence. In certain embodiments, an scFv linker comprises the sequence “AST” (the first 3 aa of a CH1 domain) upstream of a peptide linker, such as a Gly-Ser linker, e.g., (G₄S)ₙ (see, e.g., FIG. 9).

In certain embodiments, a TFcA does not comprise a first and/or a second hinge. Instead of a hinge, a TFcA may comprise a connecting linker. Such a linker may be a Gly-Ser linker as further described herein in the context of TFc linkers. An exemplary connecting linker may be shorter than a TFc linker. In certain embodiments, a connecting linker comprises a (G₄S)₃ or (G₄S)₄ sequence. In certain embodiments, a connecting linker comprises a portion of a hinge, e.g., an upper hinge, middle hinge, lower hinge, or a combination thereof or a portion of one of these and another peptide sequence, such as a (G₄S)ₙ sequence, wherein n is 1, 2, 3, 4 or 5. Other peptide sequences may also be used as a connecting linker provided that they provide the required flexibility and rigidity of certain parts of the linker.

In certain embodiments, a binding site is an antigen binding site, such as a Fab, scFv or single domain. Exemplary TFcAs comprise one or more VH and/or VL CDRs such as those from one or more of the variable regions provided herein. In certain embodiments, an anti-c-Met binding site comprises a VHCDR3 and/or a VLCDR3 sequence set forth in FIG. 9, such as those of the variable domains of the humanized antibody 5D5 (US2006/0134104) or the anti-c-Met binding site 2 (see Example 3). In certain embodiments, an anti-c-Met binding site comprises 1, 2 or 3 CDRs of one of the VH domains set forth in FIG. 9 and/or 1, 2 or 3 CDRs of the VL domain set forth in FIG. 9. In certain embodiments, an anti-EGFR binding site comprises a VHCDR3 and/or a VLCDR3 sequence set forth in FIG. 9. In certain embodiments, an anti-EGFR binding site comprises 1, 2 or 3 CDRs of one of the VH domains set forth in FIG. 9 and/or 1, 2 or 3 CDRs of one of the VL domains set forth in FIG. 9. Binding sites may also comprise one or more CDRs set forth in FIG. 9, wherein 1, 2 or 3 aas have been changed, e.g., substituted, added or deleted, provided that the binding sites are still able to bind specifically to their target.

In certain embodiments, TFcAs comprise one or more variable domains set forth in FIG. 9. For example, an anti-c-Met binding site may comprise a VH and/or VL sequence set forth in FIG. 9, such as the variable domains of the humanized antibody 5D5 (US2006/0134104) or the anti-c-Met binding site 2. Exemplary anti-EGFR binding sites comprise a VH and/or a VL sequence set forth in FIG. 9, such as those of panitumumab, 2224, cetuximab or humanized cetuximab H1L1, H1L2, H2L1 or H2L2 (see Example 3).

In certain embodiments, anti-c-Met/anti-EGFR TFcAs comprise an anti-c-Met Fab and an anti-EGFR scFv. Table 14 shows combinations of CDRs or variable domains of each of the following anti-c-Met and anti-EGFR aa sequences that may be used for forming TFcAs. The Table provides a SEQ ID NO if the sequence is provided herein or a “yes” if a combination is possible, but the resulting aa sequence is not specifically provided. A person of skill in the art will be able to create such a molecule without undue experimentation based on the fact that all the elements of such proteins and nucleotide sequences encoding such are provided herein.

TABLE 14
Heavy chains of exemplary TFcAs

| | | Anti-c-Met Fab | |
|---|---|---|---|
| | | Humanized 5D5 | Binding site 2 |
| Anti- | panitumumab | SEQ ID NO:235, | yes |
| EGFR | | 343, 225, 227 and | |
| scFv | | 229 | |
| | 2224 | SEQ ID NO:239 | yes |
| | cetuximab H1L1 | SEQ ID NO:260 | SEQ ID NO:291 |
| | cetuximab H1L2 | SEQ ID NO:281 | yes |
| | cetuximab H2L1 | SEQ ID NO:283 | yes |
| | cetuximab H2L2 | SEQ ID NO:285 | yes |

Light chains that may be used with the heavy chains in Table 14 are the light chains of the particular anti-c-Met Fab used in the TFcA. For example, a TFcA comprising a VH domain from humanized 5D5, e.g., TFcAs comprising any one of SEQ ID NOs:225, 227, 229, 235, 239, 260, 281, 283, 285 and 342, may be used with a light chain comprising the VL domain of humanized 5D5, i.e., SEQ ID NO:231. A TFcA comprising a VH domain from the anti-c-Met binding site 2, e.g., TFcAs comprising SEQ ID NO:291, may be used with a light chain comprising the VH domain of the anti-c-Met binding site 2, e.g., the VL domain of SEQ ID NO:289.

Antigen binding sites, e.g., the ones described herein, may be engineered for enhanced stability, reduced heterogeneity, enhanced expression, enhanced solubility or other desirable characteristic. Methods for engineering of antibody fragments, such as scFv, VH, VL, and Fab with enhanced stability and increased expression are described, e.g., in US 2006/0127893 US 2009/0048122 and references therein.

Variable domains may differ from those set forth herein in, or in at most, 1, 2, 3, 4, 5, 6, 7, 8, 9, 10, 15, 20, 25, 30, 50, 100 aas, provided that a binding site with a modified variable region retains its ability to bind specifically to its target antigen, e.g., a human antigen selected from c-MET, ErbB2, ErbB3, ErbB4, IGF1R, IGF2R, Insulin receptor, RON, EGFR, VEGFR1, VEGFR2, TNFR, FGFR1-4, PDGFR (alpha and beta), c-Kit, EPCAM and EphA2. Variable domains for use in TFcAs, e.g., TFcBAs, may also comprise a VH or VL aa sequence that is at least 70%, 75%, 80%, 85%, 90%, 95%, 97%, 98% or 99% identical to the VH or VL aa sequence set forth in FIG. 9, provided that a binding site with a modified variable domain retains its ability to bind specifically to its target antigen.

Anti-c-MET and/or anti-EGFR TFcBAs may also comprise a binding site that binds to the same epitope on human c-Met or human EGFR as the binding sites provided herein, such as the ones having sequences set forth in FIG. 9. Binding sites encompassed herein may also competitively block or compete with the binding of a binding site described herein, such as the ones having sequences set forth in FIG. 9. A TFcA comprising a binding site that competes with a binding site described herein for binding to a target antigen or epitope include the binding sites that are capable of displacing a reference binding site (e.g., as described herein), e.g., when added in an ELISA after the reference binding site, as well as binding sites that prevent a reference binding site from binding when the binding site is added after the reference binding site to an ELISA.

TFcAs may also comprise variable domains from anti-c-Met, anti-c-Kit, anti-ErbB2, anti-ErbB3, anti-ErbB4, anti-IGF1R, anti-IGF2R, anti-Insulin receptor, anti-RON, anti-VEGFR1, anti-VEGFR2, anti-TNFR, anti-FGFR1, anti-FGFR2, anti-FGFR3, anti-FGFR4, anti-PDGFR alpha, anti-PDGFR beta, anti-EPCAM, anti-EphA2 or anti-EGFR antibodies known in the art. Known anti-c-Met antibodies are set forth in U.S. Pat. No. 5,686,292, U.S. Pat. No. 7,476,724, WO 2004/072117, WO 2004/108766, WO 2005/016382, WO 2005/063816, WO 2006/015371, WO 2006/104911, WO 2007/126799, and WO 2009/007427. Exemplary known anti-EGFR antibodies include ABX-EGF (Abgenix) (Yang, X. D., et al., Crit. Rev. Oncol./Hematol. 38 (2001) 17-23) and humanized ICR62 (WO 2006/082515). Exemplary anti-c-Kit antibodies are set forth in U.S. Pat. No. 7,915,391 and EP 0586445B1. Exemplary anti-ErbB2 antibodies are set forth in U.S. Pat. No. 5,821,337 and U.S. Pat. No. 7,560,111. Exemplary anti-ErbB3 antibodies are set forth in U.S. Pat. No. 7,705,130, U.S. Pat. No. 7,846,440 and WO 2011/136911. Exemplary anti-ErbB4 antibodies are set forth in U.S. Pat. No. 7,332,579 and US 2010/0190964. Exemplary anti-IGF1R antibodies are set forth in U.S. Pat. No. 7,871,611 and U.S. Pat. No. 7,700,742. Exemplary anti-Insulin receptor antibodies are set forth in Bhaskar V. et al, Diabetes. 2012 May; 61(5):1263-71. Exemplary anti-RON antibodies are set forth in WO 2012/006341, US 2009/0226442, and U.S. Pat. No. 7,947,811. Exemplary anti-VEGFR1 antibodies are set forth in WO 2005/037235. Exemplary anti-VEGFR2 antibodies are set forth in U.S. Pat. No. 8,057,791 and U.S. Pat. No. 6,344,339. Exemplary anti-TNFR1 antibodies are set forth in EP 1972637B1 and US 2008/0008713. Exemplary anti-FGFR1 antibodies are set forth in Ronca R et al, Mol Cancer Ther; 9(12); 3244-53, 2010, and WO 2005/037235. Exemplary anti-FGFR2 antibodies are set forth in WO 2011/143318. Exemplary anti-FGFR3 antibodies are set forth in WO 2010/002862 and EP 1423428B1. Exemplary anti-FGFR4 antibodies are set forth in WO 03/063893, WO 2008/052796 and US 2010/0169992. Exemplary anti-PDGFR alpha antibodies are set forth in U.S. Pat. No. 8,128,929 and WO 1995/000659. Exemplary anti-PDGFR beta antibodies are set forth in U.S. Pat. No. 7,740,850. Exemplary anti-EPCAM antibodies are set forth in U.S. Pat. No. 7,976,842, US 2003/0157054, and WO 2001/007082. Exemplary anti-EphA2 antibodies are set forth in EP 1575509B1, U.S. Pat. No. 7,402,298, and U.S. Pat. No. 7,776,328. Exemplary CD-44m antibodies are set forth in U.S. Pat. No. 8,071,072, WO 2008/079246, U.S. Pat. No. 6,972,324. Exemplary CEA antibodies are set forth in U.S. Pat. No. 7,626,011. Exemplary ALK antibodies are set forth in U.S. Pat. No. 6,696,548, U.S. Pat. No. 7,902,340, and WO 2008/131575. Exemplary AXL antibodies are set forth in US 2010/0330095, US 2012/0121587, and WO 2011/159980.

In another embodiment, a binding site is a binding peptide. c-Met binding peptides are known e.g. from Matzke, A., et al., Cancer Res 65 (14) (2005) 6105-10. And Tam, Eric, M., et al., J. Mol. Biol. 385 (2009)79-90.

Binding sites preferably bind specifically to their target with Kd of 10⁻⁶, 10⁻⁷, 10⁻⁸, 10⁻⁹ M, or 10⁻¹⁰ M or an even lower Kd value, as measured, e.g., by surface plasmon resonance (e.g., using a BIAcore system).

TFcAs may bind specifically to any target protein, e.g., soluble or membrane human target proteins. Exemplary target proteins include human receptor proteins selected from the group consisting of ErbB2, ErbB3, ErbB4, IGF1R, IGF2R, Insulin receptor, RON, VEGFR1, VEGFR2, TNFR, FGFR1-4, PDGFR (alpha and beta), c-Kit, c-Met, EPCAM and EphA2.

### Exemplary Heavy and Light Chains

In one embodiment, a TFcA comprises a heavy chain and a light chain. In one embodiment, an anti-c-Met/anti-EGFR TFcBA comprises a heavy chain comprising an aa sequence set forth in FIG. 9 and/or a light chain comprising an aa sequence set forth in Example 3.

TFcBAs may also comprise a heavy chain and/or a light chain that comprise an aa sequence that differs from an aa sequence set forth in FIG. 9 in, or in at most 1, 2, 3, 4, 5, 6, 7, 8, 9, 10, 15, 20, 25, 30, 50, 100, 200, or 300 aas, provided that the TFcBA has the desired biological characteristic(s), as further described herein. A TFcBAs may also comprise a heavy chain and/or a light chain comprising an aa sequence that is at least 70%, 75%, 80%, 85%, 90%, 95%, 97%, 98% or 99% identical to the aa sequence of a heavy chain or light chain of FIG. 9, wherein the TFcBA has the desired biological characteristic(s).

TFcBAs may also comprise more than 2 binding sites, in which case, the heavy chain will comprise 1, 2, 3, 4 or more VH domains, which may be linked to the N- and/or C-terminus of any one of the following molecules: Fab-TFc-scFv; Fab-TFc-Fab; scFv-TFc-scFv; and scFv-TFc-Fab. The additional binding sites may be either Fabs or scFvs.

### Biological Activities of TFcAs

In certain embodiments, a TFcA, e.g., a TFcBA, binding to one or more target proteins inhibits signal transduction mediated by the one or more target proteins. For example, an anti-c-Met+anti-EGFR TFcBA may inhibit signal transduction mediated through either or both of c-Met and EGFR. Inhibition of signal transduction may be evidenced, e.g., by inhibition of phosphorylation of EGFR and ERK. In certain embodiments, a TFcA inhibits phosphorylation of c-Met, EGFR and/or ERK by at least 30%, 40%, 50%, 60%, 70%, 80%, 85%, 90%, 95%, 98%, 99% or more, relative to phosphorylation in the absence of the TFcA, when determined, e.g., at the end of the experiment, e.g., as set forth in the Examples. Preferred TFcAs inhibit c-Met and/or EGFR signal transduction, e.g., measured by inhibition of phosphorylation of c-Met and EGFR, nearly completely, e.g., by at least 90%, 91%, 92%, 93%, 94%, 95%, 96%, 97%, 98%, 99% or 99.5%.

Although the biological characteristics described in this section are described mostly in the context of anti-c-Met/anti-EGFR TFcBAs, the description also applies to other TFcBAs, as well as monovalent TFcAs.

Inhibition of a) ligand mediated phosphorylation of c-Met, and b) ligand-mediated phosphorylation of EGFR can be demonstrated by the ability of a TFcBA to reproducibly decrease the level of phosphorylation of a) c-Met induced by an HGF family ligand, b) EGFR induced by an EGFR ligand, e.g., EGF, or c) ERK induced by a c-Met ligand or an EGFR ligand, each relative to the phosphorylation in control cells that are not contacted with the TFcBA. The cell which expresses c-Met and/or EGFR can be a naturally occurring cell or a cell of a cell line or can be recombinantly produced by introducing nucleic acid encoding c-Met and/or EGFR into a host cell. In certain embodiments, a TFcBA inhibits a HGF family ligand mediated phosphorylation of c-Met by at least about 5%, 10%, 20%, 30%, 40%, 50%, 60%, 70%, 80%, 85%, 90%, 91%, 92%, 93%, 94%, 95%, 96%, 97%, 98% or 99%, or more, as determined, for example, by ELISA, and calculated as set forth in the Examples. In certain embodiments, a TFcBA inhibits EGF-mediated phosphorylation of EGFR by at least about 5%, 10%, 20%, 30%, 40%, 50%, 60%, 70%, 80%, 85%, 90%, 91%, 92%, 93%, 94%, 95%, 96%, 97%, 98% or 99%, or more, as determined, for example, by ELISA, and calculated as set forth in the Examples. In certain embodiments, a TFcBA inhibits EGF and/or c-Met-mediated phosphorylation of ERK by at least about 5%, 10%, 20%, 30%, 40%, 50%, 60%, 70%, 80%, 85%, 90%, 91%, 92%, 93%, 94%, 95%, 96%, 97%, 98% or 99%, or more, as determined, for example, by ELISA, and calculated as set forth in the Examples.

TFcBAs may inhibit ligand induced phosphorylation of c-Met by at least 70% or 80%, ligand induced phosphorylation of EGFR by at least 85%, 90% or 95% and optionally ligand induced phosphorylation of ERK by at least 5% or 10%. TFcBAs may also inhibit ligand induced phosphorylation of c-Met by at least 85%, ligand induced phosphorylation of EGFR by at least 85% and optionally ligand induced phosphorylation of ERK by at least 5%. In certain embodiments, TFcBAs inhibit ligand induced phosphorylation of c-Met by at least 50% and ligand induced phosphorylation of EGFR by at least 90%, and optionally ligand induced phosphorylation of ERK by at least 5%.

TFcBAs may also be defined by the EC50 (i.e. the concentration of TFcBA at which 50% of maximum inhibition is obtained) of their inhibition of phosphorylation of one or more of c-Met, EGFR and ERK, which EC50s may be determined as further described herein. For example, TFcBAs disclosed herein may inhibit phosphorylation of c-Met with an EC50 of 10⁻⁸ M, 10⁻⁹ M, 10⁻¹⁰ M or lower. They may inhibit phosphorylation of EGFR with an EC50 of 10⁻⁸ M, 10⁻⁹ M, 10⁻¹⁰ M or lower. They may inhibit phosphorylation of ERK with an EC50 of 10⁻⁷ M, 10⁻⁸ M, 10⁻⁹ M, 10⁻¹⁰ M or lower. Some TFcBAs disclosed herein inhibit phosphorylation of c-Met by at least 80% or 85% with an EC50 of 10⁻⁸ M, 10⁻⁹ M, 10⁻¹⁰ M or lower; inhibit phosphorylation of EGFR by at least 80% or 85% with an EC50 of 10⁻⁸ M, 10⁻⁹ M, 10⁻¹⁰M or lower; and optionally inhibit phosphorylation of ERK by at least 5% with an EC50 of 10⁻⁷ M, 10⁻⁸ M, 10⁻⁹ M, 10⁻¹⁰ M or lower. In some cases, essentially complete blockage of either or both of phosphorylation of c-Met and phosphorylation of EGFR will be obtainable with a TFcBA herein disclosed.

In certain embodiments, a solution comprising TFcAs at a concentration of 0.3, 0.5, 1, 2, 3, 4, 5, 6, 7, 8, 9, 10, 11, 12, 13, 14, 15 mg/ml or more (or ranges of concentrations between any of these two numbers) comprises more than 70%, 75%, 80%, 85%, 90%, 95%, 96%, 97%, 98% or 99% of TFcBAs in unaggregated form (referred to in this context as monomers) as determined e.g., by Size Exclusion Chromatography (SEC) e.g., following, a stability test as described below. The percentage of monomers may be determined in a solution after one of the following stability tests: a) incubation at 4° C. for 1, 2, 3, 4, 5, or 6 days, or 1, 2, 3 or more weeks; b) incubation at room temperature for 1, 2, 3, 4, 5, or 6 days, or 1, 2, 3 or more weeks; c) incubation at 37° C. for 1, 2, 3, 4, 5, or 6 days, or 1, 2, 3 or more weeks; d) 1, 2, 3, 4 or 5 cycles of freeze/thaw, and e) agitation, e.g., gentle agitation on the orbital shaker at room temperature, e.g., for 1, 2, 3, 4, 5 or more hours.

In certain embodiments, a TFcA exhibits a stability after 1, 2, 3, 4 or 5 days of incubation in serum at 37° C. of at least 70%, 75%, 80%, 85%, 90%, 95%, 96%, 97%, 98% or 99%, relative to its stability at day 0, where the stability of a protein is determined by, e.g., SEC or by measuring its ability to bind to one or more of its target antigens after incubation.

In certain embodiments, a TFcA has a melting temperature (Tm) as determined e.g., by Differential Scanning Fluorimetry (DSF) of at least 50° C., 55° C., 58° C. or 60° C., as described in the Examples.

TFcAs may have a combination of two or more of the characteristics set forth herein. For example, a TFcBA may inhibit ligand induced phosphorylation of c-Met by at least 70% and ligand induced phosphorylation of EGFR by at least 70%, and also exhibit one or more of the following characteristics: (i) a Tm, as determined by DSF, of at least 55 or 60° C.; and (ii) be at least 70%, 80% or 90% monomeric in PBS at 10 mg/mL after 5 or more days at at room temperature, 2 weeks at 4° C., one or more cycles of freeze-thaw or gentle agitation. In certain embodiments, TFcBAs have a Tm of at least 60° C. and stability at room temperature, 4° C. or 37° C. of at least 90% (concentration of monomer after incubation under these conditions relative to the initial concentration of monomer).

In certain embodiments, a TFcA composition comprises one or more of the following characteristics: 1) at least 50%, 60%, 70%, 80%, 90% or more of the proteins are visible on SDS PAGE after purification on protein A; 2) at least 50%, 60%, 70%, 80%, 90% or more of observed species on SDS-PAGE gels are of the correct molecular weight; 3) the thermal stability profile, as measured by Differential Scanning Fluorimetry, does not show molten globular behavior; 4) does not comprise more than 50%, 60%, 70%, 80%, 90%, 95%, 98% or 99% or more monomer as visualized by SEC; and 5) inhibits more than 95% of EGF receptor signaling activated by addition of exogenous EGF ligand, as measured by pEGFR inhibition.

Standard assays may be used for determining the biological activity and characteristics of TFcAs, such as TFcBAs. Exemplary assays are provided in the Examples.

### Nucleic Acids, Expression Vectors and Host Cells

Provided herein are nucleic acids, e.g., DNA and RNA, encoding the polypeptides described herein. Exemplary nucleotide sequences provided herein are those encoding the aa sequences set forth in the Figures. In certain embodiments, a nucleotide sequence encoding a heavy or light chain of a TFcA is linked to a sequence that enhances or promotes the expression of the nucleotide sequence in a cell to produce a protein. Such nucleic acids may be encompassed within a vector, e.g., an expression vector.

For the purposes of being secreted, a heavy and/or light chain of a TFcA preferably comprises a signal sequence, which is normally cut off after secretion to provide a mature polypeptide. The following signal sequences may be used:

(SEQ ID NO: 241)
MGFGLSWLFLVAILKGVQC:
for use, e.g., in expressing heavy chains; and
(SEQ ID NO: 243)
MGTPAQLLFLLLLWLPDTTG
for use, e.g., in expressing light chains.

An exemplary nucleotide sequence encoding SEQ ID NO:241 is atgggcttcggactgtcgtggctttttctggtggcgattcttaagggggtccagtgc (SEQ ID NO:240) and an exemplary nucleotide sequence encoding SEQ ID NO:243 is atgggcacccccgcacagctcttgttcttgctgcttctttggctccctgacacaactggt (SEQ ID NO:242).

Nucleic acids, e.g., DNA, that comprise a nucleotide sequence that is at least about 70%, 75%, 80%, 90%, 95%, 97%, 98% or 99% identical to a nucleotide sequence encoding a polypeptide described herein or a nucleotide sequence set forth herein, and which encode a heavy and or light chain of a TFcA, or portion thereof, as further described herein, are also encompassed herein. Such nucleotide sequences may encode a protein set forth herein or may encode a protein that is at least 70%, 75%, 80%, 85%, 90%, 95%, 97%, 98% or 99% identical or similar to a protein set forth herein or a portion thereof (e.g., a domain), such as an aa sequence set forth in any one of the Figures.

Also encompassed herein are cells, e.g., host cells, comprising a nucleic acid or a vector provided herein.

The TFcAs described herein may be produced by recombinant means. Methods for recombinant production are widely known in the state of the art and comprise protein expression in prokaryotic and eukaryotic cells with subsequent isolation of the antibody and usually purification to a pharmaceutically acceptable purity. For the expression of the TFcAs in a host cell, nucleic acids encoding the respective polypeptides, e.g., light and heavy chains, are inserted into expression vectors by standard methods. Expression is performed in appropriate prokaryotic or eukaryotic host cells like CHO cells, NSO cells, SP2/0 cells, HEK293 cells, COS cells, PER.C6 cells, yeast, or E. coli cells, and the TFcA is recovered from the cells (supernatant or cells after lysis). General methods for recombinant production of antibodies are well-known in the state of the art and described, for example, in the review articles of Makrides, S.C., Protein Expr. Purif 17 183-202 (1999); Geisse, S., et al, Protein Expr. Purif. 8 271-282 (1996); Kaufman, R. J., MoI. Biotechnol. 16 151-161 (2000); Werner, R. G., Drug Res. 48 870-880 (1998).

The TFcAs may be suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the TFcAs are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant TFcAs in the host cells.

Aa sequence variants (or mutants) of the TFcAs may be prepared by introducing appropriate nucleotide changes into the TFcA DNA, or by nucleotide synthesis.

“Host cell” denotes any kind of cellular system which can be engineered to generate the TFcAs described herein. In one embodiment, HEK293 cells and CHO cells are used as host cells. Expression in NSO cells is described by, e.g., Barnes, L. M., et al, Cytotechnology 32 109-123 (2000); Barnes, L. M., et al., Biotech. Bioeng. 73 261-270 (2001). Transient expression is described by, e.g., Durocher, Y., et al., Nucl. Acids. Res. 30 E9 (2002). Cloning of variable domains is described by Orlandi, R., et al., Proc. Natl. Acad. Sci. USA 86 3833-3837 (1989); Carter, P., et al., Proc. Natl. Acad. Sci. USA 89 4285-4289 (1992); and Norderhaug, L., et al., J. Immunol. Methods 204 77-87 (1997). An exemplary transient expression system (HEK 293) is described by Schlaeger, E.-J., and Christensen, K., in Cytotechnology 30 71-83 (1999) and by Schlaeger, E.-J., in J. Immunol. Methods 194 191-199 (1996).

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Purification of TFcAs may be performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxylmethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A. Appl. Biochem. Biotech. 75 93-102 (1998)).

### Methods of Using TFcAs

Provided herein are methods of using TFcAs, e.g., TFcBAs. The TFcBAs can be used for treating a disease or disorder associated with receptor dependent signaling, including a variety of cancers.

In one embodiment, a method is provided for inhibiting proliferation of a tumor cell expressing the targets of a TFcA, e.g., c-Met, ErbB2, ErbB3, ErbB4, IGF1R, IGF2R, Insulin receptor, RON, EGFR, VEGFR1, VEGFR2, TNFR, FGFR1-4, PDGFR (alpha and beta), c-Kit, EPCAM and/or EphA2. A method may comprise contacting the tumor cell with a TFcA such that proliferation of the tumor cell is inhibited, slowed down, or stopped or such that the tumor cell dies.

Provided herein are methods for treating a disease or disorder associated with the signaling pathway of the targets of a TFcBA, e.g., c-Met, ErbB2, ErbB3, ErbB4, IGF1R, IGF2R, Insulin receptor, RON, EGFR, VEGFR1, VEGFR2, TNFR, FGFR1-4, PDGFR (alpha and beta), c-Kit, EPCAM and/or EphA2, by administering to a patient a TFcBA in an amount effective to treat the disease or disorder. Suitable diseases or disorders include, for example, a variety of cancers including, but not limited to breast cancer and those set forth below. In one embodiment, a method for treating a subject having a proliferative disease, such as cancer, comprises administering to a subject in need thereof a therapeutically effective amount of one or more TFcA.

Also provided is a method for (or a TFcA, e.g., a medicament for) treating a tumor expressing the target(s) of a TFcA, e.g., a TFcBA, e.g., c-Met, ErbB2, ErbB3, ErbB4, IGF1R, IGF2R, Insulin receptor, RON, VEGFR1, VEGFR2, TNFR, FGFR1-4, PDGFR (alpha and beta), c-Kit, EPCAM, EphA2 and/or EGFR, in a patient, the method comprising administering an amount of a TFcA effective to slow down or stop tumor growth, to stop or to shrink a tumor or to slow or stop tumor invasivness or tumor metastasis). A tumor expressing c-Met, ErbB2, ErbB3, ErbB4, IGF1R, IGF2R, Insulin receptor, RON, VEGFR1, VEGFR2, TNFR, FGFR1-4, PDGFR (alpha and beta), c-Kit, EPCAM, EphA2 and/or EGFR may be treated including tumors of the following cancers: gastric, esophageal, colorectal, non-small cell lung, pancreatic, prostate, renal, and thyroid cancers, hepatocellular carcinoma, glioma/glioblastoma, and breast cancer (basal/triple-negative and HER2+).

A method of treating a tumor or a subject having a tumor can further comprise administering a second anti-cancer agent in combination with the TFcA. Thus novel compositions are contemplated comprising a TFcA, together with a second anti-cancer agent, typically a biologic agent together with at least one pharmaceutically acceptable carrier or excipient.

Also provided are kits comprising one or more TFcAs. The kits may include a label indicating the intended use of the contents of the kit and optionally including instructions for use of the kit in treating a disease or disorder associated with a target of a TFcA dependent signaling, e.g., EGFR and/or c-Met dependent signaling. The term label includes any writing, marketing materials or recorded material supplied on or with the kit, or which otherwise accompanies the kit.

### Pharmaceutical Compositions

In another aspect, a composition, e.g., a pharmaceutical composition, is provided for treatment of a tumor in a patient, as well as methods of use of each such composition to treat a tumor in a patient. The compositions provided herein contain one or more of the antibodies, e.g., TFcAs, disclosed herein, formulated together with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the antibody may be coated in a material to protect it from the action of acids and other natural conditions that may inactivate proteins.

Pharmaceutical compositions may be administered alone or in combination therapy, i.e., combined with other agents. For example, the combination therapy can include an antibody of the present disclosure with at least one additional therapeutic agent, such as an anti-cancer agent. Pharmaceutical compositions can also be administered in conjunction with another anti-cancer treatment modality, such as radiation therapy and/or surgery.

A composition of the present disclosure can be administered by a variety of methods known in the art. As will be appreciated by the skilled artisan, the route and/or mode of administration will vary depending upon the desired results.

To administer a composition provided herein by certain routes of administration, it may be necessary or desirable to coat the antibody with, or co-administer the antibody with, a material to prevent its inactivation. For example, the antibody may be administered to a patient in an appropriate carrier, for example, in liposomes, or a diluent. Pharmaceutically acceptable diluents include saline and aqueous buffer solutions. Liposomes include water-in-oil-in-water CGF emulsions as well as conventional liposomes.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any excipient, diluent or agent is incompatible with the active compound, use thereof in the pharmaceutical compositions provided herein is contemplated. Supplementary active compounds (e.g., additional anti-cancer agents) can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Saline solutions and aqueous dextrose and glycerol solutions can be employed as liquid carriers, particularly for injectable solutions. The composition, if desired, can also contain minor amounts of wetting or solubility enhancing agents, stabilizers, preservatives, or pH buffering agents. In many cases, it will be useful to include isotonic agents, for example, sodium chloride, sugars, polyalcohols such as mannitol, sorbitol, glycerol, propylene glycol, and liquid polyethylene glycol in the composition. Prolonged absorption of the injectable compositions can be brought about by including in the composition an agent that delays absorption, for example, monostearate salts and gelatin.

### EXAMPLES

The following examples should not be construed as limiting the scope of this disclosure.

Throughout the examples, the following materials and methods are used unless otherwise stated. In general, the practice of the techniques of the present disclosure employs, unless otherwise indicated, conventional techniques of chemistry, molecular biology, recombinant DNA technology, immunology (especially, e.g., antibody technology), pharmacology, pharmacy, and standard techniques in polypeptide preparation.

### Example 1

### Identification of Stable Tandem Fc Structures

This example describes the identification of stable multivalent Ab formats. In this Example and in Example 2, protein constructs were used that do not contain binding sites. Several formats were compared, and each of these formats was derived from either one of the following two tandem Fc constructs:

1) TFc “23” or “IgG1 TFc,” which comprises an IgG1 TFc comprising an IgG1 hinge; an IgG1 CH2 domain comprising the substitution N297Q; an IgG1 CH3 domain comprising the substitutions T366S/L368A/Y407V; a TFc linker consisting of (G4S)₈, an IgG1 hinge that does not comprise the upper hinge; an IgG1 CH2 domain comprising the substitution N297Q; and an IgG1 CH3 domain comprising the substitution T366W. This construct comprises the aa sequence set forth as SEQ ID NO:293 (see FIG. 11); and
2) TFc “39” or “IgG1/IgG4 TFc,” which comprises an IgG1/IgG4 tandem TFc comprising a hybrid IgG1/IgG4 hinge comprising an IgG1 upper hinge and a core and lower IgG4 hinge; an IgG4 CH2 domain comprising the substitution T299K; an IgG1 CH3 domain comprising the substitutions T366S/L368A/Y407V; a TFc linker consisting of (G4S)₈; an IgG4 hinge that does not comprise the upper hinge; an IgG4 CH2 domain comprising the substitution T299K; and an IgG1 CH3 domain comprising the substitution T366W. This construct comprises the aa sequence set forth as SEQ ID NO:319 (see FIG. 11).

Six modified versions of TFc 23 and 39 were created, and these are listed in Table 15. Briefly, a first modification was the addition of a disulfide bond in the viscinity of the knob or hole (TFc 23A). Another modification was the change of the knob hole of 23A for a smaller knob/hole (TFc 23B). Another modification introduced 1 or 2 cysteines in the upper hinge of the first hinge, to create disulfide bridges within the TFc (TFc 23 D and C, respectively). Another modification introduced a C-terminal cysteine in the CH3 domain (TFc 23E). Another modification in the TFc consisted of reducing the length of the TFc linker by 20 aas (TFc 23F, also referred to as “23G”). The aa sequences of these newly modified TFcs (shown in Table 15) are the same as those set forth in FIGS. 6 and 7, except that the TFcs used in this example did not comprise part of the upper hinge, i.e., aas EPKSC, and comprised a signal peptide. The nucleotide and aa sequences of these TFcs are shown in FIG. 11, and the identity of each of the domains or elements of the TFcs is set forth in Tables 12 and 13, with the only difference that the first hinge does not comprise EPKSC at its N-terminus.

TABLE 15
TFcs

| Name of TFc | Modifications to TFc | TFc linker | SEQ ID NO of TFc |
|---|---|---|---|
| 23 | T366S/L368A/Y407V::T366W | (G₄S) ₈ | SEQ ID NO: 293 |
| 39 | | | SEQ ID NO: 319 |
| 23A | Y349C/T366S/L368A/Y407V:: S354C/ | (G₄S) ₈ | SEQ ID NO: 295 |
| 39A | T366W | | SEQ ID NO: 321 |
| 23B | Y407T::T366Y | (G₄S) ₈ | SEQ ID NO: 297 |
| 39B | | | SEQ ID NO: 323 |
| 23C | H224C/T225C/T366S/L368A/Y407V:: | (G₄S) ₈ | SEQ ID NO: 299 |
| 39C | T366W | | SEQ ID NO: 325 |
| 23D | T223C/T366S/L368A/Y407V::T366W | (G₄S) ₈ | SEQ ID NO: 301 |
| 39D | | | SEQ ID NO: 327 |
| 23E | T366S/L368A/Y407V/C-term Cysteine:: | (G₄S) ₈ | SEQ ID NO: 303 |
| 39E | T366W/C-term Cysteine | | SEQ ID NO: 329 |
| 23G* | T366S/L368A/Y407V::T366W | (G₄S) ₄ | SEQ ID NO: 305 |

*23G contains the same TFc as that referred to elsewhere as “23F.”

The different nucleic acids (having SEQ ID NOs: 292, 294, 296, 298, 300, 302, 304, 318, 320, 322, 324, 326, or 328) were transiently transfected into Freestyle 293F cells (Invitrogen) and purified with a one step protein A purification essentially as follows. The nucleic acids encoding the proteins are cloned as single proteins into the expression plasmid using standard recombinant DNA techniques. An expression vector employed is pCEP4 (Invitrogen). Expression plasmids are transfected using Polyethylene imine (2.5 μg/ml culture) and DNA (1 μg/ml cell culture). Transfected cells are incubated at 37° C., 5% CO₂ for six days and then harvested. All proteins are purified using protein A affinity protocol, in accordance with manufacturer's instructions. The protein A affinity step is used to selectively and efficiently bind the fusion proteins out of harvested cell culture fluids (HCCF). This removes >95% of product impurities in a single step with high yields and high throughput. The portion of desired molecular form for fusion proteins after this step was in the range of 60 to 98 percent. MABSELECT from GE is used as the Protein A affinity resin. The purified material was concentrated and dialyzed into PBS.

### a) Percentage Monomers

TFc solutions were subjected to the determination of the percentage monomer present by Size Exclusion Chromatography (SEC) either in the initial solution or after incubation at 4° C., 37° C., after freeze-thaw or after gentle agitation on the orbital shaker at room temperature. SEC was performed essentially as follows. SEC is performed using Agilent 1100 Series HPLC system. 50 μg of each molecule is injected on a TSK Super SW3000 gel column (Tosoh Biosciences, P/N 18675). PBS is used as running and equilibration buffer at a flow rate of 0.35 ml/min.

Table 16 provides the percentage monomer of exemplary TFcs in initial solutions at the indicated concentrations.

TABLE 16
Percentage monomer of exemplary TFcs

| | | % monomer |
|---|---|---|
| Protein | Concentration | at 0 days |
| 23G | 3 mg/ml | 87.9 |
| 23 | 5 mg/ml | 71.4 |
| 39C | 0.5 mg/ml | 39.5 |
| 39D | 0.5 mg/ml | 50.9 |
| 39 | 12.5 mg/ml | 66.4 |

In another experiment, the percentage monomer was determined in the initial solution after concentration of the molecules essentially as described above. Table 17 provides the results.

TABLE 17
Percentage monomer in initial solution after concentration

| | mg/ml | % monomer |
|---|---|---|
| 23A | 5.2 | 67% |
| 23D | 10.0 | 78% |
| 23E | 16.8 | 74% |

A compilation of Tables 16 and 17 is shown below in Table 18

TABLE 18
Percentage monomer of exemplary TFcs

| | Initial | % monomer at | Second | % monomer |
|---|---|---|---|---|
| | Concentration | initial | concentration | at second |
| Protein | (mg/ml) | concentration | (mg/ml) | concentration |
| 23 | 5.0 | 71.4% | | |
| 23A | 0.23 | 83.7% | 5.2 | 67% |
| 23B | 1.22 | 58.2% | | |
| 23C | 5.0 | 74.6% | | |
| 23D | 0.32 | 82.0% | 10.0 | 78% |
| 23E | 0.74 | 77.5% | 16.8 | 74% |
| 23G | 0.27 | 87.9% | | |
| 39 | 12.5 | 66.4% | | |
| 39A | n/d | n/d | | |
| 39B | 0.95 | 55.0% | | |
| 39C | 0.5 | 74.6% | | |
| 39D | 0.5 | 50.9% | | |
| 39E | 0.95 | 55.0% | | |
| 39G | 0.27 | 74.8% | | |

Table 19 shows the percentage monomer of TFcs 39E and 23C in solution as determined after having been exposed to various conditions.

TABLE 19
Percentage monomer of 39E and 23C after exposure to various conditions

| | 39E | |
|---|---|---|
| | 12.9 | 23C |
| Condition | mg/ml | 5 mg/ml |
| 0 days | 89.7% | 74.6% |
| 4° C., 2 weeks | 90.6% | 82.5% |
| Room temp., 10 | 90.3% | 83.0% |
| days | | |
| 37° C., 2 weeks | 88.1% | — |
| Freeze/thaw | 90.9% | 85.0% |
| Agitate | 90.7% | — |

The results indicate that the TFcs have very different stabilities in the solution at day 0 and under the various conditions tested. 39E and 23G appear to have better stability than others.

### B) SDS PAGE Analysis

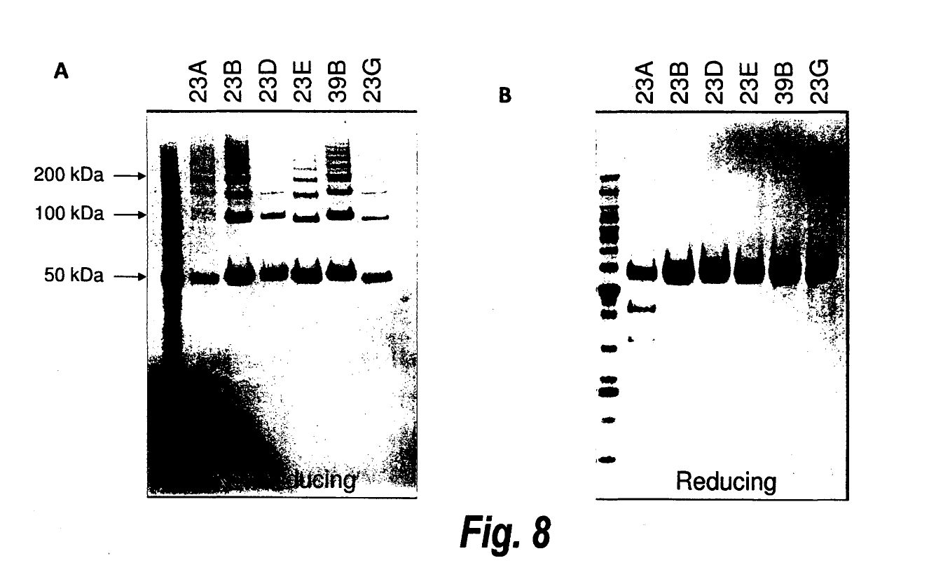

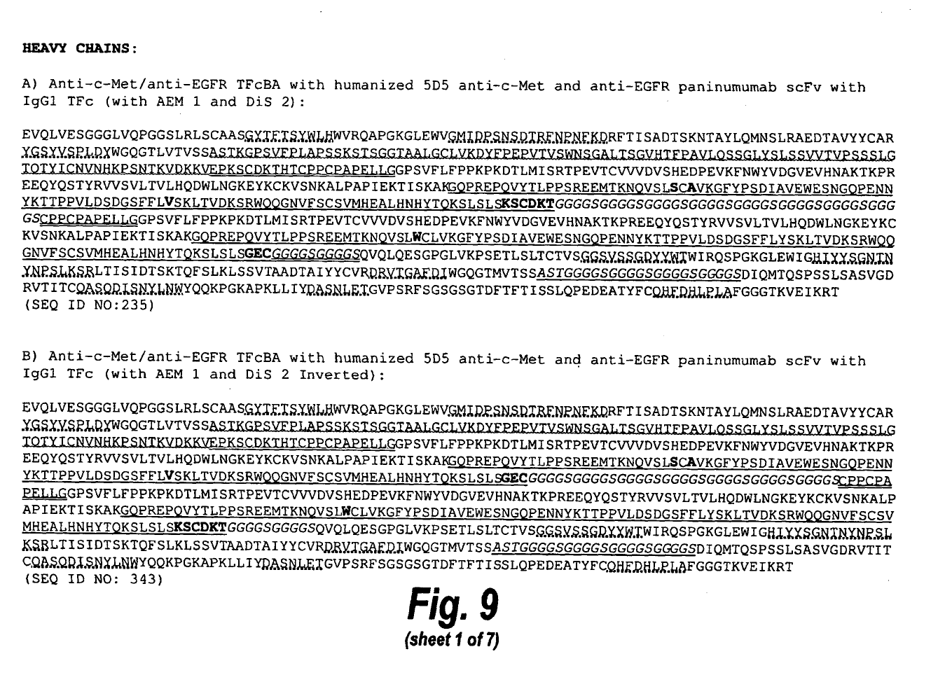

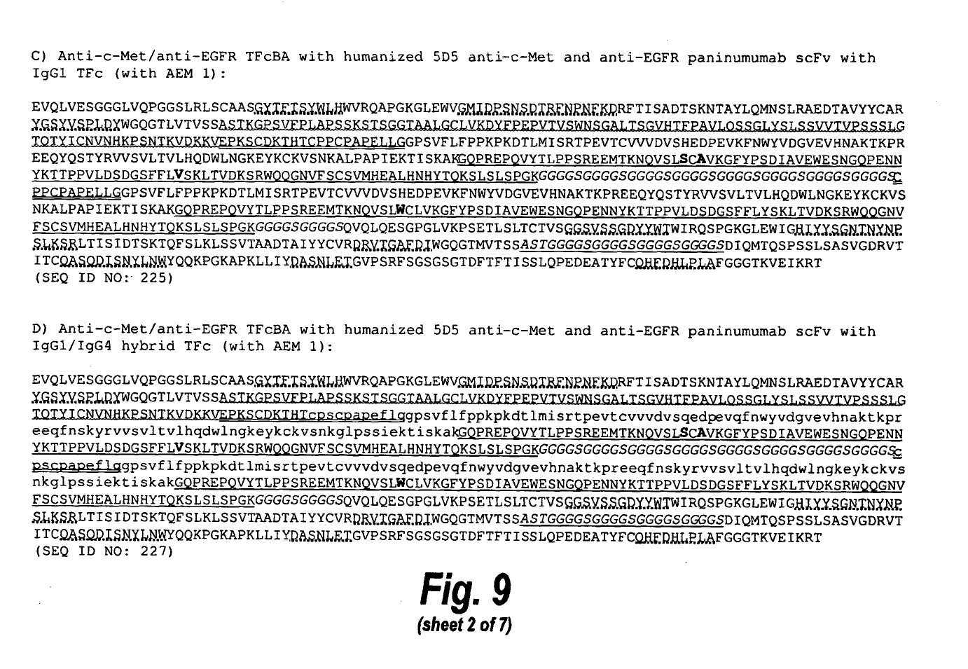

The TFcs were run on a 4-12% SDS-PAGE gel in non-denaturing conditions and visualized by Coomassie stain. The results are shown in FIG. 8.

### Example 2

### Synthesis of Second Generation TFcs

To further improve the characteristics of the TFc molecules, further modifications were made to them. The modifications included (i) varying the length of the TFc linker (“23E (35L)”, “39E (35L)”, “23E (30L)”, “39E (30L)”, “23E (25L)” and “39E (25L)”); (ii) changing the combination of AEMs and C-terminal cysteine modifications within each of the two CH3 domains (“23E (35L) Inverted” and (“39E (35L) Inverted”); and (iii) changing the mutations that enhance CH3 association (“23I”, “39I”, “23J” and “39J”). These modifications are summarized in Table 20. The aa sequences of these newly modified TFcs are set forth in FIG. 11 and the identity of each of their domains or elements is set forth in Tables 12 and 13.

TABLE 20
Second generation TFcs

| Name of TFcBA | Modifications to TFc | TFc linker | SEQ ID NO of TFc |
|---|---|---|---|
| 23E (35L) | T366S/L368A/Y407V/C-term Cysteine | (G₄S) ₇ | SEQ ID NO: 185 |
| 39E (35L) | KSCDKT::T366W/C-term Cysteine GEC | | SEQ ID NO: 211 |
| 23E (35L) | T366S/L368A/Y407V/C-term Cysteine | (G₄S) ₇ | SEQ ID NO: 187 |
| Inverted 39E | GEC::T366W/C-term Cysteine KSCDKT | | SEQ ID NO: 213 |
| (35L) Inverted | | | |
| 23E (30L) | T366S/L368A/Y407V/C-term Cysteine | (G₄S) ₆ | SEQ ID NO: 189 |
| 39E (30L) | KSCDKT::T366W/C-term Cysteine GEC | | SEQ ID NO: 215 |
| 23E (25L) | T366S/L368A/Y407V/C-term Cysteine | (G₄S) ₅ | SEQ ID NO: 191 |
| 39E (25L) | KSCDKT::T366W/C-term Cysteine GEC | | SEQ ID NO: 217 |
| 23I | S364H/F405A::Y349T/T394F | (G₄S) ₈ | SEQ ID NO: 193 |
| 39I | | | SEQ ID NO: 219 |
| 23J | K370D/K392D/K409D::E356K/E357K/D399K | (G₄S) ₈ | SEQ ID NO: 195 |
| 23J | | | SEQ ID NO: 221 |

The second generation TFcs were expressed and purified essentially as described in Example 1.

A) Percentage Monomers

TFc solutions were subjected to the determination of the percentage monomer present by SEC either in the initial solution or after 7 days at 4° C. SEC was performed essentially as described in Example 1.

Table 21 provides the percentage monomer of exemplary second generation TFcs in initial solutions and after 7 days at 4° C.

TABLE 21
Percentage monomer of exemplary TFcs

| | | Day 1 | Day 7 |
|---|---|---|---|
| Fc1 | 23E/25L | 74.8% | |
| Fc2 | 23E/35L | Not measured | 77.9% |
| Fc3 | 39E/30L | 80.2% | |
| Fc4 | 39E/35L | Not measured | 78.9% |
| Fc5 | 39E/35L inv | 79.3% | |
| Fc6 | 23E/30L | 80.2% | |
| Fc7 | 23E/35L inv | 79.1% | |
| Fc8 | 39E/25L | 78.1% | |
| Original IgG1 Fc | 23E/40L | 78.8% | |
| Original IgG1/4 Fc | 39E/40L | 88.3% | |

The results indicate that a 40 aa linker, e.g., in molecules 23E and 39E, results in a more stable TFc than a shorter linker

### Example 3

### Exemplary Anti-c-Met/Anti-EGFR TFcBAs

### a) Exemplary Anti-c-Met Binding Sites

A TFcBA may comprise an anti-c-Met binding site comprising or consisting of that of the humanized 5D5 Ab (U.S. Pat. No. 7,476,724). The heavy chain may comprise the following Fab domain or VH domain thereof:

1) Without signal peptide:

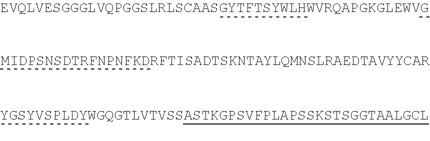

VKDYFPEPVTVSWNSGALTSGVHTFPAVLQSSGLYSLSSVVTVPSSSLG
TQTYICNVNHKPSNTKVDKKV
(SEQ ID NO: 223; the CDRs are underlined with a
dotted line, and the CH1 domain is underlined)
2) Including the exemplary signal peptide
consisting of SEQ ID NO: 241 (underlined):

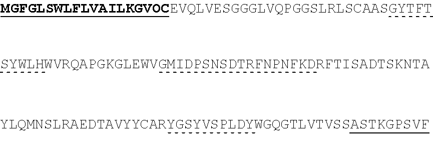

PLAPSSKSTSGGTAALGCLVKDYFPEPVTVSWNSGALTSGVHTFPAVLQ
SSGLYSLSSVVTVPSSSLGTQTYICNVNHKPSNTKVDKKV
(SEQ ID NO: 245; the signal peptide is underlined
and boldface, the CDRs are underlined with a
dotted line, and the CH1 domain is underlined)

The light chain may comprise the following Fab domain or VH domain thereof:

1) Without signal peptide:

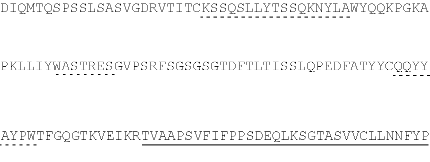

REAKVQWKVDNALQSGNSQESVTEQDSKDSTYSLSSTLTLSKADYEKHK
VYACEVTHQGLSSPVTKSFNRGEC
(SEQ ID NO: 231; the CDRs are underlined with a
dotted line, and the CL domain is underlined)
2) Including the exemplary signal peptide
consisting of SEQ ID NO: 243:

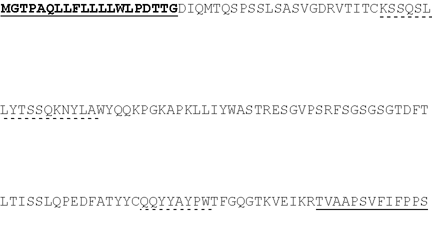

DEQLKSGTASVVCLLNNFYPREAKVQWKVDNALQSGNSQESVTEQDSKD
STYSLSSTLTLSKADYEKHKVYACEVTHQGLSSPVTKSFNRGEC
(SEQ ID NO: 247; the signal peptide is underlined
and boldface, the CDRs are underlined with a
dotted line, and the CL domain is underlined)

A TFcBA may also comprise an anti-c-Met binding site comprising or consisting of the following heavy and light chain portions, and referred to herein as “anti-c-Met binding site 2.” The heavy chain may comprise the following Fab domain or VH domain thereof:

1) Without signal peptide:

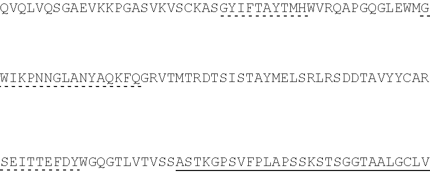

KDYFPEPVTVSWNSGALTSGVHTFPAVLQSSGLYSLSSVVTVPSSSLGT
QTYICNVNHKPSNTKVDKKV
(SEQ ID NO: 287; the CDRs are underlined with a
dotted line, and the CH1 domain is underlined)
2) Including the exemplary signal peptide
consisting of SEQ ID NO: 241 (underlined):

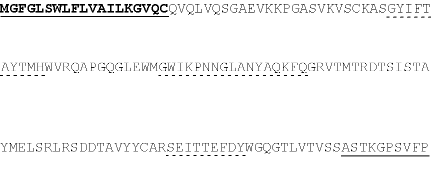

LAPSSKSTSGGTAALGCLVKDYFPEPVTVSWNSGALTSGVHTFPAVLQS
SGLYSLSSVVTVPSSSLGTQTYICNVNHKPSNTKVDKKV
(SEQ ID NO: 256; the signal peptide is underlined
and boldface; the CDRs are underlined with a
dotted line, and the CH1 domain is underlined)

The light chain may comprise the following Fab domain or VH domain thereof:

1) Without signal peptide:

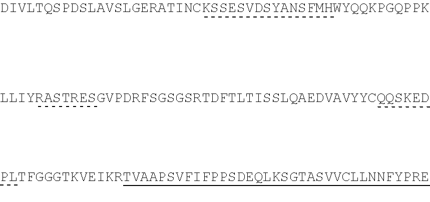

AKVQWKVDNALQSGNSQESVTEQDSKDSTYSLSSTLTLSKADYEKHKVY
ACEVTHQGLSSPVTKSFNRGEC
(SEQ ID NO: 289; the CDRs are underlined with a
dotted line, and the CL domain is underlined)
2) Including the exemplary signal peptide
consisting of SEQ ID NO: 243:

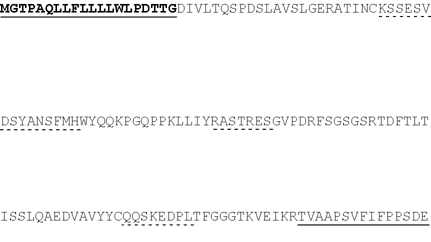

QLKSGTASVVCLLNNFYPREAKVQWKVDNALQSGNSQESVTEQDSKDST
YSLSSTLTLSKADYEKHKVYACEVTHQGLSSPVTKSFNRGEC
(SEQ ID NO: 345; the signal peptide is underlined
and boldface; the CDRs are underlined with a
dotted line, and the CL domain is underlined)

The aa sequences of the heavy chain of an exemplary mature TFcBA comprising in amino to carboxy terminal order: i) the Fab domain of the anti-c-Met binding site 5D5 (SEQ ID NO:223); ii) a TFc comprising AEM 1 and DiS 2 (SEQ ID NO:181); and iii) the panitumumab anti-EGFR scFv H1L1 having SEQ ID NO:233 (see below) is set forth as SEQ ID NO:235 (FIG. 9). The aa sequences of the heavy chain of an exemplary mature TFcBA comprising in amino to carboxy terminal order: i) the Fab domain of the anti-c-Met binding site 2 (SEQ ID NO:287), ii) a TFc comprising AEM 1 and DiS 2 (SEQ ID NO:181); and iii) the cetuximab anti-EGFR scFv H1L1 having SEQ ID NO:258 (see below) is set forth as SEQ ID NO:291 (FIG. 9). Generally, the exemplary anti-c-Met Fab heavy chain sequences provided here may be linked to any of the TFcs, or constructs comprising a TFc, disclosed herein. These proteins may be expressed with a signal sequence, which may be the signal sequence consisting of SEQ ID NO:241.

B) Exemplary Anti-EGFR scFvs
A TFcBA may comprise any of the following anti-EGFR scFvs (or variable domains or CDRs thereof):

1) Panitumumab (VECTIBIX) scFv
The aa sequence of the VH and VL domains of
panitumumab is provided in U.S. Pat. No.
6,235,883, and are assembled into an scFv having
the following aa sequence:

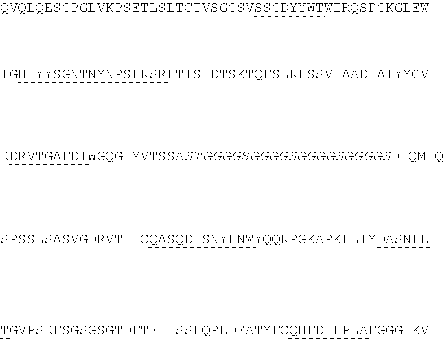

EIKRT
(SEQ ID NO: 233; the scFv linker is in italics
and the VH and VL CDRs are underlined with a
dotted line)
2) 2224 scFv
The aa sequence of the VH and VL domains of Ab
2224 is provided in US Patent Publication No
2010/0009390, and are assembled into an scFv
having the following aa sequences:

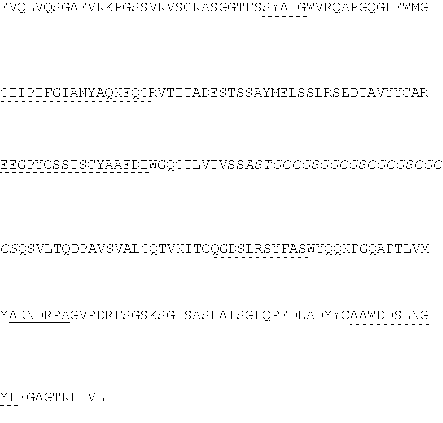

(SEQ ID NO: 237; the scFv linker is in italics
and the VH and VL CDRs are underlined with a
dotted line)
3) Humanized Cetuximab (ERBITUX) scFv
The variable regions of Cetuximab were humanized
and used for constructing the following scFvs,
wherein the CDRs are underlined with dotted
lines, the scFv linker is italicized and aa
modifications resulting from the humanization are
in lower case letters:
3.1) Cetuximab scFv H1 L1

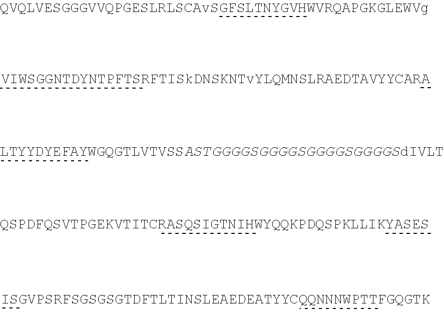

VEIKRT
(SEQ ID NO: 258)
3.2) Cetuximab scFv H1 L2

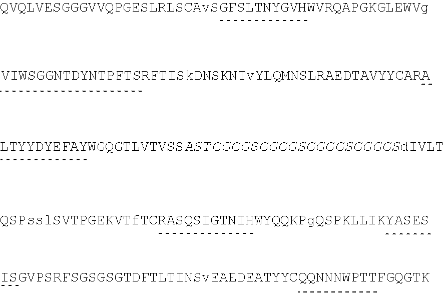

lEIKRT
(SEQ ID NO: 275)
3.3) Cetuximab scFv H2 L1

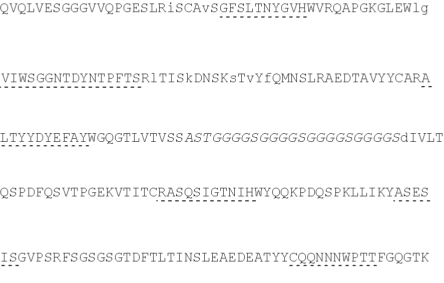

VEIKRT
(SEQ ID NO: 277)
3.4) Cetuximab scFv H2 L2

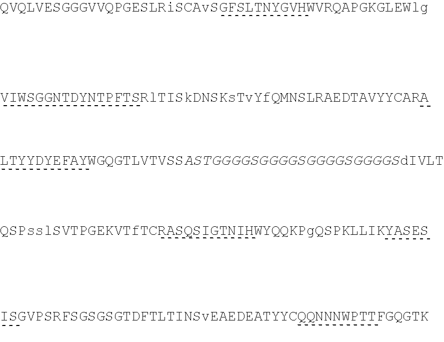

lEIKRT
(SEQ ID NO: 279)

The VH and VL domains of the humanized Cetuximab Abs may be used in any other format of Ab, e.g., an Ab having a naturally occurring structure comprising two heavy chains and two light chains.

The aa sequence of the heavy chain of an anti-c-Met/anti-EGFR TFcBA comprising i) the humanized 5D5 anti-c-Met VH domain (SEQ ID NO:223); ii) a TFc comprising AEM 1 and DiS 2 (SEQ ID NO:181); and iii) the panitumumab anti-EGFR scFv having SEQ ID NO:233 is set forth as SEQ ID NO:235 (FIG. 9). The aa sequences of the heavy chain of anti-c-Met/anti-EGFR TFcBAs comprising the same binding sites as those in SEQ ID NO:235, but comprising a different TFc are set forth in SEQ ID NOs: 343, 225, 227 and 229 (FIG. 9). The aa sequence of the heavy chain of an anti-c-Met/anti-EGFR TFcBA comprising i) the humanized 5D5 anti-c-Met VH domain (SEQ ID NO:223); ii) a TFc comprising AEM 1 and DiS 2 (SEQ ID NO:181); and iii) the 2224 anti-EGFR scFv having SEQ ID NO:237 is set forth as SEQ ID NO:239 (FIG. 9). The aa sequences of the heavy chain of an anti-c-Met/anti-EGFR TFcBA comprising i) the humanized 5D5 anti-c-Met VH domain (SEQ ID NO:223); ii) a TFc comprising AEM 1 and DiS 2 (SEQ ID NO:181); and iii) a cetuximab anti-EGFR scFv consisting of SEQ ID NO:258, 275, 277 or 279 are set forth as SEQ ID NOs:260, 281, 283 and 285, respectively (FIG. 9). Generally any of the anti-EGFR scFvs disclosed herein, or their variable or CDR sequences, may be linked to any of the TFcs, or constructs comprising a TFc, disclosed herein.

Nucleotide sequences encoding the Fab domains, scFvs and TFcBAs are provided in FIG. 10.

Other exemplary anti-c-Met/anti-EGFR TFcBAs are set forth in Table 21, wherein each of the sequences may be connected to the adjacent sequence in amino to carboxy terminal order without intervening sequence.

TABLE 21
Exemplary TFcBAs

| Anti-c-Met heavy | | Connecting | Anti-EGFR |
|---|---|---|---|
| chain Fab | TFc | linker | scFv |
| Humanized 5D5 (SEQ | IgG1 TFc (SEQ ID NO: 171, 173, 175, 177, 179, | (G4S)2 | Panitumumab |
| ID NO: 223 or 245) | 181, 183, 185, 187, 189, 191, 193 or 195) or | | (SEQ ID |
| with light chain having | IgG1/IgG4 hybrid IFc (SEQ ID NO: 197, 199, | | NO: 233) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | 2224 (SEQ ID |
| ID NO: 223 or 245) | 181, 183, 185, 187, 189, 191, 193 or 195) or | | NO: 237) |
| with light chain having | IgG1/IgG4 hybrid IFc (SEQ ID NO: 197, 199, | | |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H1L1 (SEQ ID |
| with light chain having | IgG1/IgG4 hybrid IFc (SEQ ID NO: 197, 199, | | NO: 258) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H1L2 (SEQ ID |
| with light chain having | IgG1/IgG4 hybrid IFc (SEQ ID NO: 197, 199, | | NO: 275) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H2L1 (SEQ ID |
| with light chain having | IgG1/IgG4 hybrid IFc (SEQ ID NO: 197, 199, | | NO: 277) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H2L2 (SEQ ID |
| with light chain having | IgG1/IgG4 hybrid IFc (SEQ ID NO: 197, 199, | | NO: 279) |
| SEQ ID NO: 231 or | 201, 203, 205, 207, 209, 211, 213, 215, 217, 219 | | |
| 247 | or 221) | | |
| Binding site 2 (SEQ | IgG1 TFc (SEQ ID NO: 171, 173, 175, 177, 179, | (G4S)2 | Panitumumab |
| ID NO: 287 or 256) | 181, 183, 185, 187, 189, 191, 193 or 195) or | | (SEQ ID |
| with light chain having | IgG1/IgG4 hybrid IFc (SEQ ID NO: 197, 199, | | NO: 233) |
| SEQ ID NO: 289 or | 201, 203, 205, 207, 209, 211, 213, 215, 217, 219 | | |
| 345 | or 221) | | |
| Humanized 5D5 (SEQ | IgG1 TFc (SEQ ID NO: 171, 173, 175, 177, 179, | (G4S)2 | 2224 (SEQ ID |
| ID NO: 223 or 245) | 181, 183, 185, 187, 189, 191, 193 or 195) or | | NO: 237) |
| with light chain having | IgG1/IgG4 hybrid IFc (SEQ ID NO: 197, 199, | | |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H1L1 (SEQ ID |
| with light chain having | IgG1/IgG4 hybrid IFc (SEQ ID NO: 197, 199, | | NO: 258) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H1L2 (SEQ ID |
| with light chain having | IgG1/IgG4 hybrid 11-c (SEQ ID NO: 197, 199, | | NO: 275) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H2L1 (SEQ ID |
| with light chain having | IgG1/IgG4 hybrid IFc (SEQ ID NO: 197, 199, | | NO: 277) |
| SEQ ID NO: 231 or | 201, 203, 205, 207, 209, 211, 213, 215, 217, 219 | | |
| 247 | or 221) | | |
| Humanized 5D5 (SEQ | IgG1 TFc (SEQ ID NO: 171, 173, 175, 177, 179, | (G4S)2 | Cetuximab |
| ID NO: 223 or 245) | 181, 183, 185, 187, 189, 191, 193 or 195) or | | H2L2 (SEQ ID |
| with light chain having | IgG1/IgG4 hybrid IFc (SEQ ID NO: 197, 199, | | NO: 279) |
| SEQ ID NO: 231 or | 201, 203, 205, 207, 209, 211, 213, 215, 217, 219 | | |
| 247 | or 221) | | |

### Example 4

### Methods for Preparing and Characterizing TFcAs or TFcs

### a) Protein Expression and Purification

Stable Transfection Nucleic acids are transfected into CHO-K1 cells (Chinese hamster ovary; ATCC® cat #CCL-61™) using 1:1(:1) plasmid ratio, and are purified with a one step protein A purification method, e.g., according to the following protocol. The nucleic acids encoding the TFcAs or TFcs are cloned as single proteins into the expression plasmids using standard recombinant DNA techniques. An exemplary expression vector employed is pMP 10K (SELEXIS). Expression plasmids are linearized, purified using QIAquick® purification kit (QIAGEN), and co-transfected into CHO-K1 cells using Lipofectamine® LTX (Invitrogen). Transfected cells are recovered with Ham's F12 medium (Gibco®) containing 10% FBS for 2 days without selection pressure, then with selection pressure for 4 days. After 4 days, they are changed into serum-free medium (Hyclone®) containing glutamine with selection pressure. After a week, cells are checked for expression and scaled up to desired volume. All proteins are purified using protein A affinity protocol, carried out in accordance with manufacturer's instructions. The protein A affinity step is used to selectively and efficiently bind the TFcA or TFc proteins out of harvested cell culture fluids (HCCF). This removes >95% of product impurities in a single step with high yields and high throughput. The portion of desired molecular form for TFcAs or TFcs after this step is expected to be in the range of 60 to 98 percent. MABSELECT from GE is used as the Protein A affinity resin. The purified material is concentrated and dialyzed into PBS.

Transient transfection: Nucleic acids are transiently transfected into Freestyle™ 293F cells (Invitrogen) and purified with a one-step protein A purification essentially as follows. The nucleic acids encoding the proteins are cloned as single proteins into the expression plasmid using standard recombinant DNA techniques. An exemplary expression vector employed is pCEP4 (Life Technologies cat #R790-07). Expression plasmids are transfected using Polyethylene imine (2.5 μg/ml culture) and DNA (1 μg/ml cell culture). Transfected cells are incubated at 37° C., 5% CO₂ for six days and then harvested. All proteins are purified using protein A affinity protocol, in accordance with manufacturer's instructions. The protein A affinity step is used to selectively and efficiently bind the fusion proteins out of harvested cell culture fluids (HCCF). This removes >95% of product impurities in a single step with high yields and high throughput. MABSELECT from GE is used as the Protein A affinity resin. The purified material is concentrated and dialyzed into PBS.B) SDS-PAGE analysis

TFcBAs or TFcs are run on a 4-12% SDS-PAGE gel in non-denaturing conditions and visualized by Coomassie stain. This method may be used to determine whether a TFcA or TFc is properly formed or assembled.

### C) Thermal Stability Measurement by DSF

The temperature at which a TFcA or TFc unfolds is determined by Differential Scanning Fluorimetry (DSF) essentially as follows. The DSF assay is performed in the IQ5 Real Time Detection System (Bio-Rad). 20 μl solutions of 15 uM TFcA or TFc, 1× Sypro Orange (Invitrogen Life Technologies), and 1×PBS are added to the wells of a 96 well plate. The plate is heated from 20° C. to 90° C. with a heating rate of 1° C./min. Data is transferred to GraphPad Prism® for analysis.

D) pEGFR Inhibition

Inhibition of signal transduction through EGFR, e.g., ligand-induced signal transduction, by a TFcBA may be determined by measuring the effect of the particular TFcA on the phosphorylation of EGFR.

The following protocol is used to measure inhibition of EGFR. Cells (e.g. A431 cells (ATCC® cat #CRL-1555™) or NCI-H322M (National Cancer Institute)) are maintained in DMEM medium supplemented with 10% fetal bovine serum, Penicillin/Streptomycin and L-glutamine. For signaling experiments, 3.5×10⁴ cells are plated in complete medium in 96-well tissue culture plates. The following day, complete medium is replaced with serum-free medium, and cells are incubated overnight at 37° C. Cells are pretreated for 2 hours with starting concentration of 300 nM and titrating 3-fold down for an 11 concentration dose for each TFcA or TFc, and then stimulated for 10 minutes with 8 nM EGF (Human recombinant EGF; Cat# AF-100-15; PeproTech, Inc.). Cells are washed with PBS and lysed in MPER buffer (cat #PI78505, VWR International) supplemented with protease and phosphatase inhibitors (cOmplete™ Protease Inhibitor Cocktail Tablet provided in EASY packs, cat #4693124001, Roche Diagnostics Corp; PhosSTOP® Phosphatase Inhibitor Cocktail Tablets, cat #4906837001, Roche Diagnostics Corp). ELISAs for phospho-EGFR (pEGFR) are performed according to the manufacturer's protocols (pEGFR ELISA R&D kit (cat #: DYC1095-C)), with the exception that the capture Ab is EGFR Ab-11, Clone: 199.12 (Fisher Scientific Cat# MS396P1ABX). SuperSignal® ELISA Pico Chemiluminescent Substrate (cat #PI37069, VWR International) is added and plates read on a PerkinElmer Envision plate reader. Luminescence values are plotted following normalization to the observed signal at the lowest concentration of TFcA or TFc. For data analyses, duplicate samples are averaged and error bars are used to represent the standard deviation between the two replicates Inhibition curves and corresponding IC50 values are calculated using GraphPad Prism® software (GraphPad Software, Inc.) via regression of the data to a 4 parameter logistic equation. To calculate percent inhibition, regressed values of maximal (‘max’) and minimum (‘min’) inhibitor potency can be utilized as follows:

curve_span=max−min;
baseline_span=max;
percent_inhibition=100*curve_span/baseline_span.
E) pERK Inhibition

Inhibition of signal transduction through c-Met and/or EGFR, e.g., ligand-induced signal transduction, by a TFcA may may be determined by measuring the effect of the particular TFcA on the phosphorylation of ERK. The following protocol may be used to measure inhibition of pERK. Day 1: Actively midlog (about 80% confluency) growing cells (e.g., A431 cells) are split in DMEM (+10% FBS+L/glutamine+Pen/Strep) media. Approximately 35,000 cells are seeded/well in a 96 well-plate. Day 2: The media is changed from 10% FBS to serum-free media −0.5% FBS (+L/glutamine+Pen/Strep) media. Day 3: The inhibitors/antibodies are diluted into the appropriate volume of serum media. 100 μL of each inhibitor per concentration is added/well. The inhibitor is allowed to incubate at 37° C. for 2 hours. At the end of the 2 hour period, a final concentration of 8 nM EGF (Human recombinant EGF; Cat# AF-100-15; PeproTech, Inc.) is added to each inhibitor and each concentration of inhibitor for 10 min. The cells are washed 2× with cold PBS and later lysed in 40 μL/well of SureFire® Lysis buffer (a 1:5 dilution of stock with water). The lysates are place in −80° C. usually within 5 min after lysis. Day 4: The protocol on performing the SureFire® pERK 1/2 ELISA can be found in Perkin Elmer website (ALPHASCREEN PROTEIN A 10K PTS PerkinElmer Life Sciences, Inc. Cat #: 6760617M; TGR Surefire ERK1 384 Kit for 10,000 Ass; PerkinElmer Life Sciences, Inc. Cat #: TGRES10K). Essentially, the −80° C. lysate is thawed at room temperature. In the meantime, the reaction buffer is prepared in which the Activation Buffer and Reaction Buffer are mixed according to protocol. The Protein A detection kit reagents are added last to the reaction buffer prior to adding onto the 384 well plate. 4 μL of the thawed lysate is transferred onto a ProxiPlate® 384, white shallow well plate (from Perkin Elmer; cat #6008280). After addition of the Protein A detection kit reagents, 7 μL of the final reaction buffer is transferred to each well (which already has 4 μL of the lysate in them). The plates are sealed tightly with aluminum sealer. The plate is spun down in an Eppendorf table top centrifuge at 1800 rpms for 1 minute. The plates are gently shaken at RT for 2 hours. The plates are then read in Perkin Elmer Envision® Reader. Normalization of luminescence data and calculation of IC50 occurs as described for pEGFR.

### Example 5

### Protocols for Measurement of ELISA Plate-Based Antibody Binding

Binding of Bispecific Antibodies to Soluble cMet-Fc and EGFR-his

Reacti-bind® plates (96 well) are coated with 50 μL of cMet-Fc (2 μg/mL in PBS), and incubated overnight at 4° C. Next day, the plates are washed with PBS-T (PBS+0.05% Tween-20), blocked for 1 hour at room temperature with 100 μL of blocking buffer, and washed again with PBS-T. Plates are incubated with 50 μL of bispecific antibodies at room temperature for 2 hours, and then washed with PBS-T. Antibody concentrations start at 500 nM (in PBS-T), and include ten additional two-fold dilutions and one blank (PBS-T only). Plates are then incubated with 50 μL of EGFR-his (at 1 μg/ml in PBS-T for one hour at room temperature. The plates are washed with PBS-T and then incubated with anti-his-HRP antibody diluted 1:10,000 in PBS-T for 1 hour at room temperature, and washed again with PBS-T. The plates are incubated with 100 μL of TMB substrate for 5-10 minutes at room temperature and the reaction is stopped by adding 100 μL of Stop solution. The absorbance was measured at 450 nm, and the resulting data analyzed using GraphPad Prism®.

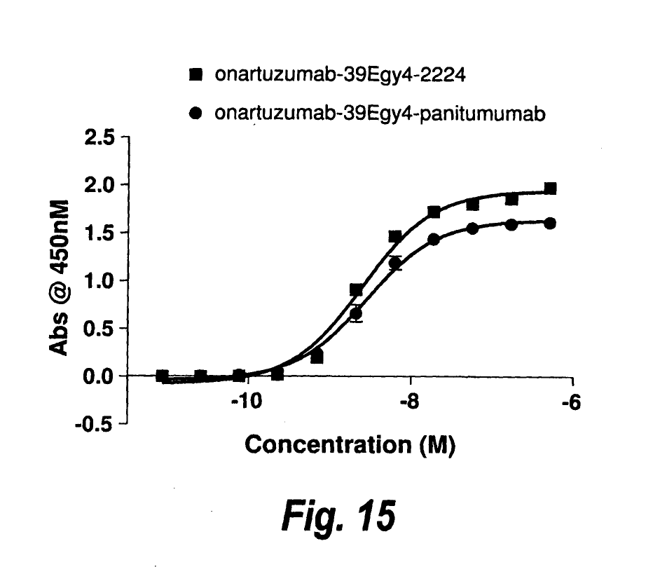

Exemplary results using TFcBAs in the method above can be seen in FIG. 15, which shows binding affinity of onartuzumab-39Egy-2224 (squares) and onartuzumab-39Egy4-panitumumab (circles).

### Example 6

### Current Technologies for Asymmetric Fc-Domains Give Molecular Weight Heterogeneity

### Stable Transfection of CHO-K1 Cells

Suspension-adapted CHO-K1 cells are grown in Hyclone® Media supplemented with 8 mM L-glutamine to a density of 2 million/mL. On the day of transfection, the cells are resuspended in a serum-free media (Opti-MEM® I) at a density of 80,000 cells/mL. The cells (500mL) are then transfected with 1 μg of total DNA (including 10 ng of pNeo vector, an in-house vector carrying the geneticin selection marker) using 2.75mL of Lipofectamin®e in a 24 well plate. After 3 hours, 1 mL of recovery media (HAMS-F12+10% FBS) is added, and the transfected cells allowed to recover for 48 hours. The cells are then expanded into a 96-well plate, and the selection marker geneticin was added to the recovery media at 500 μg/ml. After 4 more days, the media is replaced with serum free Hyclone media (supplemented with L-glutamine), and the transfected cells allowed to adapt. After a week, the selected cells form colonies, and the wells are tested for desired characteristics with western blots from the supernatant. The desired clones are expanded to a 24-well plate, then to a T-25 flask, and eventually to a shake flask. The desired clones are confirmed with SDS-PAGE, and scaled up to the desired volume. The cells are harvested by centrifugation (6000 g, 30 min) when the viability falls below 80%, and the supernatant filtered using a 0.22 μm filter.

Cells were transfected as described aboved and analyzed as follows. Results are shown in Table 22 below.

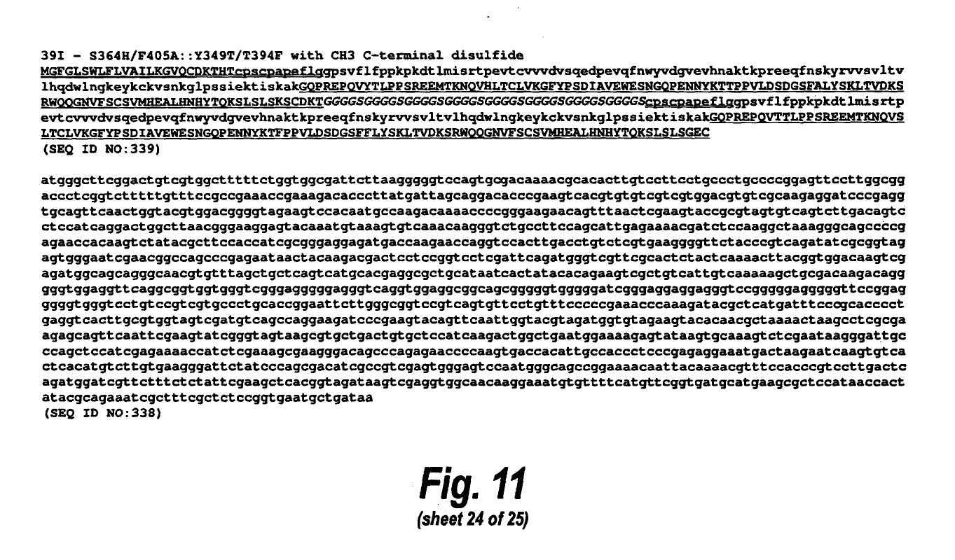

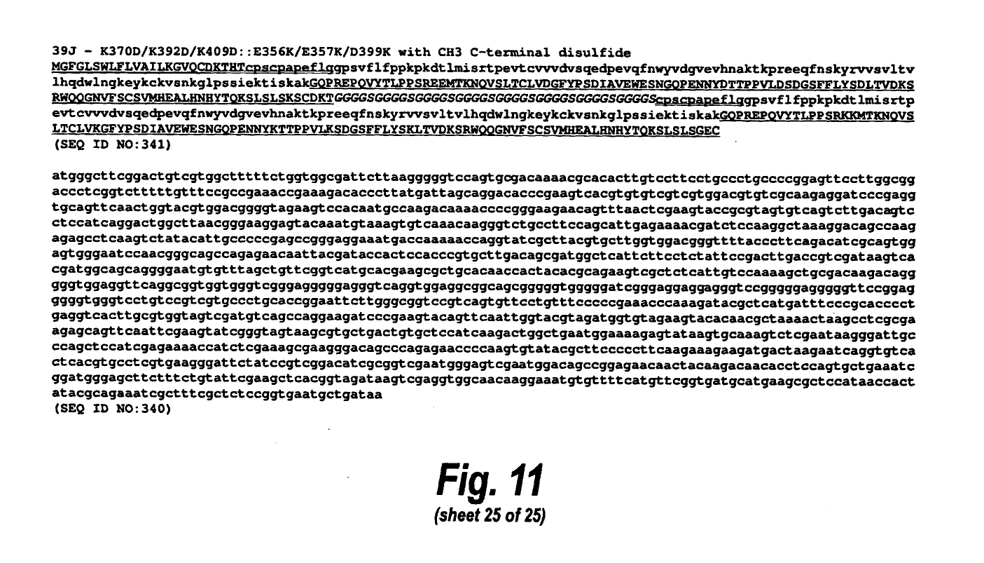

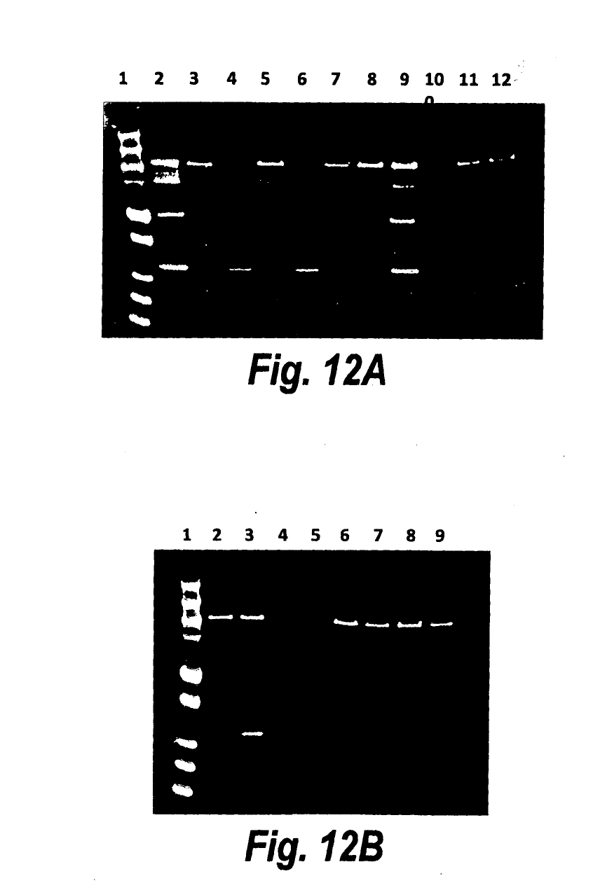

A) Western Blot Protocol
Cell supernatants expressing onartuzumab were run on a 4-12% SDS-PAGE gel in non-denaturing conditions. The proteins were transferred to nitrocellulose paper using the Invitrogen iBlot®. The blot was washed with PBS-T and then incubated for one hour with anti-human-FC conjugated to IRD700. The blot was washed three times with PBS-T and then imaged using the L1-Cor® Odyssey®. The results are shown in FIG. 12.

B) Percentage Monomers
TFc solutions were subjected to the determination of the percentage monomer present by SEC in the initial solution. SEC was performed essentially as described in Example 1.
Table 22 provides the percentage monomer of exemplary second generation TFcs in initial solutions.

TABLE 22
Percentage monomer of exemplary onartuzumab clones

| Protein | High MW species | % monomer | Low MW species |
|---|---|---|---|
| Onartuzumab1 | | 78 | 22 |
| Onartuzumab2 | | 75 | 25 |
| Onartuzumab3 | | n/d | |
| Onartuzumab4 | | 24 | 75 |
| Onartuzumab5 | | 89 | 11 |
| Onartuzumab6 | 2.2 | 98 | |

### Example 7

### Production and Analysis of Charged Aglycosylation Mutants

The identification of stable multivalent Ab formats is described below. Protein constructs were used that do not contain binding sites. Several formats were compared as shown in Table 23 and FIG. 17.

TABLE 23
TFcs

| Name of | Modifications to TFc | SEQ ID NOs of TFc |
|---|---|---|
| tandem Fc | CH2 domain | (aa/nucleotide) |
| Glyco_wt | None | 357/358 |
| Glyco_1 | N297D/T299S::N297D/T299S | 388/389 |
| Glyco_2 | T299K::N297D/T299S | 390/391 |
| Glyco_3 | N297D/T299S::T299K | 392/393 |
| Glyco_4 | N299K::N299D | 394/395 |
| Glyco_5 | N299D::N299K | 396/397 |
| Glyco_6 | N299D::N299D | 398/399 |

Nucleic acids (having SEQ ID NOs:357, and 389-399 (odd numbers) were transiently transfected into Freestyle™ 293F cells and purified with a one-step protein A purification followed by DSF essentially as described in Example 4.

TABLE 24
Percentage monomer of TFcs after exposure to various conditions

| Protein | Condition | mg/ml | % monomer |
|---|---|---|---|
| Glyco_wt | Initial purification | 10.6 | 86.6 |
| | 4 deg, 0 day | 10.6 | 79.7 |
| | Agitate | 10.6 | 79.3 |
| | 4 deg, 10 day | 10.6 | 78.6 |
| | 37 deg, 10 day | 10.6 | 75.5 |
| | 4 deg, 1 month | 10.6 | 77.5 |
| Glyco_1 | Initial purification | 3.7 | 83.2 |
| | 4 deg, 0 day | 11.2 | 83.0 |
| | Agitate | 11.2 | 82.6 |
| | 4 deg, 10 day | 11.2 | 82.4 |
| | 37 deg, 10 day | 11.2 | 80.3 |
| | 4 deg, 1 month | 11.2 | 84.7 |
| Glyco_2 | Initial purification | 1.3 | 78.6 |
| | 4 deg, 0 day | 8.4 | 78.0 |
| | Agitate | 8.4 | n/d |
| Glyco_3 | Initial purification | 3 | 76.4 |
| | 4 deg, 0 day | 12 | 75.0 |
| | Agitate | 12 | n/d |
| | 4 deg, 10 day | 12 | 73.5 |
| | 37 deg, 10 day | 12 | 71.6 |
| Glyco_4 | Initial purification | 4.2 | 84.5 |
| | 4 deg, 0 day | 10.1 | 83.5 |
| | Agitate | 10.1 | 83.6 |
| | 4 deg, 10 day | 10.1 | 83.0 |
| | 37 deg, 10 day | 10.1 | 80.8 |
| Glyco_5 | Initial purification | 9.5 | 77.1 |
| | 4 deg, 0 day | 9.5 | 76.3 |
| | Agitate | 9.5 | 76.5 |
| | 4 deg, 10 day | 9.5 | 76.6 |
| | 37 deg, 10 day | 9.5 | 73.7 |
| | 4 deg, 1 month | 9.5 | 76.5 |
| Glyco_6 | Initial purification | 6.3 | 81.7 |
| | 4 deg, 0 day | 12 | 80.6 |
| | Agitate | 12 | 81.2 |
| | 4 deg, 10 day | 12 | 81.0 |
| | 37 deg, 10 day | 12 | 77.3 |
| | 4 deg, 1 month | 12 | 80.8 |

### B) SDS PAGE Analysis

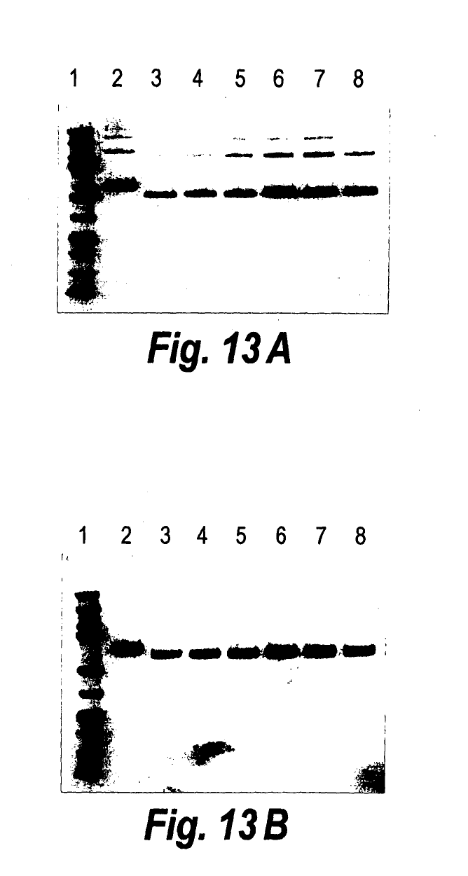

The TFcs were run on a 4-12% SDS-PAGE gel in denaturing conditions and visualized by Coomassie stain. The results are shown in FIG. 13A (non-reduced) and FIG. 13B (reduced).

### C) Thermal Stability Measurement by DSF

The temperature at which a TFcA or TFc unfolds is determined by Differential Scanning Fluorimetry (DSF) essentially as follows. The DSF assay is performed in the IQ5 Real Time Detection System (Bio-Rad). 20 μl solutions of 15 uM TFcA or TFc, 1× Sypro® Orange (Invitrogen Life Technologies), and 1×PBS are added to the wells of a 96 well plate. The plate is heated from 20° C. to 90° C. with a heating rate of 1° C./min. Data is transferred to GraphPad Prism® for analysis. Exemplary results from thermal stability determination by DSF for glycosylation site mutants are shown in Table 25.

TABLE 25
DSF of Tandem Fcs

| protein | Tm |
|---|---|
| glyco_wt | 60.5 |
| glyco_1 | 55.5 |
| glyco_2 | 57.9 |
| glyco_3 | 57.9 |
| glyco_4 | 57.3 |
| glyco_5 | 56.3 |
| glyco_6 | 51.5 |

### Example 8

### DSF Analysis of Backbone Variants

### Thermal Stability Measurement by DSF

The temperature at which a TFcA or TFc unfolds is determined by Differential Scanning Fluorimetry (DSF) as described above. Results are shown in Table 26 below. These data show that backbone modifications such as the addition of a disulfide bridge and glycosylation mutations can improve thermal stability.

TABLE 26
DSF of Tandem Fcs with backbone variations

| Protein | Tm |
|---|---|
| Onartuzumab | 57.1 |
| Onartuzumab-23 | 46.9 |
| Onartuzumab-39 | 51.2 |
| Onartuzumab-23E | 57.8 |
| Onartuzumab-39Egy4 | 60.3 |
| Onartuzumab-39Egy4-cetuximab | 57.3 |
| Onartuzumab-39Egy4-panitumumab | n/a |
| Onartuzumab-39Egy4-2224 | 55.7 |

### Example 9

### Production and Analysis of Monovalent and Bispecific tFc Molecules Using Onartuzumab Binding Site

### Percent Monomer Determination Using Size Exclusion Chromatography

50 μg of sample is injected on a TSKgel SuperSW3000 column (4.6 mm ID×30 cm) using 20 mM sodium phosphate (+300 mM NaCl) as running buffer. All measurements are performed on Agilent 1100 HPLC which is equipped with an auto sampler, a binary pump and a diode array detector. Percent monomers are determined by analyzing the data in Chemstation software. Typically, all the samples are only protein A purified and at a concentration of 5 mg/mL in 1×PBS.

TABLE 27
SEC Stability of MM131 molecules at 4° C.

| | Percent | Percent |
|---|---|---|
| | monomers | monomers |
| Molecule name | (Day 0) | (Day 7) |
| Onartuzumab-23 | 72 | 76 |
| Onartuzumab-39 | 70 | 76 |
| Onartuzumab-23E | 89 | 89 |
| Onartuzumab-39EGY4 | 81 | 81 |
| Onartuzumab-39EGY4-2224 | 88 | 88 |
| Onartuzumab-39EGY4-panitumumab | 85 | 85 |
| Onartuzumab-39EGY4-cetuximab | 82 | 83 |

### Fortebio Binding Protocol

### Materials Required:

96-well, black, round, flat bottom, polypropylene microplates (Greiner Bio-one #655209).

Octet instrument and software (version 3.0).
Protein A sensor tips (Fortebio, #18-5010)
1×PBS, antigen (his tagged cMET), antibodies

### Protocol:

All reagents are equilibrated and samples are brought to room temperature. Protein A sensor tips (Fortebio®, #18-5010) are hydrated for 10 min in 1×PBS. Kinetic assays are run using the Octet® software and procedure per manufacturer's instruction. Assay steps typically include: 1-2 min of equilibration in 1×PBS, 4 min of antibody loading (conc: 50 μg/mL in 1×PBS), 1-2 min of baseline stabilization, 4 min of antibody:antigen association, and 4 min of antibody:antigen dissociation. 1×PBS is used as the matrix throughout. Data are analyzed with Octet® Data Analysis software, processed, and fit to the curve using 1:1 binding model to determine kinetic parameters (K_{d}, Kₒₙ and K_{off})

TABLE 28
K_{d} of anti-cMet TFcs with various backbone modifications

| | Kd determined by Fortebio ® |
|---|---|
| Molecule or antibody name | (Binding to cMET.his) |
| Onartuzumab-23E | 1.3 nM |
| Onartuzumab-39EGY4 | 1.25 nM |
| Onartuzumab-23 | 1.24 nM |
| Onartuzumab-39 | 1.26 nM |
| Onartuzumab-39EGY4-224 | 1.29 nM |
| Onartuzumab-39EGY4-cetuximab | 0.9 nM |
| Onartuzumab-39EGY4-panitumumab | 1.6 nM |
| Onartuzumab | 1.2 nM |

### Example 10

### Signaling Inhibition by Onartuzumab and Bispecific Variants

To test the ability of the constructs in Table 28 to inhibit pMet, the TFc variants were tested in HGF-induced SW620 cells (ATCC® cat #: CCL-227™) as follows: On day 1, actively mid-log (about 80% confluence) growing cells (e.g., SW620 cells) are split in RPMI (+10% FBS+L/glutamine (2 mM)+Pen/Strep) media. Approximately 20,000 cells are seeded/well in a 96-well plate. On day 2, the media is changed from 10% FBS to serum-free media—RPMI+0.5% FBS (+L/glutamine+Pen/Strep) media. On day 3, the HGF (stimulated control) and various inhibitors/antibodies are diluted into the appropriate volume of serum free media. 100 μL of each inhibitor per concentration is added/well. The inhibitor is allowed to incubate at 37° C. for 2 hours. The cells are then washed 2× with cold PBS and later lysed in 50 μL/well of MPER (cat #PI78505, VWR International)+150 mM NaCl+Protease and Phosphatase Inhibitor buffer (cOmplete™ Protease Inhibitor Cocktail Tablet provided in EASY packs, cat #4693124001, Roche Diagnostics Corp; PhosSTOP® Phosphatase Inhibitor Cocktail Tablets, cat #4906837001, Roche Diagnostics Corp). The lysates are placed in −80° C. usually within 5 minutes after lysis.

For measurement of pMet signal, an ELISA kit was used (Human Phospho-HGF R/c-MET DuoSet IC Economy Pack, cat #DYC2480E, R&D Systems). A 384-well High Binding Black Solid plate from Corning® is coated with capture anti-MET antibody from R&D Systems at a final concentration of 4 μg/mL/well in PBS buffer. The plates are left at overnight at room temperature. On day 4, the −80° C. lysate is thawed at room temperature. Plates are then washed 3 times with 50 μl/well in the BIOTEK plate washer with PBST (PBS with 0.05% Tween-20®). The 384-well plates are blocked with 50 μL/well 2% BSA/PBS for 1 hour at room temperature. Duplicate lysates are pooled into one well and diluted 2-fold in 2% BSA/0.1% Tween-20/25% MPER/PBS. Recombinant standard curves are prepared by making 10×2-fold serial dilutions in 2% BSA/0.1% Tween-20®/25% MPER/PBS. ELISA plates are washed with 0.05% Tween-20/PBS. 20 μL lysates are transferred from the 96-well plate in quadruplicate to the 384-well plate. Plates are incubated at room temperature for 2 hours and washed 3 times with 0.05% Tween-20/PBS. 20 μL primary detection anti-phosphotyrosine antibody, 4G10 (cat#05-321, Millipore/Upstate), is added at a dilution of 1:1000 to the ELISA plates and incubated for 1 hour at room temperature. 20 μL of SuperSignal® ELISA Pico Chemiluminescent Substrate (cat #PI37069, VWR International) is added per manufacturer's directions and read on Envision® Plate Reader (Perkin Elmer). For data analyses, duplicate samples are averaged and error bars are used to represent the standard deviation between the two replicates Inhibition curves and corresponding IC50 values are calculated using GraphPad Prism® software (GraphPad Software, Inc.) via regression of the data to a 4 parameter logistic equation.

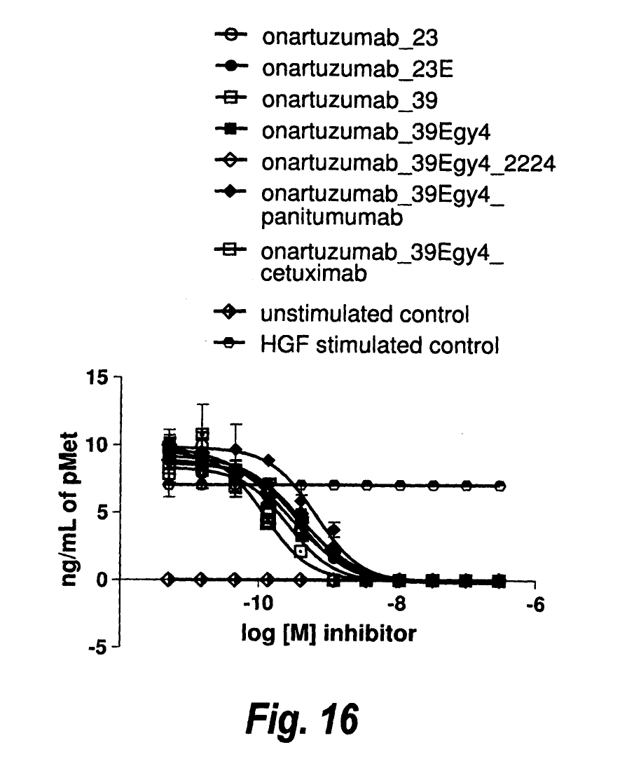

As shown in FIG. 16, all molecules tested inhibited pMet signaling in a similar manner, without regard to the identity of the TFc core region. The bivalent onartuzumab_{—}39Egy4_{—}2224, onartuzumab_{—}39Egy4_panitumumab, and onartuzumab_{—}39Egy4_cetuximab variants inhibited to a similar extent as did the monovalent onartuzumab_{—}39Egy4 variant.

### EQUIVALENTS

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure.

### INCORPORATION BY REFERENCE

The disclosure of each and every U.S. and foreign patent and pending patent application and publication referred to herein is specifically incorporated by reference herein in its entirety.

1-123. (canceled)

124. An antibody, which is a Tandem Fc Bispecific Antibody (“TFcBA”), wherein the TFcBA comprises a first binding site that is a single anti-c-Met binding site and at least one second binding site that specifically binds to a cell surface receptor other than c-Met; optionally a cell surface receptor selected from ALK, AXL, CEA, CD44, ErbB2, ErbB3, ErbB4, IGF1R, IGF2R, Insulin receptor, RON, EGFR, VEGFR1, VEGFR2, TNFR, FGFR1, FGFR2, FGFR3, FGFR4, PDGFR alpha, PDGFR beta, c-Kit, EPCAM and EphA2, wherein the anti-c-Met binding site and the second binding site are linked through a Tandem Fc (“TFc”), and
a. the TFc comprises a first Fc region and a second Fc region, or
b. the TFc comprises a first Fc region and a second Fc region which first Fc region and the second Fc region comprise a first and a second CH3 domain, or
c. the TFc comprises a first Fc region and a second Fc region which first Fc region and the second Fc region comprise a first and a second CH3 domain and a first and a second CH2 domain,
d. the TFc comprises a first Fc region and a second Fc region, and the first and the second Fc regions comprise a first and a second hinge, respectively, each said first hinge and said second hinge having a C-terminus and an N-terminus, or
e. the TFc comprises a first Fc region and a second Fc region which first Fc region and the second Fc region comprise a first and a second CH3 domain and the first and the second Fc regions comprise a first and a second hinge, respectively, each said first hinge and said second hinge having a C-terminus and an N-terminus, or
f. the TFc comprises a first Fc region and a second Fc region which first Fc region and the second Fc region comprise a first and a second CH3 domain and a first and a second CH2 domain, and the first and the second Fc regions comprise a first and a second hinge, respectively, each said first hinge and said second hinge having a C-terminus and an N-terminus; each said first Fc region and second Fc region and first and a second CH3 domain and first and second CH2 domain having a C-terminus and an N-terminus; the first Fc region and the second Fc region being linked through a TFc linker to form a contiguous polypeptide, wherein the TFc linker:
g. has a C-terminus and an N-terminus, or
h. comprises 20-50 amino acids and has a C-terminus and an N-terminus, or
i. is a Gly-Ser linker and has a C-terminus and an N-terminus, or
j. comprises (Gly4Ser)n, wherein n is 4, 5, 6, 7 or 8 and has a C-terminus and an N-terminus; and
the first and the second Fc regions associate to form an Fc dimer.

125. The TFcBA of claim 124, wherein
a. the TFcBA inhibits signal transduction induced by either or both of HGF and a cognate ligand of the receptor specifically bound by the at least one second binding site with an IC50 of 10 nM or less or 1 nM or less or 100 pM or less, or a maximal percent inhibition of at least 70% or at least 80% or at least 90%, as indicated by inhibition of phosphorylation of either or both of c-Met and the receptor specifically bound by the at least one second binding site; or
b. expression of the TFcBA in a cell produces (i) more correctly formed TFcAB molecules relative to the expression of a multivalent antibody that does not comprise a TFc or (ii) more than 80% of correctly formed TFcAB molecules as determined by Size Exclusion Chromatography (SEC).

126. The TFcBA of claim 124, wherein the second hinge does not comprise an upper hinge subdomain.

127. The TFcBA of claim 124, wherein:
a. the TFc comprised in the TFcBA comprises in amino to carboxyl terminal order: a first CH2 domain, a first CH3 domain, a TFc linker, a second CH2 domain and a second CH3 domain, or
b. the TFc comprised in the TFcBA comprises in amino to carboxyl terminal order: a first hinge, a first CH2 domain, a first CH3 domain, a TFc linker, a second CH2 domain and a second CH3 domain, or
c. the TFc comprised in the TFcBA comprises in amino to carboxyl terminal order: a first hinge, a first CH2 domain, a first CH3 domain, a TFc linker, a second hinge, a second CH2 domain and a second CH3 domain.

128. The TFcBA of claim 127, wherein the first hinge comprises an upper hinge subdomain, a core hinge subdomain and a lower hinge subdomain and the second hinge comprises a core hinge subdomain and a lower hinge subdomain, but not an upper hinge subdomain, each said hinge sub-domain having a C-terminus and an N-terminus.

129. The TFcBA of claim 124, wherein the TFc comprised by the TFcBA comprises in amino to carboxyl terminal order: a first hinge, which is linked at its C-terminus to the N-terminus of a first CH2 domain, which is linked at its C-terminus to the N-terminus of a first CH3 domain, which is linked at its C-terminus to the N-terminus of a TFc linker, which is linked at its C-terminus to the N-terminus of a second hinge, which is linked at its C-terminus to the N-terminus of a second CH2 domain, which is linked at its C-terminus to the N-terminus of a second CH3 domain.

130. The TFcBA of claim 124 wherein the TFc is an IgG1 TFc, a hybrid TFc, or an IgG1/IgG4 TFc.

131. The TFcBA of claim 129, wherein the TFc is an IgG TFc and comprises in amino to carboxyl terminal order: a first IgG1 hinge, a first IgG1 CH2 domain, a first IgG1 CH3 domain, a TFc linker, a second IgG1 hinge, a second IgG1 CH2 domain, and a second IgG1 CH3 domain.

132. The TFcBA of claim 130, wherein the TFc is a hybrid TFc that comprises in amino to carboxyl terminal order: a first IgG1/IgG4 hinge, a first IgG4 CH2 domain, a first IgG1 CH3 domain, a TFc linker, a second IgG4 hinge, a second IgG4 CH2 domain, and a second IgG1 CH3 domain.

133. The TFcBA of claim 124, wherein
a. either or both of the first CH3 domain and the second CH3 domain comprise one or more amino acid (aa) modifications that enhance or stabilize the binding between the first and the second Fc regions, or
b. each of the first CH3 domain and the second CH3 domain comprises an aa modification, which modification is an Association Enhancing Modification (“AEM”) that enhances the association of the first CH3 domain with the second CH3 domain, or
c. each of the first CH3 domain and the second CH3 domain comprises an aa modification, which modification is an AEM that enhances the association of the first CH3 domain with the second CH3 domain and the AEM is comprised by a module selected from the group consisting of AEM module 1, AEM module 2, AEM module 3 and AEM module 4, or
d. either or both of the first Fc region and the second Fc region comprises an aa modification that adds a cysteine as an insertion or replacement, which cysteine forms a disulfide bond with a cysteine in the other Fc region (a “DiS” modification), or
e. either or both of the first Fc region and the second Fc region comprises a DiS modification in a hinge, or
f. either or both of the first and the second Fc region comprise a DiS modification in a CH3 domain, or
g. either or both of the first Fc region and the second Fc region comprises a DiS modification, wherein the DiS modification is comprised by DiS module 1 or DiS module 2, or
h. either or both of the first Fc region and the second Fc region comprises a DiS modification in a hinge, wherein the DiS modification is comprised by DiS module 1 or DiS module 2, or
i. either or both of the first and the second Fc region comprise a DiS modification in a CH3 domain, wherein the DiS modification is comprised by DiS module 1 or DiS module 2.

134. The TFcBA of claim 133, wherein each of the first CH3 domain and the second CH3 domain comprises one or more AEM modifications and one or more DiS modifications, or wherein either or both of the first and the second CH3 domains comprises an aa sequence selected from the group consisting of SEQ ID NOs:27-98, or that is at least 70% identical to an aa sequence selected from the group consisting of SEQ ID NOs:27-98, or which differs therefrom in at most 30 aa additions, deletions or substitutions, or wherein the first CH3 domain or the second CH3 domain comprises an aa sequence selected from the group consisting of SEQ ID NOs:27-98.

135. The TFcBA of claim 124, wherein
a. the first CH3 and second CH3 domains together comprise a pair of two different members, each member being a CH3 amino acid (aa) sequence, each pair selected from the group of pairs consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, each member aa sequence being at least 70% identical to, or differing in at most 30 aa additions, deletions or substitutions from the each sequence of each said pair, wherein the first CH3 domain comprises a different member of the pair than is comprised by the second CH3 domain, or
b. the first and the second CH3 domains each comprise an aa sequence that identical to an aa sequence of a member of the pair of CH3 aa sequences selected from the group consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98.

136. The TFcBA of claim 124, wherein
a. the first hinge comprises an amino acid (aa) sequence that is an aa sequence selected from the group consisting of SEQ ID NOs:4, 18, 19, 20, 21, 22, 263-265 and 267-273, or the first hinge comprises an aa sequence that differs in at most 3 aa deletions, additions or substitutions from an aa sequence selected from the group consisting of SEQ ID NOs:4, 18, 19, 20, 21, 22, 263-265 and 267-273; or
b. the second hinge comprises an aa sequence that is an aa sequence selected from the group consisting of SEQ ID NOs:23, 24, 263-265 and 267-273, or the second hinge comprises an aa sequence that differs in at most 3 aa deletions, additions or substitutions from an aa sequence selected from the group consisting of SEQ ID NOs:23, 24, 263-265 and 267-273.

137. The TFcBA of claim 124, comprising a CH2 domain comprising an amino acid sequence that is at least 70% identical to SEQ ID NO:25, 26, 261 or 262, or which differs therefrom in at most 30 aa deletions, additions or substitutions.

138. The TFcBA of claim 124, wherein the TFc comprises in amino to carboxyl terminal order: a first hinge, a first CH2 domain, a first CH3 domain, a second hinge, a second CH2 domain and a second CH3 domain, wherein
a. the first hinge comprises an amino acid (aa) sequence selected from the group consisting of SEQ ID NOs:4, 18, 19, 263-265 and 267-273;
b. the first CH2 domain is aglycosylated and comprises the aa sequence set forth as SEQ ID NO:25;
c. the first CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98;
d. the second hinge comprises an aa sequence consisting of a sequence selected from the group consisting of SEQ ID NO:23, 263-265 and 267-273;
e. the second CH2 domain is aglycosylated and comprises the aa sequence set forth in SEQ ID NO:25; and
f. the second CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, wherein if the first CH3 domain comprises a first sequence of a pair of sequences, the second CH3 domain comprises the second sequence of the pair of sequences; and if the first CH3 domain comprises the second sequence of a pair of sequences, the second CH3 domain comprises the first sequence of the pair of sequences.

139. The TFcBA of claim 124, wherein the TFc comprises in amino to carboxyl terminal order: a first hinge, a first CH2 domain, a first CH3 domain, a second hinge, a second CH2 domain and a second CH3 domain, wherein
a. the first hinge comprises an amino acid (aa) sequence selected from the group consisting of SEQ ID NOs:20, 21, 22, 263-265 and 267-273;
b. the first CH2 domain is aglycosylated and comprises the aa sequence set forth in SEQ ID NO:26;
c. the first CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98;
d. the second hinge comprises an aa sequence consisting of SEQ ID NO:24, 263-265 and 267-273;
e. the second CH2 domain is aglycosylated and comprises the aa sequence set forth in SEQ ID NO:26; and
f. the second CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, wherein if the first CH3 domain comprises a first sequence of a pair of sequences, the second CH3 domain comprises the second sequence of the pair of sequences; and if the first CH3 domain comprises the second sequence of a pair of sequences, the second CH3 domain comprises the first sequence of the pair of sequences.

140. The TFcBA of claim 124, wherein the first or the second Fc region comprises an amino acid (aa) sequence selected from the group consisting of SEQ ID NOs:99-166, or the first or the second Fc region comprises an aa sequence that is at least 70% identical to an aa sequence selected from the group consisting of SEQ ID NOs:99-166, or differs therefrom in at most 50 aa deletions, additions or substitutions.

141. The TFcBA of claim 140, wherein either or both of the first and the second Fc region comprises an aa sequence that is at least 70% identical to one aa sequence of a pair of aa sequences selected from the group consisting of SEQ ID NOs:99 and 100; SEQ ID NOs:101 and 102; SEQ ID NOs:103 and 104; SEQ ID NOs:105 and 106; SEQ ID NOs:107 and 108; SEQ ID NOs:109 and 110; SEQ ID NOs:111 and 112; SEQ ID NOs:113 and 114; SEQ ID NOs:115 and 116; SEQ ID NOs:117 and 118; SEQ ID NOs:119 and 120; SEQ ID NOs:121 and 122; SEQ ID NOs:123 and 124; SEQ ID NOs:125 and 126; SEQ ID NOs:127 and 128; SEQ ID NOs:129 and 130; SEQ ID NOs:131 and 132; SEQ ID NOs:133 and 134; SEQ ID NOs:135 and 136; SEQ ID NOs:137 and 138; SEQ ID NOs:139 and 140; SEQ ID NOs:141 and 142; SEQ ID NOs:143 and 144; SEQ ID NOs:145 and 146; SEQ ID NOs:147 and 148; SEQ ID NOs:149 and 150; SEQ ID NOs:151 and 152; SEQ ID NOs:153 and 154; SEQ ID NOs:155 and 156; SEQ ID NOs:157 and 158; SEQ ID NOs:159 and 160; SEQ ID NOs:161 and 162; SEQ ID NOs:163 and 164; and SEQ ID NOs:165 and 166, or which differs therefrom in at most 50 aa deletions, additions or substitutions, and wherein the first Fc region comprises a different member of the pair than is comprised by the second Fc region.

142. The TFcBA of claim 140, wherein the first Fc region and the second Fc region together comprise a pair of two different members, each member being an Fc aa sequence, wherein each pair is selected from the group of pairs consisting of SEQ ID NOs:99 and 100; SEQ ID NOs:101 and 102; SEQ ID NOs:103 and 104; SEQ ID NOs:105 and 106; SEQ ID NOs:107 and 108; SEQ ID NOs:109 and 110; SEQ ID NOs:111 and 112; SEQ ID NOs:113 and 114; SEQ ID NOs:115 and 116; SEQ ID NOs:117 and 118; SEQ ID NOs:119 and 120; SEQ ID NOs:121 and 122; SEQ ID NOs:123 and 124; SEQ ID NOs:125 and 126; SEQ ID NOs:127 and 128; SEQ ID NOs:129 and 130; SEQ ID NOs:131 and 132; SEQ ID NOs:133 and 134; SEQ ID NOs:135 and 136; SEQ ID NOs:137 and 138; SEQ ID NOs:139 and 140; SEQ ID NOs:141 and 142; SEQ ID NOs:143 and 144; SEQ ID NOs:145 and 146; SEQ ID NOs:147 and 148; SEQ ID NOs:149 and 150; SEQ ID NOs:151 and 152; SEQ ID NOs:153 and 154; SEQ ID NOs:155 and 156; SEQ ID NOs:157 and 158; SEQ ID NOs:159 and 160; SEQ ID NOs:161 and 162; SEQ ID NOs:163 and 164; and SEQ ID NOs:165 and 166, each member aa sequence being at least 70% identical to, or differing in at most 30 aa additions, deletions or substitutions from each sequence of each said pair, wherein the first Fc region comprises a different member of the pair than is comprised by the second Fc region.

143. The TFcBA of claim 124 comprising a TFc comprising an amino acid (aa) sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221, or an aa sequence that is at least 70% identical to an aa sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221 or which differs therefrom in at most 30 aa additions, deletions or substitutions.

144. The TFcBA of claim 124, comprising an amino acid chain that comprises in amino to carboxyl terminal order:
a. a first heavy chain variable (VH) domain, a TFc, a connecting linker and a second VH domain, or
b. a first VH domain, a CH1 domain, a TFc, a connecting linker and a second VH domain, or
c. a first VH domain, a CH1 domain, a TFc, a connecting linker, a second VH domain, an scFv linker and a second light chain variable (VL) domain, wherein the second VH and VL domains associate to form a second binding site, or
d. a first VH domain, a CH1 domain, a TFc, a connecting linker, a second VH domain, an scFv linker and a second light chain variable (VL) domain, wherein the second VH domain and the VL domain associate to form a second binding site.

145. The TFcBA of claim 144, further comprising
a. a light chain that comprises a first VL domain that dimerizes with the first VH domain to form a first binding site, or
b. a light chain that comprises a first VL domain that dimerizes with the first VH domain to form a first binding site and further comprises a light chain constant (CL) domain that is linked to the carboxyl terminus of the VL domain.

146. The TFcBA of claim 124, wherein the first binding site is an N-terminal binding site and the second binding site is a C-terminal binding site.

147. The TFcBA of claim 145, wherein the anti-c-Met binding site
a. comprises a VH comprising either or both of a) the aa sequence of the VH Complementarity Determining Region (CDR)3 (VHCDR3) in SEQ ID NO:223 or 287 and b) a VLCDR3 comprising the aa sequence of the VLCDR3 in SEQ ID NO:231 or 289, or
b. comprises a VH domain comprising a set of three VH Complementarity Determining Regions (CDRs) comprising VHCDR1, VCDR2 and VHCDR3, wherein VHCDR1, VHCDR2 and VHCDR3 comprise the aa sequence of the VHCDR1, VHCDR2 and VHCDR3 in SEQ ID NO:223 or 231; and a VL domain comprising a set of three VLCDRs comprising VLCDR1, VLCDR2, and VLCDR3, wherein VLCDR1, VLCDR2 and VLCDR3 comprise the aa sequence of the VLCDR1, VLCDR2 and VLCDR3 in SEQ ID NO:287 or 289, respectively, or
c. comprises an N-terminal portion of the heavy chain and an N-terminal portion of the light chain, or
d. is comprised by either or both of a VH domain and a VL domain, wherein the VH domain comprises an aa sequence that is at least 70% identical to the VH domain set forth in SEQ ID NOs:223, 231, 287 or 289 or differs therefrom in at most 10 aas deletions, additions or substitution; and the VL domain comprises an aa sequence that is at least 70% identical to the VL domain set forth in SEQ ID NOs:223, 231, 287 or 289 or differs therefrom in at most 10 aas deletions, additions or substitution.

148. The TFcBA of claim 124, wherein the second binding site is
a. an anti-EGFR binding site that comprises either or both of a) a VHCDR3 comprising the amino acid (aa) sequence of the VHCDR3 in SEQ ID NO:233, 237, 258, 275, 277 or 279 and b) a VLCDR3 comprising the aa sequence of the VLCDR3 in SEQ ID NO:233, 237, 258, 275, 277 or 279, or
b. an anti-EGFR binding site that comprises a VH domain comprising a set of three VHCDRs comprising VHCDR1, VCDR2 and VHCDR3, wherein VHCDR1, VHCDR2 and VHCDR3 comprise the aa sequence of the VHCDR1, VHCDR2 and VHCDR3 in SEQ ID NO: 233, 237, 258, 275, 277 or 279; and a VL domain comprising a set of three VLCDRs comprising VLCDR1, VLCDR2, and VLCDR3, wherein VLCDR1, VLCDR2 and VLCDR3 comprise the aa sequence of the VLCDR1, VLCDR2 and VLCDR3 in SEQ ID NO:233, 237, 258, 275, 277 or 279, or
c. an anti-EGFR binding site that is comprised by either or both of a VH domain and a VL domain, wherein the VH domain comprises an aa sequence that is at least 70% identical to the VH domain set forth in SEQ ID NOs: 233, 237, 258, 275, 277 or 279 or differs therefrom in at most 10 aas deletions, additions or substitution; and the VL domain comprises an aa sequence that is at least 70% identical to the VL domain set forth in SEQ ID NOs: 233, 237, 258, 275, 277 or 279 or differs therefrom in at most 10 aas deletions, additions or substitutions, or
d. comprised by a C-terminal scFv that is entirely comprised by the heavy chain.

149. An Ab which is a TFcBA, wherein the TFcBA comprises a first binding site and a second binding site, wherein the first binding site binds to a first target and the second binding site binds to a second target, and wherein
the first and the second binding sites are linked through a TFc;
the TFc comprises a first Fc region and a second Fc region, each said first Fc region and second Fc region having a C-terminus and an N-terminus; the first Fc region and the second Fc region are linked through a TFc linker having a C-terminus and an N-terminus to form a contiguous polypeptide;
the first and the second Fc regions associate to form an Fc dimer; and
either or both of the first and the second Fc region comprise one or more aa modification to enhance or stabilize the binding between the first and the second Fc region.

150. The TFcBA of claim 149, wherein
a. the TFcBA inhibits signal transduction through either or both of the first and the second target; or
b. expression of the TFcBA in a cell produces (i) more correctly formed TFcAB molecules relative to the expression of a multivalent antibody that does not comprise a TFc or (ii) more than 80% of correctly formed TFcAB molecules as determined by Size Exclusion Chromatography (SEC).

151. The TFcBA of claim 149, wherein the first Fc region and the second Fc region comprise a first and a second CH3 domain, respectively, each said CH3 domain having a C-terminus and an N-terminus.

152. The TFcBA of claim 149, wherein the first and the second Fc regions comprise
a. a first and a second CH2 domain, respectively, each said CH2 domain having a C-terminus and an N-terminus, or
b. a first and a second hinge, respectively, each said first hinge and said second hinge having a C-terminus and an N-terminus, or
c. a first and a second hinge, respectively, each said first hinge and said second hinge having a C-terminus and an N-terminus and wherein the second hinge does not comprise an upper hinge subdomain.

153. The TFcBA of claim 152, wherein the TFc comprised in the TFcBA comprises in amino to carboxyl terminal order:
a. a first CH2 domain, a first CH3 domain, a TFc linker, a second CH2 domain and a second CH3 domain;
b. a first hinge, a first CH2 domain, a first CH3 domain, a TFc linker, a second CH2 domain and a second CH3 domain
c. a first hinge, a first CH2 domain, a first CH3 domain, a TFc linker, a second hinge, a second CH2 domain and a second CH3 domain, or
d. a first hinge, a first CH2 domain, a first CH3 domain, a TFc linker, a second hinge, a second CH2 domain and a second CH3 domain and wherein the first hinge comprises an upper hinge subdomain, a core hinge subdomain and a lower hinge subdomain and the second hinge comprises a core hinge subdomain and a lower hinge subdomain, but not an upper hinge subdomain, each said hinge sub-domain having a C-terminus and an N-terminus.

154. A TFcBA of claim 149, wherein the TFc comprised by the TFcBA comprises in amino to carboxyl terminal order: a first hinge, which is linked at its C-terminus to the N-terminus of a first CH2 domain, which is linked at its C-terminus to the N-terminus of a first CH3 domain, which is linked at its C-terminus to the N-terminus of a TFc linker, which is linked at its C-terminus to the N-terminus of a second hinge, which is linked at its C-terminus to the N-terminus of a second CH2 domain, which is linked at its C-terminus to the N-terminus of a second CH3 domain.

155. The TFcBA of claim 153, wherein the TFc linker
a. comprises 20-50 amino acids (aas), or
b. is a Gly-Ser linker, or
c. comprises (Gly4Ser)n, wherein n is 4, 5, 6, 7 or 8,
d. or,
e. wherein the TFc
f. is an IgG1 TFc, or
g. is an IgG1 TFc, or
h. is a hybrid TFc, or
i. is a hybrid TFc comprising in amino to carboxyl terminal order: a first IgG1/IgG4 hinge, a first IgG4 CH2 domain, a first IgG1 CH3 domain, a TFc linker, a second IgG4 hinge, a second IgG4 CH2 domain, and a second IgG1 CH3 domain, or
j. is an IgG1/IgG4 TFc
k. comprises in amino to carboxyl terminal order: a first IgG1 hinge, a first IgG1 CH2 domain, a first IgG1 CH3 domain, a TFc linker, a second IgG1 hinge, a second IgG1 CH2 domain, and a second IgG1 CH3 domain.

156. The TFcBA of claim 153, wherein
a. either or both of the first CH3 domain and the second CH3 domain comprise one or more aa modifications that enhance or stabilize the binding between the first and the second Fc regions, or
b. each of the first CH3 domain and the second CH3 domain comprises an amino acid modification, which modification is an Association Enhancing Modification (“AEM”) that enhances the association of the first CH3 domain with the second CH3 domain, or
c. each of the first CH3 domain and the second CH3 domain comprises an amino acid modification, which modification is an AEM that enhances the association of the first CH3 domain with the second CH3 domain, wherein the AEM is comprised by a module selected from the group consisting of AEM module 1, AEM module 2, AEM module 3 and AEM module 4.

157. The TFcBA of claim 153, wherein
a. the first CH3 and second CH3 domains together comprise a pair of two different members, each member being a CH3 amino acid (aa) sequence, each pair selected from the group of pairs consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, each member aa sequence being at least 70% identical to, or differing in at most 30 aa additions, deletions or substitutions from the each sequence of each said pair, wherein the first CH3 domain comprises a different member of the pair than is comprised by the second CH3 domain, or
b. the first and the second CH3 domains each comprise an aa sequence that identical to an aa sequence of a member of the pair of CH3 aa sequences selected from the group consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98.

158. The TFcBA claim 157, wherein
a. the first hinge comprises an aa sequence that differs in at most 3 aa deletions, additions or substitutions from an aa sequence selected from the group consisting of SEQ ID NOs:4, 18, 19, 20, 21, 22, 263-265 and 267-273, or
b. the first hinge comprises an aa sequence that is an aa sequence selected from the group consisting of SEQ ID NOs:4, 18, 19, 20, 21, 22, 263-265 and 267-273, or
c. the second hinge comprises an aa sequence that differs in at most 3 aa deletions, additions or substitutions from an aa sequence selected from the group consisting of SEQ ID NOs:23, 24, 263-265 and 267-273, or
d. the second hinge comprises an aa sequence that is an aa sequence selected from the group consisting of SEQ ID NOs:23, 24, 263-265 and 267-273, or
e. the TFc comprises in amino to carboxyl terminal order: a first hinge, a first CH2 domain, a first CH3 domain, a second hinge, a second CH2 domain and a second CH3 domain, wherein
i. the first hinge comprises an aa sequence selected from the group consisting of SEQ ID NOs:4, 18, 19, 263-265 and 267-273;
ii. the first CH2 domain is aglycosylated and comprises the aa sequence set forth as SEQ ID NO:25;
iii. the first CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98;
iv. the second hinge comprises an aa sequence consisting of a sequence selected from the group consisting of SEQ ID NO:23, 263-265 and 267-273;
v. the second CH2 domain is aglycosylated and comprises the aa sequence set forth in SEQ ID NO:25; and
vi. the second CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, wherein if the first CH3 domain comprises a first sequence of a pair of sequences, the second CH3 domain comprises the second sequence of the pair of sequences; and if the first CH3 domain comprises the second sequence of a pair of sequences, the second CH3 domain comprises the first sequence of the pair of sequence.

159. The TFcBA of claim 149, wherein the TFc comprises in amino to carboxyl terminal order: a first hinge, a first CH2 domain, a first CH3 domain, a second hinge, a second CH2 domain and a second CH3 domain, wherein
a.
i. the first hinge comprises an aa sequence selected from the group consisting of SEQ ID NOs:20, 21, 22, 263-265 and 267-273;
ii. the first CH2 domain is aglycosylated and comprises the aa sequence set forth in SEQ ID NO:26;
iii. the first CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98;
iv. the second hinge comprises an aa sequence consisting of SEQ ID NO:24, 263-265 and 267-273;
v. the second CH2 domain is aglycosylated and comprises the aa sequence set forth in SEQ ID NO:26; and
vi. the second CH3 domain comprises an aa sequence that is either sequence of a pair of sequences selected from the group of pairs of CH3 domain sequences consisting of SEQ ID NOs:31 and 35; SEQ ID NOs:33 and 37; SEQ ID NOs:39 and 43; SEQ ID NOs:41 and 45; SEQ ID NOs:47 and 51; SEQ ID NOs:49 and 53; SEQ ID NOs:55 and 59; SEQ ID NOs:57 and 61; SEQ ID NOs:63 and 67; SEQ ID NOs:65 and 69; SEQ ID NOs:71 and 73; SEQ ID NOs:72 and 74; SEQ ID NOs:75 and 79; SEQ ID NOs:77 and 81; SEQ ID NOs:83 and 85; SEQ ID NOs:84 and 86; SEQ ID NOs:87 and 89; SEQ ID NOs:88 and 90; SEQ ID NOs:91 and 93; SEQ ID NOs:92 and 94; SEQ ID NOs:95 and 97; and SEQ ID NOs:96 and 98, wherein if the first CH3 domain comprises a first sequence of a pair of sequences, the second CH3 domain comprises the second sequence of the pair of sequences; and if the first CH3 domain comprises the second sequence of a pair of sequences, the second CH3 domain comprises the first sequence of the pair of sequences;
or
b. the first or the second Fc region comprises an aa sequence that is at least 70% identical to an aa sequence selected from the group consisting of SEQ ID NOs:99-166, or differs therefrom in at most 50 aa deletions, additions or substitutions, or
c. the first or the second Fc region comprises an aa sequence selected from the group consisting of SEQ ID NOs:99-166, or
d. either or both of the first and the second Fc region comprises an aa sequence that is at least 70% identical to one aa sequence of a pair of aa sequences selected from the group consisting of SEQ ID NOs:99 and 100; SEQ ID NOs:101 and 102; SEQ ID NOs:103 and 104; SEQ ID NOs:105 and 106; SEQ ID NOs:107 and 108; SEQ ID NOs:109 and 110; SEQ ID NOs:111 and 112; SEQ ID NOs:113 and 114; SEQ ID NOs:115 and 116; SEQ ID NOs:117 and 118; SEQ ID NOs:119 and 120; SEQ ID NOs:121 and 122; SEQ ID NOs:123 and 124; SEQ ID NOs:125 and 126; SEQ ID NOs:127 and 128; SEQ ID NOs:129 and 130; SEQ ID NOs:131 and 132; SEQ ID NOs:133 and 134; SEQ ID NOs:135 and 136; SEQ ID NOs:137 and 138; SEQ ID NOs:139 and 140; SEQ ID NOs:141 and 142; SEQ ID NOs:143 and 144; SEQ ID NOs:145 and 146; SEQ ID NOs:147 and 148; SEQ ID NOs:149 and 150; SEQ ID NOs:151 and 152; SEQ ID NOs:153 and 154; SEQ ID NOs:155 and 156; SEQ ID NOs:157 and 158; SEQ ID NOs:159 and 160; SEQ ID NOs:161 and 162; SEQ ID NOs:163 and 164; and SEQ ID NOs:165 and 166, or which differs therefrom in at most 50 aa deletions, additions or substitutions, and wherein the first Fc region comprises a different member of the pair than is comprised by the second Fc region, or
e. the first Fc region and the second Fc region together comprise a pair of two different members, each member being an Fc aa sequence, wherein each pair is selected from the group of pairs consisting of SEQ ID NOs:99 and 100; SEQ ID NOs:101 and 102; SEQ ID NOs:103 and 104; SEQ ID NOs:105 and 106; SEQ ID NOs:107 and 108; SEQ ID NOs:109 and 110; SEQ ID NOs:111 and 112; SEQ ID NOs:113 and 114; SEQ ID NOs:115 and 116; SEQ ID NOs:117 and 118; SEQ ID NOs:119 and 120; SEQ ID NOs:121 and 122; SEQ ID NOs:123 and 124; SEQ ID NOs:125 and 126; SEQ ID NOs:127 and 128; SEQ ID NOs:129 and 130; SEQ ID NOs:131 and 132; SEQ ID NOs:133 and 134; SEQ ID NOs:135 and 136; SEQ ID NOs:137 and 138; SEQ ID NOs:139 and 140; SEQ ID NOs:141 and 142; SEQ ID NOs:143 and 144; SEQ ID NOs:145 and 146; SEQ ID NOs:147 and 148; SEQ ID NOs:149 and 150; SEQ ID NOs:151 and 152; SEQ ID NOs:153 and 154; SEQ ID NOs:155 and 156; SEQ ID NOs:157 and 158; SEQ ID NOs:159 and 160; SEQ ID NOs:161 and 162; SEQ ID NOs:163 and 164; and SEQ ID NOs:165 and 166, each member aa sequence being at least 70% identical to, or differing in at most 30 aa additions, deletions or substitutions from each sequence of each said pair, wherein the first Fc region comprises a different member of the pair than is comprised by the second Fc region, or
f. the TFcBA comprises a TFc comprises an aa sequence that is at least 70% identical to an aa sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221 or which differs therefrom in at most 30 aa additions, deletions or substitutions, or
g. the TFcBA comprises a TFc comprising an aa sequence selected from the group consisting of SEQ ID NOs:171, 173, 175, 177, 179, 181, 183, 185, 187, 189, 191, 193, 195, 197, 199, 201, 203, 205, 207, 209, 211, 213, 215, 217, 219 and 221, or
h. the TFcBA comprises a heavy chain that comprises in amino to carboxyl terminal order: a first VH domain, a TFc, a connecting linker and a second VH domain, or
i. the TFcBA comprises a heavy chain that comprises in amino to carboxyl terminal order: a first VH domain, a CH1 domain, a TFc, a connecting linker and a second VH domain, or
j. the TFcBA comprises a heavy chain that comprises in amino to carboxyl terminal order: a first VH domain, a CH1 domain, a TFc, a connecting linker, a second VH domain, an scFv linker and a second VL domain, wherein the second VH and VL domains associate to form a second binding site, or
k. the TFcBA comprises a heavy chain that comprises in amino to carboxyl terminal order: a first VH domain, a CH1 domain, a TFc, a connecting linker, a second VH domain, an scFv linker and a second light chain variable (VL) domain, wherein the second VH and VL domains associate to form a second binding site.

160. The TFcBA of claim 159, comprising a light chain that comprises a first VL domain that dimerizes with the first VH domain to form a first binding site.

161. The TFcBA of claim 160, wherein the light chain comprises a light chain constant (CL) domain that is linked to the carboxyl terminus of the VL domain.

162. The TFcBA of claim 159, wherein the first binding site is an anti-c-Met binding site and the second binding site is an anti-EGFR binding site.

163. A monovalent Tandem Fc Antibody (TFcA), comprising a binding site that is linked to a TFc comprising a first Fc region and a second Fc region linked through a TFc linker, wherein the first and the second Fc region associate to form an Fc, and wherein either or both of the first and the second Fc region comprise one or more amino acid modifications to enhance or stabilize the binding between the first and the second Fc region.

164. A TFcBA of claim 124 that is a charge-complementary paired TFcBA, wherein a charge-complementary paired TFcBA is a TFcBA that comprises a pair of charged amino acids comprising an amino acid selected from group A and an amino acid selected from group B (a charge-complementary pair)
wherein group A comprises all natural amino acids with a pI of greater than 7 and group B comprises all natural amino acids with a pI of less than 7, or optionally wherein group A comprises His, Lys, and Arg, and group B comprises Asp, Glu, Asn, Phe, Gln, Tyr, Ser, Met, Thr, Ile, Gly, Val, Trp, Leu, Ala, and Pro; and
said charge-complementary pair consists of a first amino acid residue and a second amino acid residue, and
said charge-complementary pair is either or both of a position 297 charge-complementary pair or a position 299 charge-complementary pair, wherein
a position 297 charge-complementary pair is a charge-complementary pair with said first amino acid residue located at EU position 297 of said first Fc region and said second amino acid residue located at EU position 297 of said second Fc region, and a position 299 charge-complementary pair is a charge-complementary pair with said first amino acid residue located at EU position 299 of said first Fc region and said second amino acid residue located at EU position 299 of said second Fc region.

165. The TFcBA of claim 164, wherein the first or the second binding site binds specifically to a human protein selected from the group consisting of ErbB2, ErbB3, ErbB4, IGF1R, IGF2R, Insulin receptor, Ron, c-Met, EGFR, VEGFR1, VEGFR2, TNFR, FGFR1 FGFR2, FGFR3, FGFR4, PDGFR alpha, PDGFR beta, c-Kit, EPCAM and EphA2.

166. A pharmaceutical composition comprising a TFcBA of claim 124 and a pharmaceutically acceptable carrier.

167. A nucleic acid molecule comprising at least one or at least two coding sequences, wherein said at least one coding sequence encodes a heavy chain or a light chain of TFcBA of claim 124 and wherein said at least two coding sequences comprise one coding sequence encodes a heavy chain of a TFcBA of claim 124 and a second coding sequence encodes a light chain of the TFcBA of claim 124 and wherein the nucleic acid molecule is:
a. an isolated nucleic acid molecule, or
b. comprised by a vector, or
c. comprised by a host cell, or
d. comprised by a vector that is comprised by a host cell.

168. A method of producing a TFcBA comprising culturing a host cell of claim 167 under conditions suitable for the expression of the TFcBA, and isolating the expressed TFcBA from the culture.

169. A method of treating a subject having cancer, said method comprising administering to a subject a therapeutically effective amount of a TFcBA of claim 124.
